SEQUENCE LISTING (105 sequences)

SEQ ID NO: 1 - DNA, 1881 bp, Macaca fascicularis
  misc_feature 1..1881: note = MfPV3 E1
  source 1..1881: mol_type = other DNA
atggacccttctggaattcacggcgagggcaccagatgcaccggatggttcgatgtggaagccatcgtggaaagacggaccggcgacgtggtgtccgaggacgaagatgagaacctggaagataccggcgtggacctggtggacttcatcgacgatacctgcgagtttgagcaggccggcgacgaagctcctagagcactgctgcatgcccaagagacacaggcccatgtgaacgccgtgcactacctgaagcggaagttcacatgcagccctaccgtgtctcccctgggcaatgtgcagtgttctatcgagggcgagctgagccctagactgcacgccatccaactgaatgaaggcagcggcaaggccaagcggagactgtttctgcctgatagcggctacggcaataccgaggtggaaacccagctgtctcaggtggcaggatccgatggcgaatctcaagcccctggcggcaaagaaaatgtgcagcctgctgaaggcgctggcggagatgttcgacagctgctgagaagcagcaacctgaaggccacactgctgggcaagttcaaggacgtgtacggcatcggcttcaacgaactcgtgcggtgcttcaagagcgacaagaccgtgtgcaccgattgggttgtgggagctgctggcgtgcaccattctgtggccgaaggactgaagcacctgatcgagcctttctgcacatacgcccacatgcagtgcctgacctgcaactggggagtgtttctgctgctgctcatccggttcaagtgcggcaagaacagactgaccgtggccaagtgcatctctaccctgctgaacgtgcaagagccctacctgctgatcgaacctccaaagctgagatccacagccgccgctctgtactggtacagaaccggcatcagcaacgtgtccgaagtgatcggagagcctcctgagtggatcgtgcggcagaccatgtttcagcacagcctcgaggacagcatcttcgacctgtctcagatggtgcagtgggcctacgaccacgatctggtggacgatagcgccattgcctaccagtatgcccagctggccgacgtggactctaatgccgccgctttcctgaagtctaacgctcaggccaaatacgtgaaggactgcgccaccatgtgcagacactacaagagagccgagaagcaggccatgaccatgagccagtggatcagagccagatgcgataagaccgacgacggcggagattggaggcctatcgtgcagttcctgagataccagaacgtcgagttcatcacctttctgagcgcctttaaggccttcctgaagggaatccccaagagaagctgcatcgtgctgtacggccctccaaacaccggcaagagctacttcggaatgagcctgatgcagtttctgcagggcagcatcatcagctacgtgaacagcaacagccacttttggctggcccctctggccgaagctaaagtggccatgctggacgatgccacaccacagtgctggtcctacatcgacacctacatgcggagcgtgctggacggcaaccccatgagcattgaccggaagcacaagaacctgatccagatgaagtgtccgcctctgatcatcaccagcaacaccaacgtgggcgaagatgacagatggcgctacctgcactccagagtggtggtgttcaagttcgagcagacctttccattcgaccagaacggcaatcccgtgtacgagctgtccgacaagaattggaagtccttcttcagccggacctggtccagactcgagctgcaagaggatgaggacaccgagcaggatggctctacagtgcgggcctttaagtgtgtgcccggccagaacaccaatatcctg

SEQ ID NO: 2 - protein, 627 aa, Macaca fascicularis
  REGION 1..627: note = MfPV3 E1 translated
MDPSGIHGEGTRCTGWFDVEAIVERRTGDVVSEDEDENLEDTGVDLVDFIDDTCEFEQAGDEAPRALLHAQETQAHVNAVHYLKRKFTCSPTVSPLGNVQCSIEGELSPRLHAIQLNEGSGKAKRRLFLPDSGYGNTEVETQLSQVAGSDGESQAPGGKENVQPAEGAGGDVRQLLRSSNLKATLLGKFKDVYGIGFNELVRCFKSDKTVCTDWVVGAAGVHHSVAEGLKHLIEPFCTYAHMQCLTCNWGVFLLLLIRFKCGKNRLTVAKCISTLLNVQEPYLLIEPPKLRSTAAALYWYRTGISNVSEVIGEPPEWIVRQTMFQHSLEDSIFDLSQMVQWAYDHDLVDDSAIAYQYAQLADVDSNAAAFLKSNAQAKYVKDCATMCRHYKRAEKQAMTMSQWIRARCDKTDDGGDWRPIVQFLRYQNVEFITFLSAFKAFLKGIPKRSCIVLYGPPNTGKSYFGMSLMQFLQGSIISYVNSNSHFWLAPLAEAKVAMLDDATPQCWSYIDTYMRSVLDGNPMSIDRKHKNLIQMKCPPLIITSNTNVGEDDRWRYLHSRVVVFKFEQTFPFDQNGNPVYELSDKNWKSFFSRTWSRLELQEDEDTEQDGSTVRAFKCVPGQNTNIL

SEQ ID NO: 3 - DNA, 1080 bp, Macaca fascicularis
  misc_feature 1..1080: note = MfPV3 E2
  source 1..1080: mol_type = other DNA
atggaagccctgagcgagagactgaacgcctaccaggacaagatcctgaccttctacgaagaggactccaaggacctgagggaccagatcgagcattggagatgtgtgcggggagagtgcgccgtgttctacaaggccagagagatgggccacagccacatcaaccaccagcaggttccacctctgcacgtgtccagaggcaaggcccacaatgccattgagctgcagatggccctggaaagcctgaaccagagcgacttcaacaccgaggaatggaccctgcaggacacaagcctggaaatgtggcggaccgagcctcagtgctgcttcaaaaagaaaggcgagacagtgaccgtgctgtatgactgcgacaaggacaacagcatggactacaccctgtggggccacgtgtacgtgtggggagatacaggctgggacaaagtgtgcggaaaggtggactacggcggcctgtactatgtgctgcacggcctgaaagtgtactacgttgagttccagcaagaggccgtgaagtacagcaagaccaacaagtgggaagtgcatatcggcagaacagtgatcccctacagcgagtccgtgtcctctacagccctgtgcgaaagagtgcctatcgccgagatcgccaatggcctccagcagaccagcattccctctcacacacacgccaccaccaaagagaacgcttgggtttcctctcagcctcctaccaagcggtccagaagatctgatccaggcggcgatcctgtcagagccctggatgccaatagcagaaccctgctgtctggaaccgcctgcgagaataccaccggcaactcttacagcgactgcacccctgtggtgcacctgaaaggcgaccccaattgtctgaaggccctgcggttcagattcgtgaaacacaagaatctgttcgtgaacgtgtcctccacctggcggtggaccaacaacaccgaagagaaggccatcgtgactgtgacctttaaggacgagcagcagagacagcagttcctcggcagcgtgaagatccccaactccatctgtgtgtgcaagggcgtgatgacagtt

SEQ ID NO: 4 - protein, 360 aa, Macaca fascicularis
  REGION 1..360: note = MfPV3 E2 translated
MEALSERLNAYQDKILTFYEEDSKDLRDQIEHWRCVRGECAVFYKAREMGHSHINHQQVPPLHVSRGKAHNAIELQMALESLNQSDFNTEEWTLQDTSLEMWRTEPQCCFKKKGETVTVLYDCDKDNSMDYTLWGHVYVWGDTGWDKVCGKVDYGGLYYVLHGLKVYYVEFQQEAVKYSKTNKWEVHIGRTVIPYSESVSSTALCERVPIAEIANGLQQTSIPSHTHATTKENAWVSSQPPTKRSRRSDPGGDPVRALDANSRTLLSGTACENTTGNSYSDCTPVVHLKGDPNCLKALRFRFVKHKNLFVNVSSTWRWTNNTEEKAIVTVTFKDEQQRQQFLGSVKIPNSICVCKGVMTV

SEQ ID NO: 5 - DNA, 441 bp, Macaca fascicularis
  misc_feature 1..441: note = MfPV3 E6
  source 1..441: mol_type = other DNA
atggtggaaggcagccaagagagagcccggacactgcacgaactgtgcgagcagagagaacagacccagcacgacatcgagctggactgcgtgtactgcctgaatcctctgaccagaaccgaggtgtacgacttcgccagatgggagctgagaatcgtgtacagacgggataccgcctacggcgtgtgcaagctgtgcctgaggttccacagcaaagtgcggaagtacaggcggtacaactactccatctacggccccacactggaacggcggacaaagaagcagctcgaagaactcgtgatccggtgctactgctgccagaaacctcagtgccccgtggaaaagcagcgccacgttgaccagggccagagattccacaacattgccgagcagtggaccggcagatgcctgcaatgttggaggccaacagccgctgaaacc

SEQ ID NO: 6 - protein, 147 aa, Macaca fascicularis
  REGION 1..147: note = MfPV3 E6 translated
MVEGSQERARTLHELCEQREQTQHDIELDCVYCLNPLTRTEVYDFARWELRIVYRRDTAYGVCKLCLRFHSKVRKYRRYNYSIYGPTLERRTKKQLEELVIRCYCCQKPQCPVEKQRHVDQGQRFHNIAEQWTGRCLQCWRPTAAET

SEQ ID NO: 7 - DNA, 300 bp, Macaca fascicularis
  misc_feature 1..300: note = MfPV3 E7
  source 1..300: mol_type = other DNA
atgatcggccctggacctacactgcaggatatcgtgcttgagctggaacccgagcctgtggacctgagaggatatgagcagctggactccagcgaggacgaggacgagattgaccaccaccaacagcaggacagacccgatgtgcaagaagatgacggcgagcggtttagaatcctgagcgactgtcacgtgtgccagaataccgtgtcaagagtggtgcacagcacccacgacgacctgcacattctcgaggacctgcttatgggcaccctcgagatcgtggcccctaattgtgccgtg

SEQ ID NO: 8 - protein, 100 aa, Macaca fascicularis
  REGION 1..100: note = MfPV3 E7 translated
MIGPGPTLQDIVLELEPEPVDLRGYEQLDSSEDEDEIDHHQQQDRPDVQEDDGERFRILSDCHVCQNTVSRVVHSTHDDLHILEDLLMGTLEIVAPNCAV

SEQ ID NO: 9 - DNA, 1947 bp, Human papillomavirus type 16
  misc_feature 1..1947: note = HPV16 E1
  source 1..1947: mol_type = other DNA
atggccgatcctgctggcacaaacggcgaagaaggcacaggctgcaacggctggttttacgtggaagccgtggtggaaaagaaaaccggcgacgccatcagcgacgacgagaacgagaatgacagcgacaccggcgaggacctggtggacttcatcgtgaacgacaacgactacctgacacaggccgagacagagacagcccacgctctgtttacagcccaagaggccaagcagcacagagatgccgttcaggtgctgaagagaaagtacctgggcagccctctgagcgacatcagcggctgtgtggacaacaacatcagccccagactgaaggccatctgcatcgagaagcagagcagagccgccaagcggagactgtttgagagcgaggatagcggctacggcaataccgaggtggaaacccagcaaatgctgcaggtcgagggcagacacgaaaccgagacaccttgcagccagtacagcggaggatctggcggaggctgtagccagtactcttctggaagtggcggagagggcgtgtccgagagacacaccatttgtcagacccctctgaccaacatcctgaacgtgctgaaaaccagcaacgccaaggccgccatgctggccaagttcaaagagctgtacggcgtcagcttcagcgaactcgtgcggcccttcaagagcaacaagtccacctgttgcgactggtgcattgccgcctttggcctgacaccttctatcgccgacagcatcaagaccctgctccagcagtactgtctgtacctgcacattcagagcctggcctgctcttggggaatggtggtgctgctgctcgtgcggtacaagtgcggcaagaacagagagacaattgagaagctgctgagcaaactgctgtgcgtgtcccctatgtgcatgatgatcgagcctccaaagctgcggtctacagccgccgctctgtactggtacaagaccggcatcagcaatatctccgaggtgtacggcgacacccctgagtggattcagagacagaccgtcctgcagcacagcttcaacgactgcaccttcgagctgtcccagatggtgcagtgggcctacgacaatgacatcgtggacgactccgagatcgcctataagtacgcccagctggccgacaccaacagcaatgccagcgccttcctgaagtccaacagccaggccaagatcgtgaaggactgcgccaccatgtgcagacactacaagcgggccgagaagaaacagatgagcatgagccagtggatcaagtacagatgcgacagagtggacgacggcggcgattggaagcagatcgtgatgttcctgagataccagggcgtcgagttcatgagctttctgacagccctgaagcggttcctgcagggcatccccaagaagaactgcatcctgctgtatggcgccgctaacaccggcaagagcctgttcggaatgagcctgatgaagtttctgcagggctccgtgatctgctttgtgaacagcaagagccacttctggctccagccactggccgatgccaagattggcatgctggacgatgccaccgtgccttgctggaactacatcgacgacaacctgagaaacgccctggacggcaacctggtttctatggacgtgaagcacagacccctggtgcagctgaagtgtcctccactgctgatcaccagcaacatcaacgccggcaccgattccagatggccctatctgcacaaccggctggtggtgtttacctttcctaacgagttccccttcgacgaaaacggcaaccccgtgtacgagctgaacgataagaattggaagtccttcttcagccggacctggtccagactgagcctgcacgaggatgaggacaaagagaacgacggcgacagcctgcctaccttcaaatgtgtcagcggccagaacaccaacacactg

SEQ ID NO: 10 - protein, 649 aa, Human papillomavirus type 16
  REGION 1..649: note = HPV16 E1 translated
MADPAGTNGEEGTGCNGWFYVEAVVEKKTGDAISDDENENDSDTGEDLVDFIVNDNDYLTQAETETAHALFTAQEAKQHRDAVQVLKRKYLGSPLSDISGCVDNNISPRLKAICIEKQSRAAKRRLFESEDSGYGNTEVETQQMLQVEGRHETETPCSQYSGGSGGGCSQYSSGSGGEGVSERHTICQTPLTNILNVLKTSNAKAAMLAKFKELYGVSFSELVRPFKSNKSTCCDWCIAAFGLTPSIADSIKTLLQQYCLYLHIQSLACSWGMVVLLLVRYKCGKNRETIEKLLSKLLCVSPMCMMIEPPKLRSTAAALYWYKTGISNISEVYGDTPEWIQRQTVLQHSFNDCTFELSQMVQWAYDNDIVDDSEIAYKYAQLADTNSNASAFLKSNSQAKIVKDCATMCRHYKRAEKKQMSMSQWIKYRCDRVDDGGDWKQIVMFLRYQGVEFMSFLTALKRFLQGIPKKNCILLYGAANTGKSLFGMSLMKFLQGSVICFVNSKSHFWLQPLADAKIGMLDDATVPCWNYIDDNLRNALDGNLVSMDVKHRPLVQLKCPPLLITSNINAGTDSRWPYLHNRLVVFTFPNEFPFDENGNPVYELNDKNWKSFFSRTWSRLSLHEDEDKENDGDSLPTFKCVSGQNTNTL

SEQ ID NO: 11 - DNA, 1095 bp, Human papillomavirus type 16
  misc_feature 1..1095: note = HPV16 E2
  source 1..1095: mol_type = other DNA
atggaaacactgtgccagcggctgaacgtgtgccaggacaagattctgacccactatgagaacgacagcaccgacctgagggaccacatcgactactggaagcacatgcggctggaatgcgccatctactacaaggctagagagatgggcttcaagcacatcaaccaccaggtggtgcctacactggccgtgtctaagaacaaggctctgcaggccatcgaactgcagctgaccctggaaacaatctacaactcccagtactccaacgagaagtggaccctccaggacgtgtcactggaagtgtacctgaccgctcctaccggctgcatcaagaaacacggctacaccgtggaagtgcagtttgacggcgatatctgcaataccatgcactacaccaactggacccacatctacatctgcgaagaggccagcgtgacagtggtggaaggccaggtcgactactacggcctgtactacgtgcacgagggcatcagaacctacttcgtgcagttcaaggatgacgccgagaagtacagcaagaacaaagtgtgggaagtgcacgctggcggccaagtgatcctgtgtcctacaagcgtgttcagcagcaacgaagtgtctagccccgagatcatcagacagcacctggccaatcatcctgccgccacacacacaaaagctgtggccctgggcaccgaggaaacacagaccacaattcagaggcccagaagcgagcccgataccggcaatccttgtcacaccaccaagctgctgcaccgggattccgtggattctgcccctatcctgaccgccttcaacagctcccacaaaggccggatcaactgcaacagcaacaccacacctatcgtgcacctgaaaggggacgccaacactctgaaggccctgcggtacagattcaagaagcactgtaccctgtataccgccgtgtccagcacatggcactggaccggacacaacgtgaaacacaagagcgccatcgtgaccctgacctacgatagcgagtggcagagggaccagttcctgagccaagtgaagatccctaagaccatcaccgtgtccaccggctttatgagcatt

SEQ ID NO: 12 - protein, 365 aa, Human papillomavirus type 16
  REGION 1..365: note = HPV16 E2 translated
METLCQRLNVCQDKILTHYENDSTDLRDHIDYWKHMRLECAIYYKAREMGFKHINHQVVPTLAVSKNKALQAIELQLTLETIYNSQYSNEKWTLQDVSLEVYLTAPTGCIKKHGYTVEVQFDGDICNTMHYTNWTHIYICEEASVTVVEGQVDYYGLYYVHEGIRTYFVQFKDDAEKYSKNKVWEVHAGGQVILCPTSVFSSNEVSSPEIIRQHLANHPAATHTKAVALGTEETQTTIQRPRSEPDTGNPCHTTKLLHRDSVDSAPILTAFNSSHKGRINCNSNTTPIVHLKGDANTLKALRYRFKKHCTLYTAVSSTWHWTGHNVKHKSAIVTLTYDSEWQRDQFLSQVKIPKTITVSTGFMSI

SEQ ID NO: 13 - DNA, 462 bp, Human papillomavirus type 16
  misc_feature 1..462: note = HPV16 E6
  source 1..462: mol_type = other DNA
atgcaccagaaacggacagccatgttccaggatcctcaagagcggccacggaagctgcctcagctgtgtacagaactgcagaccaccatccacgacatcatcctcgagtgcgtgtactgcaagcagcagctcctgcggagagaagtgtacgacttcgccttccgggatctgtgcatcgtgtacagagatggcaacccctacgccgtgtgcgacaagtgcctgaagttctactccaagatcagcgagtaccggcactactgctactccctgtacggcaccacacttgagcagcagtacaacaagcccctgtgcgatctgctgatccggtgcatcaactgccagaaacctcagtgccccgaggaaaagcagagacacctggacaagaagcagcggttccacaacattagaggcagatggacaggccggtgcatgagctgctgtagaagcagcagaacaagacgg

SEQ ID NO: 14 - protein, 154 aa, Human papillomavirus type 16
  REGION 1..154: note = HPV16 E6 translated
MHQKRTAMFQDPQERPRKLPQLCTELQTTIHDIILECVYCKQQLLRREVYDFAFRDLCIVYRDGNPYAVCDKCLKFYSKISEYRHYCYSLYGTTLEQQYNKPLCDLLIRCINCQKPQCPEEKQRHLDKKQRFHNIRGRWTGRCMSCCRSSRTRR

SEQ ID NO: 15 - DNA, 294 bp, Human papillomavirus type 16
  misc_feature 1..294: note = HPV16 E7
  source 1..294: mol_type = other DNA
atgcacggcgataccccaacactgcacgagtacatgctggatctgcagcccgagacaaccgatctgtacggctatgagcagctgaatgactccagcgaggaagaggacgagatcgacggacctgctggacaggctgaacctgatagagcccactacaatatcgtgaccttctgctgcaagtgcgatagcaccctgagaagatgcgtgcagagcacccacgtggacatcaggaccctcgaggatctgctcatgggcaccctgggaattgtggccccaatctgctctcagaagcct

SEQ ID NO: 16 - protein, 98 aa, Human papillomavirus type 16
  REGION 1..98: note = HPV16 E7 translated
MHGDTPTLHEYMLDLQPETTDLYGYEQLNDSSEEEDEIDGPAGQAEPDRAHYNIVTFCCKCDSTLRRCVQSTHVDIRTLEDLLMGTLGIVAPICSQKP

SEQ ID NO: 17 - DNA, 1971 bp, Human papillomavirus type 18
  misc_feature 1..1971: note = HPV18 E1
  source 1..1971: mol_type = other DNA
atggccgatcctgagggaacagatggcgaaggcacaggctgcaacggctggttttacgtgcaagccatcgtggacaagaagaccggcgacgtcatcagcgacgacgaggatgagaatgccaccgacaccggcagcgacatggtggactttatcgatacccagggcacgttctgcgagcaggccgaactggaaacagcccaggcactgtttcacgcccaagaggtgcacaacgatgcccaggtgctgcacgtgctgaagagaaagtttgccggcggaagcaccgagaacagcccactgggagaaagactggaagtggacaccgagctgagccccagactgcaagagatcagcctgaacagcggccagaagaaggccaagcggagactgttcaccatctccgacagcggctacggctgtagcgaagtggaagccacacagatccaagtgaccaccaatggcgagcacggcggcaatgtttgttctggcggatctaccgaggccatcgacaatggcggaaccgagggcaacaatagctccgtggatggcaccagcgacaacagcaacatcgagaacgtgaaccctcagtgcacaatcgcccagctgaaggacctgctgaaagtgaacaacaagcagggcgccatgctggccgtgtttaaggatacctacggcctgagcttcaccgacctcgtgcggaacttcaagagcgacaagaccacctgtaccgactgggtcaccgccatctttggcgtgaacccaacaatcgccgagggcttcaagaccctgatccagcctttcatcctgtacgcccacatccagtgtctggactgcaaatggggcgtgctgattctggccctgctgagatacaagtgcggcaagagcagactgaccgtggccaagggactgagcactctgctgcatgtgcccgagacatgcatgctgattcagcctccaaagctgcggtctagcgtggccgctctgtactggtacagaaccggcatcagcaatatcagcgaagtgatgggcgacacccctgagtggattcagcggctgacaatcatccagcacggcatcgacgactccaacttcgacctgagcgagatggtgcagtgggccttcgataacgagctgaccgacgagtccgacatggcctttgaatacgccctgctggcagacagcaactccaatgccgccgctttcctgaagtccaactgccaggccaagtacctgaaggactgcgccaccatgtgcaagcactaccgcagagcacagaaacggcagatgaacatgagccagtggatccggttccggtgcagcaagatcgatgaaggtggcgactggcggcctatcgtgcagttcctgagataccagcagatcgagttcatcacctttctgggcgccctgaagtcattcctgaagggaacccctaaaaagaactgcctggtgttctgcggccctgccaataccggcaagtcctacttcggcatgagctttatccacttcatccagggcgccgtgatcagcttcgtgaacagcaccagccacttttggctggaacccctgaccgataccaaggtggcaatgctggacgacgccaccaccacctgttggacctacttcgacacctacatgcggaacgccctggacggcaaccccatcagcatcgacagaaagcacaagcccctcatccagctgaaatgccctccaatcctgctgaccacaaacattcaccccgccaaggacaatcggtggccctacctggaaagccggatcaccgtgttcgagttccccaacgcctttccattcgacaagaacggcaatcccgtgtacgagatcaacgacaagaattggaagtgcttcttcgagcggacctggtccagactggatctgcacgaggaagaagaggacgccgacacagagggcaatcccttcggcacctttaagtgtgtggccgggcagaatcacagacccctg

SEQ ID NO: 18 - protein, 657 aa, Human papillomavirus type 18
  REGION 1..657: note = HPV18 E1 translated
MADPEGTDGEGTGCNGWFYVQAIVDKKTGDVISDDEDENATDTGSDMVDFIDTQGTFCEQAELETAQALFHAQEVHNDAQVLHVLKRKFAGGSTENSPLGERLEVDTELSPRLQEISLNSGQKKAKRRLFTISDSGYGCSEVEATQIQVTTNGEHGGNVCSGGSTEAIDNGGTEGNNSSVDGTSDNSNIENVNPQCTIAQLKDLLKVNNKQGAMLAVFKDTYGLSFTDLVRNFKSDKTTCTDWVTAIFGVNPTIAEGFKTLIQPFILYAHIQCLDCKWGVLILALLRYKCGKSRLTVAKGLSTLLHVPETCMLIQPPKLRSSVAALYWYRTGISNISEVMGDTPEWIQRLTIIQHGIDDSNFDLSEMVQWAFDNELTDESDMAFEYALLADSNSNAAAFLKSNCQAKYLKDCATMCKHYRRAQKRQMNMSQWIRFRCSKIDEGGDWRPIVQFLRYQQIEFITFLGALKSFLKGTPKKNCLVFCGPANTGKSYFGMSFIHFIQGAVISFVNSTSHFWLEPLTDTKVAMLDDATTTCWTYFDTYMRNALDGNPISIDRKHKPLIQLKCPPILLTTNIHPAKDNRWPYLESRITVFEFPNAFPFDKNGNPVYEINDKNWKCFFERTWSRLDLHEEEEDADTEGNPFGTFKCVAGQNHRPL

SEQ ID NO: 19 - DNA, 1095 bp, Human papillomavirus type 18
  misc_feature 1..1095: note = HPV18 E2
  source 1..1095: mol_type = other DNA
atgcagaccccaaaagagacactgagcgagcggctgagcgccctccaggacaagatcatcgaccactacgagaacgacagcaaggacatcgacagccagattcagtactggcagctgatcagatgggagaacgccatcttcttcgccgctagagagcacggaattcagaccctgaaccaccaggtggtgcccgcctacaacatcagcaagagcaaggcccacaaggccattgaactccagatggccctgcaaggactggctcagagcgcctacaagaccgaggattggacactccaggatacctgcgaggaactgtggaacacagagcccacacactgcttcaagaaaggcggccagaccgtgcaggtctacttcgatggcaacaaggataactgcatgacctacgtggcctgggacagcgtgtactacatgacagatgccggcacctgggacaagaccgccacctgtgtttctcaccggggcctgtactacgtgaaagagggctacaacaccttctacatcgagtttaagagcgagtgcgagaagtatggcaacaccggcacatgggaagtgcatttcggcaacaacgtgatcgactgcaacgactccatgtgctccacctccgatgacaccgtgtctgccacacagctggtcaagcagctccagcacaccccatctccatacagctctacagtgtctgtgggcaccgccaagacctatggccaaacaagcgctgccacaagacctggccattgtggactggccgagaagcagcactgtggccctgttaatcctctgctgggagccgccacacctacaggcaacaacaagagaagaaagctgtgcagcggcaacaccacacctatcattcacctgaagggcgacagaaacagcctgaagtgcctgcggtacagactgaggaagcacagcgatcactaccgggacatcagctccacctggcactggacaggtgctggcaacgaaaagaccggcatcctgacagtgacctaccacagcgagactcagcggaccaagttcctgaacaccgtggctatccccgacagcgtgcagattctcgtgggctacatgaccatg

SEQ ID NO: 20 - protein, 365 aa, Human papillomavirus type 18
  REGION 1..365: note = HPV18 E2 translated
MQTPKETLSERLSALQDKIIDHYENDSKDIDSQIQYWQLIRWENAIFFAAREHGIQTLNHQVVPAYNISKSKAHKAIELQMALQGLAQSAYKTEDWTLQDTCEELWNTEPTHCFKKGGQTVQVYFDGNKDNCMTYVAWDSVYYMTDAGTWDKTATCVSHRGLYYVKEGYNTFYIEFKSECEKYGNTGTWEVHFGNNVIDCNDSMCSTSDDTVSATQLVKQLQHTPSPYSSTVSVGTAKTYGQTSAATRPGHCGLAEKQHCGPVNPLLGAATPTGNNKRRKLCSGNTTPIIHLKGDRNSLKCLRYRLRKHSDHYRDISSTWHWTGAGNEKTGILTVTYHSETQRTKFLNTVAIPDSVQILVGYMTM

SEQ ID NO: 21 - DNA, 462 bp, Human papillomavirus type 18
  misc_feature 1..462: note = HPV18 E6
  source 1..462: mol_type = other DNA
atggccagattcgaggaccctaccagacggccctacaagctgcctgatctgtgcaccgaactgaacaccagccttcaggatatcgagatcacctgtgtgtactgcaagaccgtgctcgagctgacagaagtgtttgagttcgccttcaaggacctgttcgtggtgtacagagacagcatccctcacgccgcctgtcacaagtgcatcgacttctacagcagaatcagagagctgcggcactacagcgactccgtgtacggcgataccctggaaaaactgaccaacaccggactgtacaacctgctgatccggtgcctgagatgccagaaacctcagaaccccgccgagaaactgagacatctgaacgagaagcggcggttccacaatatcgccggccactacagaggccagtgccacagctgttgcaaccgggccagacaagaaagactccagcggagaaga

SEQ ID NO: 22 - protein, 154 aa, Human papillomavirus type 18
  REGION 1..154: note = HPV18 E6 translated
MARFEDPTRRPYKLPDLCTELNTSLQDIEITCVYCKTVLELTEVFEFAFKDLFVVYRDSIPHAACHKCIDFYSRIRELRHYSDSVYGDTLEKLTNTGLYNLLIRCLRCQKPQNPAEKLRHLNEKRRFHNIAGHYRGQCHSCCNRARQERLQRRR

SEQ ID NO: 23 - DNA, 315 bp, Human papillomavirus type 18
  misc_feature 1..315: note = HPV18 E7
  source 1..315: mol_type = other DNA
atgcacggacctaaagccacacttcaggacatcgtgctgcacctcgagcctcagaatgagatccccgtggatctgctcggccatgagcagctgagcgatagcgaggaagagaacgacgagatcgacggcgtcaaccaccagcatctgcctgccagaagggccgaacctcagagacacaccatgctgtgcatgtgctgcaagtgcgaggcccggatcgagagagtggtggaatctagcgccgacgacctgagagctttccagcagctgttcctgaatactctgagcttcgtggccccttggtgcgctagtcagcag

SEQ ID NO: 24 - protein, 105 aa, Human papillomavirus type 18
  REGION 1..105: note = HPV18 E7 translated
MHGPKATLQDIVLHLEPQNEIPVDLLGHEQLSDSEEENDEIDGVNHQHLPARRAEPQRHTMLCMCCKCEARIERVVESSADDLRAFQQLFLNTLSFVAPWCASQQ

SEQ ID NO: 25 - DNA, 4455 bp, Macaca fascicularis
  misc_feature 1..4455: note = MfPV3 Ii-E1E2E6E7
  source 1..4455: mol_type = other DNA
atgcacagaaggcggagcagaagctgccgcgaggatcagaaacccgtgatggacgaccagcgggacctgatcagcaacaacgagcagctgcctatgctcggcagaaggcctggcgctcctgagagcaaatgttctagaggcgccctgtacaccggcttcagcatcctggttacactgctgctggccggacaggctaccaccgcctactttctgtatcagcagcagggcagactggacaagctgaccgtgaccagccagaacctgcagctggaaaacctgcggatgaagctgcccaagcctcctaagcctgtgtccaagatgcggatggccacacctctgctgatgcaggctttgcctatgggagcactgcctcagggccctatgcagaacgccaccaaatacggcaacatgaccgaggaccacgtgatgcatctgctgcagaatgccgatcctctgaaggtgtaccctccactgaagggcagctttcccgagaacctgcggcacctgaagaacaccatggaaaccatcgactggaaggtgttcgagagctggatgcaccactggctgctgttcgagatgagccggcacagcctggaacagaagcctacagacgcccctcctaaagagtccctggaactggaagatcccagctctggcctgggcgtgacaaagcaagatctgggccctgtgcctatgatggacccttctggaattcacggcgagggcaccagatgcaccggatggttcgatgtggaagccatcgtggaaagacggaccggcgacgtggtgtccgaggacgaagatgagaacctggaagataccggcgtggacctggtggacttcatcgacgatacctgcgagtttgagcaggccggcgacgaagctcctagagcactgctgcatgcccaagagacacaggcccatgtgaacgccgtgcactacctgaagcggaagttcacatgcagccctaccgtgtctcccctgggcaatgtgcagtgttctatcgagggcgagctgagccctagactgcacgccatccaactgaatgaaggcagcggcaaggccaagcggagactgtttctgcctgatagcggctacggcaataccgaggtggaaacccagctgtctcaggtggcaggatccgatggcgaatctcaagcccctggcggcaaagaaaatgtgcagcctgctgaaggcgctggcggagatgttcgacagctgctgagaagcagcaacctgaaggccacactgctgggcaagttcaaggacgtgtacggcatcggcttcaacgaactcgtgcggtgcttcaagagcgacaagaccgtgtgcaccgattgggttgtgggagctgctggcgtgcaccattctgtggccgaaggactgaagcacctgatcgagcctttctgcacatacgcccacatgcagtgcctgacctgcaactggggagtgtttctgctgctgctcatccggttcaagtgcggcaagaacagactgaccgtggccaagtgcatctctaccctgctgaacgtgcaagagccctacctgctgatcgaacctccaaagctgagatccacagccgccgctctgtactggtacagaaccggcatcagcaacgtgtccgaagtgatcggagagcctcctgagtggatcgtgcggcagaccatgtttcagcacagcctcgaggacagcatcttcgacctgtctcagatggtgcagtgggcctacgaccacgatctggtggacgatagcgccattgcctaccagtatgcccagctggccgacgtggactctaatgccgccgctttcctgaagtctaacgctcaggccaaatacgtgaaggactgcgccaccatgtgcagacactacaagagagccgagaagcaggccatgaccatgagccagtggatcagagccagatgcgataagaccgacgacggcggagattggaggcctatcgtgcagttcctgagataccagaacgtcgagttcatcacctttctgagcgcctttaaggccttcctgaagggaatccccaagagaagctgcatcgtgctgtacggccctccaaacaccggcaagagctacttcggaatgagcctgatgcagtttctgcagggcagcatcatcagctacgtgaacagcaacagccacttttggctggcccctctggccgaagctaaagtggccatgctggacgatgccacaccacagtgctggtcctacatcgacacctacatgcggagcgtgctggacggcaaccccatgagcattgaccggaagcacaagaacctgatccagatgaagtgtccgcctctgatcatcaccagcaacaccaacgtgggcgaagatgacagatggcgctacctgcactccagagtggtggtgttcaagttcgagcagacctttccattcgaccagaacggcaatcccgtgtacgagctgtccgacaagaattggaagtccttcttcagccggacctggtccagactcgagctgcaagaggatgaggacaccgagcaggatggctctacagtgcgggcctttaagtgtgtgcccggccagaacaccaatatcctgggctctatggaagccctgagcgagagactgaacgcctaccaggacaagatcctgaccttctacgaagaggactccaaggacctgagggaccagatcgagcattggagatgtgtgcggggagagtgcgccgtgttctacaaggccagagagatgggccacagccacatcaaccaccagcaggttccacctctgcacgtgtccagaggcaaggcccacaatgccattgagctgcagatggccctggaaagcctgaaccagagcgacttcaacaccgaggaatggaccctgcaggacacaagcctggaaatgtggcggaccgagcctcagtgctgcttcaaaaagaaaggcgagacagtgaccgtgctgtatgactgcgacaaggacaacagcatggactacaccctgtggggccacgtgtacgtgtggggagatacaggctgggacaaagtgtgcggaaaggtggactacggcggcctgtactatgtgctgcacggcctgaaagtgtactacgttgagttccagcaagaggccgtgaagtacagcaagaccaacaagtgggaagtgcatatcggcagaacagtgatcccctacagcgagtccgtgtcctctacagccctgtgcgaaagagtgcctatcgccgagatcgccaatggcctccagcagaccagcattccctctcacacacacgccaccaccaaagagaacgcttgggtttcctctcagcctcctaccaagcggtccagaagatctgatccaggcggcgatcctgtcagagccctggatgccaatagcagaaccctgctgtctggaaccgcctgcgagaataccaccggcaactcttacagcgactgcacccctgtggtgcacctgaaaggcgaccccaattgtctgaaggccctgcggttcagattcgtgaaacacaagaatctgttcgtgaacgtgtcctccacctggcggtggaccaacaacaccgaagagaaggccatcgtgactgtgacctttaaggacgagcagcagagacagcagttcctcggcagcgtgaagatccccaactccatctgtgtgtgcaagggcgtgatgacagttggcagcatggtggaaggcagccaagagagagcccggacactgcacgaactgtgcgagcagagagaacagacccagcacgacatcgagctggactgcgtgtactgcctgaatcctctgaccagaaccgaggtgtacgacttcgccagatgggagctgagaatcgtgtacagacgggataccgcctacggcgtgtgcaagctgtgcctgaggttccacagcaaagtgcggaagtacaggcggtacaactactccatctacggccccacactggaacggcggacaaagaagcagctcgaagaactcgtgatccggtgctactgctgccagaaacctcagtgccccgtggaaaagcagcgccacgttgaccagggccagagattccacaacattgccgagcagtggaccggcagatgcctgcaatgttggaggccaacagccgctgaaaccggctctatgatcggccctggacctacactgcaggatatcgtgcttgagctggaacccgagcctgtggacctgagaggatatgagcagctggactccagcgaggacgaggacgagattgaccaccaccaacagcaggacagacccgatgtgcaagaagatgacggcgagcggtttagaatcctgagcgactgtcacgtgtgccagaataccgtgtcaagagtggtgcacagcacccacgacgacctgcacattctcgaggacctgcttatgggcaccctcgagatcgtggcccctaattgtgccgtgggcagcgaacagaaactgatctccgaggaagatctctga

SEQ ID NO: 26 - protein, 1484 aa, Macaca fascicularis
  REGION 1..1484: note = MfPV3 Ii-E1E2E6E7 translated
MHRRRSRSCREDQKPVMDDQRDLISNNEQLPMLGRRPGAPESKCSRGALYTGFSILVTLLLAGQATTAYFLYQQQGRLDKLTVTSQNLQLENLRMKLPKPPKPVSKMRMATPLLMQALPMGALPQGPMQNATKYGNMTEDHVMHLLQNADPLKVYPPLKGSFPENLRHLKNTMETIDWKVFESWMHHWLLFEMSRHSLEQKPTDAPPKESLELEDPSSGLGVTKQDLGPVPMMDPSGIHGEGTRCTGWFDVEAIVERRTGDVVSEDEDENLEDTGVDLVDFIDDTCEFEQAGDEAPRALLHAQETQAHVNAVHYLKRKFTCSPTVSPLGNVQCSIEGELSPRLHAIQLNEGSGKAKRRLFLPDSGYGNTEVETQLSQVAGSDGESQAPGGKENVQPAEGAGGDVRQLLRSSNLKATLLGKFKDVYGIGFNELVRCFKSDKTVCTDWVVGAAGVHHSVAEGLKHLIEPFCTYAHMQCLTCNWGVFLLLLIRFKCGKNRLTVAKCISTLLNVQEPYLLIEPPKLRSTAAALYWYRTGISNVSEVIGEPPEWIVRQTMFQHSLEDSIFDLSQMVQWAYDHDLVDDSAIAYQYAQLADVDSNAAAFLKSNAQAKYVKDCATMCRHYKRAEKQAMTMSQWIRARCDKTDDGGDWRPIVQFLRYQNVEFITFLSAFKAFLKGIPKRSCIVLYGPPNTGKSYFGMSLMQFLQGSIISYVNSNSHFWLAPLAEAKVAMLDDATPQCWSYIDTYMRSVLDGNPMSIDRKHKNLIQMKCPPLIITSNTNVGEDDRWRYLHSRVVVFKFEQTFPFDQNGNPVYELSDKNWKSFFSRTWSRLELQEDEDTEQDGSTVRAFKCVPGQNTNILGSMEALSERLNAYQDKILTFYEEDSKDLRDQIEHWRCVRGECAVFYKAREMGHSHINHQQVPPLHVSRGKAHNAIELQMALESLNQSDFNTEEWTLQDTSLEMWRTEPQCCFKKKGETVTVLYDCDKDNSMDYTLWGHVYVWGDTGWDKVCGKVDYGGLYYVLHGLKVYYVEFQQEAVKYSKTNKWEVHIGRTVIPYSESVSSTALCERVPIAEIANGLQQTSIPSHTHATTKENAWVSSQPPTKRSRRSDPGGDPVRALDANSRTLLSGTACENTTGNSYSDCTPVVHLKGDPNCLKALRFRFVKHKNLFVNVSSTWRWTNNTEEKAIVTVTFKDEQQRQQFLGSVKIPNSICVCKGVMTVGSMVEGSQERARTLHELCEQREQTQHDIELDCVYCLNPLTRTEVYDFARWELRIVYRRDTAYGVCKLCLRFHSKVRKYRRYNYSIYGPTLERRTKKQLEELVIRCYCCQKPQCPVEKQRHVDQGQRFHNIAEQWTGRCLQCWRPTAAETGSMIGPGPTLQDIVLELEPEPVDLRGYEQLDSSEDEDEIDHHQQQDRPDVQEDDGERFRILSDCHVCQNTVSRVVHSTHDDLHILEDLLMGTLEIVAPNCAVGSEQKLISEEDL

SEQ ID NO: 27 - DNA, 5211 bp, Macaca fascicularis
  misc_feature 1..5211: note = MfPV3 Ii-E1E2-p2a-Ii-E6E7
  source 1..5211: mol_type = other DNA
atgcacagaaggcggagcagaagctgccgcgaggatcagaaacccgtgatggacgaccagcgggacctgatcagcaacaacgagcagctgcctatgctcggcagaaggcctggcgctcctgagagcaaatgttctagaggcgccctgtacaccggcttcagcatcctggttacactgctgctggccggacaggctaccaccgcctactttctgtatcagcagcagggcagactggacaagctgaccgtgaccagccagaacctgcagctggaaaacctgcggatgaagctgcccaagcctcctaagcctgtgtccaagatgcggatggccacacctctgctgatgcaggctttgcctatgggagcactgcctcagggccctatgcagaacgccaccaaatacggcaacatgaccgaggaccacgtgatgcatctgctgcagaatgccgatcctctgaaggtgtaccctccactgaagggcagctttcccgagaacctgcggcacctgaagaacaccatggaaaccatcgactggaaggtgttcgagagctggatgcaccactggctgctgttcgagatgagccggcacagcctggaacagaagcctacagacgcccctcctaaagagtccctggaactggaagatcccagctctggcctgggcgtgacaaagcaagatctgggccctgtgcctatgatggacccttctggaattcacggcgagggcaccagatgcaccggatggttcgatgtggaagccatcgtggaaagacggaccggcgacgtggtgtccgaggacgaagatgagaacctggaagataccggcgtggacctggtggacttcatcgacgatacctgcgagtttgagcaggccggcgacgaagctcctagagcactgctgcatgcccaagagacacaggcccatgtgaacgccgtgcactacctgaagcggaagttcacatgcagccctaccgtgtctcccctgggcaatgtgcagtgttctatcgagggcgagctgagccctagactgcacgccatccaactgaatgaaggcagcggcaaggccaagcggagactgtttctgcctgatagcggctacggcaataccgaggtggaaacccagctgtctcaggtggcaggatccgatggcgaatctcaagcccctggcggcaaagaaaatgtgcagcctgctgaaggcgctggcggagatgttcgacagctgctgagaagcagcaacctgaaggccacactgctgggcaagttcaaggacgtgtacggcatcggcttcaacgaactcgtgcggtgcttcaagagcgacaagaccgtgtgcaccgattgggttgtgggagctgctggcgtgcaccattctgtggccgaaggactgaagcacctgatcgagcctttctgcacatacgcccacatgcagtgcctgacctgcaactggggagtgtttctgctgctgctcatccggttcaagtgcggcaagaacagactgaccgtggccaagtgcatctctaccctgctgaacgtgcaagagccctacctgctgatcgaacctccaaagctgagatccacagccgccgctctgtactggtacagaaccggcatcagcaacgtgtccgaagtgatcggagagcctcctgagtggatcgtgcggcagaccatgtttcagcacagcctcgaggacagcatcttcgacctgtctcagatggtgcagtgggcctacgaccacgatctggtggacgatagcgccattgcctaccagtatgcccagctggccgacgtggactctaatgccgccgctttcctgaagtctaacgctcaggccaaatacgtgaaggactgcgccaccatgtgcagacactacaagagagccgagaagcaggccatgaccatgagccagtggatcagagccagatgcgataagaccgacgacggcggagattggaggcctatcgtgcagttcctgagataccagaacgtcgagttcatcacctttctgagcgcctttaaggccttcctgaagggaatccccaagagaagctgcatcgtgctgtacggccctccaaacaccggcaagagctacttcggaatgagcctgatgcagtttctgcagggcagcatcatcagctacgtgaacagcaacagccacttttggctggcccctctggccgaagctaaagtggccatgctggacgatgccacaccacagtgctggtcctacatcgacacctacatgcggagcgtgctggacggcaaccccatgagcattgaccggaagcacaagaacctgatccagatgaagtgtccgcctctgatcatcaccagcaacaccaacgtgggcgaagatgacagatggcgctacctgcactccagagtggtggtgttcaagttcgagcagacctttccattcgaccagaacggcaatcccgtgtacgagctgtccgacaagaattggaagtccttcttcagccggacctggtccagactcgagctgcaagaggatgaggacaccgagcaggatggctctacagtgcgggcctttaagtgtgtgcccggccagaacaccaatatcctgggctctatggaagccctgagcgagagactgaacgcctaccaggacaagatcctgaccttctacgaagaggactccaaggacctgagggaccagatcgagcattggagatgtgtgcggggagagtgcgccgtgttctacaaggccagagagatgggccacagccacatcaaccaccagcaggttccacctctgcacgtgtccagaggcaaggcccacaatgccattgagctgcagatggccctggaaagcctgaaccagagcgacttcaacaccgaggaatggaccctgcaggacacaagcctggaaatgtggcggaccgagcctcagtgctgcttcaaaaagaaaggcgagacagtgaccgtgctgtatgactgcgacaaggacaacagcatggactacaccctgtggggccacgtgtacgtgtggggagatacaggctgggacaaagtgtgcggaaaggtggactacggcggcctgtactatgtgctgcacggcctgaaagtgtactacgttgagttccagcaagaggccgtgaagtacagcaagaccaacaagtgggaagtgcatatcggcagaacagtgatcccctacagcgagtccgtgtcctctacagccctgtgcgaaagagtgcctatcgccgagatcgccaatggcctccagcagaccagcattccctctcacacacacgccaccaccaaagagaacgcttgggtttcctctcagcctcctaccaagcggtccagaagatctgatccaggcggcgatcctgtcagagccctggatgccaatagcagaaccctgctgtctggaaccgcctgcgagaataccaccggcaactcttacagcgactgcacccctgtggtgcacctgaaaggcgaccccaattgtctgaaggccctgcggttcagattcgtgaaacacaagaatctgttcgtgaacgtgtcctccacctggcggtggaccaacaacaccgaagagaaggccatcgtgactgtgacctttaaggacgagcagcagagacagcagttcctcggcagcgtgaagatccccaactccatctgtgtgtgcaagggcgtgatgacagttggcagcggcgccaccaatttcagtctgcttaaacaggccggggacgtcgaagagaaccccggacctatgcatcggcgcagatctcggtcttgtagagaagaccaaaagcctgtcatggatgatcaaagagatttaatttctaataatgaacaattacccatgttgggacggcgccccggagcccccgaatctaagtgcagccggggagctttatatacaggattttctattttagtgaccttattattagctggccaagccacaacagcttatttcttataccaacaacaaggacggttagataaattaacagtcacatctcaaaatttacaattagagaatttaagaatgaaattacctaaaccacccaaacccgtcagtaaaatgagaatggctacccccttattaatgcaagccctccccatgggcgccttaccccaaggacccatgcaaaatgctacaaagtatggaaatatgacagaagatcatgtcatgcacttattacaaaacgctgaccccttaaaagtctatccccctttaaaaggatctttccctgaaaatttaagacatttaaaaaatacaatggagacaattgattggaaagtctttgaatcttggatgcatcattggttattatttgaaatgtctaggcattctttagagcaaaaacccaccgatgctccacccaaggaaagtttagagttagaggacccttctagcggattaggagtcaccaaacaggacttaggacccgtccccatgatggtggaaggcagccaagagagagcccggacactgcacgaactgtgcgagcagagagaacagacccagcacgacatcgagctggactgcgtgtactgcctgaatcctctgaccagaaccgaggtgtacgacttcgccagatgggagctgagaatcgtgtacagacgggataccgcctacggcgtgtgcaagctgtgcctgaggttccacagcaaagtgcggaagtacaggcggtacaactactccatctacggccccacactggaacggcggacaaagaagcagctcgaagaactcgtgatccggtgctactgctgccagaaacctcagtgccccgtggaaaagcagcgccacgttgaccagggccagagattccacaacattgccgagcagtggaccggcagatgcctgcaatgttggaggccaacagccgctgaaaccggctctatgatcggccctggacctacactgcaggatatcgtgcttgagctggaacccgagcctgtggacctgagaggatatgagcagctggactccagcgaggacgaggacgagattgaccaccaccaacagcaggacagacccgatgtgcaagaagatgacggcgagcggtttagaatcctgagcgactgtcacgtgtgccagaataccgtgtcaagagtggtgcacagcacccacgacgacctgcacattctcgaggacctgcttatgggcaccctcgagatcgtggcccctaattgtgccgtgggcagcgaacagaaactgatctccgaggaagatctctga

SEQ ID NO: 28 - protein, 1736 aa, Macaca fascicularis
  REGION 1..1736: note = MfPV3 Ii-E1E2-p2a-Ii-E6E7 translated
MHRRRSRSCREDQKPVMDDQRDLISNNEQLPMLGRRPGAPESKCSRGALYTGFSILVTLLLAGQATTAYFLYQQQGRLDKLTVTSQNLQLENLRMKLPKPPKPVSKMRMATPLLMQALPMGALPQGPMQNATKYGNMTEDHVMHLLQNADPLKVYPPLKGSFPENLRHLKNTMETIDWKVFESWMHHWLLFEMSRHSLEQKPTDAPPKESLELEDPSSGLGVTKQDLGPVPMMDPSGIHGEGTRCTGWFDVEAIVERRTGDVVSEDEDENLEDTGVDLVDFIDDTCEFEQAGDEAPRALLHAQETQAHVNAVHYLKRKFTCSPTVSPLGNVQCSIEGELSPRLHAIQLNEGSGKAKRRLFLPDSGYGNTEVETQLSQVAGSDGESQAPGGKENVQPAEGAGGDVRQLLRSSNLKATLLGKFKDVYGIGFNELVRCFKSDKTVCTDWVVGAAGVHHSVAEGLKHLIEPFCTYAHMQCLTCNWGVFLLLLIRFKCGKNRLTVAKCISTLLNVQEPYLLIEPPKLRSTAAALYWYRTGISNVSEVIGEPPEWIVRQTMFQHSLEDSIFDLSQMVQWAYDHDLVDDSAIAYQYAQLADVDSNAAAFLKSNAQAKYVKDCATMCRHYKRAEKQAMTMSQWIRARCDKTDDGGDWRPIVQFLRYQNVEFITFLSAFKAFLKGIPKRSCIVLYGPPNTGKSYFGMSLMQFLQGSIISYVNSNSHFWLAPLAEAKVAMLDDATPQCWSYIDTYMRSVLDGNPMSIDRKHKNLIQMKCPPLIITSNTNVGEDDRWRYLHSRVVVFKFEQTFPFDQNGNPVYELSDKNWKSFFSRTWSRLELQEDEDTEQDGSTVRAFKCVPGQNTNILGSMEALSERLNAYQDKILTFYEEDSKDLRDQIEHWRCVRGECAVFYKAREMGHSHINHQQVPPLHVSRGKAHNAIELQMALESLNQSDFNTEEWTLQDTSLEMWRTEPQCCFKKKGETVTVLYDCDKDNSMDYTLWGHVYVWGDTGWDKVCGKVDYGGLYYVLHGLKVYYVEFQQEAVKYSKTNKWEVHIGRTVIPYSESVSSTALCERVPIAEIANGLQQTSIPSHTHATTKENAWVSSQPPTKRSRRSDPGGDPVRALDANSRTLLSGTACENTTGNSYSDCTPVVHLKGDPNCLKALRFRFVKHKNLFVNVSSTWRWTNNTEEKAIVTVTFKDEQQRQQFLGSVKIPNSICVCKGVMTVGSGATNFSLLKQAGDVEENPGPMHRRRSRSCREDQKPVMDDQRDLISNNEQLPMLGRRPGAPESKCSRGALYTGFSILVTLLLAGQATTAYFLYQQQGRLDKLTVTSQNLQLENLRMKLPKPPKPVSKMRMATPLLMQALPMGALPQGPMQNATKYGNMTEDHVMHLLQNADPLKVYPPLKGSFPENLRHLKNTMETIDWKVFESWMHHWLLFEMSRHSLEQKPTDAPPKESLELEDPSSGLGVTKQDLGPVPMMVEGSQERARTLHELCEQREQTQHDIELDCVYCLNPLTRTEVYDFARWELRIVYRRDTAYGVCKLCLRFHSKVRKYRRYNYSIYGPTLERRTKKQLEELVIRCYCCQKPQCPVEKQRHVDQGQRFHNIAEQWTGRCLQCWRPTAAETGSMIGPGPTLQDIVLELEPEPVDLRGYEQLDSSEDEDEIDHHQQQDRPDVQEDDGERFRILSDCHVCQNTVSRVVHSTHDDLHILEDLLMGTLEIVAPNCAVGSEQKLISEEDL

SEQ ID NO: 29 - DNA, 3759 bp, Macaca fascicularis
  misc_feature 1..3759: note = MfPV3 E1E2E6E7
  source 1..3759: mol_type = other DNA
atggacccttctggaattcacggcgagggcaccagatgcaccggatggttcgatgtggaagccatcgtggaaagacggaccggcgacgtggtgtccgaggacgaagatgagaacctggaagataccggcgtggacctggtggacttcatcgacgatacctgcgagtttgagcaggccggcgacgaagctcctagagcactgctgcatgcccaagagacacaggcccatgtgaacgccgtgcactacctgaagcggaagttcacatgcagccctaccgtgtctcccctgggcaatgtgcagtgttctatcgagggcgagctgagccctagactgcacgccatccaactgaatgaaggcagcggcaaggccaagcggagactgtttctgcctgatagcggctacggcaataccgaggtggaaacccagctgtctcaggtggcaggatccgatggcgaatctcaagcccctggcggcaaagaaaatgtgcagcctgctgaaggcgctggcggagatgttcgacagctgctgagaagcagcaacctgaaggccacactgctgggcaagttcaaggacgtgtacggcatcggcttcaacgaactcgtgcggtgcttcaagagcgacaagaccgtgtgcaccgattgggttgtgggagctgctggcgtgcaccattctgtggccgaaggactgaagcacctgatcgagcctttctgcacatacgcccacatgcagtgcctgacctgcaactggggagtgtttctgctgctgctcatccggttcaagtgcggcaagaacagactgaccgtggccaagtgcatctctaccctgctgaacgtgcaagagccctacctgctgatcgaacctccaaagctgagatccacagccgccgctctgtactggtacagaaccggcatcagcaacgtgtccgaagtgatcggagagcctcctgagtggatcgtgcggcagaccatgtttcagcacagcctcgaggacagcatcttcgacctgtctcagatggtgcagtgggcctacgaccacgatctggtggacgatagcgccattgcctaccagtatgcccagctggccgacgtggactctaatgccgccgctttcctgaagtctaacgctcaggccaaatacgtgaaggactgcgccaccatgtgcagacactacaagagagccgagaagcaggccatgaccatgagccagtggatcagagccagatgcgataagaccgacgacggcggagattggaggcctatcgtgcagttcctgagataccagaacgtcgagttcatcacctttctgagcgcctttaaggccttcctgaagggaatccccaagagaagctgcatcgtgctgtacggccctccaaacaccggcaagagctacttcggaatgagcctgatgcagtttctgcagggcagcatcatcagctacgtgaacagcaacagccacttttggctggcccctctggccgaagctaaagtggccatgctggacgatgccacaccacagtgctggtcctacatcgacacctacatgcggagcgtgctggacggcaaccccatgagcattgaccggaagcacaagaacctgatccagatgaagtgtccgcctctgatcatcaccagcaacaccaacgtgggcgaagatgacagatggcgctacctgcactccagagtggtggtgttcaagttcgagcagacctttccattcgaccagaacggcaatcccgtgtacgagctgtccgacaagaattggaagtccttcttcagccggacctggtccagactcgagctgcaagaggatgaggacaccgagcaggatggctctacagtgcgggcctttaagtgtgtgcccggccagaacaccaatatcctgggctctatggaagccctgagcgagagactgaacgcctaccaggacaagatcctgaccttctacgaagaggactccaaggacctgagggaccagatcgagcattggagatgtgtgcggggagagtgcgccgtgttctacaaggccagagagatgggccacagccacatcaaccaccagcaggttccacctctgcacgtgtccagaggcaaggcccacaatgccattgagctgcagatggccctggaaagcctgaaccagagcgacttcaacaccgaggaatggaccctgcaggacacaagcctggaaatgtggcggaccgagcctcagtgctgcttcaaaaagaaaggcgagacagtgaccgtgctgtatgactgcgacaaggacaacagcatggactacaccctgtggggccacgtgtacgtgtggggagatacaggctgggacaaagtgtgcggaaaggtggactacggcggcctgtactatgtgctgcacggcctgaaagtgtactacgttgagttccagcaagaggccgtgaagtacagcaagaccaacaagtgggaagtgcatatcggcagaacagtgatcccctacagcgagtccgtgtcctctacagccctgtgcgaaagagtgcctatcgccgagatcgccaatggcctccagcagaccagcattccctctcacacacacgccaccaccaaagagaacgcttgggtttcctctcagcctcctaccaagcggtccagaagatctgatccaggcggcgatcctgtcagagccctggatgccaatagcagaaccctgctgtctggaaccgcctgcgagaataccaccggcaactcttacagcgactgcacccctgtggtgcacctgaaaggcgaccccaattgtctgaaggccctgcggttcagattcgtgaaacacaagaatctgttcgtgaacgtgtcctccacctggcggtggaccaacaacaccgaagagaaggccatcgtgactgtgacctttaaggacgagcagcagagacagcagttcctcggcagcgtgaagatccccaactccatctgtgtgtgcaagggcgtgatgacagttggcagcatggtggaaggcagccaagagagagcccggacactgcacgaactgtgcgagcagagagaacagacccagcacgacatcgagctggactgcgtgtactgcctgaatcctctgaccagaaccgaggtgtacgacttcgccagatgggagctgagaatcgtgtacagacgggataccgcctacggcgtgtgcaagctgtgcctgaggttccacagcaaagtgcggaagtacaggcggtacaactactccatctacggccccacactggaacggcggacaaagaagcagctcgaagaactcgtgatccggtgctactgctgccagaaacctcagtgccccgtggaaaagcagcgccacgttgaccagggccagagattccacaacattgccgagcagtggaccggcagatgcctgcaatgttggaggccaacagccgctgaaaccggctctatgatcggccctggacctacactgcaggatatcgtgcttgagctggaacccgagcctgtggacctgagaggatatgagcagctggactccagcgaggacgaggacgagattgaccaccaccaacagcaggacagacccgatgtgcaagaagatgacggcgagcggtttagaatcctgagcgactgtcacgtgtgccagaataccgtgtcaagagtggtgcacagcacccacgacgacctgcacattctcgaggacctgcttatgggcaccctcgagatcgtggcccctaattgtgccgtgggcagcgaacagaaactgatctccgaggaagatctctga

SEQ ID NO: 30 - protein, 1252 aa, Macaca fascicularis
  REGION 1..1252: note = MfPV3 E1E2E6E7 translated
MDPSGIHGEGTRCTGWFDVEAIVERRTGDVVSEDEDENLEDTGVDLVDFIDDTCEFEQAGDEAPRALLHAQETQAHVNAVHYLKRKFTCSPTVSPLGNVQCSIEGELSPRLHAIQLNEGSGKAKRRLFLPDSGYGNTEVETQLSQVAGSDGESQAPGGKENVQPAEGAGGDVRQLLRSSNLKATLLGKFKDVYGIGFNELVRCFKSDKTVCTDWVVGAAGVHHSVAEGLKHLIEPFCTYAHMQCLTCNWGVFLLLLIRFKCGKNRLTVAKCISTLLNVQEPYLLIEPPKLRSTAAALYWYRTGISNVSEVIGEPPEWIVRQTMFQHSLEDSIFDLSQMVQWAYDHDLVDDSAIAYQYAQLADVDSNAAAFLKSNAQAKYVKDCATMCRHYKRAEKQAMTMSQWIRARCDKTDDGGDWRPIVQFLRYQNVEFITFLSAFKAFLKGIPKRSCIVLYGPPNTGKSYFGMSLMQFLQGSIISYVNSNSHFWLAPLAEAKVAMLDDATPQCWSYIDTYMRSVLDGNPMSIDRKHKNLIQMKCPPLIITSNTNVGEDDRWRYLHSRVVVFKFEQTFPFDQNGNPVYELSDKNWKSFFSRTWSRLELQEDEDTEQDGSTVRAFKCVPGQNTNILGSMEALSERLNAYQDKILTFYEEDSKDLRDQIEHWRCVRGECAVFYKAREMGHSHINHQQVPPLHVSRGKAHNAIELQMALESLNQSDFNTEEWTLQDTSLEMWRTEPQCCFKKKGETVTVLYDCDKDNSMDYTLWGHVYVWGDTGWDKVCGKVDYGGLYYVLHGLKVYYVEFQQEAVKYSKTNKWEVHIGRTVIPYSESVSSTALCERVPIAEIANGLQQTSIPSHTHATTKENAWVSSQPPTKRSRRSDPGGDPVRALDANSRTLLSGTACENTTGNSYSDCTPVVHLKGDPNCLKALRFRFVKHKNLFVNVSSTWRWTNNTEEKAIVTVTFKDEQQRQQFLGSVKIPNSICVCKGVMTVGSMVEGSQERARTLHELCEQREQTQHDIELDCVYCLNPLTRTEVYDFARWELRIVYRRDTAYGVCKLCLRFHSKVRKYRRYNYSIYGPTLERRTKKQLEELVIRCYCCQKPQCPVEKQRHVDQGQRFHNIAEQWTGRCLQCWRPTAAETGSMIGPGPTLQDIVLELEPEPVDLRGYEQLDSSEDEDEIDHHQQQDRPDVQEDDGERFRILSDCHVCQNTVSRVVHSTHDDLHILEDLLMGTLEIVAPNCAVGSEQKLISEEDL

SEQ ID NO: 31 - DNA, 5211 bp, Macaca fascicularis
  misc_feature 1..5211: note = MfPV3 Ii-E1E6E7-p2a-Ii-E2
  source 1..5211: mol_type = other DNA
atgcacagaaggcggagcagaagctgccgcgaggatcagaaacccgtgatggacgaccagcgggacctgatcagcaacaacgagcagctgcctatgctcggcagaaggcctggcgctcctgagagcaaatgttctagaggcgccctgtacaccggcttcagcatcctggttacactgctgctggccggacaggctaccaccgcctactttctgtatcagcagcagggcagactggacaagctgaccgtgaccagccagaacctgcagctggaaaacctgcggatgaagctgcccaagcctcctaagcctgtgtccaagatgcggatggccacacctctgctgatgcaggctttgcctatgggagcactgcctcagggccctatgcagaacgccaccaaatacggcaacatgaccgaggaccacgtgatgcatctgctgcagaatgccgatcctctgaaggtgtaccctccactgaagggcagctttcccgagaacctgcggcacctgaagaacaccatggaaaccatcgactggaaggtgttcgagagctggatgcaccactggctgctgttcgagatgagccggcacagcctggaacagaagcctacagacgcccctcctaaagagtccctggaactggaagatcccagctctggcctgggcgtgacaaagcaagatctgggccctgtgcctatgatggacccttctggaattcacggcgagggcaccagatgcaccggatggttcgatgtggaagccatcgtggaaagacggaccggcgacgtggtgtccgaggacgaagatgagaacctggaagataccggcgtggacctggtggacttcatcgacgatacctgcgagtttgagcaggccggcgacgaagctcctagagcactgctgcatgcccaagagacacaggcccatgtgaacgccgtgcactacctgaagcggaagttcacatgcagccctaccgtgtctcccctgggcaatgtgcagtgttctatcgagggcgagctgagccctagactgcacgccatccaactgaatgaaggcagcggcaaggccaagcggagactgtttctgcctgatagcggctacggcaataccgaggtggaaacccagctgtctcaggtggcaggatccgatggcgaatctcaagcccctggcggcaaagaaaatgtgcagcctgctgaaggcgctggcggagatgttcgacagctgctgagaagcagcaacctgaaggccacactgctgggcaagttcaaggacgtgtacggcatcggcttcaacgaactcgtgcggtgcttcaagagcgacaagaccgtgtgcaccgattgggttgtgggagctgctggcgtgcaccattctgtggccgaaggactgaagcacctgatcgagcctttctgcacatacgcccacatgcagtgcctgacctgcaactggggagtgtttctgctgctgctcatccggttcaagtgcggcaagaacagactgaccgtggccaagtgcatctctaccctgctgaacgtgcaagagccctacctgctgatcgaacctccaaagctgagatccacagccgccgctctgtactggtacagaaccggcatcagcaacgtgtccgaagtgatcggagagcctcctgagtggatcgtgcggcagaccatgtttcagcacagcctcgaggacagcatcttcgacctgtctcagatggtgcagtgggcctacgaccacgatctggtggacgatagcgccattgcctaccagtatgcccagctggccgacgtggactctaatgccgccgctttcctgaagtctaacgctcaggccaaatacgtgaaggactgcgccaccatgtgcagacactacaagagagccgagaagcaggccatgaccatgagccagtggatcagagccagatgcgataagaccgacgacggcggagattggaggcctatcgtgcagttcctgagataccagaacgtcgagttcatcacctttctgagcgcctttaaggccttcctgaagggaatccccaagagaagctgcatcgtgctgtacggccctccaaacaccggcaagagctacttcggaatgagcctgatgcagtttctgcagggcagcatcatcagctacgtgaacagcaacagccacttttggctggcccctctggccgaagctaaagtggccatgctggacgatgccacaccacagtgctggtcctacatcgacacctacatgcggagcgtgctggacggcaaccccatgagcattgaccggaagcacaagaacctgatccagatgaagtgtccgcctctgatcatcaccagcaacaccaacgtgggcgaagatgacagatggcgctacctgcactccagagtggtggtgttcaagttcgagcagacctttccattcgaccagaacggcaatcccgtgtacgagctgtccgacaagaattggaagtccttcttcagccggacctggtccagactcgagctgcaagaggatgaggacaccgagcaggatggctctacagtgcgggcctttaagtgtgtgcccggccagaacaccaatatcctgggctctatggtggaaggcagccaagagagagcccggacactgcacgaactgtgcgagcagagagaacagacccagcacgacatcgagctggactgcgtgtactgcctgaatcctctgaccagaaccgaggtgtacgacttcgccagatgggagctgagaatcgtgtacagacgggataccgcctacggcgtgtgcaagctgtgcctgaggttccacagcaaagtgcggaagtacaggcggtacaactactccatctacggccccacactggaacggcggacaaagaagcagctcgaagaactcgtgatccggtgctactgctgccagaaacctcagtgccccgtggaaaagcagcgccacgttgaccagggccagagattccacaacattgccgagcagtggaccggcagatgcctgcaatgttggaggccaacagccgctgaaaccggctctatgatcggccctggacctacactgcaggatatcgtgcttgagctggaacccgagcctgtggacctgagaggatatgagcagctggactccagcgaggacgaggacgagattgaccaccaccaacagcaggacagacccgatgtgcaagaagatgacggcgagcggtttagaatcctgagcgactgtcacgtgtgccagaataccgtgtcaagagtggtgcacagcacccacgacgacctgcacattctcgaggacctgcttatgggcaccctcgagatcgtggcccctaattgtgccgtgggcagcggcgccaccaatttcagtctgcttaaacaggccggggacgtcgaagagaaccccggacctatgcatcggcgcagatctcggtcttgtagagaagaccaaaagcctgtcatggatgatcaaagagatttaatttctaataatgaacaattacccatgttgggacggcgccccggagcccccgaatctaagtgcagccggggagctttatatacaggattttctattttagtgaccttattattagctggccaagccacaacagcttatttcttataccaacaacaaggacggttagataaattaacagtcacatctcaaaatttacaattagagaatttaagaatgaaattacctaaaccacccaaacccgtcagtaaaatgagaatggctacccccttattaatgcaagccctccccatgggcgccttaccccaaggacccatgcaaaatgctacaaagtatggaaatatgacagaagatcatgtcatgcacttattacaaaacgctgaccccttaaaagtctatccccctttaaaaggatctttccctgaaaatttaagacatttaaaaaatacaatggagacaattgattggaaagtctttgaatcttggatgcatcattggttattatttgaaatgtctaggcattctttagagcaaaaacccaccgatgctccacccaaggaaagtttagagttagaggacccttctagcggattaggagtcaccaaacaggacttaggacccgtccccatgatggaagccctgagcgagagactgaacgcctaccaggacaagatcctgaccttctacgaagaggactccaaggacctgagggaccagatcgagcattggagatgtgtgcggggagagtgcgccgtgttctacaaggccagagagatgggccacagccacatcaaccaccagcaggttccacctctgcacgtgtccagaggcaaggcccacaatgccattgagctgcagatggccctggaaagcctgaaccagagcgacttcaacaccgaggaatggaccctgcaggacacaagcctggaaatgtggcggaccgagcctcagtgctgcttcaaaaagaaaggcgagacagtgaccgtgctgtatgactgcgacaaggacaacagcatggactacaccctgtggggccacgtgtacgtgtggggagatacaggctgggacaaagtgtgcggaaaggtggactacggcggcctgtactatgtgctgcacggcctgaaagtgtactacgttgagttccagcaagaggccgtgaagtacagcaagaccaacaagtgggaagtgcatatcggcagaacagtgatcccctacagcgagtccgtgtcctctacagccctgtgcgaaagagtgcctatcgccgagatcgccaatggcctccagcagaccagcattccctctcacacacacgccaccaccaaagagaacgcttgggtttcctctcagcctcctaccaagcggtccagaagatctgatccaggcggcgatcctgtcagagccctggatgccaatagcagaaccctgctgtctggaaccgcctgcgagaataccaccggcaactcttacagcgactgcacccctgtggtgcacctgaaaggcgaccccaattgtctgaaggccctgcggttcagattcgtgaaacacaagaatctgttcgtgaacgtgtcctccacctggcggtggaccaacaacaccgaagagaaggccatcgtgactgtgacctttaaggacgagcagcagagacagcagttcctcggcagcgtgaagatccccaactccatctgtgtgtgcaagggcgtgatgacagttggcagcgaacagaaactgatctccgaggaagatctctga

SEQ ID NO: 32 - protein, 1736 aa, Macaca fascicularis
  REGION 1..1736: note = MfPV3 Ii-E1E6E7-p2a-Ii-2 translated
MHRRRSRSCREDQKPVMDDQRDLISNNEQLPMLGRRPGAPESKCSRGALYTGFSILVTLLLAGQATTAYFLYQQQGRLDKLTVTSQNLQLENLRMKLPKPPKPVSKMRMATPLLMQALPMGALPQGPMQNATKYGNMTEDHVMHLLQNADPLKVYPPLKGSFPENLRHLKNTMETIDWKVFESWMHHWLLFEMSRHSLEQKPTDAPPKESLELEDPSSGLGVTKQDLGPVPMMDPSGIHGEGTRCTGWFDVEAIVERRTGDVVSEDEDENLEDTGVDLVDFIDDTCEFEQAGDEAPRALLHAQETQAHVNAVHYLKRKFTCSPTVSPLGNVQCSIEGELSPRLHAIQLNEGSGKAKRRLFLPDSGYGNTEVETQLSQVAGSDGESQAPGGKENVQPAEGAGGDVRQLLRSSNLKATLLGKFKDVYGIGFNELVRCFKSDKTVCTDWVVGAAGVHHSVAEGLKHLIEPFCTYAHMQCLTCNWGVFLLLLIRFKCGKNRLTVAKCISTLLNVQEPYLLIEPPKLRSTAAALYWYRTGISNVSEVIGEPPEWIVRQTMFQHSLEDSIFDLSQMVQWAYDHDLVDDSAIAYQYAQLADVDSNAAAFLKSNAQAKYVKDCATMCRHYKRAEKQAMTMSQWIRARCDKTDDGGDWRPIVQFLRYQNVEFITFLSAFKAFLKGIPKRSCIVLYGPPNTGKSYFGMSLMQFLQGSIISYVNSNSHFWLAPLAEAKVAMLDDATPQCWSYIDTYMRSVLDGNPMSIDRKHKNLIQMKCPPLIITSNTNVGEDDRWRYLHSRVVVFKFEQTFPFDQNGNPVYELSDKNWKSFFSRTWSRLELQEDEDTEQDGSTVRAFKCVPGQNTNILGSMVEGSQERARTLHELCEQREQTQHDIELDCVYCLNPLTRTEVYDFARWELRIVYRRDTAYGVCKLCLRFHSKVRKYRRYNYSIYGPTLERRTKKQLEELVIRCYCCQKPQCPVEKQRHVDQGQRFHNIAEQWTGRCLQCWRPTAAETGSMIGPGPTLQDIVLELEPEPVDLRGYEQLDSSEDEDEIDHHQQQDRPDVQEDDGERFRILSDCHVCQNTVSRVVHSTHDDLHILEDLLMGTLEIVAPNCAVGSGATNFSLLKQAGDVEENPGPMHRRRSRSCREDQKPVMDDQRDLISNNEQLPMLGRRPGAPESKCSRGALYTGFSILVTLLLAGQATTAYFLYQQQGRLDKLTVTSQNLQLENLRMKLPKPPKPVSKMRMATPLLMQALPMGALPQGPMQNATKYGNMTEDHVMHLLQNADPLKVYPPLKGSFPENLRHLKNTMETIDWKVFESWMHHWLLFEMSRHSLEQKPTDAPPKESLELEDPSSGLGVTKQDLGPVPMMEALSERLNAYQDKILTFYEEDSKDLRDQIEHWRCVRGECAVFYKAREMGHSHINHQQVPPLHVSRGKAHNAIELQMALESLNQSDFNTEEWTLQDTSLEMWRTEPQCCFKKKGETVTVLYDCDKDNSMDYTLWGHVYVWGDTGWDKVCGKVDYGGLYYVLHGLKVYYVEFQQEAVKYSKTNKWEVHIGRTVIPYSESVSSTALCERVPIAEIANGLQQTSIPSHTHATTKENAWVSSQPPTKRSRRSDPGGDPVRALDANSRTLLSGTACENTTGNSYSDCTPVVHLKGDPNCLKALRFRFVKHKNLFVNVSSTWRWTNNTEEKAIVTVTFKDEQQRQQFLGSVKIPNSICVCKGVMTVGSEQKLISEEDL

SEQ ID NO: 33 - DNA, 3369 bp, Macaca fascicularis
  misc_feature 1..3369: note = MfPV3 Ii-E1E6E7
  source 1..3369: mol_type = other DNA
atgcacagaaggcggagcagaagctgccgcgaggatcagaaacccgtgatggacgaccagcgggacctgatcagcaacaacgagcagctgcctatgctcggcagaaggcctggcgctcctgagagcaaatgttctagaggcgccctgtacaccggcttcagcatcctggttacactgctgctggccggacaggctaccaccgcctactttctgtatcagcagcagggcagactggacaagctgaccgtgaccagccagaacctgcagctggaaaacctgcggatgaagctgcccaagcctcctaagcctgtgtccaagatgcggatggccacacctctgctgatgcaggctttgcctatgggagcactgcctcagggccctatgcagaacgccaccaaatacggcaacatgaccgaggaccacgtgatgcatctgctgcagaatgccgatcctctgaaggtgtaccctccactgaagggcagctttcccgagaacctgcggcacctgaagaacaccatggaaaccatcgactggaaggtgttcgagagctggatgcaccactggctgctgttcgagatgagccggcacagcctggaacagaagcctacagacgcccctcctaaagagtccctggaactggaagatcccagctctggcctgggcgtgacaaagcaagatctgggccctgtgcctatgatggacccttctggaattcacggcgagggcaccagatgcaccggatggttcgatgtggaagccatcgtggaaagacggaccggcgacgtggtgtccgaggacgaagatgagaacctggaagataccggcgtggacctggtggacttcatcgacgatacctgcgagtttgagcaggccggcgacgaagctcctagagcactgctgcatgcccaagagacacaggcccatgtgaacgccgtgcactacctgaagcggaagttcacatgcagccctaccgtgtctcccctgggcaatgtgcagtgttctatcgagggcgagctgagccctagactgcacgccatccaactgaatgaaggcagcggcaaggccaagcggagactgtttctgcctgatagcggctacggcaataccgaggtggaaacccagctgtctcaggtggcaggatccgatggcgaatctcaagcccctggcggcaaagaaaatgtgcagcctgctgaaggcgctggcggagatgttcgacagctgctgagaagcagcaacctgaaggccacactgctgggcaagttcaaggacgtgtacggcatcggcttcaacgaactcgtgcggtgcttcaagagcgacaagaccgtgtgcaccgattgggttgtgggagctgctggcgtgcaccattctgtggccgaaggactgaagcacctgatcgagcctttctgcacatacgcccacatgcagtgcctgacctgcaactggggagtgtttctgctgctgctcatccggttcaagtgcggcaagaacagactgaccgtggccaagtgcatctctaccctgctgaacgtgcaagagccctacctgctgatcgaacctccaaagctgagatccacagccgccgctctgtactggtacagaaccggcatcagcaacgtgtccgaagtgatcggagagcctcctgagtggatcgtgcggcagaccatgtttcagcacagcctcgaggacagcatcttcgacctgtctcagatggtgcagtgggcctacgaccacgatctggtggacgatagcgccattgcctaccagtatgcccagctggccgacgtggactctaatgccgccgctttcctgaagtctaacgctcaggccaaatacgtgaaggactgcgccaccatgtgcagacactacaagagagccgagaagcaggccatgaccatgagccagtggatcagagccagatgcgataagaccgacgacggcggagattggaggcctatcgtgcagttcctgagataccagaacgtcgagttcatcacctttctgagcgcctttaaggccttcctgaagggaatccccaagagaagctgcatcgtgctgtacggccctccaaacaccggcaagagctacttcggaatgagcctgatgcagtttctgcagggcagcatcatcagctacgtgaacagcaacagccacttttggctggcccctctggccgaagctaaagtggccatgctggacgatgccacaccacagtgctggtcctacatcgacacctacatgcggagcgtgctggacggcaaccccatgagcattgaccggaagcacaagaacctgatccagatgaagtgtccgcctctgatcatcaccagcaacaccaacgtgggcgaagatgacagatggcgctacctgcactccagagtggtggtgttcaagttcgagcagacctttccattcgaccagaacggcaatcccgtgtacgagctgtccgacaagaattggaagtccttcttcagccggacctggtccagactcgagctgcaagaggatgaggacaccgagcaggatggctctacagtgcgggcctttaagtgtgtgcccggccagaacaccaatatcctgggctctatggtggaaggcagccaagagagagcccggacactgcacgaactgtgcgagcagagagaacagacccagcacgacatcgagctggactgcgtgtactgcctgaatcctctgaccagaaccgaggtgtacgacttcgccagatgggagctgagaatcgtgtacagacgggataccgcctacggcgtgtgcaagctgtgcctgaggttccacagcaaagtgcggaagtacaggcggtacaactactccatctacggccccacactggaacggcggacaaagaagcagctcgaagaactcgtgatccggtgctactgctgccagaaacctcagtgccccgtggaaaagcagcgccacgttgaccagggccagagattccacaacattgccgagcagtggaccggcagatgcctgcaatgttggaggccaacagccgctgaaaccggctctatgatcggccctggacctacactgcaggatatcgtgcttgagctggaacccgagcctgtggacctgagaggatatgagcagctggactccagcgaggacgaggacgagattgaccaccaccaacagcaggacagacccgatgtgcaagaagatgacggcgagcggtttagaatcctgagcgactgtcacgtgtgccagaataccgtgtcaagagtggtgcacagcacccacgacgacctgcacattctcgaggacctgcttatgggcaccctcgagatcgtggcccctaattgtgccgtgggcagcgaacagaaactgatctccgaggaagatctctga

SEQ ID NO: 34 - protein, 1122 aa, Macaca fascicularis
  REGION 1..1122: note = MfPV3 Ii-E1E6E7 translated
MHRRRSRSCREDQKPVMDDQRDLISNNEQLPMLGRRPGAPESKCSRGALYTGFSILVTLLLAGQATTAYFLYQQQGRLDKLTVTSQNLQLENLRMKLPKPPKPVSKMRMATPLLMQALPMGALPQGPMQNATKYGNMTEDHVMHLLQNADPLKVYPPLKGSFPENLRHLKNTMETIDWKVFESWMHHWLLFEMSRHSLEQKPTDAPPKESLELEDPSSGLGVTKQDLGPVPMMDPSGIHGEGTRCTGWFDVEAIVERRTGDVVSEDEDENLEDTGVDLVDFIDDTCEFEQAGDEAPRALLHAQETQAHVNAVHYLKRKFTCSPTVSPLGNVQCSIEGELSPRLHAIQLNEGSGKAKRRLFLPDSGYGNTEVETQLSQVAGSDGESQAPGGKENVQPAEGAGGDVRQLLRSSNLKATLLGKFKDVYGIGFNELVRCFKSDKTVCTDWVVGAAGVHHSVAEGLKHLIEPFCTYAHMQCLTCNWGVFLLLLIRFKCGKNRLTVAKCISTLLNVQEPYLLIEPPKLRSTAAALYWYRTGISNVSEVIGEPPEWIVRQTMFQHSLEDSIFDLSQMVQWAYDHDLVDDSAIAYQYAQLADVDSNAAAFLKSNAQAKYVKDCATMCRHYKRAEKQAMTMSQWIRARCDKTDDGGDWRPIVQFLRYQNVEFITFLSAFKAFLKGIPKRSCIVLYGPPNTGKSYFGMSLMQFLQGSIISYVNSNSHFWLAPLAEAKVAMLDDATPQCWSYIDTYMRSVLDGNPMSIDRKHKNLIQMKCPPLIITSNTNVGEDDRWRYLHSRVVVFKFEQTFPFDQNGNPVYELSDKNWKSFFSRTWSRLELQEDEDTEQDGSTVRAFKCVPGQNTNILGSMVEGSQERARTLHELCEQREQTQHDIELDCVYCLNPLTRTEVYDFARWELRIVYRRDTAYGVCKLCLRFHSKVRKYRRYNYSIYGPTLERRTKKQLEELVIRCYCCQKPQCPVEKQRHVDQGQRFHNIAEQWTGRCLQCWRPTAAETGSMIGPGPTLQDIVLELEPEPVDLRGYEQLDSSEDEDEIDHHQQQDRPDVQEDDGERFRILSDCHVCQNTVSRVVHSTHDDLHILEDLLMGTLEIVAPNCAVGSEQKLISEEDL

SEQ ID NO: 35 - DNA, 4515 bp, Human papillomavirus type 16
  misc_feature 1..4515: note = HPV16 Ii-E1E2E6E7
  source 1..4515: mol_type = other DNA
atgcacagaaggcggagcagaagctgccgcgaggatcagaaacccgtgatggacgaccagcgggacctgatcagcaacaacgagcagctgcctatgctcggcagaaggcctggcgctcctgagagcaaatgttctagaggcgccctgtacaccggcttcagcatcctggttacactgctgctggccggacaggctaccaccgcctactttctgtatcagcagcagggcagactggacaagctgaccgtgaccagccagaacctgcagctggaaaacctgcggatgaagctgcccaagcctcctaagcctgtgtccaagatgcggatggccacacctctgctgatgcaggctttgcctatgggagcactgcctcagggccctatgcagaacgccaccaaatacggcaacatgaccgaggaccacgtgatgcatctgctgcagaatgccgatcctctgaaggtgtaccctccactgaagggcagctttcccgagaacctgcggcacctgaagaacaccatggaaaccatcgactggaaggtgttcgagagctggatgcaccactggctgctgttcgagatgagccggcacagcctggaacagaagcctacagacgcccctcctaaagagtccctggaactggaagatcccagctctggcctgggcgtgacaaagcaagatctgggccctgtgcctatgatggccgatcctgctggcacaaacggcgaagaaggcacaggctgcaacggctggttttacgtggaagccgtggtggaaaagaaaaccggcgacgccatcagcgacgacgagaacgagaatgacagcgacaccggcgaggacctggtggacttcatcgtgaacgacaacgactacctgacacaggccgagacagagacagcccacgctctgtttacagcccaagaggccaagcagcacagagatgccgttcaggtgctgaagagaaagtacctgggcagccctctgagcgacatcagcggctgtgtggacaacaacatcagccccagactgaaggccatctgcatcgagaagcagagcagagccgccaagcggagactgtttgagagcgaggatagcggctacggcaataccgaggtggaaacccagcaaatgctgcaggtcgagggcagacacgaaaccgagacaccttgcagccagtacagcggaggatctggcggaggctgtagccagtactcttctggaagtggcggagagggcgtgtccgagagacacaccatttgtcagacccctctgaccaacatcctgaacgtgctgaaaaccagcaacgccaaggccgccatgctggccaagttcaaagagctgtacggcgtcagcttcagcgaactcgtgcggcccttcaagagcaacaagtccacctgttgcgactggtgcattgccgcctttggcctgacaccttctatcgccgacagcatcaagaccctgctccagcagtactgtctgtacctgcacattcagagcctggcctgctcttggggaatggtggtgctgctgctcgtgcggtacaagtgcggcaagaacagagagacaattgagaagctgctgagcaaactgctgtgcgtgtcccctatgtgcatgatgatcgagcctccaaagctgcggtctacagccgccgctctgtactggtacaagaccggcatcagcaatatctccgaggtgtacggcgacacccctgagtggattcagagacagaccgtcctgcagcacagcttcaacgactgcaccttcgagctgtcccagatggtgcagtgggcctacgacaatgacatcgtggacgactccgagatcgcctataagtacgcccagctggccgacaccaacagcaatgccagcgccttcctgaagtccaacagccaggccaagatcgtgaaggactgcgccaccatgtgcagacactacaagcgggccgagaagaaacagatgagcatgagccagtggatcaagtacagatgcgacagagtggacgacggcggcgattggaagcagatcgtgatgttcctgagataccagggcgtcgagttcatgagctttctgacagccctgaagcggttcctgcagggcatccccaagaagaactgcatcctgctgtatggcgccgctaacaccggcaagagcctgttcggaatgagcctgatgaagtttctgcagggctccgtgatctgctttgtgaacagcaagagccacttctggctccagccactggccgatgccaagattggcatgctggacgatgccaccgtgccttgctggaactacatcgacgacaacctgagaaacgccctggacggcaacctggtttctatggacgtgaagcacagacccctggtgcagctgaagtgtcctccactgctgatcaccagcaacatcaacgccggcaccgattccagatggccctatctgcacaaccggctggtggtgtttacctttcctaacgagttccccttcgacgaaaacggcaaccccgtgtacgagctgaacgataagaattggaagtccttcttcagccggacctggtccagactgagcctgcacgaggatgaggacaaagagaacgacggcgacagcctgcctaccttcaaatgtgtcagcggccagaacaccaacacactgggcagcatggaaacactgtgccagcggctgaacgtgtgccaggacaagattctgacccactatgagaacgacagcaccgacctgagggaccacatcgactactggaagcacatgcggctggaatgcgccatctactacaaggctagagagatgggcttcaagcacatcaaccaccaggtggtgcctacactggccgtgtctaagaacaaggctctgcaggccatcgaactgcagctgaccctggaaacaatctacaactcccagtactccaacgagaagtggaccctccaggacgtgtcactggaagtgtacctgaccgctcctaccggctgcatcaagaaacacggctacaccgtggaagtgcagtttgacggcgatatctgcaataccatgcactacaccaactggacccacatctacatctgcgaagaggccagcgtgacagtggtggaaggccaggtcgactactacggcctgtactacgtgcacgagggcatcagaacctacttcgtgcagttcaaggatgacgccgagaagtacagcaagaacaaagtgtgggaagtgcacgctggcggccaagtgatcctgtgtcctacaagcgtgttcagcagcaacgaagtgtctagccccgagatcatcagacagcacctggccaatcatcctgccgccacacacacaaaagctgtggccctgggcaccgaggaaacacagaccacaattcagaggcccagaagcgagcccgataccggcaatccttgtcacaccaccaagctgctgcaccgggattccgtggattctgcccctatcctgaccgccttcaacagctcccacaaaggccggatcaactgcaacagcaacaccacacctatcgtgcacctgaaaggggacgccaacactctgaaggccctgcggtacagattcaagaagcactgtaccctgtataccgccgtgtccagcacatggcactggaccggacacaacgtgaaacacaagagcgccatcgtgaccctgacctacgatagcgagtggcagagggaccagttcctgagccaagtgaagatccctaagaccatcaccgtgtccaccggctttatgagcattggcagcatgcaccagaaacggacagccatgttccaggatcctcaagagcggccacggaagctgcctcagctgtgtacagaactgcagaccaccatccacgacatcatcctcgagtgcgtgtactgcaagcagcagctcctgcggagagaagtgtacgacttcgccttccgggatctgtgcatcgtgtacagagatggcaacccctacgccgtgtgcgacaagtgcctgaagttctactccaagatcagcgagtaccggcactactgctactccctgtacggcaccacacttgagcagcagtacaacaagcccctgtgcgatctgctgatccggtgcatcaactgccagaaacctcagtgccccgaggaaaagcagagacacctggacaagaagcagcggttccacaacattagaggcagatggacaggccggtgcatgagctgctgtagaagcagcagaacaagacggggctccatgcacggcgataccccaacactgcacgagtacatgctggatctgcagcccgagacaaccgatctgtacggctatgagcagctgaatgactccagcgaggaagaggacgagatcgacggacctgctggacaggctgaacctgatagagcccactacaatatcgtgaccttctgctgcaagtgcgatagcaccctgagaagatgcgtgcagagcacccacgtggacatcaggaccctcgaggatctgctcatgggcaccctgggaattgtggccccaatctgctctcagaagccttga

SEQ ID NO: 36 - protein, 1504 aa, Human papillomavirus type 16
  REGION 1..1504: note = HPV16 Ii-E1E2E6E7 translated
MHRRRSRSCREDQKPVMDDQRDLISNNEQLPMLGRRPGAPESKCSRGALYTGFSILVTLLLAGQATTAYFLYQQQGRLDKLTVTSQNLQLENLRMKLPKPPKPVSKMRMATPLLMQALPMGALPQGPMQNATKYGNMTEDHVMHLLQNADPLKVYPPLKGSFPENLRHLKNTMETIDWKVFESWMHHWLLFEMSRHSLEQKPTDAPPKESLELEDPSSGLGVTKQDLGPVPMMADPAGTNGEEGTGCNGWFYVEAVVEKKTGDAISDDENENDSDTGEDLVDFIVNDNDYLTQAETETAHALFTAQEAKQHRDAVQVLKRKYLGSPLSDISGCVDNNISPRLKAICIEKQSRAAKRRLFESEDSGYGNTEVETQQMLQVEGRHETETPCSQYSGGSGGGCSQYSSGSGGEGVSERHTICQTPLTNILNVLKTSNAKAAMLAKFKELYGVSFSELVRPFKSNKSTCCDWCIAAFGLTPSIADSIKTLLQQYCLYLHIQSLACSWGMVVLLLVRYKCGKNRETIEKLLSKLLCVSPMCMMIEPPKLRSTAAALYWYKTGISNISEVYGDTPEWIQRQTVLQHSFNDCTFELSQMVQWAYDNDIVDDSEIAYKYAQLADTNSNASAFLKSNSQAKIVKDCATMCRHYKRAEKKQMSMSQWIKYRCDRVDDGGDWKQIVMFLRYQGVEFMSFLTALKRFLQGIPKKNCILLYGAANTGKSLFGMSLMKFLQGSVICFVNSKSHFWLQPLADAKIGMLDDATVPCWNYIDDNLRNALDGNLVSMDVKHRPLVQLKCPPLLITSNINAGTDSRWPYLHNRLVVFTFPNEFPFDENGNPVYELNDKNWKSFFSRTWSRLSLHEDEDKENDGDSLPTFKCVSGQNTNTLGSMETLCQRLNVCQDKILTHYENDSTDLRDHIDYWKHMRLECAIYYKAREMGFKHINHQVVPTLAVSKNKALQAIELQLTLETIYNSQYSNEKWTLQDVSLEVYLTAPTGCIKKHGYTVEVQFDGDICNTMHYTNWTHIYICEEASVTVVEGQVDYYGLYYVHEGIRTYFVQFKDDAEKYSKNKVWEVHAGGQVILCPTSVFSSNEVSSPEIIRQHLANHPAATHTKAVALGTEETQTTIQRPRSEPDTGNPCHTTKLLHRDSVDSAPILTAFNSSHKGRINCNSNTTPIVHLKGDANTLKALRYRFKKHCTLYTAVSSTWHWTGHNVKHKSAIVTLTYDSEWQRDQFLSQVKIPKTITVSTGFMSIGSMHQKRTAMFQDPQERPRKLPQLCTELQTTIHDIILECVYCKQQLLRREVYDFAFRDLCIVYRDGNPYAVCDKCLKFYSKISEYRHYCYSLYGTTLEQQYNKPLCDLLIRCINCQKPQCPEEKQRHLDKKQRFHNIRGRWTGRCMSCCRSSRTRRGSMHGDTPTLHEYMLDLQPETTDLYGYEQLNDSSEEEDEIDGPAGQAEPDRAHYNIVTFCCKCDSTLRRCVQSTHVDIRTLEDLLMGTLGIVAPICSQKP

SEQ ID NO: 37 - DNA, 5277 bp, Human papillomavirus type 16
  misc_feature 1..5277: note = HPV16 Ii-E1E2-p2a-Ii-E6E7
  source 1..5277: mol_type = other DNA
atgcacagaaggcggagcagaagctgccgcgaggatcagaaacccgtgatggacgaccagcgggacctgatcagcaacaacgagcagctgcctatgctcggcagaaggcctggcgctcctgagagcaaatgttctagaggcgccctgtacaccggcttcagcatcctggttacactgctgctggccggacaggctaccaccgcctactttctgtatcagcagcagggcagactggacaagctgaccgtgaccagccagaacctgcagctggaaaacctgcggatgaagctgcccaagcctcctaagcctgtgtccaagatgcggatggccacacctctgctgatgcaggctttgcctatgggagcactgcctcagggccctatgcagaacgccaccaaatacggcaacatgaccgaggaccacgtgatgcatctgctgcagaatgccgatcctctgaaggtgtaccctccactgaagggcagctttcccgagaacctgcggcacctgaagaacaccatggaaaccatcgactggaaggtgttcgagagctggatgcaccactggctgctgttcgagatgagccggcacagcctggaacagaagcctacagacgcccctcctaaagagtccctggaactggaagatcccagctctggcctgggcgtgacaaagcaagatctgggccctgtgcctatgatggccgatcctgctggcacaaacggcgaagaaggcacaggctgcaacggctggttttacgtggaagccgtggtggaaaagaaaaccggcgacgccatcagcgacgacgagaacgagaatgacagcgacaccggcgaggacctggtggacttcatcgtgaacgacaacgactacctgacacaggccgagacagagacagcccacgctctgtttacagcccaagaggccaagcagcacagagatgccgttcaggtgctgaagagaaagtacctgggcagccctctgagcgacatcagcggctgtgtggacaacaacatcagccccagactgaaggccatctgcatcgagaagcagagcagagccgccaagcggagactgtttgagagcgaggatagcggctacggcaataccgaggtggaaacccagcaaatgctgcaggtcgagggcagacacgaaaccgagacaccttgcagccagtacagcggaggatctggcggaggctgtagccagtactcttctggaagtggcggagagggcgtgtccgagagacacaccatttgtcagacccctctgaccaacatcctgaacgtgctgaaaaccagcaacgccaaggccgccatgctggccaagttcaaagagctgtacggcgtcagcttcagcgaactcgtgcggcccttcaagagcaacaagtccacctgttgcgactggtgcattgccgcctttggcctgacaccttctatcgccgacagcatcaagaccctgctccagcagtactgtctgtacctgcacattcagagcctggcctgctcttggggaatggtggtgctgctgctcgtgcggtacaagtgcggcaagaacagagagacaattgagaagctgctgagcaaactgctgtgcgtgtcccctatgtgcatgatgatcgagcctccaaagctgcggtctacagccgccgctctgtactggtacaagaccggcatcagcaatatctccgaggtgtacggcgacacccctgagtggattcagagacagaccgtcctgcagcacagcttcaacgactgcaccttcgagctgtcccagatggtgcagtgggcctacgacaatgacatcgtggacgactccgagatcgcctataagtacgcccagctggccgacaccaacagcaatgccagcgccttcctgaagtccaacagccaggccaagatcgtgaaggactgcgccaccatgtgcagacactacaagcgggccgagaagaaacagatgagcatgagccagtggatcaagtacagatgcgacagagtggacgacggcggcgattggaagcagatcgtgatgttcctgagataccagggcgtcgagttcatgagctttctgacagccctgaagcggttcctgcagggcatccccaagaagaactgcatcctgctgtatggcgccgctaacaccggcaagagcctgttcggaatgagcctgatgaagtttctgcagggctccgtgatctgctttgtgaacagcaagagccacttctggctccagccactggccgatgccaagattggcatgctggacgatgccaccgtgccttgctggaactacatcgacgacaacctgagaaacgccctggacggcaacctggtttctatggacgtgaagcacagacccctggtgcagctgaagtgtcctccactgctgatcaccagcaacatcaacgccggcaccgattccagatggccctatctgcacaaccggctggtggtgtttacctttcctaacgagttccccttcgacgaaaacggcaaccccgtgtacgagctgaacgataagaattggaagtccttcttcagccggacctggtccagactgagcctgcacgaggatgaggacaaagagaacgacggcgacagcctgcctaccttcaaatgtgtcagcggccagaacaccaacacactgggcagcatggaaacactgtgccagcggctgaacgtgtgccaggacaagattctgacccactatgagaacgacagcaccgacctgagggaccacatcgactactggaagcacatgcggctggaatgcgccatctactacaaggctagagagatgggcttcaagcacatcaaccaccaggtggtgcctacactggccgtgtctaagaacaaggctctgcaggccatcgaactgcagctgaccctggaaacaatctacaactcccagtactccaacgagaagtggaccctccaggacgtgtcactggaagtgtacctgaccgctcctaccggctgcatcaagaaacacggctacaccgtggaagtgcagtttgacggcgatatctgcaataccatgcactacaccaactggacccacatctacatctgcgaagaggccagcgtgacagtggtggaaggccaggtcgactactacggcctgtactacgtgcacgagggcatcagaacctacttcgtgcagttcaaggatgacgccgagaagtacagcaagaacaaagtgtgggaagtgcacgctggcggccaagtgatcctgtgtcctacaagcgtgttcagcagcaacgaagtgtctagccccgagatcatcagacagcacctggccaatcatcctgccgccacacacacaaaagctgtggccctgggcaccgaggaaacacagaccacaattcagaggcccagaagcgagcccgataccggcaatccttgtcacaccaccaagctgctgcaccgggattccgtggattctgcccctatcctgaccgccttcaacagctcccacaaaggccggatcaactgcaacagcaacaccacacctatcgtgcacctgaaaggggacgccaacactctgaaggccctgcggtacagattcaagaagcactgtaccctgtataccgccgtgtccagcacatggcactggaccggacacaacgtgaaacacaagagcgccatcgtgaccctgacctacgatagcgagtggcagagggaccagttcctgagccaagtgaagatccctaagaccatcaccgtgtccaccggctttatgagcattggcagcggcagcggcgccaccaatttcagtctgcttaaacaggccggggacgtcgaagagaaccccggacctatgcatcggcgcagatctcggtcttgtagagaagaccaaaagcctgtcatggatgatcaaagagatttaatttctaataatgaacaattacccatgttgggacggcgccccggagcccccgaatctaagtgcagccggggagctttatatacaggattttctattttagtgaccttattattagctggccaagccacaacagcttatttcttataccaacaacaaggacggttagataaattaacagtcacatctcaaaatttacaattagagaatttaagaatgaaattacctaaaccacccaaacccgtcagtaaaatgagaatggctacccccttattaatgcaagccctccccatgggcgccttaccccaaggacccatgcaaaatgctacaaagtatggaaatatgacagaagatcatgtcatgcacttattacaaaacgctgaccccttaaaagtctatccccctttaaaaggatctttccctgaaaatttaagacatttaaaaaatacaatggagacaattgattggaaagtctttgaatcttggatgcatcattggttattatttgaaatgtctaggcattctttagagcaaaaacccaccgatgctccacccaaggaaagtttagagttagaggacccttctagcggattaggagtcaccaaacaggacttaggacccgtccccatgatgcaccagaaacggacagccatgttccaggatcctcaagagcggccacggaagctgcctcagctgtgtacagaactgcagaccaccatccacgacatcatcctcgagtgcgtgtactgcaagcagcagctcctgcggagagaagtgtacgacttcgccttccgggatctgtgcatcgtgtacagagatggcaacccctacgccgtgtgcgacaagtgcctgaagttctactccaagatcagcgagtaccggcactactgctactccctgtacggcaccacacttgagcagcagtacaacaagcccctgtgcgatctgctgatccggtgcatcaactgccagaaacctcagtgccccgaggaaaagcagagacacctggacaagaagcagcggttccacaacattagaggcagatggacaggccggtgcatgagctgctgtagaagcagcagaacaagacggggctccatgcacggcgataccccaacactgcacgagtacatgctggatctgcagcccgagacaaccgatctgtacggctatgagcagctgaatgactccagcgaggaagaggacgagatcgacggacctgctggacaggctgaacctgatagagcccactacaatatcgtgaccttctgctgcaagtgcgatagcaccctgagaagatgcgtgcagagcacccacgtggacatcaggaccctcgaggatctgctcatgggcaccctgggaattgtggccccaatctgctctcagaagccttga

SEQ ID NO: 38 - protein, 1758 aa, Human papillomavirus type 16
  REGION 1..1758: note = HPV16 Ii-E1E2-p2a-Ii-E6E7 translated
MHRRRSRSCREDQKPVMDDQRDLISNNEQLPMLGRRPGAPESKCSRGALYTGFSILVTLLLAGQATTAYFLYQQQGRLDKLTVTSQNLQLENLRMKLPKPPKPVSKMRMATPLLMQALPMGALPQGPMQNATKYGNMTEDHVMHLLQNADPLKVYPPLKGSFPENLRHLKNTMETIDWKVFESWMHHWLLFEMSRHSLEQKPTDAPPKESLELEDPSSGLGVTKQDLGPVPMMADPAGTNGEEGTGCNGWFYVEAVVEKKTGDAISDDENENDSDTGEDLVDFIVNDNDYLTQAETETAHALFTAQEAKQHRDAVQVLKRKYLGSPLSDISGCVDNNISPRLKAICIEKQSRAAKRRLFESEDSGYGNTEVETQQMLQVEGRHETETPCSQYSGGSGGGCSQYSSGSGGEGVSERHTICQTPLTNILNVLKTSNAKAAMLAKFKELYGVSFSELVRPFKSNKSTCCDWCIAAFGLTPSIADSIKTLLQQYCLYLHIQSLACSWGMVVLLLVRYKCGKNRETIEKLLSKLLCVSPMCMMIEPPKLRSTAAALYWYKTGISNISEVYGDTPEWIQRQTVLQHSFNDCTFELSQMVQWAYDNDIVDDSEIAYKYAQLADTNSNASAFLKSNSQAKIVKDCATMCRHYKRAEKKQMSMSQWIKYRCDRVDDGGDWKQIVMFLRYQGVEFMSFLTALKRFLQGIPKKNCILLYGAANTGKSLFGMSLMKFLQGSVICFVNSKSHFWLQPLADAKIGMLDDATVPCWNYIDDNLRNALDGNLVSMDVKHRPLVQLKCPPLLITSNINAGTDSRWPYLHNRLVVFTFPNEFPFDENGNPVYELNDKNWKSFFSRTWSRLSLHEDEDKENDGDSLPTFKCVSGQNTNTLGSMETLCQRLNVCQDKILTHYENDSTDLRDHIDYWKHMRLECAIYYKAREMGFKHINHQVVPTLAVSKNKALQAIELQLTLETIYNSQYSNEKWTLQDVSLEVYLTAPTGCIKKHGYTVEVQFDGDICNTMHYTNWTHIYICEEASVTVVEGQVDYYGLYYVHEGIRTYFVQFKDDAEKYSKNKVWEVHAGGQVILCPTSVFSSNEVSSPEIIRQHLANHPAATHTKAVALGTEETQTTIQRPRSEPDTGNPCHTTKLLHRDSVDSAPILTAFNSSHKGRINCNSNTTPIVHLKGDANTLKALRYRFKKHCTLYTAVSSTWHWTGHNVKHKSAIVTLTYDSEWQRDQFLSQVKIPKTITVSTGFMSIGSGSGATNFSLLKQAGDVEENPGPMHRRRSRSCREDQKPVMDDQRDLISNNEQLPMLGRRPGAPESKCSRGALYTGFSILVTLLLAGQATTAYFLYQQQGRLDKLTVTSQNLQLENLRMKLPKPPKPVSKMRMATPLLMQALPMGALPQGPMQNATKYGNMTEDHVMHLLQNADPLKVYPPLKGSFPENLRHLKNTMETIDWKVFESWMHHWLLFEMSRHSLEQKPTDAPPKESLELEDPSSGLGVTKQDLGPVPMMHQKRTAMFQDPQERPRKLPQLCTELQTTIHDIILECVYCKQQLLRREVYDFAFRDLCIVYRDGNPYAVCDKCLKFYSKISEYRHYCYSLYGTTLEQQYNKPLCDLLIRCINCQKPQCPEEKQRHLDKKQRFHNIRGRWTGRCMSCCRSSRTRRGSMHGDTPTLHEYMLDLQPETTDLYGYEQLNDSSEEEDEIDGPAGQAEPDRAHYNIVTFCCKCDSTLRRCVQSTHVDIRTLEDLLMGTLGIVAPICSQKP

SEQ ID NO: 39 - DNA, 3819 bp, Human papillomavirus type 16
  misc_feature 1..3819: note = HPV16 E1E2E6E7
  source 1..3819: mol_type = other DNA
atggccgatcctgctggcacaaacggcgaagaaggcacaggctgcaacggctggttttacgtggaagccgtggtggaaaagaaaaccggcgacgccatcagcgacgacgagaacgagaatgacagcgacaccggcgaggacctggtggacttcatcgtgaacgacaacgactacctgacacaggccgagacagagacagcccacgctctgtttacagcccaagaggccaagcagcacagagatgccgttcaggtgctgaagagaaagtacctgggcagccctctgagcgacatcagcggctgtgtggacaacaacatcagccccagactgaaggccatctgcatcgagaagcagagcagagccgccaagcggagactgtttgagagcgaggatagcggctacggcaataccgaggtggaaacccagcaaatgctgcaggtcgagggcagacacgaaaccgagacaccttgcagccagtacagcggaggatctggcggaggctgtagccagtactcttctggaagtggcggagagggcgtgtccgagagacacaccatttgtcagacccctctgaccaacatcctgaacgtgctgaaaaccagcaacgccaaggccgccatgctggccaagttcaaagagctgtacggcgtcagcttcagcgaactcgtgcggcccttcaagagcaacaagtccacctgttgcgactggtgcattgccgcctttggcctgacaccttctatcgccgacagcatcaagaccctgctccagcagtactgtctgtacctgcacattcagagcctggcctgctcttggggaatggtggtgctgctgctcgtgcggtacaagtgcggcaagaacagagagacaattgagaagctgctgagcaaactgctgtgcgtgtcccctatgtgcatgatgatcgagcctccaaagctgcggtctacagccgccgctctgtactggtacaagaccggcatcagcaatatctccgaggtgtacggcgacacccctgagtggattcagagacagaccgtcctgcagcacagcttcaacgactgcaccttcgagctgtcccagatggtgcagtgggcctacgacaatgacatcgtggacgactccgagatcgcctataagtacgcccagctggccgacaccaacagcaatgccagcgccttcctgaagtccaacagccaggccaagatcgtgaaggactgcgccaccatgtgcagacactacaagcgggccgagaagaaacagatgagcatgagccagtggatcaagtacagatgcgacagagtggacgacggcggcgattggaagcagatcgtgatgttcctgagataccagggcgtcgagttcatgagctttctgacagccctgaagcggttcctgcagggcatccccaagaagaactgcatcctgctgtatggcgccgctaacaccggcaagagcctgttcggaatgagcctgatgaagtttctgcagggctccgtgatctgctttgtgaacagcaagagccacttctggctccagccactggccgatgccaagattggcatgctggacgatgccaccgtgccttgctggaactacatcgacgacaacctgagaaacgccctggacggcaacctggtttctatggacgtgaagcacagacccctggtgcagctgaagtgtcctccactgctgatcaccagcaacatcaacgccggcaccgattccagatggccctatctgcacaaccggctggtggtgtttacctttcctaacgagttccccttcgacgaaaacggcaaccccgtgtacgagctgaacgataagaattggaagtccttcttcagccggacctggtccagactgagcctgcacgaggatgaggacaaagagaacgacggcgacagcctgcctaccttcaaatgtgtcagcggccagaacaccaacacactgggcagcatggaaacactgtgccagcggctgaacgtgtgccaggacaagattctgacccactatgagaacgacagcaccgacctgagggaccacatcgactactggaagcacatgcggctggaatgcgccatctactacaaggctagagagatgggcttcaagcacatcaaccaccaggtggtgcctacactggccgtgtctaagaacaaggctctgcaggccatcgaactgcagctgaccctggaaacaatctacaactcccagtactccaacgagaagtggaccctccaggacgtgtcactggaagtgtacctgaccgctcctaccggctgcatcaagaaacacggctacaccgtggaagtgcagtttgacggcgatatctgcaataccatgcactacaccaactggacccacatctacatctgcgaagaggccagcgtgacagtggtggaaggccaggtcgactactacggcctgtactacgtgcacgagggcatcagaacctacttcgtgcagttcaaggatgacgccgagaagtacagcaagaacaaagtgtgggaagtgcacgctggcggccaagtgatcctgtgtcctacaagcgtgttcagcagcaacgaagtgtctagccccgagatcatcagacagcacctggccaatcatcctgccgccacacacacaaaagctgtggccctgggcaccgaggaaacacagaccacaattcagaggcccagaagcgagcccgataccggcaatccttgtcacaccaccaagctgctgcaccgggattccgtggattctgcccctatcctgaccgccttcaacagctcccacaaaggccggatcaactgcaacagcaacaccacacctatcgtgcacctgaaaggggacgccaacactctgaaggccctgcggtacagattcaagaagcactgtaccctgtataccgccgtgtccagcacatggcactggaccggacacaacgtgaaacacaagagcgccatcgtgaccctgacctacgatagcgagtggcagagggaccagttcctgagccaagtgaagatccctaagaccatcaccgtgtccaccggctttatgagcattggcagcatgcaccagaaacggacagccatgttccaggatcctcaagagcggccacggaagctgcctcagctgtgtacagaactgcagaccaccatccacgacatcatcctcgagtgcgtgtactgcaagcagcagctcctgcggagagaagtgtacgacttcgccttccgggatctgtgcatcgtgtacagagatggcaacccctacgccgtgtgcgacaagtgcctgaagttctactccaagatcagcgagtaccggcactactgctactccctgtacggcaccacacttgagcagcagtacaacaagcccctgtgcgatctgctgatccggtgcatcaactgccagaaacctcagtgccccgaggaaaagcagagacacctggacaagaagcagcggttccacaacattagaggcagatggacaggccggtgcatgagctgctgtagaagcagcagaacaagacggggctccatgcacggcgataccccaacactgcacgagtacatgctggatctgcagcccgagacaaccgatctgtacggctatgagcagctgaatgactccagcgaggaagaggacgagatcgacggacctgctggacaggctgaacctgatagagcccactacaatatcgtgaccttctgctgcaagtgcgatagcaccctgagaagatgcgtgcagagcacccacgtggacatcaggaccctcgaggatctgctcatgggcaccctgggaattgtggccccaatctgctctcagaagccttga

SEQ ID NO: 40 - protein, 1272 aa, Human papillomavirus type 16
  REGION 1..1272: note = HPV16 E1E2E6E7 translated
MADPAGTNGEEGTGCNGWFYVEAVVEKKTGDAISDDENENDSDTGEDLVDFIVNDNDYLTQAETETAHALFTAQEAKQHRDAVQVLKRKYLGSPLSDISGCVDNNISPRLKAICIEKQSRAAKRRLFESEDSGYGNTEVETQQMLQVEGRHETETPCSQYSGGSGGGCSQYSSGSGGEGVSERHTICQTPLTNILNVLKTSNAKAAMLAKFKELYGVSFSELVRPFKSNKSTCCDWCIAAFGLTPSIADSIKTLLQQYCLYLHIQSLACSWGMVVLLLVRYKCGKNRETIEKLLSKLLCVSPMCMMIEPPKLRSTAAALYWYKTGISNISEVYGDTPEWIQRQTVLQHSFNDCTFELSQMVQWAYDNDIVDDSEIAYKYAQLADTNSNASAFLKSNSQAKIVKDCATMCRHYKRAEKKQMSMSQWIKYRCDRVDDGGDWKQIVMFLRYQGVEFMSFLTALKRFLQGIPKKNCILLYGAANTGKSLFGMSLMKFLQGSVICFVNSKSHFWLQPLADAKIGMLDDATVPCWNYIDDNLRNALDGNLVSMDVKHRPLVQLKCPPLLITSNINAGTDSRWPYLHNRLVVFTFPNEFPFDENGNPVYELNDKNWKSFFSRTWSRLSLHEDEDKENDGDSLPTFKCVSGQNTNTLGSMETLCQRLNVCQDKILTHYENDSTDLRDHIDYWKHMRLECAIYYKAREMGFKHINHQVVPTLAVSKNKALQAIELQLTLETIYNSQYSNEKWTLQDVSLEVYLTAPTGCIKKHGYTVEVQFDGDICNTMHYTNWTHIYICEEASVTVVEGQVDYYGLYYVHEGIRTYFVQFKDDAEKYSKNKVWEVHAGGQVILCPTSVFSSNEVSSPEIIRQHLANHPAATHTKAVALGTEETQTTIQRPRSEPDTGNPCHTTKLLHRDSVDSAPILTAFNSSHKGRINCNSNTTPIVHLKGDANTLKALRYRFKKHCTLYTAVSSTWHWTGHNVKHKSAIVTLTYDSEWQRDQFLSQVKIPKTITVSTGFMSIGSMHQKRTAMFQDPQERPRKLPQLCTELQTTIHDIILECVYCKQQLLRREVYDFAFRDLCIVYRDGNPYAVCDKCLKFYSKISEYRHYCYSLYGTTLEQQYNKPLCDLLIRCINCQKPQCPEEKQRHLDKKQRFHNIRGRWTGRCMSCCRSSRTRRGSMHGDTPTLHEYMLDLQPETTDLYGYEQLNDSSEEEDEIDGPAGQAEPDRAHYNIVTFCCKCDSTLRRCVQSTHVDIRTLEDLLMGTLGIVAPICSQKP

SEQ ID NO: 41 - DNA, 5271 bp, Human papillomavirus type 16
  misc_feature 1..5271: note = HPV16 Ii-E1E6E7-p2a-Ii-E2
  source 1..5271: mol_type = other DNA
atgcacagaaggcggagcagaagctgccgcgaggatcagaaacccgtgatggacgaccagcgggacctgatcagcaacaacgagcagctgcctatgctcggcagaaggcctggcgctcctgagagcaaatgttctagaggcgccctgtacaccggcttcagcatcctggttacactgctgctggccggacaggctaccaccgcctactttctgtatcagcagcagggcagactggacaagctgaccgtgaccagccagaacctgcagctggaaaacctgcggatgaagctgcccaagcctcctaagcctgtgtccaagatgcggatggccacacctctgctgatgcaggctttgcctatgggagcactgcctcagggccctatgcagaacgccaccaaatacggcaacatgaccgaggaccacgtgatgcatctgctgcagaatgccgatcctctgaaggtgtaccctccactgaagggcagctttcccgagaacctgcggcacctgaagaacaccatggaaaccatcgactggaaggtgttcgagagctggatgcaccactggctgctgttcgagatgagccggcacagcctggaacagaagcctacagacgcccctcctaaagagtccctggaactggaagatcccagctctggcctgggcgtgacaaagcaagatctgggccctgtgcctatgatggccgatcctgctggcacaaacggcgaagaaggcacaggctgcaacggctggttttacgtggaagccgtggtggaaaagaaaaccggcgacgccatcagcgacgacgagaacgagaatgacagcgacaccggcgaggacctggtggacttcatcgtgaacgacaacgactacctgacacaggccgagacagagacagcccacgctctgtttacagcccaagaggccaagcagcacagagatgccgttcaggtgctgaagagaaagtacctgggcagccctctgagcgacatcagcggctgtgtggacaacaacatcagccccagactgaaggccatctgcatcgagaagcagagcagagccgccaagcggagactgtttgagagcgaggatagcggctacggcaataccgaggtggaaacccagcaaatgctgcaggtcgagggcagacacgaaaccgagacaccttgcagccagtacagcggaggatctggcggaggctgtagccagtactcttctggaagtggcggagagggcgtgtccgagagacacaccatttgtcagacccctctgaccaacatcctgaacgtgctgaaaaccagcaacgccaaggccgccatgctggccaagttcaaagagctgtacggcgtcagcttcagcgaactcgtgcggcccttcaagagcaacaagtccacctgttgcgactggtgcattgccgcctttggcctgacaccttctatcgccgacagcatcaagaccctgctccagcagtactgtctgtacctgcacattcagagcctggcctgctcttggggaatggtggtgctgctgctcgtgcggtacaagtgcggcaagaacagagagacaattgagaagctgctgagcaaactgctgtgcgtgtcccctatgtgcatgatgatcgagcctccaaagctgcggtctacagccgccgctctgtactggtacaagaccggcatcagcaatatctccgaggtgtacggcgacacccctgagtggattcagagacagaccgtcctgcagcacagcttcaacgactgcaccttcgagctgtcccagatggtgcagtgggcctacgacaatgacatcgtggacgactccgagatcgcctataagtacgcccagctggccgacaccaacagcaatgccagcgccttcctgaagtccaacagccaggccaagatcgtgaaggactgcgccaccatgtgcagacactacaagcgggccgagaagaaacagatgagcatgagccagtggatcaagtacagatgcgacagagtggacgacggcggcgattggaagcagatcgtgatgttcctgagataccagggcgtcgagttcatgagctttctgacagccctgaagcggttcctgcagggcatccccaagaagaactgcatcctgctgtatggcgccgctaacaccggcaagagcctgttcggaatgagcctgatgaagtttctgcagggctccgtgatctgctttgtgaacagcaagagccacttctggctccagccactggccgatgccaagattggcatgctggacgatgccaccgtgccttgctggaactacatcgacgacaacctgagaaacgccctggacggcaacctggtttctatggacgtgaagcacagacccctggtgcagctgaagtgtcctccactgctgatcaccagcaacatcaacgccggcaccgattccagatggccctatctgcacaaccggctggtggtgtttacctttcctaacgagttccccttcgacgaaaacggcaaccccgtgtacgagctgaacgataagaattggaagtccttcttcagccggacctggtccagactgagcctgcacgaggatgaggacaaagagaacgacggcgacagcctgcctaccttcaaatgtgtcagcggccagaacaccaacacactgggcagcatgcaccagaaacggacagccatgttccaggatcctcaagagcggccacggaagctgcctcagctgtgtacagaactgcagaccaccatccacgacatcatcctcgagtgcgtgtactgcaagcagcagctcctgcggagagaagtgtacgacttcgccttccgggatctgtgcatcgtgtacagagatggcaacccctacgccgtgtgcgacaagtgcctgaagttctactccaagatcagcgagtaccggcactactgctactccctgtacggcaccacacttgagcagcagtacaacaagcccctgtgcgatctgctgatccggtgcatcaactgccagaaacctcagtgccccgaggaaaagcagagacacctggacaagaagcagcggttccacaacattagaggcagatggacaggccggtgcatgagctgctgtagaagcagcagaacaagacggggctccatgcacggcgataccccaacactgcacgagtacatgctggatctgcagcccgagacaaccgatctgtacggctatgagcagctgaatgactccagcgaggaagaggacgagatcgacggacctgctggacaggctgaacctgatagagcccactacaatatcgtgaccttctgctgcaagtgcgatagcaccctgagaagatgcgtgcagagcacccacgtggacatcaggaccctcgaggatctgctcatgggcaccctgggaattgtggccccaatctgctctcagaagcctggcagcggcgccaccaatttcagtctgcttaaacaggccggggacgtcgaagagaaccccggacctatgcatcggcgcagatctcggtcttgtagagaagaccaaaagcctgtcatggatgatcaaagagatttaatttctaataatgaacaattacccatgttgggacggcgccccggagcccccgaatctaagtgcagccggggagctttatatacaggattttctattttagtgaccttattattagctggccaagccacaacagcttatttcttataccaacaacaaggacggttagataaattaacagtcacatctcaaaatttacaattagagaatttaagaatgaaattacctaaaccacccaaacccgtcagtaaaatgagaatggctacccccttattaatgcaagccctccccatgggcgccttaccccaaggacccatgcaaaatgctacaaagtatggaaatatgacagaagatcatgtcatgcacttattacaaaacgctgaccccttaaaagtctatccccctttaaaaggatctttccctgaaaatttaagacatttaaaaaatacaatggagacaattgattggaaagtctttgaatcttggatgcatcattggttattatttgaaatgtctaggcattctttagagcaaaaacccaccgatgctccacccaaggaaagtttagagttagaggacccttctagcggattaggagtcaccaaacaggacttaggacccgtccccatgatggaaacactgtgccagcggctgaacgtgtgccaggacaagattctgacccactatgagaacgacagcaccgacctgagggaccacatcgactactggaagcacatgcggctggaatgcgccatctactacaaggctagagagatgggcttcaagcacatcaaccaccaggtggtgcctacactggccgtgtctaagaacaaggctctgcaggccatcgaactgcagctgaccctggaaacaatctacaactcccagtactccaacgagaagtggaccctccaggacgtgtcactggaagtgtacctgaccgctcctaccggctgcatcaagaaacacggctacaccgtggaagtgcagtttgacggcgatatctgcaataccatgcactacaccaactggacccacatctacatctgcgaagaggccagcgtgacagtggtggaaggccaggtcgactactacggcctgtactacgtgcacgagggcatcagaacctacttcgtgcagttcaaggatgacgccgagaagtacagcaagaacaaagtgtgggaagtgcacgctggcggccaagtgatcctgtgtcctacaagcgtgttcagcagcaacgaagtgtctagccccgagatcatcagacagcacctggccaatcatcctgccgccacacacacaaaagctgtggccctgggcaccgaggaaacacagaccacaattcagaggcccagaagcgagcccgataccggcaatccttgtcacaccaccaagctgctgcaccgggattccgtggattctgcccctatcctgaccgccttcaacagctcccacaaaggccggatcaactgcaacagcaacaccacacctatcgtgcacctgaaaggggacgccaacactctgaaggccctgcggtacagattcaagaagcactgtaccctgtataccgccgtgtccagcacatggcactggaccggacacaacgtgaaacacaagagcgccatcgtgaccctgacctacgatagcgagtggcagagggaccagttcctgagccaagtgaagatccctaagaccatcaccgtgtccaccggctttatgagcatttga

SEQ ID NO: 42 - protein, 1756 aa, Human papillomavirus type 16
  REGION 1..1756: note = HPV16 Ii-E1E6E7-p2a-Ii-E2 translated
MHRRRSRSCREDQKPVMDDQRDLISNNEQLPMLGRRPGAPESKCSRGALYTGFSILVTLLLAGQATTAYFLYQQQGRLDKLTVTSQNLQLENLRMKLPKPPKPVSKMRMATPLLMQALPMGALPQGPMQNATKYGNMTEDHVMHLLQNADPLKVYPPLKGSFPENLRHLKNTMETIDWKVFESWMHHWLLFEMSRHSLEQKPTDAPPKESLELEDPSSGLGVTKQDLGPVPMMADPAGTNGEEGTGCNGWFYVEAVVEKKTGDAISDDENENDSDTGEDLVDFIVNDNDYLTQAETETAHALFTAQEAKQHRDAVQVLKRKYLGSPLSDISGCVDNNISPRLKAICIEKQSRAAKRRLFESEDSGYGNTEVETQQMLQVEGRHETETPCSQYSGGSGGGCSQYSSGSGGEGVSERHTICQTPLTNILNVLKTSNAKAAMLAKFKELYGVSFSELVRPFKSNKSTCCDWCIAAFGLTPSIADSIKTLLQQYCLYLHIQSLACSWGMVVLLLVRYKCGKNRETIEKLLSKLLCVSPMCMMIEPPKLRSTAAALYWYKTGISNISEVYGDTPEWIQRQTVLQHSFNDCTFELSQMVQWAYDNDIVDDSEIAYKYAQLADTNSNASAFLKSNSQAKIVKDCATMCRHYKRAEKKQMSMSQWIKYRCDRVDDGGDWKQIVMFLRYQGVEFMSFLTALKRFLQGIPKKNCILLYGAANTGKSLFGMSLMKFLQGSVICFVNSKSHFWLQPLADAKIGMLDDATVPCWNYIDDNLRNALDGNLVSMDVKHRPLVQLKCPPLLITSNINAGTDSRWPYLHNRLVVFTFPNEFPFDENGNPVYELNDKNWKSFFSRTWSRLSLHEDEDKENDGDSLPTFKCVSGQNTNTLGSMHQKRTAMFQDPQERPRKLPQLCTELQTTIHDIILECVYCKQQLLRREVYDFAFRDLCIVYRDGNPYAVCDKCLKFYSKISEYRHYCYSLYGTTLEQQYNKPLCDLLIRCINCQKPQCPEEKQRHLDKKQRFHNIRGRWTGRCMSCCRSSRTRRGSMHGDTPTLHEYMLDLQPETTDLYGYEQLNDSSEEEDEIDGPAGQAEPDRAHYNIVTFCCKCDSTLRRCVQSTHVDIRTLEDLLMGTLGIVAPICSQKPGSGATNFSLLKQAGDVEENPGPMHRRRSRSCREDQKPVMDDQRDLISNNEQLPMLGRRPGAPESKCSRGALYTGFSILVTLLLAGQATTAYFLYQQQGRLDKLTVTSQNLQLENLRMKLPKPPKPVSKMRMATPLLMQALPMGALPQGPMQNATKYGNMTEDHVMHLLQNADPLKVYPPLKGSFPENLRHLKNTMETIDWKVFESWMHHWLLFEMSRHSLEQKPTDAPPKESLELEDPSSGLGVTKQDLGPVPMMETLCQRLNVCQDKILTHYENDSTDLRDHIDYWKHMRLECAIYYKAREMGFKHINHQVVPTLAVSKNKALQAIELQLTLETIYNSQYSNEKWTLQDVSLEVYLTAPTGCIKKHGYTVEVQFDGDICNTMHYTNWTHIYICEEASVTVVEGQVDYYGLYYVHEGIRTYFVQFKDDAEKYSKNKVWEVHAGGQVILCPTSVFSSNEVSSPEIIRQHLANHPAATHTKAVALGTEETQTTIQRPRSEPDTGNPCHTTKLLHRDSVDSAPILTAFNSSHKGRINCNSNTTPIVHLKGDANTLKALRYRFKKHCTLYTAVSSTWHWTGHNVKHKSAIVTLTYDSEWQRDQFLSQVKIPKTITVSTGFMSI

SEQ ID NO: 43 - DNA, 3414 bp, Human papillomavirus type 16
  misc_feature 1..3414: note = HPV16 Ii-E1E6E7
  source 1..3414: mol_type = other DNA
atgcacagaaggcggagcagaagctgccgcgaggatcagaaacccgtgatggacgaccagcgggacctgatcagcaacaacgagcagctgcctatgctcggcagaaggcctggcgctcctgagagcaaatgttctagaggcgccctgtacaccggcttcagcatcctggttacactgctgctggccggacaggctaccaccgcctactttctgtatcagcagcagggcagactggacaagctgaccgtgaccagccagaacctgcagctggaaaacctgcggatgaagctgcccaagcctcctaagcctgtgtccaagatgcggatggccacacctctgctgatgcaggctttgcctatgggagcactgcctcagggccctatgcagaacgccaccaaatacggcaacatgaccgaggaccacgtgatgcatctgctgcagaatgccgatcctctgaaggtgtaccctccactgaagggcagctttcccgagaacctgcggcacctgaagaacaccatggaaaccatcgactggaaggtgttcgagagctggatgcaccactggctgctgttcgagatgagccggcacagcctggaacagaagcctacagacgcccctcctaaagagtccctggaactggaagatcccagctctggcctgggcgtgacaaagcaagatctgggccctgtgcctatgatggccgatcctgctggcacaaacggcgaagaaggcacaggctgcaacggctggttttacgtggaagccgtggtggaaaagaaaaccggcgacgccatcagcgacgacgagaacgagaatgacagcgacaccggcgaggacctggtggacttcatcgtgaacgacaacgactacctgacacaggccgagacagagacagcccacgctctgtttacagcccaagaggccaagcagcacagagatgccgttcaggtgctgaagagaaagtacctgggcagccctctgagcgacatcagcggctgtgtggacaacaacatcagccccagactgaaggccatctgcatcgagaagcagagcagagccgccaagcggagactgtttgagagcgaggatagcggctacggcaataccgaggtggaaacccagcaaatgctgcaggtcgagggcagacacgaaaccgagacaccttgcagccagtacagcggaggatctggcggaggctgtagccagtactcttctggaagtggcggagagggcgtgtccgagagacacaccatttgtcagacccctctgaccaacatcctgaacgtgctgaaaaccagcaacgccaaggccgccatgctggccaagttcaaagagctgtacggcgtcagcttcagcgaactcgtgcggcccttcaagagcaacaagtccacctgttgcgactggtgcattgccgcctttggcctgacaccttctatcgccgacagcatcaagaccctgctccagcagtactgtctgtacctgcacattcagagcctggcctgctcttggggaatggtggtgctgctgctcgtgcggtacaagtgcggcaagaacagagagacaattgagaagctgctgagcaaactgctgtgcgtgtcccctatgtgcatgatgatcgagcctccaaagctgcggtctacagccgccgctctgtactggtacaagaccggcatcagcaatatctccgaggtgtacggcgacacccctgagtggattcagagacagaccgtcctgcagcacagcttcaacgactgcaccttcgagctgtcccagatggtgcagtgggcctacgacaatgacatcgtggacgactccgagatcgcctataagtacgcccagctggccgacaccaacagcaatgccagcgccttcctgaagtccaacagccaggccaagatcgtgaaggactgcgccaccatgtgcagacactacaagcgggccgagaagaaacagatgagcatgagccagtggatcaagtacagatgcgacagagtggacgacggcggcgattggaagcagatcgtgatgttcctgagataccagggcgtcgagttcatgagctttctgacagccctgaagcggttcctgcagggcatccccaagaagaactgcatcctgctgtatggcgccgctaacaccggcaagagcctgttcggaatgagcctgatgaagtttctgcagggctccgtgatctgctttgtgaacagcaagagccacttctggctccagccactggccgatgccaagattggcatgctggacgatgccaccgtgccttgctggaactacatcgacgacaacctgagaaacgccctggacggcaacctggtttctatggacgtgaagcacagacccctggtgcagctgaagtgtcctccactgctgatcaccagcaacatcaacgccggcaccgattccagatggccctatctgcacaaccggctggtggtgtttacctttcctaacgagttccccttcgacgaaaacggcaaccccgtgtacgagctgaacgataagaattggaagtccttcttcagccggacctggtccagactgagcctgcacgaggatgaggacaaagagaacgacggcgacagcctgcctaccttcaaatgtgtcagcggccagaacaccaacacactgggcagcatgcaccagaaacggacagccatgttccaggatcctcaagagcggccacggaagctgcctcagctgtgtacagaactgcagaccaccatccacgacatcatcctcgagtgcgtgtactgcaagcagcagctcctgcggagagaagtgtacgacttcgccttccgggatctgtgcatcgtgtacagagatggcaacccctacgccgtgtgcgacaagtgcctgaagttctactccaagatcagcgagtaccggcactactgctactccctgtacggcaccacacttgagcagcagtacaacaagcccctgtgcgatctgctgatccggtgcatcaactgccagaaacctcagtgccccgaggaaaagcagagacacctggacaagaagcagcggttccacaacattagaggcagatggacaggccggtgcatgagctgctgtagaagcagcagaacaagacggggctccatgcacggcgataccccaacactgcacgagtacatgctggatctgcagcccgagacaaccgatctgtacggctatgagcagctgaatgactccagcgaggaagaggacgagatcgacggacctgctggacaggctgaacctgatagagcccactacaatatcgtgaccttctgctgcaagtgcgatagcaccctgagaagatgcgtgcagagcacccacgtggacatcaggaccctcgaggatctgctcatgggcaccctgggaattgtggccccaatctgctctcagaagccttga

SEQ ID NO: 44 - protein, 1137 aa, Human papillomavirus type 16
  REGION 1..1137: note = HPV16 Ii-E1E6E7 translated
MHRRRSRSCREDQKPVMDDQRDLISNNEQLPMLGRRPGAPESKCSRGALYTGFSILVTLLLAGQATTAYFLYQQQGRLDKLTVTSQNLQLENLRMKLPKPPKPVSKMRMATPLLMQALPMGALPQGPMQNATKYGNMTEDHVMHLLQNADPLKVYPPLKGSFPENLRHLKNTMETIDWKVFESWMHHWLLFEMSRHSLEQKPTDAPPKESLELEDPSSGLGVTKQDLGPVPMMADPAGTNGEEGTGCNGWFYVEAVVEKKTGDAISDDENENDSDTGEDLVDFIVNDNDYLTQAETETAHALFTAQEAKQHRDAVQVLKRKYLGSPLSDISGCVDNNISPRLKAICIEKQSRAAKRRLFESEDSGYGNTEVETQQMLQVEGRHETETPCSQYSGGSGGGCSQYSSGSGGEGVSERHTICQTPLTNILNVLKTSNAKAAMLAKFKELYGVSFSELVRPFKSNKSTCCDWCIAAFGLTPSIADSIKTLLQQYCLYLHIQSLACSWGMVVLLLVRYKCGKNRETIEKLLSKLLCVSPMCMMIEPPKLRSTAAALYWYKTGISNISEVYGDTPEWIQRQTVLQHSFNDCTFELSQMVQWAYDNDIVDDSEIAYKYAQLADTNSNASAFLKSNSQAKIVKDCATMCRHYKRAEKKQMSMSQWIKYRCDRVDDGGDWKQIVMFLRYQGVEFMSFLTALKRFLQGIPKKNCILLYGAANTGKSLFGMSLMKFLQGSVICFVNSKSHFWLQPLADAKIGMLDDATVPCWNYIDDNLRNALDGNLVSMDVKHRPLVQLKCPPLLITSNINAGTDSRWPYLHNRLVVFTFPNEFPFDENGNPVYELNDKNWKSFFSRTWSRLSLHEDEDKENDGDSLPTFKCVSGQNTNTLGSMHQKRTAMFQDPQERPRKLPQLCTELQTTIHDIILECVYCKQQLLRREVYDFAFRDLCIVYRDGNPYAVCDKCLKFYSKISEYRHYCYSLYGTTLEQQYNKPLCDLLIRCINCQKPQCPEEKQRHLDKKQRFHNIRGRWTGRCMSCCRSSRTRRGSMHGDTPTLHEYMLDLQPETTDLYGYEQLNDSSEEEDEIDGPAGQAEPDRAHYNIVTFCCKCDSTLRRCVQSTHVDIRTLEDLLMGTLGIVAPICSQKP

SEQ ID NO: 45 - DNA, 4560 bp, Human papillomavirus type 18
  misc_feature 1..4560: note = HPV18 Ii-E1E2E6E7
  source 1..4560: mol_type = other DNA
atgcacagaaggcggagcagaagctgccgcgaggatcagaaacccgtgatggacgaccagcgggacctgatcagcaacaacgagcagctgcctatgctcggcagaaggcctggcgctcctgagagcaaatgttctagaggcgccctgtacaccggcttcagcatcctggttacactgctgctggccggacaggctaccaccgcctactttctgtatcagcagcagggcagactggacaagctgaccgtgaccagccagaacctgcagctggaaaacctgcggatgaagctgcccaagcctcctaagcctgtgtccaagatgcggatggccacacctctgctgatgcaggctttgcctatgggagcactgcctcagggccctatgcagaacgccaccaaatacggcaacatgaccgaggaccacgtgatgcatctgctgcagaatgccgatcctctgaaggtgtaccctccactgaagggcagctttcccgagaacctgcggcacctgaagaacaccatggaaaccatcgactggaaggtgttcgagagctggatgcaccactggctgctgttcgagatgagccggcacagcctggaacagaagcctacagacgcccctcctaaagagtccctggaactggaagatcccagctctggcctgggcgtgacaaagcaagatctgggccctgtgcctatgatggccgatcctgagggaacagatggcgaaggcacaggctgcaacggctggttttacgtgcaagccatcgtggacaagaagaccggcgacgtcatcagcgacgacgaggatgagaatgccaccgacaccggcagcgacatggtggactttatcgatacccagggcacgttctgcgagcaggccgaactggaaacagcccaggcactgtttcacgcccaagaggtgcacaacgatgcccaggtgctgcacgtgctgaagagaaagtttgccggcggaagcaccgagaacagcccactgggagaaagactggaagtggacaccgagctgagccccagactgcaagagatcagcctgaacagcggccagaagaaggccaagcggagactgttcaccatctccgacagcggctacggctgtagcgaagtggaagccacacagatccaagtgaccaccaatggcgagcacggcggcaatgtttgttctggcggatctaccgaggccatcgacaatggcggaaccgagggcaacaatagctccgtggatggcaccagcgacaacagcaacatcgagaacgtgaaccctcagtgcacaatcgcccagctgaaggacctgctgaaagtgaacaacaagcagggcgccatgctggccgtgtttaaggatacctacggcctgagcttcaccgacctcgtgcggaacttcaagagcgacaagaccacctgtaccgactgggtcaccgccatctttggcgtgaacccaacaatcgccgagggcttcaagaccctgatccagcctttcatcctgtacgcccacatccagtgtctggactgcaaatggggcgtgctgattctggccctgctgagatacaagtgcggcaagagcagactgaccgtggccaagggactgagcactctgctgcatgtgcccgagacatgcatgctgattcagcctccaaagctgcggtctagcgtggccgctctgtactggtacagaaccggcatcagcaatatcagcgaagtgatgggcgacacccctgagtggattcagcggctgacaatcatccagcacggcatcgacgactccaacttcgacctgagcgagatggtgcagtgggccttcgataacgagctgaccgacgagtccgacatggcctttgaatacgccctgctggcagacagcaactccaatgccgccgctttcctgaagtccaactgccaggccaagtacctgaaggactgcgccaccatgtgcaagcactaccgcagagcacagaaacggcagatgaacatgagccagtggatccggttccggtgcagcaagatcgatgaaggtggcgactggcggcctatcgtgcagttcctgagataccagcagatcgagttcatcacctttctgggcgccctgaagtcattcctgaagggaacccctaaaaagaactgcctggtgttctgcggccctgccaataccggcaagtcctacttcggcatgagctttatccacttcatccagggcgccgtgatcagcttcgtgaacagcaccagccacttttggctggaacccctgaccgataccaaggtggcaatgctggacgacgccaccaccacctgttggacctacttcgacacctacatgcggaacgccctggacggcaaccccatcagcatcgacagaaagcacaagcccctcatccagctgaaatgccctccaatcctgctgaccacaaacattcaccccgccaaggacaatcggtggccctacctggaaagccggatcaccgtgttcgagttccccaacgcctttccattcgacaagaacggcaatcccgtgtacgagatcaacgacaagaattggaagtgcttcttcgagcggacctggtccagactggatctgcacgaggaagaagaggacgccgacacagagggcaatcccttcggcacctttaagtgtgtggccgggcagaatcacagacccctgggatctatgcagaccccaaaagagacactgagcgagcggctgagcgccctccaggacaagatcatcgaccactacgagaacgacagcaaggacatcgacagccagattcagtactggcagctgatcagatgggagaacgccatcttcttcgccgctagagagcacggaattcagaccctgaaccaccaggtggtgcccgcctacaacatcagcaagagcaaggcccacaaggccattgaactccagatggccctgcaaggactggctcagagcgcctacaagaccgaggattggacactccaggatacctgcgaggaactgtggaacacagagcccacacactgcttcaagaaaggcggccagaccgtgcaggtctacttcgatggcaacaaggataactgcatgacctacgtggcctgggacagcgtgtactacatgacagatgccggcacctgggacaagaccgccacctgtgtttctcaccggggcctgtactacgtgaaagagggctacaacaccttctacatcgagtttaagagcgagtgcgagaagtatggcaacaccggcacatgggaagtgcatttcggcaacaacgtgatcgactgcaacgactccatgtgctccacctccgatgacaccgtgtctgccacacagctggtcaagcagctccagcacaccccatctccatacagctctacagtgtctgtgggcaccgccaagacctatggccaaacaagcgctgccacaagacctggccattgtggactggccgagaagcagcactgtggccctgttaatcctctgctgggagccgccacacctacaggcaacaacaagagaagaaagctgtgcagcggcaacaccacacctatcattcacctgaagggcgacagaaacagcctgaagtgcctgcggtacagactgaggaagcacagcgatcactaccgggacatcagctccacctggcactggacaggtgctggcaacgaaaagaccggcatcctgacagtgacctaccacagcgagactcagcggaccaagttcctgaacaccgtggctatccccgacagcgtgcagattctcgtgggctacatgaccatgggcagcatggccagattcgaggaccctaccagacggccctacaagctgcctgatctgtgcaccgaactgaacaccagccttcaggatatcgagatcacctgtgtgtactgcaagaccgtgctcgagctgacagaagtgtttgagttcgccttcaaggacctgttcgtggtgtacagagacagcatccctcacgccgcctgtcacaagtgcatcgacttctacagcagaatcagagagctgcggcactacagcgactccgtgtacggcgataccctggaaaaactgaccaacaccggactgtacaacctgctgatccggtgcctgagatgccagaaacctcagaaccccgccgagaaactgagacatctgaacgagaagcggcggttccacaatatcgccggccactacagaggccagtgccacagctgttgcaaccgggccagacaagaaagactccagcggagaagaggcagcatgcacggacctaaagccacacttcaggacatcgtgctgcacctcgagcctcagaatgagatccccgtggatctgctcggccatgagcagctgagcgatagcgaggaagagaacgacgagatcgacggcgtcaaccaccagcatctgcctgccagaagggccgaacctcagagacacaccatgctgtgcatgtgctgcaagtgcgaggcccggatcgagagagtggtggaatctagcgccgacgacctgagagctttccagcagctgttcctgaatactctgagcttcgtggccccttggtgcgctagtcagcagtga

SEQ ID NO: 46 - protein, 1519 aa, Human papillomavirus type 18
  REGION 1..1519: note = HPV18 Ii-E1E2E6E7 translated
MHRRRSRSCREDQKPVMDDQRDLISNNEQLPMLGRRPGAPESKCSRGALYTGFSILVTLLLAGQATTAYFLYQQQGRLDKLTVTSQNLQLENLRMKLPKPPKPVSKMRMATPLLMQALPMGALPQGPMQNATKYGNMTEDHVMHLLQNADPLKVYPPLKGSFPENLRHLKNTMETIDWKVFESWMHHWLLFEMSRHSLEQKPTDAPPKESLELEDPSSGLGVTKQDLGPVPMMADPEGTDGEGTGCNGWFYVQAIVDKKTGDVISDDEDENATDTGSDMVDFIDTQGTFCEQAELETAQALFHAQEVHNDAQVLHVLKRKFAGGSTENSPLGERLEVDTELSPRLQEISLNSGQKKAKRRLFTISDSGYGCSEVEATQIQVTTNGEHGGNVCSGGSTEAIDNGGTEGNNSSVDGTSDNSNIENVNPQCTIAQLKDLLKVNNKQGAMLAVFKDTYGLSFTDLVRNFKSDKTTCTDWVTAIFGVNPTIAEGFKTLIQPFILYAHIQCLDCKWGVLILALLRYKCGKSRLTVAKGLSTLLHVPETCMLIQPPKLRSSVAALYWYRTGISNISEVMGDTPEWIQRLTIIQHGIDDSNFDLSEMVQWAFDNELTDESDMAFEYALLADSNSNAAAFLKSNCQAKYLKDCATMCKHYRRAQKRQMNMSQWIRFRCSKIDEGGDWRPIVQFLRYQQIEFITFLGALKSFLKGTPKKNCLVFCGPANTGKSYFGMSFIHFIQGAVISFVNSTSHFWLEPLTDTKVAMLDDATTTCWTYFDTYMRNALDGNPISIDRKHKPLIQLKCPPILLTTNIHPAKDNRWPYLESRITVFEFPNAFPFDKNGNPVYEINDKNWKCFFERTWSRLDLHEEEEDADTEGNPFGTFKCVAGQNHRPLGSMQTPKETLSERLSALQDKIIDHYENDSKDIDSQIQYWQLIRWENAIFFAAREHGIQTLNHQVVPAYNISKSKAHKAIELQMALQGLAQSAYKTEDWTLQDTCEELWNTEPTHCFKKGGQTVQVYFDGNKDNCMTYVAWDSVYYMTDAGTWDKTATCVSHRGLYYVKEGYNTFYIEFKSECEKYGNTGTWEVHFGNNVIDCNDSMCSTSDDTVSATQLVKQLQHTPSPYSSTVSVGTAKTYGQTSAATRPGHCGLAEKQHCGPVNPLLGAATPTGNNKRRKLCSGNTTPIIHLKGDRNSLKCLRYRLRKHSDHYRDISSTWHWTGAGNEKTGILTVTYHSETQRTKFLNTVAIPDSVQILVGYMTMGSMARFEDPTRRPYKLPDLCTELNTSLQDIEITCVYCKTVLELTEVFEFAFKDLFVVYRDSIPHAACHKCIDFYSRIRELRHYSDSVYGDTLEKLTNTGLYNLLIRCLRCQKPQNPAEKLRHLNEKRRFHNIAGHYRGQCHSCCNRARQERLQRRRGSMHGPKATLQDIVLHLEPQNEIPVDLLGHEQLSDSEEENDEIDGVNHQHLPARRAEPQRHTMLCMCCKCEARIERVVESSADDLRAFQQLFLNTLSFVAPWCASQQ

SEQ ID NO: 47 - DNA, 5316 bp, Human papillomavirus type 18
  misc_feature 1..5316: note = HPV18 Ii-E1E2-p2a-Ii-E6E7
  source 1..5316: mol_type = other DNA
atgcacagaaggcggagcagaagctgccgcgaggatcagaaacccgtgatggacgaccagcgggacctgatcagcaacaacgagcagctgcctatgctcggcagaaggcctggcgctcctgagagcaaatgttctagaggcgccctgtacaccggcttcagcatcctggttacactgctgctggccggacaggctaccaccgcctactttctgtatcagcagcagggcagactggacaagctgaccgtgaccagccagaacctgcagctggaaaacctgcggatgaagctgcccaagcctcctaagcctgtgtccaagatgcggatggccacacctctgctgatgcaggctttgcctatgggagcactgcctcagggccctatgcagaacgccaccaaatacggcaacatgaccgaggaccacgtgatgcatctgctgcagaatgccgatcctctgaaggtgtaccctccactgaagggcagctttcccgagaacctgcggcacctgaagaacaccatggaaaccatcgactggaaggtgttcgagagctggatgcaccactggctgctgttcgagatgagccggcacagcctggaacagaagcctacagacgcccctcctaaagagtccctggaactggaagatcccagctctggcctgggcgtgacaaagcaagatctgggccctgtgcctatgatggccgatcctgagggaacagatggcgaaggcacaggctgcaacggctggttttacgtgcaagccatcgtggacaagaagaccggcgacgtcatcagcgacgacgaggatgagaatgccaccgacaccggcagcgacatggtggactttatcgatacccagggcacgttctgcgagcaggccgaactggaaacagcccaggcactgtttcacgcccaagaggtgcacaacgatgcccaggtgctgcacgtgctgaagagaaagtttgccggcggaagcaccgagaacagcccactgggagaaagactggaagtggacaccgagctgagccccagactgcaagagatcagcctgaacagcggccagaagaaggccaagcggagactgttcaccatctccgacagcggctacggctgtagcgaagtggaagccacacagatccaagtgaccaccaatggcgagcacggcggcaatgtttgttctggcggatctaccgaggccatcgacaatggcggaaccgagggcaacaatagctccgtggatggcaccagcgacaacagcaacatcgagaacgtgaaccctcagtgcacaatcgcccagctgaaggacctgctgaaagtgaacaacaagcagggcgccatgctggccgtgtttaaggatacctacggcctgagcttcaccgacctcgtgcggaacttcaagagcgacaagaccacctgtaccgactgggtcaccgccatctttggcgtgaacccaacaatcgccgagggcttcaagaccctgatccagcctttcatcctgtacgcccacatccagtgtctggactgcaaatggggcgtgctgattctggccctgctgagatacaagtgcggcaagagcagactgaccgtggccaagggactgagcactctgctgcatgtgcccgagacatgcatgctgattcagcctccaaagctgcggtctagcgtggccgctctgtactggtacagaaccggcatcagcaatatcagcgaagtgatgggcgacacccctgagtggattcagcggctgacaatcatccagcacggcatcgacgactccaacttcgacctgagcgagatggtgcagtgggccttcgataacgagctgaccgacgagtccgacatggcctttgaatacgccctgctggcagacagcaactccaatgccgccgctttcctgaagtccaactgccaggccaagtacctgaaggactgcgccaccatgtgcaagcactaccgcagagcacagaaacggcagatgaacatgagccagtggatccggttccggtgcagcaagatcgatgaaggtggcgactggcggcctatcgtgcagttcctgagataccagcagatcgagttcatcacctttctgggcgccctgaagtcattcctgaagggaacccctaaaaagaactgcctggtgttctgcggccctgccaataccggcaagtcctacttcggcatgagctttatccacttcatccagggcgccgtgatcagcttcgtgaacagcaccagccacttttggctggaacccctgaccgataccaaggtggcaatgctggacgacgccaccaccacctgttggacctacttcgacacctacatgcggaacgccctggacggcaaccccatcagcatcgacagaaagcacaagcccctcatccagctgaaatgccctccaatcctgctgaccacaaacattcaccccgccaaggacaatcggtggccctacctggaaagccggatcaccgtgttcgagttccccaacgcctttccattcgacaagaacggcaatcccgtgtacgagatcaacgacaagaattggaagtgcttcttcgagcggacctggtccagactggatctgcacgaggaagaagaggacgccgacacagagggcaatcccttcggcacctttaagtgtgtggccgggcagaatcacagacccctgggatctatgcagaccccaaaagagacactgagcgagcggctgagcgccctccaggacaagatcatcgaccactacgagaacgacagcaaggacatcgacagccagattcagtactggcagctgatcagatgggagaacgccatcttcttcgccgctagagagcacggaattcagaccctgaaccaccaggtggtgcccgcctacaacatcagcaagagcaaggcccacaaggccattgaactccagatggccctgcaaggactggctcagagcgcctacaagaccgaggattggacactccaggatacctgcgaggaactgtggaacacagagcccacacactgcttcaagaaaggcggccagaccgtgcaggtctacttcgatggcaacaaggataactgcatgacctacgtggcctgggacagcgtgtactacatgacagatgccggcacctgggacaagaccgccacctgtgtttctcaccggggcctgtactacgtgaaagagggctacaacaccttctacatcgagtttaagagcgagtgcgagaagtatggcaacaccggcacatgggaagtgcatttcggcaacaacgtgatcgactgcaacgactccatgtgctccacctccgatgacaccgtgtctgccacacagctggtcaagcagctccagcacaccccatctccatacagctctacagtgtctgtgggcaccgccaagacctatggccaaacaagcgctgccacaagacctggccattgtggactggccgagaagcagcactgtggccctgttaatcctctgctgggagccgccacacctacaggcaacaacaagagaagaaagctgtgcagcggcaacaccacacctatcattcacctgaagggcgacagaaacagcctgaagtgcctgcggtacagactgaggaagcacagcgatcactaccgggacatcagctccacctggcactggacaggtgctggcaacgaaaagaccggcatcctgacagtgacctaccacagcgagactcagcggaccaagttcctgaacaccgtggctatccccgacagcgtgcagattctcgtgggctacatgaccatgggcagcggcgccaccaatttcagtctgcttaaacaggccggggacgtcgaagagaaccccggacctatgcatcggcgcagatctcggtcttgtagagaagaccaaaagcctgtcatggatgatcaaagagatttaatttctaataatgaacaattacccatgttgggacggcgccccggagcccccgaatctaagtgcagccggggagctttatatacaggattttctattttagtgaccttattattagctggccaagccacaacagcttatttcttataccaacaacaaggacggttagataaattaacagtcacatctcaaaatttacaattagagaatttaagaatgaaattacctaaaccacccaaacccgtcagtaaaatgagaatggctacccccttattaatgcaagccctccccatgggcgccttaccccaaggacccatgcaaaatgctacaaagtatggaaatatgacagaagatcatgtcatgcacttattacaaaacgctgaccccttaaaagtctatccccctttaaaaggatctttccctgaaaatttaagacatttaaaaaatacaatggagacaattgattggaaagtctttgaatcttggatgcatcattggttattatttgaaatgtctaggcattctttagagcaaaaacccaccgatgctccacccaaggaaagtttagagttagaggacccttctagcggattaggagtcaccaaacaggacttaggacccgtccccatgatggccagattcgaggaccctaccagacggccctacaagctgcctgatctgtgcaccgaactgaacaccagccttcaggatatcgagatcacctgtgtgtactgcaagaccgtgctcgagctgacagaagtgtttgagttcgccttcaaggacctgttcgtggtgtacagagacagcatccctcacgccgcctgtcacaagtgcatcgacttctacagcagaatcagagagctgcggcactacagcgactccgtgtacggcgataccctggaaaaactgaccaacaccggactgtacaacctgctgatccggtgcctgagatgccagaaacctcagaaccccgccgagaaactgagacatctgaacgagaagcggcggttccacaatatcgccggccactacagaggccagtgccacagctgttgcaaccgggccagacaagaaagactccagcggagaagaggcagcatgcacggacctaaagccacacttcaggacatcgtgctgcacctcgagcctcagaatgagatccccgtggatctgctcggccatgagcagctgagcgatagcgaggaagagaacgacgagatcgacggcgtcaaccaccagcatctgcctgccagaagggccgaacctcagagacacaccatgctgtgcatgtgctgcaagtgcgaggcccggatcgagagagtggtggaatctagcgccgacgacctgagagctttccagcagctgttcctgaatactctgagcttcgtggccccttggtgcgctagtcagcagtga

SEQ ID NO: 48 - protein, 1771 aa, Human papillomavirus type 18
  REGION 1..1771: note = HPV18 Ii-E1E2-p2a-Ii-E6E7 translated
MHRRRSRSCREDQKPVMDDQRDLISNNEQLPMLGRRPGAPESKCSRGALYTGFSILVTLLLAGQATTAYFLYQQQGRLDKLTVTSQNLQLENLRMKLPKPPKPVSKMRMATPLLMQALPMGALPQGPMQNATKYGNMTEDHVMHLLQNADPLKVYPPLKGSFPENLRHLKNTMETIDWKVFESWMHHWLLFEMSRHSLEQKPTDAPPKESLELEDPSSGLGVTKQDLGPVPMMADPEGTDGEGTGCNGWFYVQAIVDKKTGDVISDDEDENATDTGSDMVDFIDTQGTFCEQAELETAQALFHAQEVHNDAQVLHVLKRKFAGGSTENSPLGERLEVDTELSPRLQEISLNSGQKKAKRRLFTISDSGYGCSEVEATQIQVTTNGEHGGNVCSGGSTEAIDNGGTEGNNSSVDGTSDNSNIENVNPQCTIAQLKDLLKVNNKQGAMLAVFKDTYGLSFTDLVRNFKSDKTTCTDWVTAIFGVNPTIAEGFKTLIQPFILYAHIQCLDCKWGVLILALLRYKCGKSRLTVAKGLSTLLHVPETCMLIQPPKLRSSVAALYWYRTGISNISEVMGDTPEWIQRLTIIQHGIDDSNFDLSEMVQWAFDNELTDESDMAFEYALLADSNSNAAAFLKSNCQAKYLKDCATMCKHYRRAQKRQMNMSQWIRFRCSKIDEGGDWRPIVQFLRYQQIEFITFLGALKSFLKGTPKKNCLVFCGPANTGKSYFGMSFIHFIQGAVISFVNSTSHFWLEPLTDTKVAMLDDATTTCWTYFDTYMRNALDGNPISIDRKHKPLIQLKCPPILLTTNIHPAKDNRWPYLESRITVFEFPNAFPFDKNGNPVYEINDKNWKCFFERTWSRLDLHEEEEDADTEGNPFGTFKCVAGQNHRPLGSMQTPKETLSERLSALQDKIIDHYENDSKDIDSQIQYWQLIRWENAIFFAAREHGIQTLNHQVVPAYNISKSKAHKAIELQMALQGLAQSAYKTEDWTLQDTCEELWNTEPTHCFKKGGQTVQVYFDGNKDNCMTYVAWDSVYYMTDAGTWDKTATCVSHRGLYYVKEGYNTFYIEFKSECEKYGNTGTWEVHFGNNVIDCNDSMCSTSDDTVSATQLVKQLQHTPSPYSSTVSVGTAKTYGQTSAATRPGHCGLAEKQHCGPVNPLLGAATPTGNNKRRKLCSGNTTPIIHLKGDRNSLKCLRYRLRKHSDHYRDISSTWHWTGAGNEKTGILTVTYHSETQRTKFLNTVAIPDSVQILVGYMTMGSGATNFSLLKQAGDVEENPGPMHRRRSRSCREDQKPVMDDQRDLISNNEQLPMLGRRPGAPESKCSRGALYTGFSILVTLLLAGQATTAYFLYQQQGRLDKLTVTSQNLQLENLRMKLPKPPKPVSKMRMATPLLMQALPMGALPQGPMQNATKYGNMTEDHVMHLLQNADPLKVYPPLKGSFPENLRHLKNTMETIDWKVFESWMHHWLLFEMSRHSLEQKPTDAPPKESLELEDPSSGLGVTKQDLGPVPMMARFEDPTRRPYKLPDLCTELNTSLQDIEITCVYCKTVLELTEVFEFAFKDLFVVYRDSIPHAACHKCIDFYSRIRELRHYSDSVYGDTLEKLTNTGLYNLLIRCLRCQKPQNPAEKLRHLNEKRRFHNIAGHYRGQCHSCCNRARQERLQRRRGSMHGPKATLQDIVLHLEPQNEIPVDLLGHEQLSDSEEENDEIDGVNHQHLPARRAEPQRHTMLCMCCKCEARIERVVESSADDLRAFQQLFLNTLSFVAPWCASQQ

SEQ ID NO: 49 - DNA, 3864 bp, Human papillomavirus type 18
  misc_feature 1..3864: note = HPV18 #4 E1E2E6E7
  source 1..3864: mol_type = other DNA
atggccgatcctgagggaacagatggcgaaggcacaggctgcaacggctggttttacgtgcaagccatcgtggacaagaagaccggcgacgtcatcagcgacgacgaggatgagaatgccaccgacaccggcagcgacatggtggactttatcgatacccagggcacgttctgcgagcaggccgaactggaaacagcccaggcactgtttcacgcccaagaggtgcacaacgatgcccaggtgctgcacgtgctgaagagaaagtttgccggcggaagcaccgagaacagcccactgggagaaagactggaagtggacaccgagctgagccccagactgcaagagatcagcctgaacagcggccagaagaaggccaagcggagactgttcaccatctccgacagcggctacggctgtagcgaagtggaagccacacagatccaagtgaccaccaatggcgagcacggcggcaatgtttgttctggcggatctaccgaggccatcgacaatggcggaaccgagggcaacaatagctccgtggatggcaccagcgacaacagcaacatcgagaacgtgaaccctcagtgcacaatcgcccagctgaaggacctgctgaaagtgaacaacaagcagggcgccatgctggccgtgtttaaggatacctacggcctgagcttcaccgacctcgtgcggaacttcaagagcgacaagaccacctgtaccgactgggtcaccgccatctttggcgtgaacccaacaatcgccgagggcttcaagaccctgatccagcctttcatcctgtacgcccacatccagtgtctggactgcaaatggggcgtgctgattctggccctgctgagatacaagtgcggcaagagcagactgaccgtggccaagggactgagcactctgctgcatgtgcccgagacatgcatgctgattcagcctccaaagctgcggtctagcgtggccgctctgtactggtacagaaccggcatcagcaatatcagcgaagtgatgggcgacacccctgagtggattcagcggctgacaatcatccagcacggcatcgacgactccaacttcgacctgagcgagatggtgcagtgggccttcgataacgagctgaccgacgagtccgacatggcctttgaatacgccctgctggcagacagcaactccaatgccgccgctttcctgaagtccaactgccaggccaagtacctgaaggactgcgccaccatgtgcaagcactaccgcagagcacagaaacggcagatgaacatgagccagtggatccggttccggtgcagcaagatcgatgaaggtggcgactggcggcctatcgtgcagttcctgagataccagcagatcgagttcatcacctttctgggcgccctgaagtcattcctgaagggaacccctaaaaagaactgcctggtgttctgcggccctgccaataccggcaagtcctacttcggcatgagctttatccacttcatccagggcgccgtgatcagcttcgtgaacagcaccagccacttttggctggaacccctgaccgataccaaggtggcaatgctggacgacgccaccaccacctgttggacctacttcgacacctacatgcggaacgccctggacggcaaccccatcagcatcgacagaaagcacaagcccctcatccagctgaaatgccctccaatcctgctgaccacaaacattcaccccgccaaggacaatcggtggccctacctggaaagccggatcaccgtgttcgagttccccaacgcctttccattcgacaagaacggcaatcccgtgtacgagatcaacgacaagaattggaagtgcttcttcgagcggacctggtccagactggatctgcacgaggaagaagaggacgccgacacagagggcaatcccttcggcacctttaagtgtgtggccgggcagaatcacagacccctgggatctatgcagaccccaaaagagacactgagcgagcggctgagcgccctccaggacaagatcatcgaccactacgagaacgacagcaaggacatcgacagccagattcagtactggcagctgatcagatgggagaacgccatcttcttcgccgctagagagcacggaattcagaccctgaaccaccaggtggtgcccgcctacaacatcagcaagagcaaggcccacaaggccattgaactccagatggccctgcaaggactggctcagagcgcctacaagaccgaggattggacactccaggatacctgcgaggaactgtggaacacagagcccacacactgcttcaagaaaggcggccagaccgtgcaggtctacttcgatggcaacaaggataactgcatgacctacgtggcctgggacagcgtgtactacatgacagatgccggcacctgggacaagaccgccacctgtgtttctcaccggggcctgtactacgtgaaagagggctacaacaccttctacatcgagtttaagagcgagtgcgagaagtatggcaacaccggcacatgggaagtgcatttcggcaacaacgtgatcgactgcaacgactccatgtgctccacctccgatgacaccgtgtctgccacacagctggtcaagcagctccagcacaccccatctccatacagctctacagtgtctgtgggcaccgccaagacctatggccaaacaagcgctgccacaagacctggccattgtggactggccgagaagcagcactgtggccctgttaatcctctgctgggagccgccacacctacaggcaacaacaagagaagaaagctgtgcagcggcaacaccacacctatcattcacctgaagggcgacagaaacagcctgaagtgcctgcggtacagactgaggaagcacagcgatcactaccgggacatcagctccacctggcactggacaggtgctggcaacgaaaagaccggcatcctgacagtgacctaccacagcgagactcagcggaccaagttcctgaacaccgtggctatccccgacagcgtgcagattctcgtgggctacatgaccatgggcagcatggccagattcgaggaccctaccagacggccctacaagctgcctgatctgtgcaccgaactgaacaccagccttcaggatatcgagatcacctgtgtgtactgcaagaccgtgctcgagctgacagaagtgtttgagttcgccttcaaggacctgttcgtggtgtacagagacagcatccctcacgccgcctgtcacaagtgcatcgacttctacagcagaatcagagagctgcggcactacagcgactccgtgtacggcgataccctggaaaaactgaccaacaccggactgtacaacctgctgatccggtgcctgagatgccagaaacctcagaaccccgccgagaaactgagacatctgaacgagaagcggcggttccacaatatcgccggccactacagaggccagtgccacagctgttgcaaccgggccagacaagaaagactccagcggagaagaggcagcatgcacggacctaaagccacacttcaggacatcgtgctgcacctcgagcctcagaatgagatccccgtggatctgctcggccatgagcagctgagcgatagcgaggaagagaacgacgagatcgacggcgtcaaccaccagcatctgcctgccagaagggccgaacctcagagacacaccatgctgtgcatgtgctgcaagtgcgaggcccggatcgagagagtggtggaatctagcgccgacgacctgagagctttccagcagctgttcctgaatactctgagcttcgtggccccttggtgcgctagtcagcagtga

SEQ ID NO: 50 - protein, 1287 aa, Human papillomavirus type 18
  REGION 1..1287: note = HPV18 #4 E1E2E6E7 translated
MADPEGTDGEGTGCNGWFYVQAIVDKKTGDVISDDEDENATDTGSDMVDFIDTQGTFCEQAELETAQALFHAQEVHNDAQVLHVLKRKFAGGSTENSPLGERLEVDTELSPRLQEISLNSGQKKAKRRLFTISDSGYGCSEVEATQIQVTTNGEHGGNVCSGGSTEAIDNGGTEGNNSSVDGTSDNSNIENVNPQCTIAQLKDLLKVNNKQGAMLAVFKDTYGLSFTDLVRNFKSDKTTCTDWVTAIFGVNPTIAEGFKTLIQPFILYAHIQCLDCKWGVLILALLRYKCGKSRLTVAKGLSTLLHVPETCMLIQPPKLRSSVAALYWYRTGISNISEVMGDTPEWIQRLTIIQHGIDDSNFDLSEMVQWAFDNELTDESDMAFEYALLADSNSNAAAFLKSNCQAKYLKDCATMCKHYRRAQKRQMNMSQWIRFRCSKIDEGGDWRPIVQFLRYQQIEFITFLGALKSFLKGTPKKNCLVFCGPANTGKSYFGMSFIHFIQGAVISFVNSTSHFWLEPLTDTKVAMLDDATTTCWTYFDTYMRNALDGNPISIDRKHKPLIQLKCPPILLTTNIHPAKDNRWPYLESRITVFEFPNAFPFDKNGNPVYEINDKNWKCFFERTWSRLDLHEEEEDADTEGNPFGTFKCVAGQNHRPLGSMQTPKETLSERLSALQDKIIDHYENDSKDIDSQIQYWQLIRWENAIFFAAREHGIQTLNHQVVPAYNISKSKAHKAIELQMALQGLAQSAYKTEDWTLQDTCEELWNTEPTHCFKKGGQTVQVYFDGNKDNCMTYVAWDSVYYMTDAGTWDKTATCVSHRGLYYVKEGYNTFYIEFKSECEKYGNTGTWEVHFGNNVIDCNDSMCSTSDDTVSATQLVKQLQHTPSPYSSTVSVGTAKTYGQTSAATRPGHCGLAEKQHCGPVNPLLGAATPTGNNKRRKLCSGNTTPIIHLKGDRNSLKCLRYRLRKHSDHYRDISSTWHWTGAGNEKTGILTVTYHSETQRTKFLNTVAIPDSVQILVGYMTMGSMARFEDPTRRPYKLPDLCTELNTSLQDIEITCVYCKTVLELTEVFEFAFKDLFVVYRDSIPHAACHKCIDFYSRIRELRHYSDSVYGDTLEKLTNTGLYNLLIRCLRCQKPQNPAEKLRHLNEKRRFHNIAGHYRGQCHSCCNRARQERLQRRRGSMHGPKATLQDIVLHLEPQNEIPVDLLGHEQLSDSEEENDEIDGVNHQHLPARRAEPQRHTMLCMCCKCEARIERVVESSADDLRAFQQLFLNTLSFVAPWCASQQ

SEQ ID NO: 51 - DNA, 5316 bp, Human papillomavirus type 18
  misc_feature 1..5316: note = HPV18 Ii-E1E6E7-p2a-Ii-E2
  source 1..5316: mol_type = other DNA
atgcacagaaggcggagcagaagctgccgcgaggatcagaaacccgtgatggacgaccagcgggacctgatcagcaacaacgagcagctgcctatgctcggcagaaggcctggcgctcctgagagcaaatgttctagaggcgccctgtacaccggcttcagcatcctggttacactgctgctggccggacaggctaccaccgcctactttctgtatcagcagcagggcagactggacaagctgaccgtgaccagccagaacctgcagctggaaaacctgcggatgaagctgcccaagcctcctaagcctgtgtccaagatgcggatggccacacctctgctgatgcaggctttgcctatgggagcactgcctcagggccctatgcagaacgccaccaaatacggcaacatgaccgaggaccacgtgatgcatctgctgcagaatgccgatcctctgaaggtgtaccctccactgaagggcagctttcccgagaacctgcggcacctgaagaacaccatggaaaccatcgactggaaggtgttcgagagctggatgcaccactggctgctgttcgagatgagccggcacagcctggaacagaagcctacagacgcccctcctaaagagtccctggaactggaagatcccagctctggcctgggcgtgacaaagcaagatctgggccctgtgcctatgatggccgatcctgagggaacagatggcgaaggcacaggctgcaacggctggttttacgtgcaagccatcgtggacaagaagaccggcgacgtcatcagcgacgacgaggatgagaatgccaccgacaccggcagcgacatggtggactttatcgatacccagggcacgttctgcgagcaggccgaactggaaacagcccaggcactgtttcacgcccaagaggtgcacaacgatgcccaggtgctgcacgtgctgaagagaaagtttgccggcggaagcaccgagaacagcccactgggagaaagactggaagtggacaccgagctgagccccagactgcaagagatcagcctgaacagcggccagaagaaggccaagcggagactgttcaccatctccgacagcggctacggctgtagcgaagtggaagccacacagatccaagtgaccaccaatggcgagcacggcggcaatgtttgttctggcggatctaccgaggccatcgacaatggcggaaccgagggcaacaatagctccgtggatggcaccagcgacaacagcaacatcgagaacgtgaaccctcagtgcacaatcgcccagctgaaggacctgctgaaagtgaacaacaagcagggcgccatgctggccgtgtttaaggatacctacggcctgagcttcaccgacctcgtgcggaacttcaagagcgacaagaccacctgtaccgactgggtcaccgccatctttggcgtgaacccaacaatcgccgagggcttcaagaccctgatccagcctttcatcctgtacgcccacatccagtgtctggactgcaaatggggcgtgctgattctggccctgctgagatacaagtgcggcaagagcagactgaccgtggccaagggactgagcactctgctgcatgtgcccgagacatgcatgctgattcagcctccaaagctgcggtctagcgtggccgctctgtactggtacagaaccggcatcagcaatatcagcgaagtgatgggcgacacccctgagtggattcagcggctgacaatcatccagcacggcatcgacgactccaacttcgacctgagcgagatggtgcagtgggccttcgataacgagctgaccgacgagtccgacatggcctttgaatacgccctgctggcagacagcaactccaatgccgccgctttcctgaagtccaactgccaggccaagtacctgaaggactgcgccaccatgtgcaagcactaccgcagagcacagaaacggcagatgaacatgagccagtggatccggttccggtgcagcaagatcgatgaaggtggcgactggcggcctatcgtgcagttcctgagataccagcagatcgagttcatcacctttctgggcgccctgaagtcattcctgaagggaacccctaaaaagaactgcctggtgttctgcggccctgccaataccggcaagtcctacttcggcatgagctttatccacttcatccagggcgccgtgatcagcttcgtgaacagcaccagccacttttggctggaacccctgaccgataccaaggtggcaatgctggacgacgccaccaccacctgttggacctacttcgacacctacatgcggaacgccctggacggcaaccccatcagcatcgacagaaagcacaagcccctcatccagctgaaatgccctccaatcctgctgaccacaaacattcaccccgccaaggacaatcggtggccctacctggaaagccggatcaccgtgttcgagttccccaacgcctttccattcgacaagaacggcaatcccgtgtacgagatcaacgacaagaattggaagtgcttcttcgagcggacctggtccagactggatctgcacgaggaagaagaggacgccgacacagagggcaatcccttcggcacctttaagtgtgtggccgggcagaatcacagacccctgggcagcatggccagattcgaggaccctaccagacggccctacaagctgcctgatctgtgcaccgaactgaacaccagccttcaggatatcgagatcacctgtgtgtactgcaagaccgtgctcgagctgacagaagtgtttgagttcgccttcaaggacctgttcgtggtgtacagagacagcatccctcacgccgcctgtcacaagtgcatcgacttctacagcagaatcagagagctgcggcactacagcgactccgtgtacggcgataccctggaaaaactgaccaacaccggactgtacaacctgctgatccggtgcctgagatgccagaaacctcagaaccccgccgagaaactgagacatctgaacgagaagcggcggttccacaatatcgccggccactacagaggccagtgccacagctgttgcaaccgggccagacaagaaagactccagcggagaagaggcagcatgcacggacctaaagccacacttcaggacatcgtgctgcacctcgagcctcagaatgagatccccgtggatctgctcggccatgagcagctgagcgatagcgaggaagagaacgacgagatcgacggcgtcaaccaccagcatctgcctgccagaagggccgaacctcagagacacaccatgctgtgcatgtgctgcaagtgcgaggcccggatcgagagagtggtggaatctagcgccgacgacctgagagctttccagcagctgttcctgaatactctgagcttcgtggccccttggtgcgctagtcagcagggcagcggcgccaccaatttcagtctgcttaaacaggccggggacgtcgaagagaaccccggacctatgcatcggcgcagatctcggtcttgtagagaagaccaaaagcctgtcatggatgatcaaagagatttaatttctaataatgaacaattacccatgttgggacggcgccccggagcccccgaatctaagtgcagccggggagctttatatacaggattttctattttagtgaccttattattagctggccaagccacaacagcttatttcttataccaacaacaaggacggttagataaattaacagtcacatctcaaaatttacaattagagaatttaagaatgaaattacctaaaccacccaaacccgtcagtaaaatgagaatggctacccccttattaatgcaagccctccccatgggcgccttaccccaaggacccatgcaaaatgctacaaagtatggaaatatgacagaagatcatgtcatgcacttattacaaaacgctgaccccttaaaagtctatccccctttaaaaggatctttccctgaaaatttaagacatttaaaaaatacaatggagacaattgattggaaagtctttgaatcttggatgcatcattggttattatttgaaatgtctaggcattctttagagcaaaaacccaccgatgctccacccaaggaaagtttagagttagaggacccttctagcggattaggagtcaccaaacaggacttaggacccgtccccatgatgcagaccccaaaagagacactgagcgagcggctgagcgccctccaggacaagatcatcgaccactacgagaacgacagcaaggacatcgacagccagattcagtactggcagctgatcagatgggagaacgccatcttcttcgccgctagagagcacggaattcagaccctgaaccaccaggtggtgcccgcctacaacatcagcaagagcaaggcccacaaggccattgaactccagatggccctgcaaggactggctcagagcgcctacaagaccgaggattggacactccaggatacctgcgaggaactgtggaacacagagcccacacactgcttcaagaaaggcggccagaccgtgcaggtctacttcgatggcaacaaggataactgcatgacctacgtggcctgggacagcgtgtactacatgacagatgccggcacctgggacaagaccgccacctgtgtttctcaccggggcctgtactacgtgaaagagggctacaacaccttctacatcgagtttaagagcgagtgcgagaagtatggcaacaccggcacatgggaagtgcatttcggcaacaacgtgatcgactgcaacgactccatgtgctccacctccgatgacaccgtgtctgccacacagctggtcaagcagctccagcacaccccatctccatacagctctacagtgtctgtgggcaccgccaagacctatggccaaacaagcgctgccacaagacctggccattgtggactggccgagaagcagcactgtggccctgttaatcctctgctgggagccgccacacctacaggcaacaacaagagaagaaagctgtgcagcggcaacaccacacctatcattcacctgaagggcgacagaaacagcctgaagtgcctgcggtacagactgaggaagcacagcgatcactaccgggacatcagctccacctggcactggacaggtgctggcaacgaaaagaccggcatcctgacagtgacctaccacagcgagactcagcggaccaagttcctgaacaccgtggctatccccgacagcgtgcagattctcgtgggctacatgaccatgtga

SEQ ID NO: 52 - protein, 1771 aa, Human papillomavirus type 18
  REGION 1..1771: note = HPV18 Ii-E1E6E7-p2a-Ii-E2 translated
MHRRRSRSCREDQKPVMDDQRDLISNNEQLPMLGRRPGAPESKCSRGALYTGFSILVTLLLAGQATTAYFLYQQQGRLDKLTVTSQNLQLENLRMKLPKPPKPVSKMRMATPLLMQALPMGALPQGPMQNATKYGNMTEDHVMHLLQNADPLKVYPPLKGSFPENLRHLKNTMETIDWKVFESWMHHWLLFEMSRHSLEQKPTDAPPKESLELEDPSSGLGVTKQDLGPVPMMADPEGTDGEGTGCNGWFYVQAIVDKKTGDVISDDEDENATDTGSDMVDFIDTQGTFCEQAELETAQALFHAQEVHNDAQVLHVLKRKFAGGSTENSPLGERLEVDTELSPRLQEISLNSGQKKAKRRLFTISDSGYGCSEVEATQIQVTTNGEHGGNVCSGGSTEAIDNGGTEGNNSSVDGTSDNSNIENVNPQCTIAQLKDLLKVNNKQGAMLAVFKDTYGLSFTDLVRNFKSDKTTCTDWVTAIFGVNPTIAEGFKTLIQPFILYAHIQCLDCKWGVLILALLRYKCGKSRLTVAKGLSTLLHVPETCMLIQPPKLRSSVAALYWYRTGISNISEVMGDTPEWIQRLTIIQHGIDDSNFDLSEMVQWAFDNELTDESDMAFEYALLADSNSNAAAFLKSNCQAKYLKDCATMCKHYRRAQKRQMNMSQWIRFRCSKIDEGGDWRPIVQFLRYQQIEFITFLGALKSFLKGTPKKNCLVFCGPANTGKSYFGMSFIHFIQGAVISFVNSTSHFWLEPLTDTKVAMLDDATTTCWTYFDTYMRNALDGNPISIDRKHKPLIQLKCPPILLTTNIHPAKDNRWPYLESRITVFEFPNAFPFDKNGNPVYEINDKNWKCFFERTWSRLDLHEEEEDADTEGNPFGTFKCVAGQNHRPLGSMARFEDPTRRPYKLPDLCTELNTSLQDIEITCVYCKTVLELTEVFEFAFKDLFVVYRDSIPHAACHKCIDFYSRIRELRHYSDSVYGDTLEKLTNTGLYNLLIRCLRCQKPQNPAEKLRHLNEKRRFHNIAGHYRGQCHSCCNRARQERLQRRRGSMHGPKATLQDIVLHLEPQNEIPVDLLGHEQLSDSEEENDEIDGVNHQHLPARRAEPQRHTMLCMCCKCEARIERVVESSADDLRAFQQLFLNTLSFVAPWCASQQGSGATNFSLLKQAGDVEENPGPMHRRRSRSCREDQKPVMDDQRDLISNNEQLPMLGRRPGAPESKCSRGALYTGFSILVTLLLAGQATTAYFLYQQQGRLDKLTVTSQNLQLENLRMKLPKPPKPVSKMRMATPLLMQALPMGALPQGPMQNATKYGNMTEDHVMHLLQNADPLKVYPPLKGSFPENLRHLKNTMETIDWKVFESWMHHWLLFEMSRHSLEQKPTDAPPKESLELEDPSSGLGVTKQDLGPVPMMQTPKETLSERLSALQDKIIDHYENDSKDIDSQIQYWQLIRWENAIFFAAREHGIQTLNHQVVPAYNISKSKAHKAIELQMALQGLAQSAYKTEDWTLQDTCEELWNTEPTHCFKKGGQTVQVYFDGNKDNCMTYVAWDSVYYMTDAGTWDKTATCVSHRGLYYVKEGYNTFYIEFKSECEKYGNTGTWEVHFGNNVIDCNDSMCSTSDDTVSATQLVKQLQHTPSPYSSTVSVGTAKTYGQTSAATRPGHCGLAEKQHCGPVNPLLGAATPTGNNKRRKLCSGNTTPIIHLKGDRNSLKCLRYRLRKHSDHYRDISSTWHWTGAGNEKTGILTVTYHSETQRTKFLNTVAIPDSVQILVGYMTM

SEQ ID NO: 53 - DNA, 3459 bp, Human papillomavirus type 18
  misc_feature 1..3459: note = HPV18 Ii-E1E6E7
  source 1..3459: mol_type = other DNA
atgcacagaaggcggagcagaagctgccgcgaggatcagaaacccgtgatggacgaccagcgggacctgatcagcaacaacgagcagctgcctatgctcggcagaaggcctggcgctcctgagagcaaatgttctagaggcgccctgtacaccggcttcagcatcctggttacactgctgctggccggacaggctaccaccgcctactttctgtatcagcagcagggcagactggacaagctgaccgtgaccagccagaacctgcagctggaaaacctgcggatgaagctgcccaagcctcctaagcctgtgtccaagatgcggatggccacacctctgctgatgcaggctttgcctatgggagcactgcctcagggccctatgcagaacgccaccaaatacggcaacatgaccgaggaccacgtgatgcatctgctgcagaatgccgatcctctgaaggtgtaccctccactgaagggcagctttcccgagaacctgcggcacctgaagaacaccatggaaaccatcgactggaaggtgttcgagagctggatgcaccactggctgctgttcgagatgagccggcacagcctggaacagaagcctacagacgcccctcctaaagagtccctggaactggaagatcccagctctggcctgggcgtgacaaagcaagatctgggccctgtgcctatgatggccgatcctgagggaacagatggcgaaggcacaggctgcaacggctggttttacgtgcaagccatcgtggacaagaagaccggcgacgtcatcagcgacgacgaggatgagaatgccaccgacaccggcagcgacatggtggactttatcgatacccagggcacgttctgcgagcaggccgaactggaaacagcccaggcactgtttcacgcccaagaggtgcacaacgatgcccaggtgctgcacgtgctgaagagaaagtttgccggcggaagcaccgagaacagcccactgggagaaagactggaagtggacaccgagctgagccccagactgcaagagatcagcctgaacagcggccagaagaaggccaagcggagactgttcaccatctccgacagcggctacggctgtagcgaagtggaagccacacagatccaagtgaccaccaatggcgagcacggcggcaatgtttgttctggcggatctaccgaggccatcgacaatggcggaaccgagggcaacaatagctccgtggatggcaccagcgacaacagcaacatcgagaacgtgaaccctcagtgcacaatcgcccagctgaaggacctgctgaaagtgaacaacaagcagggcgccatgctggccgtgtttaaggatacctacggcctgagcttcaccgacctcgtgcggaacttcaagagcgacaagaccacctgtaccgactgggtcaccgccatctttggcgtgaacccaacaatcgccgagggcttcaagaccctgatccagcctttcatcctgtacgcccacatccagtgtctggactgcaaatggggcgtgctgattctggccctgctgagatacaagtgcggcaagagcagactgaccgtggccaagggactgagcactctgctgcatgtgcccgagacatgcatgctgattcagcctccaaagctgcggtctagcgtggccgctctgtactggtacagaaccggcatcagcaatatcagcgaagtgatgggcgacacccctgagtggattcagcggctgacaatcatccagcacggcatcgacgactccaacttcgacctgagcgagatggtgcagtgggccttcgataacgagctgaccgacgagtccgacatggcctttgaatacgccctgctggcagacagcaactccaatgccgccgctttcctgaagtccaactgccaggccaagtacctgaaggactgcgccaccatgtgcaagcactaccgcagagcacagaaacggcagatgaacatgagccagtggatccggttccggtgcagcaagatcgatgaaggtggcgactggcggcctatcgtgcagttcctgagataccagcagatcgagttcatcacctttctgggcgccctgaagtcattcctgaagggaacccctaaaaagaactgcctggtgttctgcggccctgccaataccggcaagtcctacttcggcatgagctttatccacttcatccagggcgccgtgatcagcttcgtgaacagcaccagccacttttggctggaacccctgaccgataccaaggtggcaatgctggacgacgccaccaccacctgttggacctacttcgacacctacatgcggaacgccctggacggcaaccccatcagcatcgacagaaagcacaagcccctcatccagctgaaatgccctccaatcctgctgaccacaaacattcaccccgccaaggacaatcggtggccctacctggaaagccggatcaccgtgttcgagttccccaacgcctttccattcgacaagaacggcaatcccgtgtacgagatcaacgacaagaattggaagtgcttcttcgagcggacctggtccagactggatctgcacgaggaagaagaggacgccgacacagagggcaatcccttcggcacctttaagtgtgtggccgggcagaatcacagacccctgggcagcatggccagattcgaggaccctaccagacggccctacaagctgcctgatctgtgcaccgaactgaacaccagccttcaggatatcgagatcacctgtgtgtactgcaagaccgtgctcgagctgacagaagtgtttgagttcgccttcaaggacctgttcgtggtgtacagagacagcatccctcacgccgcctgtcacaagtgcatcgacttctacagcagaatcagagagctgcggcactacagcgactccgtgtacggcgataccctggaaaaactgaccaacaccggactgtacaacctgctgatccggtgcctgagatgccagaaacctcagaaccccgccgagaaactgagacatctgaacgagaagcggcggttccacaatatcgccggccactacagaggccagtgccacagctgttgcaaccgggccagacaagaaagactccagcggagaagaggcagcatgcacggacctaaagccacacttcaggacatcgtgctgcacctcgagcctcagaatgagatccccgtggatctgctcggccatgagcagctgagcgatagcgaggaagagaacgacgagatcgacggcgtcaaccaccagcatctgcctgccagaagggccgaacctcagagacacaccatgctgtgcatgtgctgcaagtgcgaggcccggatcgagagagtggtggaatctagcgccgacgacctgagagctttccagcagctgttcctgaatactctgagcttcgtggccccttggtgcgctagtcagcagtga

SEQ ID NO: 54 - protein, 1152 aa, Human papillomavirus type 18
  REGION 1..1152: note = HPV18 Ii-E1E6E7 translated
MHRRRSRSCREDQKPVMDDQRDLISNNEQLPMLGRRPGAPESKCSRGALYTGFSILVTLLLAGQATTAYFLYQQQGRLDKLTVTSQNLQLENLRMKLPKPPKPVSKMRMATPLLMQALPMGALPQGPMQNATKYGNMTEDHVMHLLQNADPLKVYPPLKGSFPENLRHLKNTMETIDWKVFESWMHHWLLFEMSRHSLEQKPTDAPPKESLELEDPSSGLGVTKQDLGPVPMMADPEGTDGEGTGCNGWFYVQAIVDKKTGDVISDDEDENATDTGSDMVDFIDTQGTFCEQAELETAQALFHAQEVHNDAQVLHVLKRKFAGGSTENSPLGERLEVDTELSPRLQEISLNSGQKKAKRRLFTISDSGYGCSEVEATQIQVTTNGEHGGNVCSGGSTEAIDNGGTEGNNSSVDGTSDNSNIENVNPQCTIAQLKDLLKVNNKQGAMLAVFKDTYGLSFTDLVRNFKSDKTTCTDWVTAIFGVNPTIAEGFKTLIQPFILYAHIQCLDCKWGVLILALLRYKCGKSRLTVAKGLSTLLHVPETCMLIQPPKLRSSVAALYWYRTGISNISEVMGDTPEWIQRLTIIQHGIDDSNFDLSEMVQWAFDNELTDESDMAFEYALLADSNSNAAAFLKSNCQAKYLKDCATMCKHYRRAQKRQMNMSQWIRFRCSKIDEGGDWRPIVQFLRYQQIEFITFLGALKSFLKGTPKKNCLVFCGPANTGKSYFGMSFIHFIQGAVISFVNSTSHFWLEPLTDTKVAMLDDATTTCWTYFDTYMRNALDGNPISIDRKHKPLIQLKCPPILLTTNIHPAKDNRWPYLESRITVFEFPNAFPFDKNGNPVYEINDKNWKCFFERTWSRLDLHEEEEDADTEGNPFGTFKCVAGQNHRPLGSMARFEDPTRRPYKLPDLCTELNTSLQDIEITCVYCKTVLELTEVFEFAFKDLFVVYRDSIPHAACHKCIDFYSRIRELRHYSDSVYGDTLEKLTNTGLYNLLIRCLRCQKPQNPAEKLRHLNEKRRFHNIAGHYRGQCHSCCNRARQERLQRRRGSMHGPKATLQDIVLHLEPQNEIPVDLLGHEQLSDSEEENDEIDGVNHQHLPARRAEPQRHTMLCMCCKCEARIERVVESSADDLRAFQQLFLNTLSFVAPWCASQQ

SEQ ID NO: 55 - DNA, 5033 bp, synthetic construct
  misc_feature 1..5033: note = MfPV3 Ii-E1E2E6E7 as carried by Adenoviral vector
  source 1..5033: mol_type = other DNA
gtgatgcggttttggcagtacatcaatgggcgtggatagcggtttgactcacggggatttccaagtctccaccccattgacgtcaatgggagtttgttttggaaccaaaatcaacgggactttccaaaatgtcgtaacaactccgccccattgacgcaaatgggcggtaggcgtgtacggtgggaggtctatataagcagagctctccctatcagtgatagagatctccctatcagtgatagagatcgtcgacgagctcgtttagtgaaccgtcagatcgcctggagacgccatccacgctgttttgacctctagcaccggtgccaccatgcacagaaggcggagcagaagctgccgcgaggatcagaaacccgtgatggacgaccagcgggacctgatcagcaacaacgagcagctgcctatgctcggcagaaggcctggcgctcctgagagcaaatgttctagaggcgccctgtacaccggcttcagcatcctggttacactgctgctggccggacaggctaccaccgcctactttctgtatcagcagcagggcagactggacaagctgaccgtgaccagccagaacctgcagctggaaaacctgcggatgaagctgcccaagcctcctaagcctgtgtccaagatgcggatggccacacctctgctgatgcaggctttgcctatgggagcactgcctcagggccctatgcagaacgccaccaaatacggcaacatgaccgaggaccacgtgatgcatctgctgcagaatgccgatcctctgaaggtgtaccctccactgaagggcagctttcccgagaacctgcggcacctgaagaacaccatggaaaccatcgactggaaggtgttcgagagctggatgcaccactggctgctgttcgagatgagccggcacagcctggaacagaagcctacagacgcccctcctaaagagtccctggaactggaagatcccagctctggcctgggcgtgacaaagcaagatctgggccctgtgcctatgatggacccttctggaattcacggcgagggcaccagatgcaccggatggttcgatgtggaagccatcgtggaaagacggaccggcgacgtggtgtccgaggacgaagatgagaacctggaagataccggcgtggacctggtggacttcatcgacgatacctgcgagtttgagcaggccggcgacgaagctcctagagcactgctgcatgcccaagagacacaggcccatgtgaacgccgtgcactacctgaagcggaagttcacatgcagccctaccgtgtctcccctgggcaatgtgcagtgttctatcgagggcgagctgagccctagactgcacgccatccaactgaatgaaggcagcggcaaggccaagcggagactgtttctgcctgatagcggctacggcaataccgaggtggaaacccagctgtctcaggtggcaggatccgatggcgaatctcaagcccctggcggcaaagaaaatgtgcagcctgctgaaggcgctggcggagatgttcgacagctgctgagaagcagcaacctgaaggccacactgctgggcaagttcaaggacgtgtacggcatcggcttcaacgaactcgtgcggtgcttcaagagcgacaagaccgtgtgcaccgattgggttgtgggagctgctggcgtgcaccattctgtggccgaaggactgaagcacctgatcgagcctttctgcacatacgcccacatgcagtgcctgacctgcaactggggagtgtttctgctgctgctcatccggttcaagtgcggcaagaacagactgaccgtggccaagtgcatctctaccctgctgaacgtgcaagagccctacctgctgatcgaacctccaaagctgagatccacagccgccgctctgtactggtacagaaccggcatcagcaacgtgtccgaagtgatcggagagcctcctgagtggatcgtgcggcagaccatgtttcagcacagcctcgaggacagcatcttcgacctgtctcagatggtgcagtgggcctacgaccacgatctggtggacgatagcgccattgcctaccagtatgcccagctggccgacgtggactctaatgccgccgctttcctgaagtctaacgctcaggccaaatacgtgaaggactgcgccaccatgtgcagacactacaagagagccgagaagcaggccatgaccatgagccagtggatcagagccagatgcgataagaccgacgacggcggagattggaggcctatcgtgcagttcctgagataccagaacgtcgagttcatcacctttctgagcgcctttaaggccttcctgaagggaatccccaagagaagctgcatcgtgctgtacggccctccaaacaccggcaagagctacttcggaatgagcctgatgcagtttctgcagggcagcatcatcagctacgtgaacagcaacagccacttttggctggcccctctggccgaagctaaagtggccatgctggacgatgccacaccacagtgctggtcctacatcgacacctacatgcggagcgtgctggacggcaaccccatgagcattgaccggaagcacaagaacctgatccagatgaagtgtccgcctctgatcatcaccagcaacaccaacgtgggcgaagatgacagatggcgctacctgcactccagagtggtggtgttcaagttcgagcagacctttccattcgaccagaacggcaatcccgtgtacgagctgtccgacaagaattggaagtccttcttcagccggacctggtccagactcgagctgcaagaggatgaggacaccgagcaggatggctctacagtgcgggcctttaagtgtgtgcccggccagaacaccaatatcctgggctctatggaagccctgagcgagagactgaacgcctaccaggacaagatcctgaccttctacgaagaggactccaaggacctgagggaccagatcgagcattggagatgtgtgcggggagagtgcgccgtgttctacaaggccagagagatgggccacagccacatcaaccaccagcaggttccacctctgcacgtgtccagaggcaaggcccacaatgccattgagctgcagatggccctggaaagcctgaaccagagcgacttcaacaccgaggaatggaccctgcaggacacaagcctggaaatgtggcggaccgagcctcagtgctgcttcaaaaagaaaggcgagacagtgaccgtgctgtatgactgcgacaaggacaacagcatggactacaccctgtggggccacgtgtacgtgtggggagatacaggctgggacaaagtgtgcggaaaggtggactacggcggcctgtactatgtgctgcacggcctgaaagtgtactacgttgagttccagcaagaggccgtgaagtacagcaagaccaacaagtgggaagtgcatatcggcagaacagtgatcccctacagcgagtccgtgtcctctacagccctgtgcgaaagagtgcctatcgccgagatcgccaatggcctccagcagaccagcattccctctcacacacacgccaccaccaaagagaacgcttgggtttcctctcagcctcctaccaagcggtccagaagatctgatccaggcggcgatcctgtcagagccctggatgccaatagcagaaccctgctgtctggaaccgcctgcgagaataccaccggcaactcttacagcgactgcacccctgtggtgcacctgaaaggcgaccccaattgtctgaaggccctgcggttcagattcgtgaaacacaagaatctgttcgtgaacgtgtcctccacctggcggtggaccaacaacaccgaagagaaggccatcgtgactgtgacctttaaggacgagcagcagagacagcagttcctcggcagcgtgaagatccccaactccatctgtgtgtgcaagggcgtgatgacagttggcagcatggtggaaggcagccaagagagagcccggacactgcacgaactgtgcgagcagagagaacagacccagcacgacatcgagctggactgcgtgtactgcctgaatcctctgaccagaaccgaggtgtacgacttcgccagatgggagctgagaatcgtgtacagacgggataccgcctacggcgtgtgcaagctgtgcctgaggttccacagcaaagtgcggaagtacaggcggtacaactactccatctacggccccacactggaacggcggacaaagaagcagctcgaagaactcgtgatccggtgctactgctgccagaaacctcagtgccccgtggaaaagcagcgccacgttgaccagggccagagattccacaacattgccgagcagtggaccggcagatgcctgcaatgttggaggccaacagccgctgaaaccggctctatgatcggccctggacctacactgcaggatatcgtgcttgagctggaacccgagcctgtggacctgagaggatatgagcagctggactccagcgaggacgaggacgagattgaccaccaccaacagcaggacagacccgatgtgcaagaagatgacggcgagcggtttagaatcctgagcgactgtcacgtgtgccagaataccgtgtcaagagtggtgcacagcacccacgacgacctgcacattctcgaggacctgcttatgggcaccctcgagatcgtggcccctaattgtgccgtgggcagcgaacagaaactgatctccgaggaagatctctgaggaagaatgtgcacagtgctaaccagaaaattcaggacaagactagtgcggccgcgactctagatcataatcagccataccacatttgtagaggttttacttgctttaaaaaacctcccacacctccccctgaacctgaaacataaaatgaatgcaattgttgttgttaacttgtttattgcagcttataatggttacaaataaagcaatagcatcacaaatttcacaaataaagcatttttttcactgc

SEQ ID NO: 56 - DNA, 5789 bp, synthetic construct
  misc_feature 1..5789: note = MfPV3 Ii-E1E2-p2a-Ii-E6E7 as carried by Adenoviral vector
  source 1..5789: mol_type = other DNA
gtgatgcggttttggcagtacatcaatgggcgtggatagcggtttgactcacggggatttccaagtctccaccccattgacgtcaatgggagtttgttttggaaccaaaatcaacgggactttccaaaatgtcgtaacaactccgccccattgacgcaaatgggcggtaggcgtgtacggtgggaggtctatataagcagagctctccctatcagtgatagagatctccctatcagtgatagagatcgtcgacgagctcgtttagtgaaccgtcagatcgcctggagacgccatccacgctgttttgacctctagcaccggtgccaccatgcacagaaggcggagcagaagctgccgcgaggatcagaaacccgtgatggacgaccagcgggacctgatcagcaacaacgagcagctgcctatgctcggcagaaggcctggcgctcctgagagcaaatgttctagaggcgccctgtacaccggcttcagcatcctggttacactgctgctggccggacaggctaccaccgcctactttctgtatcagcagcagggcagactggacaagctgaccgtgaccagccagaacctgcagctggaaaacctgcggatgaagctgcccaagcctcctaagcctgtgtccaagatgcggatggccacacctctgctgatgcaggctttgcctatgggagcactgcctcagggccctatgcagaacgccaccaaatacggcaacatgaccgaggaccacgtgatgcatctgctgcagaatgccgatcctctgaaggtgtaccctccactgaagggcagctttcccgagaacctgcggcacctgaagaacaccatggaaaccatcgactggaaggtgttcgagagctggatgcaccactggctgctgttcgagatgagccggcacagcctggaacagaagcctacagacgcccctcctaaagagtccctggaactggaagatcccagctctggcctgggcgtgacaaagcaagatctgggccctgtgcctatgatggacccttctggaattcacggcgagggcaccagatgcaccggatggttcgatgtggaagccatcgtggaaagacggaccggcgacgtggtgtccgaggacgaagatgagaacctggaagataccggcgtggacctggtggacttcatcgacgatacctgcgagtttgagcaggccggcgacgaagctcctagagcactgctgcatgcccaagagacacaggcccatgtgaacgccgtgcactacctgaagcggaagttcacatgcagccctaccgtgtctcccctgggcaatgtgcagtgttctatcgagggcgagctgagccctagactgcacgccatccaactgaatgaaggcagcggcaaggccaagcggagactgtttctgcctgatagcggctacggcaataccgaggtggaaacccagctgtctcaggtggcaggatccgatggcgaatctcaagcccctggcggcaaagaaaatgtgcagcctgctgaaggcgctggcggagatgttcgacagctgctgagaagcagcaacctgaaggccacactgctgggcaagttcaaggacgtgtacggcatcggcttcaacgaactcgtgcggtgcttcaagagcgacaagaccgtgtgcaccgattgggttgtgggagctgctggcgtgcaccattctgtggccgaaggactgaagcacctgatcgagcctttctgcacatacgcccacatgcagtgcctgacctgcaactggggagtgtttctgctgctgctcatccggttcaagtgcggcaagaacagactgaccgtggccaagtgcatctctaccctgctgaacgtgcaagagccctacctgctgatcgaacctccaaagctgagatccacagccgccgctctgtactggtacagaaccggcatcagcaacgtgtccgaagtgatcggagagcctcctgagtggatcgtgcggcagaccatgtttcagcacagcctcgaggacagcatcttcgacctgtctcagatggtgcagtgggcctacgaccacgatctggtggacgatagcgccattgcctaccagtatgcccagctggccgacgtggactctaatgccgccgctttcctgaagtctaacgctcaggccaaatacgtgaaggactgcgccaccatgtgcagacactacaagagagccgagaagcaggccatgaccatgagccagtggatcagagccagatgcgataagaccgacgacggcggagattggaggcctatcgtgcagttcctgagataccagaacgtcgagttcatcacctttctgagcgcctttaaggccttcctgaagggaatccccaagagaagctgcatcgtgctgtacggccctccaaacaccggcaagagctacttcggaatgagcctgatgcagtttctgcagggcagcatcatcagctacgtgaacagcaacagccacttttggctggcccctctggccgaagctaaagtggccatgctggacgatgccacaccacagtgctggtcctacatcgacacctacatgcggagcgtgctggacggcaaccccatgagcattgaccggaagcacaagaacctgatccagatgaagtgtccgcctctgatcatcaccagcaacaccaacgtgggcgaagatgacagatggcgctacctgcactccagagtggtggtgttcaagttcgagcagacctttccattcgaccagaacggcaatcccgtgtacgagctgtccgacaagaattggaagtccttcttcagccggacctggtccagactcgagctgcaagaggatgaggacaccgagcaggatggctctacagtgcgggcctttaagtgtgtgcccggccagaacaccaatatcctgggctctatggaagccctgagcgagagactgaacgcctaccaggacaagatcctgaccttctacgaagaggactccaaggacctgagggaccagatcgagcattggagatgtgtgcggggagagtgcgccgtgttctacaaggccagagagatgggccacagccacatcaaccaccagcaggttccacctctgcacgtgtccagaggcaaggcccacaatgccattgagctgcagatggccctggaaagcctgaaccagagcgacttcaacaccgaggaatggaccctgcaggacacaagcctggaaatgtggcggaccgagcctcagtgctgcttcaaaaagaaaggcgagacagtgaccgtgctgtatgactgcgacaaggacaacagcatggactacaccctgtggggccacgtgtacgtgtggggagatacaggctgggacaaagtgtgcggaaaggtggactacggcggcctgtactatgtgctgcacggcctgaaagtgtactacgttgagttccagcaagaggccgtgaagtacagcaagaccaacaagtgggaagtgcatatcggcagaacagtgatcccctacagcgagtccgtgtcctctacagccctgtgcgaaagagtgcctatcgccgagatcgccaatggcctccagcagaccagcattccctctcacacacacgccaccaccaaagagaacgcttgggtttcctctcagcctcctaccaagcggtccagaagatctgatccaggcggcgatcctgtcagagccctggatgccaatagcagaaccctgctgtctggaaccgcctgcgagaataccaccggcaactcttacagcgactgcacccctgtggtgcacctgaaaggcgaccccaattgtctgaaggccctgcggttcagattcgtgaaacacaagaatctgttcgtgaacgtgtcctccacctggcggtggaccaacaacaccgaagagaaggccatcgtgactgtgacctttaaggacgagcagcagagacagcagttcctcggcagcgtgaagatccccaactccatctgtgtgtgcaagggcgtgatgacagttggcagcggcgccaccaatttcagtctgcttaaacaggccggggacgtcgaagagaaccccggacctatgcatcggcgcagatctcggtcttgtagagaagaccaaaagcctgtcatggatgatcaaagagatttaatttctaataatgaacaattacccatgttgggacggcgccccggagcccccgaatctaagtgcagccggggagctttatatacaggattttctattttagtgaccttattattagctggccaagccacaacagcttatttcttataccaacaacaaggacggttagataaattaacagtcacatctcaaaatttacaattagagaatttaagaatgaaattacctaaaccacccaaacccgtcagtaaaatgagaatggctacccccttattaatgcaagccctccccatgggcgccttaccccaaggacccatgcaaaatgctacaaagtatggaaatatgacagaagatcatgtcatgcacttattacaaaacgctgaccccttaaaagtctatccccctttaaaaggatctttccctgaaaatttaagacatttaaaaaatacaatggagacaattgattggaaagtctttgaatcttggatgcatcattggttattatttgaaatgtctaggcattctttagagcaaaaacccaccgatgctccacccaaggaaagtttagagttagaggacccttctagcggattaggagtcaccaaacaggacttaggacccgtccccatgatggtggaaggcagccaagagagagcccggacactgcacgaactgtgcgagcagagagaacagacccagcacgacatcgagctggactgcgtgtactgcctgaatcctctgaccagaaccgaggtgtacgacttcgccagatgggagctgagaatcgtgtacagacgggataccgcctacggcgtgtgcaagctgtgcctgaggttccacagcaaagtgcggaagtacaggcggtacaactactccatctacggccccacactggaacggcggacaaagaagcagctcgaagaactcgtgatccggtgctactgctgccagaaacctcagtgccccgtggaaaagcagcgccacgttgaccagggccagagattccacaacattgccgagcagtggaccggcagatgcctgcaatgttggaggccaacagccgctgaaaccggctctatgatcggccctggacctacactgcaggatatcgtgcttgagctggaacccgagcctgtggacctgagaggatatgagcagctggactccagcgaggacgaggacgagattgaccaccaccaacagcaggacagacccgatgtgcaagaagatgacggcgagcggtttagaatcctgagcgactgtcacgtgtgccagaataccgtgtcaagagtggtgcacagcacccacgacgacctgcacattctcgaggacctgcttatgggcaccctcgagatcgtggcccctaattgtgccgtgggcagcgaacagaaactgatctccgaggaagatctctgaggaagaatgtgcacagtgctaaccagaaaattcaggacaagactagtgcggccgcgactctagatcataatcagccataccacatttgtagaggttttacttgctttaaaaaacctcccacacctccccctgaacctgaaacataaaatgaatgcaattgttgttgttaacttgtttattgcagcttataatggttacaaataaagcaatagcatcacaaatttcacaaataaagcatttttttcactgc

SEQ ID NO: 57 - DNA, 4337 bp, synthetic construct
  misc_feature 1..4337: note = MfPV3 E1E2E6E7 as carried by Adenoviral vector
  source 1..4337: mol_type = other DNA
gtgatgcggttttggcagtacatcaatgggcgtggatagcggtttgactcacggggatttccaagtctccaccccattgacgtcaatgggagtttgttttggaaccaaaatcaacgggactttccaaaatgtcgtaacaactccgccccattgacgcaaatgggcggtaggcgtgtacggtgggaggtctatataagcagagctctccctatcagtgatagagatctccctatcagtgatagagatcgtcgacgagctcgtttagtgaaccgtcagatcgcctggagacgccatccacgctgttttgacctctagcaccggtgccaccatggacccttctggaattcacggcgagggcaccagatgcaccggatggttcgatgtggaagccatcgtggaaagacggaccggcgacgtggtgtccgaggacgaagatgagaacctggaagataccggcgtggacctggtggacttcatcgacgatacctgcgagtttgagcaggccggcgacgaagctcctagagcactgctgcatgcccaagagacacaggcccatgtgaacgccgtgcactacctgaagcggaagttcacatgcagccctaccgtgtctcccctgggcaatgtgcagtgttctatcgagggcgagctgagccctagactgcacgccatccaactgaatgaaggcagcggcaaggccaagcggagactgtttctgcctgatagcggctacggcaataccgaggtggaaacccagctgtctcaggtggcaggatccgatggcgaatctcaagcccctggcggcaaagaaaatgtgcagcctgctgaaggcgctggcggagatgttcgacagctgctgagaagcagcaacctgaaggccacactgctgggcaagttcaaggacgtgtacggcatcggcttcaacgaactcgtgcggtgcttcaagagcgacaagaccgtgtgcaccgattgggttgtgggagctgctggcgtgcaccattctgtggccgaaggactgaagcacctgatcgagcctttctgcacatacgcccacatgcagtgcctgacctgcaactggggagtgtttctgctgctgctcatccggttcaagtgcggcaagaacagactgaccgtggccaagtgcatctctaccctgctgaacgtgcaagagccctacctgctgatcgaacctccaaagctgagatccacagccgccgctctgtactggtacagaaccggcatcagcaacgtgtccgaagtgatcggagagcctcctgagtggatcgtgcggcagaccatgtttcagcacagcctcgaggacagcatcttcgacctgtctcagatggtgcagtgggcctacgaccacgatctggtggacgatagcgccattgcctaccagtatgcccagctggccgacgtggactctaatgccgccgctttcctgaagtctaacgctcaggccaaatacgtgaaggactgcgccaccatgtgcagacactacaagagagccgagaagcaggccatgaccatgagccagtggatcagagccagatgcgataagaccgacgacggcggagattggaggcctatcgtgcagttcctgagataccagaacgtcgagttcatcacctttctgagcgcctttaaggccttcctgaagggaatccccaagagaagctgcatcgtgctgtacggccctccaaacaccggcaagagctacttcggaatgagcctgatgcagtttctgcagggcagcatcatcagctacgtgaacagcaacagccacttttggctggcccctctggccgaagctaaagtggccatgctggacgatgccacaccacagtgctggtcctacatcgacacctacatgcggagcgtgctggacggcaaccccatgagcattgaccggaagcacaagaacctgatccagatgaagtgtccgcctctgatcatcaccagcaacaccaacgtgggcgaagatgacagatggcgctacctgcactccagagtggtggtgttcaagttcgagcagacctttccattcgaccagaacggcaatcccgtgtacgagctgtccgacaagaattggaagtccttcttcagccggacctggtccagactcgagctgcaagaggatgaggacaccgagcaggatggctctacagtgcgggcctttaagtgtgtgcccggccagaacaccaatatcctgggctctatggaagccctgagcgagagactgaacgcctaccaggacaagatcctgaccttctacgaagaggactccaaggacctgagggaccagatcgagcattggagatgtgtgcggggagagtgcgccgtgttctacaaggccagagagatgggccacagccacatcaaccaccagcaggttccacctctgcacgtgtccagaggcaaggcccacaatgccattgagctgcagatggccctggaaagcctgaaccagagcgacttcaacaccgaggaatggaccctgcaggacacaagcctggaaatgtggcggaccgagcctcagtgctgcttcaaaaagaaaggcgagacagtgaccgtgctgtatgactgcgacaaggacaacagcatggactacaccctgtggggccacgtgtacgtgtggggagatacaggctgggacaaagtgtgcggaaaggtggactacggcggcctgtactatgtgctgcacggcctgaaagtgtactacgttgagttccagcaagaggccgtgaagtacagcaagaccaacaagtgggaagtgcatatcggcagaacagtgatcccctacagcgagtccgtgtcctctacagccctgtgcgaaagagtgcctatcgccgagatcgccaatggcctccagcagaccagcattccctctcacacacacgccaccaccaaagagaacgcttgggtttcctctcagcctcctaccaagcggtccagaagatctgatccaggcggcgatcctgtcagagccctggatgccaatagcagaaccctgctgtctggaaccgcctgcgagaataccaccggcaactcttacagcgactgcacccctgtggtgcacctgaaaggcgaccccaattgtctgaaggccctgcggttcagattcgtgaaacacaagaatctgttcgtgaacgtgtcctccacctggcggtggaccaacaacaccgaagagaaggccatcgtgactgtgacctttaaggacgagcagcagagacagcagttcctcggcagcgtgaagatccccaactccatctgtgtgtgcaagggcgtgatgacagttggcagcatggtggaaggcagccaagagagagcccggacactgcacgaactgtgcgagcagagagaacagacccagcacgacatcgagctggactgcgtgtactgcctgaatcctctgaccagaaccgaggtgtacgacttcgccagatgggagctgagaatcgtgtacagacgggataccgcctacggcgtgtgcaagctgtgcctgaggttccacagcaaagtgcggaagtacaggcggtacaactactccatctacggccccacactggaacggcggacaaagaagcagctcgaagaactcgtgatccggtgctactgctgccagaaacctcagtgccccgtggaaaagcagcgccacgttgaccagggccagagattccacaacattgccgagcagtggaccggcagatgcctgcaatgttggaggccaacagccgctgaaaccggctctatgatcggccctggacctacactgcaggatatcgtgcttgagctggaacccgagcctgtggacctgagaggatatgagcagctggactccagcgaggacgaggacgagattgaccaccaccaacagcaggacagacccgatgtgcaagaagatgacggcgagcggtttagaatcctgagcgactgtcacgtgtgccagaataccgtgtcaagagtggtgcacagcacccacgacgacctgcacattctcgaggacctgcttatgggcaccctcgagatcgtggcccctaattgtgccgtgggcagcgaacagaaactgatctccgaggaagatctctgaggaagaatgtgcacagtgctaaccagaaaattcaggacaagactagtgcggccgcgactctagatcataatcagccataccacatttgtagaggttttacttgctttaaaaaacctcccacacctccccctgaacctgaaacataaaatgaatgcaattgttgttgttaacttgtttattgcagcttataatggttacaaataaagcaatagcatcacaaatttcacaaataaagcatttttttcactgc

SEQ ID NO: 58 - DNA, 5789 bp, synthetic construct
  misc_feature 1..5789: note = MfPV3 Ii-E1E6E7-p2a-Ii-E2 as carried by Adenoviral vector
  source 1..5789: mol_type = other DNA
gtgatgcggttttggcagtacatcaatgggcgtggatagcggtttgactcacggggatttccaagtctccaccccattgacgtcaatgggagtttgttttggaaccaaaatcaacgggactttccaaaatgtcgtaacaactccgccccattgacgcaaatgggcggtaggcgtgtacggtgggaggtctatataagcagagctctccctatcagtgatagagatctccctatcagtgatagagatcgtcgacgagctcgtttagtgaaccgtcagatcgcctggagacgccatccacgctgttttgacctctagcaccggtgccaccatgcacagaaggcggagcagaagctgccgcgaggatcagaaacccgtgatggacgaccagcgggacctgatcagcaacaacgagcagctgcctatgctcggcagaaggcctggcgctcctgagagcaaatgttctagaggcgccctgtacaccggcttcagcatcctggttacactgctgctggccggacaggctaccaccgcctactttctgtatcagcagcagggcagactggacaagctgaccgtgaccagccagaacctgcagctggaaaacctgcggatgaagctgcccaagcctcctaagcctgtgtccaagatgcggatggccacacctctgctgatgcaggctttgcctatgggagcactgcctcagggccctatgcagaacgccaccaaatacggcaacatgaccgaggaccacgtgatgcatctgctgcagaatgccgatcctctgaaggtgtaccctccactgaagggcagctttcccgagaacctgcggcacctgaagaacaccatggaaaccatcgactggaaggtgttcgagagctggatgcaccactggctgctgttcgagatgagccggcacagcctggaacagaagcctacagacgcccctcctaaagagtccctggaactggaagatcccagctctggcctgggcgtgacaaagcaagatctgggccctgtgcctatgatggacccttctggaattcacggcgagggcaccagatgcaccggatggttcgatgtggaagccatcgtggaaagacggaccggcgacgtggtgtccgaggacgaagatgagaacctggaagataccggcgtggacctggtggacttcatcgacgatacctgcgagtttgagcaggccggcgacgaagctcctagagcactgctgcatgcccaagagacacaggcccatgtgaacgccgtgcactacctgaagcggaagttcacatgcagccctaccgtgtctcccctgggcaatgtgcagtgttctatcgagggcgagctgagccctagactgcacgccatccaactgaatgaaggcagcggcaaggccaagcggagactgtttctgcctgatagcggctacggcaataccgaggtggaaacccagctgtctcaggtggcaggatccgatggcgaatctcaagcccctggcggcaaagaaaatgtgcagcctgctgaaggcgctggcggagatgttcgacagctgctgagaagcagcaacctgaaggccacactgctgggcaagttcaaggacgtgtacggcatcggcttcaacgaactcgtgcggtgcttcaagagcgacaagaccgtgtgcaccgattgggttgtgggagctgctggcgtgcaccattctgtggccgaaggactgaagcacctgatcgagcctttctgcacatacgcccacatgcagtgcctgacctgcaactggggagtgtttctgctgctgctcatccggttcaagtgcggcaagaacagactgaccgtggccaagtgcatctctaccctgctgaacgtgcaagagccctacctgctgatcgaacctccaaagctgagatccacagccgccgctctgtactggtacagaaccggcatcagcaacgtgtccgaagtgatcggagagcctcctgagtggatcgtgcggcagaccatgtttcagcacagcctcgaggacagcatcttcgacctgtctcagatggtgcagtgggcctacgaccacgatctggtggacgatagcgccattgcctaccagtatgcccagctggccgacgtggactctaatgccgccgctttcctgaagtctaacgctcaggccaaatacgtgaaggactgcgccaccatgtgcagacactacaagagagccgagaagcaggccatgaccatgagccagtggatcagagccagatgcgataagaccgacgacggcggagattggaggcctatcgtgcagttcctgagataccagaacgtcgagttcatcacctttctgagcgcctttaaggccttcctgaagggaatccccaagagaagctgcatcgtgctgtacggccctccaaacaccggcaagagctacttcggaatgagcctgatgcagtttctgcagggcagcatcatcagctacgtgaacagcaacagccacttttggctggcccctctggccgaagctaaagtggccatgctggacgatgccacaccacagtgctggtcctacatcgacacctacatgcggagcgtgctggacggcaaccccatgagcattgaccggaagcacaagaacctgatccagatgaagtgtccgcctctgatcatcaccagcaacaccaacgtgggcgaagatgacagatggcgctacctgcactccagagtggtggtgttcaagttcgagcagacctttccattcgaccagaacggcaatcccgtgtacgagctgtccgacaagaattggaagtccttcttcagccggacctggtccagactcgagctgcaagaggatgaggacaccgagcaggatggctctacagtgcgggcctttaagtgtgtgcccggccagaacaccaatatcctgggctctatggtggaaggcagccaagagagagcccggacactgcacgaactgtgcgagcagagagaacagacccagcacgacatcgagctggactgcgtgtactgcctgaatcctctgaccagaaccgaggtgtacgacttcgccagatgggagctgagaatcgtgtacagacgggataccgcctacggcgtgtgcaagctgtgcctgaggttccacagcaaagtgcggaagtacaggcggtacaactactccatctacggccccacactggaacggcggacaaagaagcagctcgaagaactcgtgatccggtgctactgctgccagaaacctcagtgccccgtggaaaagcagcgccacgttgaccagggccagagattccacaacattgccgagcagtggaccggcagatgcctgcaatgttggaggccaacagccgctgaaaccggctctatgatcggccctggacctacactgcaggatatcgtgcttgagctggaacccgagcctgtggacctgagaggatatgagcagctggactccagcgaggacgaggacgagattgaccaccaccaacagcaggacagacccgatgtgcaagaagatgacggcgagcggtttagaatcctgagcgactgtcacgtgtgccagaataccgtgtcaagagtggtgcacagcacccacgacgacctgcacattctcgaggacctgcttatgggcaccctcgagatcgtggcccctaattgtgccgtgggcagcggcgccaccaatttcagtctgcttaaacaggccggggacgtcgaagagaaccccggacctatgcatcggcgcagatctcggtcttgtagagaagaccaaaagcctgtcatggatgatcaaagagatttaatttctaataatgaacaattacccatgttgggacggcgccccggagcccccgaatctaagtgcagccggggagctttatatacaggattttctattttagtgaccttattattagctggccaagccacaacagcttatttcttataccaacaacaaggacggttagataaattaacagtcacatctcaaaatttacaattagagaatttaagaatgaaattacctaaaccacccaaacccgtcagtaaaatgagaatggctacccccttattaatgcaagccctccccatgggcgccttaccccaaggacccatgcaaaatgctacaaagtatggaaatatgacagaagatcatgtcatgcacttattacaaaacgctgaccccttaaaagtctatccccctttaaaaggatctttccctgaaaatttaagacatttaaaaaatacaatggagacaattgattggaaagtctttgaatcttggatgcatcattggttattatttgaaatgtctaggcattctttagagcaaaaacccaccgatgctccacccaaggaaagtttagagttagaggacccttctagcggattaggagtcaccaaacaggacttaggacccgtccccatgatggaagccctgagcgagagactgaacgcctaccaggacaagatcctgaccttctacgaagaggactccaaggacctgagggaccagatcgagcattggagatgtgtgcggggagagtgcgccgtgttctacaaggccagagagatgggccacagccacatcaaccaccagcaggttccacctctgcacgtgtccagaggcaaggcccacaatgccattgagctgcagatggccctggaaagcctgaaccagagcgacttcaacaccgaggaatggaccctgcaggacacaagcctggaaatgtggcggaccgagcctcagtgctgcttcaaaaagaaaggcgagacagtgaccgtgctgtatgactgcgacaaggacaacagcatggactacaccctgtggggccacgtgtacgtgtggggagatacaggctgggacaaagtgtgcggaaaggtggactacggcggcctgtactatgtgctgcacggcctgaaagtgtactacgttgagttccagcaagaggccgtgaagtacagcaagaccaacaagtgggaagtgcatatcggcagaacagtgatcccctacagcgagtccgtgtcctctacagccctgtgcgaaagagtgcctatcgccgagatcgccaatggcctccagcagaccagcattccctctcacacacacgccaccaccaaagagaacgcttgggtttcctctcagcctcctaccaagcggtccagaagatctgatccaggcggcgatcctgtcagagccctggatgccaatagcagaaccctgctgtctggaaccgcctgcgagaataccaccggcaactcttacagcgactgcacccctgtggtgcacctgaaaggcgaccccaattgtctgaaggccctgcggttcagattcgtgaaacacaagaatctgttcgtgaacgtgtcctccacctggcggtggaccaacaacaccgaagagaaggccatcgtgactgtgacctttaaggacgagcagcagagacagcagttcctcggcagcgtgaagatccccaactccatctgtgtgtgcaagggcgtgatgacagttggcagcgaacagaaactgatctccgaggaagatctctgaggaagaatgtgcacagtgctaaccagaaaattcaggacaagactagtgcggccgcgactctagatcataatcagccataccacatttgtagaggttttacttgctttaaaaaacctcccacacctccccctgaacctgaaacataaaatgaatgcaattgttgttgttaacttgtttattgcagcttataatggttacaaataaagcaatagcatcacaaatttcacaaataaagcatttttttcactgc

SEQ ID NO: 59 - DNA, 3947 bp, synthetic construct
  misc_feature 1..3947: note = MfPV3 Ii-E1E6E7 as carried by Adenoviral vector
  source 1..3947: mol_type = other DNA
gtgatgcggttttggcagtacatcaatgggcgtggatagcggtttgactcacggggatttccaagtctccaccccattgacgtcaatgggagtttgttttggaaccaaaatcaacgggactttccaaaatgtcgtaacaactccgccccattgacgcaaatgggcggtaggcgtgtacggtgggaggtctatataagcagagctctccctatcagtgatagagatctccctatcagtgatagagatcgtcgacgagctcgtttagtgaaccgtcagatcgcctggagacgccatccacgctgttttgacctctagcaccggtgccaccatgcacagaaggcggagcagaagctgccgcgaggatcagaaacccgtgatggacgaccagcgggacctgatcagcaacaacgagcagctgcctatgctcggcagaaggcctggcgctcctgagagcaaatgttctagaggcgccctgtacaccggcttcagcatcctggttacactgctgctggccggacaggctaccaccgcctactttctgtatcagcagcagggcagactggacaagctgaccgtgaccagccagaacctgcagctggaaaacctgcggatgaagctgcccaagcctcctaagcctgtgtccaagatgcggatggccacacctctgctgatgcaggctttgcctatgggagcactgcctcagggccctatgcagaacgccaccaaatacggcaacatgaccgaggaccacgtgatgcatctgctgcagaatgccgatcctctgaaggtgtaccctccactgaagggcagctttcccgagaacctgcggcacctgaagaacaccatggaaaccatcgactggaaggtgttcgagagctggatgcaccactggctgctgttcgagatgagccggcacagcctggaacagaagcctacagacgcccctcctaaagagtccctggaactggaagatcccagctctggcctgggcgtgacaaagcaagatctgggccctgtgcctatgatggacccttctggaattcacggcgagggcaccagatgcaccggatggttcgatgtggaagccatcgtggaaagacggaccggcgacgtggtgtccgaggacgaagatgagaacctggaagataccggcgtggacctggtggacttcatcgacgatacctgcgagtttgagcaggccggcgacgaagctcctagagcactgctgcatgcccaagagacacaggcccatgtgaacgccgtgcactacctgaagcggaagttcacatgcagccctaccgtgtctcccctgggcaatgtgcagtgttctatcgagggcgagctgagccctagactgcacgccatccaactgaatgaaggcagcggcaaggccaagcggagactgtttctgcctgatagcggctacggcaataccgaggtggaaacccagctgtctcaggtggcaggatccgatggcgaatctcaagcccctggcggcaaagaaaatgtgcagcctgctgaaggcgctggcggagatgttcgacagctgctgagaagcagcaacctgaaggccacactgctgggcaagttcaaggacgtgtacggcatcggcttcaacgaactcgtgcggtgcttcaagagcgacaagaccgtgtgcaccgattgggttgtgggagctgctggcgtgcaccattctgtggccgaaggactgaagcacctgatcgagcctttctgcacatacgcccacatgcagtgcctgacctgcaactggggagtgtttctgctgctgctcatccggttcaagtgcggcaagaacagactgaccgtggccaagtgcatctctaccctgctgaacgtgcaagagccctacctgctgatcgaacctccaaagctgagatccacagccgccgctctgtactggtacagaaccggcatcagcaacgtgtccgaagtgatcggagagcctcctgagtggatcgtgcggcagaccatgtttcagcacagcctcgaggacagcatcttcgacctgtctcagatggtgcagtgggcctacgaccacgatctggtggacgatagcgccattgcctaccagtatgcccagctggccgacgtggactctaatgccgccgctttcctgaagtctaacgctcaggccaaatacgtgaaggactgcgccaccatgtgcagacactacaagagagccgagaagcaggccatgaccatgagccagtggatcagagccagatgcgataagaccgacgacggcggagattggaggcctatcgtgcagttcctgagataccagaacgtcgagttcatcacctttctgagcgcctttaaggccttcctgaagggaatccccaagagaagctgcatcgtgctgtacggccctccaaacaccggcaagagctacttcggaatgagcctgatgcagtttctgcagggcagcatcatcagctacgtgaacagcaacagccacttttggctggcccctctggccgaagctaaagtggccatgctggacgatgccacaccacagtgctggtcctacatcgacacctacatgcggagcgtgctggacggcaaccccatgagcattgaccggaagcacaagaacctgatccagatgaagtgtccgcctctgatcatcaccagcaacaccaacgtgggcgaagatgacagatggcgctacctgcactccagagtggtggtgttcaagttcgagcagacctttccattcgaccagaacggcaatcccgtgtacgagctgtccgacaagaattggaagtccttcttcagccggacctggtccagactcgagctgcaagaggatgaggacaccgagcaggatggctctacagtgcgggcctttaagtgtgtgcccggccagaacaccaatatcctgggctctatggtggaaggcagccaagagagagcccggacactgcacgaactgtgcgagcagagagaacagacccagcacgacatcgagctggactgcgtgtactgcctgaatcctctgaccagaaccgaggtgtacgacttcgccagatgggagctgagaatcgtgtacagacgggataccgcctacggcgtgtgcaagctgtgcctgaggttccacagcaaagtgcggaagtacaggcggtacaactactccatctacggccccacactggaacggcggacaaagaagcagctcgaagaactcgtgatccggtgctactgctgccagaaacctcagtgccccgtggaaaagcagcgccacgttgaccagggccagagattccacaacattgccgagcagtggaccggcagatgcctgcaatgttggaggccaacagccgctgaaaccggctctatgatcggccctggacctacactgcaggatatcgtgcttgagctggaacccgagcctgtggacctgagaggatatgagcagctggactccagcgaggacgaggacgagattgaccaccaccaacagcaggacagacccgatgtgcaagaagatgacggcgagcggtttagaatcctgagcgactgtcacgtgtgccagaataccgtgtcaagagtggtgcacagcacccacgacgacctgcacattctcgaggacctgcttatgggcaccctcgagatcgtggcccctaattgtgccgtgggcagcgaacagaaactgatctccgaggaagatctctgaggaagaatgtgcacagtgctaaccagaaaattcaggacaagactagtgcggccgcgactctagatcataatcagccataccacatttgtagaggttttacttgctttaaaaaacctcccacacctccccctgaacctgaaacataaaatgaatgcaattgttgttgttaacttgtttattgcagcttataatggttacaaataaagcaatagcatcacaaatttcacaaataaagcatttttttcactgc

SEQ ID NO: 60 - DNA, 5095 bp, synthetic construct
  misc_feature 1..5095: note = HPV16 Ii-E1E2E6E7 as carried by Adenoviral vector
  source 1..5095: mol_type = other DNA
gtgatgcggttttggcagtacatcaatgggcgtggatagcggtttgactcacggggatttccaagtctccaccccattgacgtcaatgggagtttgttttggaaccaaaatcaacgggactttccaaaatgtcgtaacaactccgccccattgacgcaaatgggcggtaggcgtgtacggtgggaggtctatataagcagagctctccctatcagtgatagagatctccctatcagtgatagagatcgtcgacgagctcgtttagtgaaccgtcagatcgcctggagacgccatccacgctgttttgacctctagcaccggtgccaccatgcacagaaggcggagcagaagctgccgcgaggatcagaaacccgtgatggacgaccagcgggacctgatcagcaacaacgagcagctgcctatgctcggcagaaggcctggcgctcctgagagcaaatgttctagaggcgccctgtacaccggcttcagcatcctggttacactgctgctggccggacaggctaccaccgcctactttctgtatcagcagcagggcagactggacaagctgaccgtgaccagccagaacctgcagctggaaaacctgcggatgaagctgcccaagcctcctaagcctgtgtccaagatgcggatggccacacctctgctgatgcaggctttgcctatgggagcactgcctcagggccctatgcagaacgccaccaaatacggcaacatgaccgaggaccacgtgatgcatctgctgcagaatgccgatcctctgaaggtgtaccctccactgaagggcagctttcccgagaacctgcggcacctgaagaacaccatggaaaccatcgactggaaggtgttcgagagctggatgcaccactggctgctgttcgagatgagccggcacagcctggaacagaagcctacagacgcccctcctaaagagtccctggaactggaagatcccagctctggcctgggcgtgacaaagcaagatctgggccctgtgcctatgatggccgatcctgctggcacaaacggcgaagaaggcacaggctgcaacggctggttttacgtggaagccgtggtggaaaagaaaaccggcgacgccatcagcgacgacgagaacgagaatgacagcgacaccggcgaggacctggtggacttcatcgtgaacgacaacgactacctgacacaggccgagacagagacagcccacgctctgtttacagcccaagaggccaagcagcacagagatgccgttcaggtgctgaagagaaagtacctgggcagccctctgagcgacatcagcggctgtgtggacaacaacatcagccccagactgaaggccatctgcatcgagaagcagagcagagccgccaagcggagactgtttgagagcgaggatagcggctacggcaataccgaggtggaaacccagcaaatgctgcaggtcgagggcagacacgaaaccgagacaccttgcagccagtacagcggaggatctggcggaggctgtagccagtactcttctggaagtggcggagagggcgtgtccgagagacacaccatttgtcagacccctctgaccaacatcctgaacgtgctgaaaaccagcaacgccaaggccgccatgctggccaagttcaaagagctgtacggcgtcagcttcagcgaactcgtgcggcccttcaagagcaacaagtccacctgttgcgactggtgcattgccgcctttggcctgacaccttctatcgccgacagcatcaagaccctgctccagcagtactgtctgtacctgcacattcagagcctggcctgctcttggggaatggtggtgctgctgctcgtgcggtacaagtgcggcaagaacagagagacaattgagaagctgctgagcaaactgctgtgcgtgtcccctatgtgcatgatgatcgagcctccaaagctgcggtctacagccgccgctctgtactggtacaagaccggcatcagcaatatctccgaggtgtacggcgacacccctgagtggattcagagacagaccgtcctgcagcacagcttcaacgactgcaccttcgagctgtcccagatggtgcagtgggcctacgacaatgacatcgtggacgactccgagatcgcctataagtacgcccagctggccgacaccaacagcaatgccagcgccttcctgaagtccaacagccaggccaagatcgtgaaggactgcgccaccatgtgcagacactacaagcgggccgagaagaaacagatgagcatgagccagtggatcaagtacagatgcgacagagtggacgacggcggcgattggaagcagatcgtgatgttcctgagataccagggcgtcgagttcatgagctttctgacagccctgaagcggttcctgcagggcatccccaagaagaactgcatcctgctgtatggcgccgctaacaccggcaagagcctgttcggaatgagcctgatgaagtttctgcagggctccgtgatctgctttgtgaacagcaagagccacttctggctccagccactggccgatgccaagattggcatgctggacgatgccaccgtgccttgctggaactacatcgacgacaacctgagaaacgccctggacggcaacctggtttctatggacgtgaagcacagacccctggtgcagctgaagtgtcctccactgctgatcaccagcaacatcaacgccggcaccgattccagatggccctatctgcacaaccggctggtggtgtttacctttcctaacgagttccccttcgacgaaaacggcaaccccgtgtacgagctgaacgataagaattggaagtccttcttcagccggacctggtccagactgagcctgcacgaggatgaggacaaagagaacgacggcgacagcctgcctaccttcaaatgtgtcagcggccagaacaccaacacactgggcagcatggaaacactgtgccagcggctgaacgtgtgccaggacaagattctgacccactatgagaacgacagcaccgacctgagggaccacatcgactactggaagcacatgcggctggaatgcgccatctactacaaggctagagagatgggcttcaagcacatcaaccaccaggtggtgcctacactggccgtgtctaagaacaaggctctgcaggccatcgaactgcagctgaccctggaaacaatctacaactcccagtactccaacgagaagtggaccctccaggacgtgtcactggaagtgtacctgaccgctcctaccggctgcatcaagaaacacggctacaccgtggaagtgcagtttgacggcgatatctgcaataccatgcactacaccaactggacccacatctacatctgcgaagaggccagcgtgacagtggtggaaggccaggtcgactactacggcctgtactacgtgcacgagggcatcagaacctacttcgtgcagttcaaggatgacgccgagaagtacagcaagaacaaagtgtgggaagtgcacgctggcggccaagtgatcctgtgtcctacaagcgtgttcagcagcaacgaagtgtctagccccgagatcatcagacagcacctggccaatcatcctgccgccacacacacaaaagctgtggccctgggcaccgaggaaacacagaccacaattcagaggcccagaagcgagcccgataccggcaatccttgtcacaccaccaagctgctgcaccgggattccgtggattctgcccctatcctgaccgccttcaacagctcccacaaaggccggatcaactgcaacagcaacaccacacctatcgtgcacctgaaaggggacgccaacactctgaaggccctgcggtacagattcaagaagcactgtaccctgtataccgccgtgtccagcacatggcactggaccggacacaacgtgaaacacaagagcgccatcgtgaccctgacctacgatagcgagtggcagagggaccagttcctgagccaagtgaagatccctaagaccatcaccgtgtccaccggctttatgagcattggcagcatgcaccagaaacggacagccatgttccaggatcctcaagagcggccacggaagctgcctcagctgtgtacagaactgcagaccaccatccacgacatcatcctcgagtgcgtgtactgcaagcagcagctcctgcggagagaagtgtacgacttcgccttccgggatctgtgcatcgtgtacagagatggcaacccctacgccgtgtgcgacaagtgcctgaagttctactccaagatcagcgagtaccggcactactgctactccctgtacggcaccacacttgagcagcagtacaacaagcccctgtgcgatctgctgatccggtgcatcaactgccagaaacctcagtgccccgaggaaaagcagagacacctggacaagaagcagcggttccacaacattagaggcagatggacaggccggtgcatgagctgctgtagaagcagcagaacaagacggggctccatgcacggcgataccccaacactgcacgagtacatgctggatctgcagcccgagacaaccgatctgtacggctatgagcagctgaatgactccagcgaggaagaggacgagatcgacggacctgctggacaggctgaacctgatagagcccactacaatatcgtgaccttctgctgcaagtgcgatagcaccctgagaagatgcgtgcagagcacccacgtggacatcaggaccctcgaggatctgctcatgggcaccctgggaattgtggccccaatctgctctcagaagccttgagcgcggccgcggaagaatgtgcacagtgctaaccagaaaattcaggacaagactagtgactctagatcataatcagccataccacatttgtagaggttttacttgctttaaaaaacctcccacacctccccctgaacctgaaacataaaatgaatgcaattgttgttgttaacttgtttattgcagcttataatggttacaaataaagcaatagcatcacaaatttcacaaataaagcatttttttcactgc

SEQ ID NO: 61 - DNA, 5855 bp, synthetic construct
  misc_feature 1..5855: note = HPV16 Ii-E1E2-p2a-Ii-E6E7 as carried by Adenoviral vector
  source 1..5855: mol_type = other DNA
gtgatgcggttttggcagtacatcaatgggcgtggatagcggtttgactcacggggatttccaagtctccaccccattgacgtcaatgggagtttgttttggaaccaaaatcaacgggactttccaaaatgtcgtaacaactccgccccattgacgcaaatgggcggtaggcgtgtacggtgggaggtctatataagcagagctctccctatcagtgatagagatctccctatcagtgatagagatcgtcgacgagctcgtttagtgaaccgtcagatcgcctggagacgccatccacgctgttttgacctctagcaccggtgccaccatgcacagaaggcggagcagaagctgccgcgaggatcagaaacccgtgatggacgaccagcgggacctgatcagcaacaacgagcagctgcctatgctcggcagaaggcctggcgctcctgagagcaaatgttctagaggcgccctgtacaccggcttcagcatcctggttacactgctgctggccggacaggctaccaccgcctactttctgtatcagcagcagggcagactggacaagctgaccgtgaccagccagaacctgcagctggaaaacctgcggatgaagctgcccaagcctcctaagcctgtgtccaagatgcggatggccacacctctgctgatgcaggctttgcctatgggagcactgcctcagggccctatgcagaacgccaccaaatacggcaacatgaccgaggaccacgtgatgcatctgctgcagaatgccgatcctctgaaggtgtaccctccactgaagggcagctttcccgagaacctgcggcacctgaagaacaccatggaaaccatcgactggaaggtgttcgagagctggatgcaccactggctgctgttcgagatgagccggcacagcctggaacagaagcctacagacgcccctcctaaagagtccctggaactggaagatcccagctctggcctgggcgtgacaaagcaagatctgggccctgtgcctatgatggccgatcctgctggcacaaacggcgaagaaggcacaggctgcaacggctggttttacgtggaagccgtggtggaaaagaaaaccggcgacgccatcagcgacgacgagaacgagaatgacagcgacaccggcgaggacctggtggacttcatcgtgaacgacaacgactacctgacacaggccgagacagagacagcccacgctctgtttacagcccaagaggccaagcagcacagagatgccgttcaggtgctgaagagaaagtacctgggcagccctctgagcgacatcagcggctgtgtggacaacaacatcagccccagactgaaggccatctgcatcgagaagcagagcagagccgccaagcggagactgtttgagagcgaggatagcggctacggcaataccgaggtggaaacccagcaaatgctgcaggtcgagggcagacacgaaaccgagacaccttgcagccagtacagcggaggatctggcggaggctgtagccagtactcttctggaagtggcggagagggcgtgtccgagagacacaccatttgtcagacccctctgaccaacatcctgaacgtgctgaaaaccagcaacgccaaggccgccatgctggccaagttcaaagagctgtacggcgtcagcttcagcgaactcgtgcggcccttcaagagcaacaagtccacctgttgcgactggtgcattgccgcctttggcctgacaccttctatcgccgacagcatcaagaccctgctccagcagtactgtctgtacctgcacattcagagcctggcctgctcttggggaatggtggtgctgctgctcgtgcggtacaagtgcggcaagaacagagagacaattgagaagctgctgagcaaactgctgtgcgtgtcccctatgtgcatgatgatcgagcctccaaagctgcggtctacagccgccgctctgtactggtacaagaccggcatcagcaatatctccgaggtgtacggcgacacccctgagtggattcagagacagaccgtcctgcagcacagcttcaacgactgcaccttcgagctgtcccagatggtgcagtgggcctacgacaatgacatcgtggacgactccgagatcgcctataagtacgcccagctggccgacaccaacagcaatgccagcgccttcctgaagtccaacagccaggccaagatcgtgaaggactgcgccaccatgtgcagacactacaagcgggccgagaagaaacagatgagcatgagccagtggatcaagtacagatgcgacagagtggacgacggcggcgattggaagcagatcgtgatgttcctgagataccagggcgtcgagttcatgagctttctgacagccctgaagcggttcctgcagggcatccccaagaagaactgcatcctgctgtatggcgccgctaacaccggcaagagcctgttcggaatgagcctgatgaagtttctgcagggctccgtgatctgctttgtgaacagcaagagccacttctggctccagccactggccgatgccaagattggcatgctggacgatgccaccgtgccttgctggaactacatcgacgacaacctgagaaacgccctggacggcaacctggtttctatggacgtgaagcacagacccctggtgcagctgaagtgtcctccactgctgatcaccagcaacatcaacgccggcaccgattccagatggccctatctgcacaaccggctggtggtgtttacctttcctaacgagttccccttcgacgaaaacggcaaccccgtgtacgagctgaacgataagaattggaagtccttcttcagccggacctggtccagactgagcctgcacgaggatgaggacaaagagaacgacggcgacagcctgcctaccttcaaatgtgtcagcggccagaacaccaacacactgggcagcatggaaacactgtgccagcggctgaacgtgtgccaggacaagattctgacccactatgagaacgacagcaccgacctgagggaccacatcgactactggaagcacatgcggctggaatgcgccatctactacaaggctagagagatgggcttcaagcacatcaaccaccaggtggtgcctacactggccgtgtctaagaacaaggctctgcaggccatcgaactgcagctgaccctggaaacaatctacaactcccagtactccaacgagaagtggaccctccaggacgtgtcactggaagtgtacctgaccgctcctaccggctgcatcaagaaacacggctacaccgtggaagtgcagtttgacggcgatatctgcaataccatgcactacaccaactggacccacatctacatctgcgaagaggccagcgtgacagtggtggaaggccaggtcgactactacggcctgtactacgtgcacgagggcatcagaacctacttcgtgcagttcaaggatgacgccgagaagtacagcaagaacaaagtgtgggaagtgcacgctggcggccaagtgatcctgtgtcctacaagcgtgttcagcagcaacgaagtgtctagccccgagatcatcagacagcacctggccaatcatcctgccgccacacacacaaaagctgtggccctgggcaccgaggaaacacagaccacaattcagaggcccagaagcgagcccgataccggcaatccttgtcacaccaccaagctgctgcaccgggattccgtggattctgcccctatcctgaccgccttcaacagctcccacaaaggccggatcaactgcaacagcaacaccacacctatcgtgcacctgaaaggggacgccaacactctgaaggccctgcggtacagattcaagaagcactgtaccctgtataccgccgtgtccagcacatggcactggaccggacacaacgtgaaacacaagagcgccatcgtgaccctgacctacgatagcgagtggcagagggaccagttcctgagccaagtgaagatccctaagaccatcaccgtgtccaccggctttatgagcattggcagcggcagcggcgccaccaatttcagtctgcttaaacaggccggggacgtcgaagagaaccccggacctatgcatcggcgcagatctcggtcttgtagagaagaccaaaagcctgtcatggatgatcaaagagatttaatttctaataatgaacaattacccatgttgggacggcgccccggagcccccgaatctaagtgcagccggggagctttatatacaggattttctattttagtgaccttattattagctggccaagccacaacagcttatttcttataccaacaacaaggacggttagataaattaacagtcacatctcaaaatttacaattagagaatttaagaatgaaattacctaaaccacccaaacccgtcagtaaaatgagaatggctacccccttattaatgcaagccctccccatgggcgccttaccccaaggacccatgcaaaatgctacaaagtatggaaatatgacagaagatcatgtcatgcacttattacaaaacgctgaccccttaaaagtctatccccctttaaaaggatctttccctgaaaatttaagacatttaaaaaatacaatggagacaattgattggaaagtctttgaatcttggatgcatcattggttattatttgaaatgtctaggcattctttagagcaaaaacccaccgatgctccacccaaggaaagtttagagttagaggacccttctagcggattaggagtcaccaaacaggacttaggacccgtccccatgatgcaccagaaacggacagccatgttccaggatcctcaagagcggccacggaagctgcctcagctgtgtacagaactgcagaccaccatccacgacatcatcctcgagtgcgtgtactgcaagcagcagctcctgcggagagaagtgtacgacttcgccttccgggatctgtgcatcgtgtacagagatggcaacccctacgccgtgtgcgacaagtgcctgaagttctactccaagatcagcgagtaccggcactactgctactccctgtacggcaccacacttgagcagcagtacaacaagcccctgtgcgatctgctgatccggtgcatcaactgccagaaacctcagtgccccgaggaaaagcagagacacctggacaagaagcagcggttccacaacattagaggcagatggacaggccggtgcatgagctgctgtagaagcagcagaacaagacggggctccatgcacggcgataccccaacactgcacgagtacatgctggatctgcagcccgagacaaccgatctgtacggctatgagcagctgaatgactccagcgaggaagaggacgagatcgacggacctgctggacaggctgaacctgatagagcccactacaatatcgtgaccttctgctgcaagtgcgatagcaccctgagaagatgcgtgcagagcacccacgtggacatcaggaccctcgaggatctgctcatgggcaccctgggaattgtggccccaatctgctctcagaagccttgagcggccgcggaagaatgtgcacagtgctaaccagaaaattcaggacaagactagtgactctagatcataatcagccataccacatttgtagaggttttacttgctttaaaaaacctcccacacctccccctgaacctgaaacataaaatgaatgcaattgttgttgttaacttgtttattgcagcttataatggttacaaataaagcaatagcatcacaaatttcacaaataaagcatttttttcactgc

SEQ ID NO: 62 - DNA, 4397 bp, synthetic construct
  misc_feature 1..4397: note = HPV16 E1E2E6E7 as carried by Adenoviral vector
  source 1..4397: mol_type = other DNA
gtgatgcggttttggcagtacatcaatgggcgtggatagcggtttgactcacggggatttccaagtctccaccccattgacgtcaatgggagtttgttttggaaccaaaatcaacgggactttccaaaatgtcgtaacaactccgccccattgacgcaaatgggcggtaggcgtgtacggtgggaggtctatataagcagagctctccctatcagtgatagagatctccctatcagtgatagagatcgtcgacgagctcgtttagtgaaccgtcagatcgcctggagacgccatccacgctgttttgacctctagcaccggtgccaccatggccgatcctgctggcacaaacggcgaagaaggcacaggctgcaacggctggttttacgtggaagccgtggtggaaaagaaaaccggcgacgccatcagcgacgacgagaacgagaatgacagcgacaccggcgaggacctggtggacttcatcgtgaacgacaacgactacctgacacaggccgagacagagacagcccacgctctgtttacagcccaagaggccaagcagcacagagatgccgttcaggtgctgaagagaaagtacctgggcagccctctgagcgacatcagcggctgtgtggacaacaacatcagccccagactgaaggccatctgcatcgagaagcagagcagagccgccaagcggagactgtttgagagcgaggatagcggctacggcaataccgaggtggaaacccagcaaatgctgcaggtcgagggcagacacgaaaccgagacaccttgcagccagtacagcggaggatctggcggaggctgtagccagtactcttctggaagtggcggagagggcgtgtccgagagacacaccatttgtcagacccctctgaccaacatcctgaacgtgctgaaaaccagcaacgccaaggccgccatgctggccaagttcaaagagctgtacggcgtcagcttcagcgaactcgtgcggcccttcaagagcaacaagtccacctgttgcgactggtgcattgccgcctttggcctgacaccttctatcgccgacagcatcaagaccctgctccagcagtactgtctgtacctgcacattcagagcctggcctgctcttggggaatggtggtgctgctgctcgtgcggtacaagtgcggcaagaacagagagacaattgagaagctgctgagcaaactgctgtgcgtgtcccctatgtgcatgatgatcgagcctccaaagctgcggtctacagccgccgctctgtactggtacaagaccggcatcagcaatatctccgaggtgtacggcgacacccctgagtggattcagagacagaccgtcctgcagcacagcttcaacgactgcaccttcgagctgtcccagatggtgcagtgggcctacgacaatgacatcgtggacgactccgagatcgcctataagtacgcccagctggccgacaccaacagcaatgccagcgccttcctgaagtccaacagccaggccaagatcgtgaaggactgcgccaccatgtgcagacactacaagcgggccgagaagaaacagatgagcatgagccagtggatcaagtacagatgcgacagagtggacgacggcggcgattggaagcagatcgtgatgttcctgagataccagggcgtcgagttcatgagctttctgacagccctgaagcggttcctgcagggcatccccaagaagaactgcatcctgctgtatggcgccgctaacaccggcaagagcctgttcggaatgagcctgatgaagtttctgcagggctccgtgatctgctttgtgaacagcaagagccacttctggctccagccactggccgatgccaagattggcatgctggacgatgccaccgtgccttgctggaactacatcgacgacaacctgagaaacgccctggacggcaacctggtttctatggacgtgaagcacagacccctggtgcagctgaagtgtcctccactgctgatcaccagcaacatcaacgccggcaccgattccagatggccctatctgcacaaccggctggtggtgtttacctttcctaacgagttccccttcgacgaaaacggcaaccccgtgtacgagctgaacgataagaattggaagtccttcttcagccggacctggtccagactgagcctgcacgaggatgaggacaaagagaacgacggcgacagcctgcctaccttcaaatgtgtcagcggccagaacaccaacacactgggcagcatggaaacactgtgccagcggctgaacgtgtgccaggacaagattctgacccactatgagaacgacagcaccgacctgagggaccacatcgactactggaagcacatgcggctggaatgcgccatctactacaaggctagagagatgggcttcaagcacatcaaccaccaggtggtgcctacactggccgtgtctaagaacaaggctctgcaggccatcgaactgcagctgaccctggaaacaatctacaactcccagtactccaacgagaagtggaccctccaggacgtgtcactggaagtgtacctgaccgctcctaccggctgcatcaagaaacacggctacaccgtggaagtgcagtttgacggcgatatctgcaataccatgcactacaccaactggacccacatctacatctgcgaagaggccagcgtgacagtggtggaaggccaggtcgactactacggcctgtactacgtgcacgagggcatcagaacctacttcgtgcagttcaaggatgacgccgagaagtacagcaagaacaaagtgtgggaagtgcacgctggcggccaagtgatcctgtgtcctacaagcgtgttcagcagcaacgaagtgtctagccccgagatcatcagacagcacctggccaatcatcctgccgccacacacacaaaagctgtggccctgggcaccgaggaaacacagaccacaattcagaggcccagaagcgagcccgataccggcaatccttgtcacaccaccaagctgctgcaccgggattccgtggattctgcccctatcctgaccgccttcaacagctcccacaaaggccggatcaactgcaacagcaacaccacacctatcgtgcacctgaaaggggacgccaacactctgaaggccctgcggtacagattcaagaagcactgtaccctgtataccgccgtgtccagcacatggcactggaccggacacaacgtgaaacacaagagcgccatcgtgaccctgacctacgatagcgagtggcagagggaccagttcctgagccaagtgaagatccctaagaccatcaccgtgtccaccggctttatgagcattggcagcatgcaccagaaacggacagccatgttccaggatcctcaagagcggccacggaagctgcctcagctgtgtacagaactgcagaccaccatccacgacatcatcctcgagtgcgtgtactgcaagcagcagctcctgcggagagaagtgtacgacttcgccttccgggatctgtgcatcgtgtacagagatggcaacccctacgccgtgtgcgacaagtgcctgaagttctactccaagatcagcgagtaccggcactactgctactccctgtacggcaccacacttgagcagcagtacaacaagcccctgtgcgatctgctgatccggtgcatcaactgccagaaacctcagtgccccgaggaaaagcagagacacctggacaagaagcagcggttccacaacattagaggcagatggacaggccggtgcatgagctgctgtagaagcagcagaacaagacggggctccatgcacggcgataccccaacactgcacgagtacatgctggatctgcagcccgagacaaccgatctgtacggctatgagcagctgaatgactccagcgaggaagaggacgagatcgacggacctgctggacaggctgaacctgatagagcccactacaatatcgtgaccttctgctgcaagtgcgatagcaccctgagaagatgcgtgcagagcacccacgtggacatcaggaccctcgaggatctgctcatgggcaccctgggaattgtggccccaatctgctctcagaagccttgagcggccgcggaagaatgtgcacagtgctaaccagaaaattcaggacaagactagtgactctagatcataatcagccataccacatttgtagaggttttacttgctttaaaaaacctcccacacctccccctgaacctgaaacataaaatgaatgcaattgttgttgttaacttgtttattgcagcttataatggttacaaataaagcaatagcatcacaaatttcacaaataaagcatttttttcactgc

SEQ ID NO: 63 - DNA, 5849 bp, synthetic construct
  misc_feature 1..5849: note = HPV16 Ii-E1E6E7-p2a-Ii-E2 as carried by Adenoviral vector
  source 1..5849: mol_type = other DNA
gtgatgcggttttggcagtacatcaatgggcgtggatagcggtttgactcacggggatttccaagtctccaccccattgacgtcaatgggagtttgttttggaaccaaaatcaacgggactttccaaaatgtcgtaacaactccgccccattgacgcaaatgggcggtaggcgtgtacggtgggaggtctatataagcagagctctccctatcagtgatagagatctccctatcagtgatagagatcgtcgacgagctcgtttagtgaaccgtcagatcgcctggagacgccatccacgctgttttgacctctagcaccggtgccaccatgcacagaaggcggagcagaagctgccgcgaggatcagaaacccgtgatggacgaccagcgggacctgatcagcaacaacgagcagctgcctatgctcggcagaaggcctggcgctcctgagagcaaatgttctagaggcgccctgtacaccggcttcagcatcctggttacactgctgctggccggacaggctaccaccgcctactttctgtatcagcagcagggcagactggacaagctgaccgtgaccagccagaacctgcagctggaaaacctgcggatgaagctgcccaagcctcctaagcctgtgtccaagatgcggatggccacacctctgctgatgcaggctttgcctatgggagcactgcctcagggccctatgcagaacgccaccaaatacggcaacatgaccgaggaccacgtgatgcatctgctgcagaatgccgatcctctgaaggtgtaccctccactgaagggcagctttcccgagaacctgcggcacctgaagaacaccatggaaaccatcgactggaaggtgttcgagagctggatgcaccactggctgctgttcgagatgagccggcacagcctggaacagaagcctacagacgcccctcctaaagagtccctggaactggaagatcccagctctggcctgggcgtgacaaagcaagatctgggccctgtgcctatgatggccgatcctgctggcacaaacggcgaagaaggcacaggctgcaacggctggttttacgtggaagccgtggtggaaaagaaaaccggcgacgccatcagcgacgacgagaacgagaatgacagcgacaccggcgaggacctggtggacttcatcgtgaacgacaacgactacctgacacaggccgagacagagacagcccacgctctgtttacagcccaagaggccaagcagcacagagatgccgttcaggtgctgaagagaaagtacctgggcagccctctgagcgacatcagcggctgtgtggacaacaacatcagccccagactgaaggccatctgcatcgagaagcagagcagagccgccaagcggagactgtttgagagcgaggatagcggctacggcaataccgaggtggaaacccagcaaatgctgcaggtcgagggcagacacgaaaccgagacaccttgcagccagtacagcggaggatctggcggaggctgtagccagtactcttctggaagtggcggagagggcgtgtccgagagacacaccatttgtcagacccctctgaccaacatcctgaacgtgctgaaaaccagcaacgccaaggccgccatgctggccaagttcaaagagctgtacggcgtcagcttcagcgaactcgtgcggcccttcaagagcaacaagtccacctgttgcgactggtgcattgccgcctttggcctgacaccttctatcgccgacagcatcaagaccctgctccagcagtactgtctgtacctgcacattcagagcctggcctgctcttggggaatggtggtgctgctgctcgtgcggtacaagtgcggcaagaacagagagacaattgagaagctgctgagcaaactgctgtgcgtgtcccctatgtgcatgatgatcgagcctccaaagctgcggtctacagccgccgctctgtactggtacaagaccggcatcagcaatatctccgaggtgtacggcgacacccctgagtggattcagagacagaccgtcctgcagcacagcttcaacgactgcaccttcgagctgtcccagatggtgcagtgggcctacgacaatgacatcgtggacgactccgagatcgcctataagtacgcccagctggccgacaccaacagcaatgccagcgccttcctgaagtccaacagccaggccaagatcgtgaaggactgcgccaccatgtgcagacactacaagcgggccgagaagaaacagatgagcatgagccagtggatcaagtacagatgcgacagagtggacgacggcggcgattggaagcagatcgtgatgttcctgagataccagggcgtcgagttcatgagctttctgacagccctgaagcggttcctgcagggcatccccaagaagaactgcatcctgctgtatggcgccgctaacaccggcaagagcctgttcggaatgagcctgatgaagtttctgcagggctccgtgatctgctttgtgaacagcaagagccacttctggctccagccactggccgatgccaagattggcatgctggacgatgccaccgtgccttgctggaactacatcgacgacaacctgagaaacgccctggacggcaacctggtttctatggacgtgaagcacagacccctggtgcagctgaagtgtcctccactgctgatcaccagcaacatcaacgccggcaccgattccagatggccctatctgcacaaccggctggtggtgtttacctttcctaacgagttccccttcgacgaaaacggcaaccccgtgtacgagctgaacgataagaattggaagtccttcttcagccggacctggtccagactgagcctgcacgaggatgaggacaaagagaacgacggcgacagcctgcctaccttcaaatgtgtcagcggccagaacaccaacacactgggcagcatgcaccagaaacggacagccatgttccaggatcctcaagagcggccacggaagctgcctcagctgtgtacagaactgcagaccaccatccacgacatcatcctcgagtgcgtgtactgcaagcagcagctcctgcggagagaagtgtacgacttcgccttccgggatctgtgcatcgtgtacagagatggcaacccctacgccgtgtgcgacaagtgcctgaagttctactccaagatcagcgagtaccggcactactgctactccctgtacggcaccacacttgagcagcagtacaacaagcccctgtgcgatctgctgatccggtgcatcaactgccagaaacctcagtgccccgaggaaaagcagagacacctggacaagaagcagcggttccacaacattagaggcagatggacaggccggtgcatgagctgctgtagaagcagcagaacaagacggggctccatgcacggcgataccccaacactgcacgagtacatgctggatctgcagcccgagacaaccgatctgtacggctatgagcagctgaatgactccagcgaggaagaggacgagatcgacggacctgctggacaggctgaacctgatagagcccactacaatatcgtgaccttctgctgcaagtgcgatagcaccctgagaagatgcgtgcagagcacccacgtggacatcaggaccctcgaggatctgctcatgggcaccctgggaattgtggccccaatctgctctcagaagcctggcagcggcgccaccaatttcagtctgcttaaacaggccggggacgtcgaagagaaccccggacctatgcatcggcgcagatctcggtcttgtagagaagaccaaaagcctgtcatggatgatcaaagagatttaatttctaataatgaacaattacccatgttgggacggcgccccggagcccccgaatctaagtgcagccggggagctttatatacaggattttctattttagtgaccttattattagctggccaagccacaacagcttatttcttataccaacaacaaggacggttagataaattaacagtcacatctcaaaatttacaattagagaatttaagaatgaaattacctaaaccacccaaacccgtcagtaaaatgagaatggctacccccttattaatgcaagccctccccatgggcgccttaccccaaggacccatgcaaaatgctacaaagtatggaaatatgacagaagatcatgtcatgcacttattacaaaacgctgaccccttaaaagtctatccccctttaaaaggatctttccctgaaaatttaagacatttaaaaaatacaatggagacaattgattggaaagtctttgaatcttggatgcatcattggttattatttgaaatgtctaggcattctttagagcaaaaacccaccgatgctccacccaaggaaagtttagagttagaggacccttctagcggattaggagtcaccaaacaggacttaggacccgtccccatgatggaaacactgtgccagcggctgaacgtgtgccaggacaagattctgacccactatgagaacgacagcaccgacctgagggaccacatcgactactggaagcacatgcggctggaatgcgccatctactacaaggctagagagatgggcttcaagcacatcaaccaccaggtggtgcctacactggccgtgtctaagaacaaggctctgcaggccatcgaactgcagctgaccctggaaacaatctacaactcccagtactccaacgagaagtggaccctccaggacgtgtcactggaagtgtacctgaccgctcctaccggctgcatcaagaaacacggctacaccgtggaagtgcagtttgacggcgatatctgcaataccatgcactacaccaactggacccacatctacatctgcgaagaggccagcgtgacagtggtggaaggccaggtcgactactacggcctgtactacgtgcacgagggcatcagaacctacttcgtgcagttcaaggatgacgccgagaagtacagcaagaacaaagtgtgggaagtgcacgctggcggccaagtgatcctgtgtcctacaagcgtgttcagcagcaacgaagtgtctagccccgagatcatcagacagcacctggccaatcatcctgccgccacacacacaaaagctgtggccctgggcaccgaggaaacacagaccacaattcagaggcccagaagcgagcccgataccggcaatccttgtcacaccaccaagctgctgcaccgggattccgtggattctgcccctatcctgaccgccttcaacagctcccacaaaggccggatcaactgcaacagcaacaccacacctatcgtgcacctgaaaggggacgccaacactctgaaggccctgcggtacagattcaagaagcactgtaccctgtataccgccgtgtccagcacatggcactggaccggacacaacgtgaaacacaagagcgccatcgtgaccctgacctacgatagcgagtggcagagggaccagttcctgagccaagtgaagatccctaagaccatcaccgtgtccaccggctttatgagcatttgagcggccgcggaagaatgtgcacagtgctaaccagaaaattcaggacaagactagtgactctagatcataatcagccataccacatttgtagaggttttacttgctttaaaaaacctcccacacctccccctgaacctgaaacataaaatgaatgcaattgttgttgttaacttgtttattgcagcttataatggttacaaataaagcaatagcatcacaaatttcacaaataaagcatttttttcactgc

SEQ ID NO: 64 - DNA, 3992 bp, synthetic construct
  misc_feature 1..3992: note = HPV16 Ii-E1E6E7 as carried by Adenoviral vector
  source 1..3992: mol_type = other DNA
gtgatgcggttttggcagtacatcaatgggcgtggatagcggtttgactcacggggatttccaagtctccaccccattgacgtcaatgggagtttgttttggaaccaaaatcaacgggactttccaaaatgtcgtaacaactccgccccattgacgcaaatgggcggtaggcgtgtacggtgggaggtctatataagcagagctctccctatcagtgatagagatctccctatcagtgatagagatcgtcgacgagctcgtttagtgaaccgtcagatcgcctggagacgccatccacgctgttttgacctctagcaccggtgccaccatgcacagaaggcggagcagaagctgccgcgaggatcagaaacccgtgatggacgaccagcgggacctgatcagcaacaacgagcagctgcctatgctcggcagaaggcctggcgctcctgagagcaaatgttctagaggcgccctgtacaccggcttcagcatcctggttacactgctgctggccggacaggctaccaccgcctactttctgtatcagcagcagggcagactggacaagctgaccgtgaccagccagaacctgcagctggaaaacctgcggatgaagctgcccaagcctcctaagcctgtgtccaagatgcggatggccacacctctgctgatgcaggctttgcctatgggagcactgcctcagggccctatgcagaacgccaccaaatacggcaacatgaccgaggaccacgtgatgcatctgctgcagaatgccgatcctctgaaggtgtaccctccactgaagggcagctttcccgagaacctgcggcacctgaagaacaccatggaaaccatcgactggaaggtgttcgagagctggatgcaccactggctgctgttcgagatgagccggcacagcctggaacagaagcctacagacgcccctcctaaagagtccctggaactggaagatcccagctctggcctgggcgtgacaaagcaagatctgggccctgtgcctatgatggccgatcctgctggcacaaacggcgaagaaggcacaggctgcaacggctggttttacgtggaagccgtggtggaaaagaaaaccggcgacgccatcagcgacgacgagaacgagaatgacagcgacaccggcgaggacctggtggacttcatcgtgaacgacaacgactacctgacacaggccgagacagagacagcccacgctctgtttacagcccaagaggccaagcagcacagagatgccgttcaggtgctgaagagaaagtacctgggcagccctctgagcgacatcagcggctgtgtggacaacaacatcagccccagactgaaggccatctgcatcgagaagcagagcagagccgccaagcggagactgtttgagagcgaggatagcggctacggcaataccgaggtggaaacccagcaaatgctgcaggtcgagggcagacacgaaaccgagacaccttgcagccagtacagcggaggatctggcggaggctgtagccagtactcttctggaagtggcggagagggcgtgtccgagagacacaccatttgtcagacccctctgaccaacatcctgaacgtgctgaaaaccagcaacgccaaggccgccatgctggccaagttcaaagagctgtacggcgtcagcttcagcgaactcgtgcggcccttcaagagcaacaagtccacctgttgcgactggtgcattgccgcctttggcctgacaccttctatcgccgacagcatcaagaccctgctccagcagtactgtctgtacctgcacattcagagcctggcctgctcttggggaatggtggtgctgctgctcgtgcggtacaagtgcggcaagaacagagagacaattgagaagctgctgagcaaactgctgtgcgtgtcccctatgtgcatgatgatcgagcctccaaagctgcggtctacagccgccgctctgtactggtacaagaccggcatcagcaatatctccgaggtgtacggcgacacccctgagtggattcagagacagaccgtcctgcagcacagcttcaacgactgcaccttcgagctgtcccagatggtgcagtgggcctacgacaatgacatcgtggacgactccgagatcgcctataagtacgcccagctggccgacaccaacagcaatgccagcgccttcctgaagtccaacagccaggccaagatcgtgaaggactgcgccaccatgtgcagacactacaagcgggccgagaagaaacagatgagcatgagccagtggatcaagtacagatgcgacagagtggacgacggcggcgattggaagcagatcgtgatgttcctgagataccagggcgtcgagttcatgagctttctgacagccctgaagcggttcctgcagggcatccccaagaagaactgcatcctgctgtatggcgccgctaacaccggcaagagcctgttcggaatgagcctgatgaagtttctgcagggctccgtgatctgctttgtgaacagcaagagccacttctggctccagccactggccgatgccaagattggcatgctggacgatgccaccgtgccttgctggaactacatcgacgacaacctgagaaacgccctggacggcaacctggtttctatggacgtgaagcacagacccctggtgcagctgaagtgtcctccactgctgatcaccagcaacatcaacgccggcaccgattccagatggccctatctgcacaaccggctggtggtgtttacctttcctaacgagttccccttcgacgaaaacggcaaccccgtgtacgagctgaacgataagaattggaagtccttcttcagccggacctggtccagactgagcctgcacgaggatgaggacaaagagaacgacggcgacagcctgcctaccttcaaatgtgtcagcggccagaacaccaacacactgggcagcatgcaccagaaacggacagccatgttccaggatcctcaagagcggccacggaagctgcctcagctgtgtacagaactgcagaccaccatccacgacatcatcctcgagtgcgtgtactgcaagcagcagctcctgcggagagaagtgtacgacttcgccttccgggatctgtgcatcgtgtacagagatggcaacccctacgccgtgtgcgacaagtgcctgaagttctactccaagatcagcgagtaccggcactactgctactccctgtacggcaccacacttgagcagcagtacaacaagcccctgtgcgatctgctgatccggtgcatcaactgccagaaacctcagtgccccgaggaaaagcagagacacctggacaagaagcagcggttccacaacattagaggcagatggacaggccggtgcatgagctgctgtagaagcagcagaacaagacggggctccatgcacggcgataccccaacactgcacgagtacatgctggatctgcagcccgagacaaccgatctgtacggctatgagcagctgaatgactccagcgaggaagaggacgagatcgacggacctgctggacaggctgaacctgatagagcccactacaatatcgtgaccttctgctgcaagtgcgatagcaccctgagaagatgcgtgcagagcacccacgtggacatcaggaccctcgaggatctgctcatgggcaccctgggaattgtggccccaatctgctctcagaagccttgagcggccgcggaagaatgtgcacagtgctaaccagaaaattcaggacaagactagtgactctagatcataatcagccataccacatttgtagaggttttacttgctttaaaaaacctcccacacctccccctgaacctgaaacataaaatgaatgcaattgttgttgttaacttgtttattgcagcttataatggttacaaataaagcaatagcatcacaaatttcacaaataaagcatttttttcactgc

SEQ ID NO: 65 - DNA, 5138 bp, synthetic construct
  misc_feature 1..5138: note = HPV18 Ii-E1E2E6E7 as carried by Adenoviral vector
  source 1..5138: mol_type = other DNA
gtgatgcggttttggcagtacatcaatgggcgtggatagcggtttgactcacggggatttccaagtctccaccccattgacgtcaatgggagtttgttttggaaccaaaatcaacgggactttccaaaatgtcgtaacaactccgccccattgacgcaaatgggcggtaggcgtgtacggtgggaggtctatataagcagagctctccctatcagtgatagagatctccctatcagtgatagagatcgtcgacgagctcgtttagtgaaccgtcagatcgcctggagacgccatccacgctgttttgacctctagcaccggtgccaccatgcacagaaggcggagcagaagctgccgcgaggatcagaaacccgtgatggacgaccagcgggacctgatcagcaacaacgagcagctgcctatgctcggcagaaggcctggcgctcctgagagcaaatgttctagaggcgccctgtacaccggcttcagcatcctggttacactgctgctggccggacaggctaccaccgcctactttctgtatcagcagcagggcagactggacaagctgaccgtgaccagccagaacctgcagctggaaaacctgcggatgaagctgcccaagcctcctaagcctgtgtccaagatgcggatggccacacctctgctgatgcaggctttgcctatgggagcactgcctcagggccctatgcagaacgccaccaaatacggcaacatgaccgaggaccacgtgatgcatctgctgcagaatgccgatcctctgaaggtgtaccctccactgaagggcagctttcccgagaacctgcggcacctgaagaacaccatggaaaccatcgactggaaggtgttcgagagctggatgcaccactggctgctgttcgagatgagccggcacagcctggaacagaagcctacagacgcccctcctaaagagtccctggaactggaagatcccagctctggcctgggcgtgacaaagcaagatctgggccctgtgcctatgatggccgatcctgagggaacagatggcgaaggcacaggctgcaacggctggttttacgtgcaagccatcgtggacaagaagaccggcgacgtcatcagcgacgacgaggatgagaatgccaccgacaccggcagcgacatggtggactttatcgatacccagggcacgttctgcgagcaggccgaactggaaacagcccaggcactgtttcacgcccaagaggtgcacaacgatgcccaggtgctgcacgtgctgaagagaaagtttgccggcggaagcaccgagaacagcccactgggagaaagactggaagtggacaccgagctgagccccagactgcaagagatcagcctgaacagcggccagaagaaggccaagcggagactgttcaccatctccgacagcggctacggctgtagcgaagtggaagccacacagatccaagtgaccaccaatggcgagcacggcggcaatgtttgttctggcggatctaccgaggccatcgacaatggcggaaccgagggcaacaatagctccgtggatggcaccagcgacaacagcaacatcgagaacgtgaaccctcagtgcacaatcgcccagctgaaggacctgctgaaagtgaacaacaagcagggcgccatgctggccgtgtttaaggatacctacggcctgagcttcaccgacctcgtgcggaacttcaagagcgacaagaccacctgtaccgactgggtcaccgccatctttggcgtgaacccaacaatcgccgagggcttcaagaccctgatccagcctttcatcctgtacgcccacatccagtgtctggactgcaaatggggcgtgctgattctggccctgctgagatacaagtgcggcaagagcagactgaccgtggccaagggactgagcactctgctgcatgtgcccgagacatgcatgctgattcagcctccaaagctgcggtctagcgtggccgctctgtactggtacagaaccggcatcagcaatatcagcgaagtgatgggcgacacccctgagtggattcagcggctgacaatcatccagcacggcatcgacgactccaacttcgacctgagcgagatggtgcagtgggccttcgataacgagctgaccgacgagtccgacatggcctttgaatacgccctgctggcagacagcaactccaatgccgccgctttcctgaagtccaactgccaggccaagtacctgaaggactgcgccaccatgtgcaagcactaccgcagagcacagaaacggcagatgaacatgagccagtggatccggttccggtgcagcaagatcgatgaaggtggcgactggcggcctatcgtgcagttcctgagataccagcagatcgagttcatcacctttctgggcgccctgaagtcattcctgaagggaacccctaaaaagaactgcctggtgttctgcggccctgccaataccggcaagtcctacttcggcatgagctttatccacttcatccagggcgccgtgatcagcttcgtgaacagcaccagccacttttggctggaacccctgaccgataccaaggtggcaatgctggacgacgccaccaccacctgttggacctacttcgacacctacatgcggaacgccctggacggcaaccccatcagcatcgacagaaagcacaagcccctcatccagctgaaatgccctccaatcctgctgaccacaaacattcaccccgccaaggacaatcggtggccctacctggaaagccggatcaccgtgttcgagttccccaacgcctttccattcgacaagaacggcaatcccgtgtacgagatcaacgacaagaattggaagtgcttcttcgagcggacctggtccagactggatctgcacgaggaagaagaggacgccgacacagagggcaatcccttcggcacctttaagtgtgtggccgggcagaatcacagacccctgggatctatgcagaccccaaaagagacactgagcgagcggctgagcgccctccaggacaagatcatcgaccactacgagaacgacagcaaggacatcgacagccagattcagtactggcagctgatcagatgggagaacgccatcttcttcgccgctagagagcacggaattcagaccctgaaccaccaggtggtgcccgcctacaacatcagcaagagcaaggcccacaaggccattgaactccagatggccctgcaaggactggctcagagcgcctacaagaccgaggattggacactccaggatacctgcgaggaactgtggaacacagagcccacacactgcttcaagaaaggcggccagaccgtgcaggtctacttcgatggcaacaaggataactgcatgacctacgtggcctgggacagcgtgtactacatgacagatgccggcacctgggacaagaccgccacctgtgtttctcaccggggcctgtactacgtgaaagagggctacaacaccttctacatcgagtttaagagcgagtgcgagaagtatggcaacaccggcacatgggaagtgcatttcggcaacaacgtgatcgactgcaacgactccatgtgctccacctccgatgacaccgtgtctgccacacagctggtcaagcagctccagcacaccccatctccatacagctctacagtgtctgtgggcaccgccaagacctatggccaaacaagcgctgccacaagacctggccattgtggactggccgagaagcagcactgtggccctgttaatcctctgctgggagccgccacacctacaggcaacaacaagagaagaaagctgtgcagcggcaacaccacacctatcattcacctgaagggcgacagaaacagcctgaagtgcctgcggtacagactgaggaagcacagcgatcactaccgggacatcagctccacctggcactggacaggtgctggcaacgaaaagaccggcatcctgacagtgacctaccacagcgagactcagcggaccaagttcctgaacaccgtggctatccccgacagcgtgcagattctcgtgggctacatgaccatgggcagcatggccagattcgaggaccctaccagacggccctacaagctgcctgatctgtgcaccgaactgaacaccagccttcaggatatcgagatcacctgtgtgtactgcaagaccgtgctcgagctgacagaagtgtttgagttcgccttcaaggacctgttcgtggtgtacagagacagcatccctcacgccgcctgtcacaagtgcatcgacttctacagcagaatcagagagctgcggcactacagcgactccgtgtacggcgataccctggaaaaactgaccaacaccggactgtacaacctgctgatccggtgcctgagatgccagaaacctcagaaccccgccgagaaactgagacatctgaacgagaagcggcggttccacaatatcgccggccactacagaggccagtgccacagctgttgcaaccgggccagacaagaaagactccagcggagaagaggcagcatgcacggacctaaagccacacttcaggacatcgtgctgcacctcgagcctcagaatgagatccccgtggatctgctcggccatgagcagctgagcgatagcgaggaagagaacgacgagatcgacggcgtcaaccaccagcatctgcctgccagaagggccgaacctcagagacacaccatgctgtgcatgtgctgcaagtgcgaggcccggatcgagagagtggtggaatctagcgccgacgacctgagagctttccagcagctgttcctgaatactctgagcttcgtggccccttggtgcgctagtcagcagtgagcggccgcggaagaatgtgcacagtgctaaccagaaaattcaggacaagactagtgactctagatcataatcagccataccacatttgtagaggttttacttgctttaaaaaacctcccacacctccccctgaacctgaaacataaaatgaatgcaattgttgttgttaacttgtttattgcagcttataatggttacaaataaagcaatagcatcacaaatttcacaaataaagcatttttttcactgc

SEQ ID NO: 66 - DNA, 5894 bp, synthetic construct
  misc_feature 1..5894: note = HPV18 Ii-E1E2-p2a-Ii-E6E7 as carried by Adenoviral vector
  source 1..5894: mol_type = other DNA
gtgatgcggttttggcagtacatcaatgggcgtggatagcggtttgactcacggggatttccaagtctccaccccattgacgtcaatgggagtttgttttggaaccaaaatcaacgggactttccaaaatgtcgtaacaactccgccccattgacgcaaatgggcggtaggcgtgtacggtgggaggtctatataagcagagctctccctatcagtgatagagatctccctatcagtgatagagatcgtcgacgagctcgtttagtgaaccgtcagatcgcctggagacgccatccacgctgttttgacctctagcaccggtgccaccatgcacagaaggcggagcagaagctgccgcgaggatcagaaacccgtgatggacgaccagcgggacctgatcagcaacaacgagcagctgcctatgctcggcagaaggcctggcgctcctgagagcaaatgttctagaggcgccctgtacaccggcttcagcatcctggttacactgctgctggccggacaggctaccaccgcctactttctgtatcagcagcagggcagactggacaagctgaccgtgaccagccagaacctgcagctggaaaacctgcggatgaagctgcccaagcctcctaagcctgtgtccaagatgcggatggccacacctctgctgatgcaggctttgcctatgggagcactgcctcagggccctatgcagaacgccaccaaatacggcaacatgaccgaggaccacgtgatgcatctgctgcagaatgccgatcctctgaaggtgtaccctccactgaagggcagctttcccgagaacctgcggcacctgaagaacaccatggaaaccatcgactggaaggtgttcgagagctggatgcaccactggctgctgttcgagatgagccggcacagcctggaacagaagcctacagacgcccctcctaaagagtccctggaactggaagatcccagctctggcctgggcgtgacaaagcaagatctgggccctgtgcctatgatggccgatcctgagggaacagatggcgaaggcacaggctgcaacggctggttttacgtgcaagccatcgtggacaagaagaccggcgacgtcatcagcgacgacgaggatgagaatgccaccgacaccggcagcgacatggtggactttatcgatacccagggcacgttctgcgagcaggccgaactggaaacagcccaggcactgtttcacgcccaagaggtgcacaacgatgcccaggtgctgcacgtgctgaagagaaagtttgccggcggaagcaccgagaacagcccactgggagaaagactggaagtggacaccgagctgagccccagactgcaagagatcagcctgaacagcggccagaagaaggccaagcggagactgttcaccatctccgacagcggctacggctgtagcgaagtggaagccacacagatccaagtgaccaccaatggcgagcacggcggcaatgtttgttctggcggatctaccgaggccatcgacaatggcggaaccgagggcaacaatagctccgtggatggcaccagcgacaacagcaacatcgagaacgtgaaccctcagtgcacaatcgcccagctgaaggacctgctgaaagtgaacaacaagcagggcgccatgctggccgtgtttaaggatacctacggcctgagcttcaccgacctcgtgcggaacttcaagagcgacaagaccacctgtaccgactgggtcaccgccatctttggcgtgaacccaacaatcgccgagggcttcaagaccctgatccagcctttcatcctgtacgcccacatccagtgtctggactgcaaatggggcgtgctgattctggccctgctgagatacaagtgcggcaagagcagactgaccgtggccaagggactgagcactctgctgcatgtgcccgagacatgcatgctgattcagcctccaaagctgcggtctagcgtggccgctctgtactggtacagaaccggcatcagcaatatcagcgaagtgatgggcgacacccctgagtggattcagcggctgacaatcatccagcacggcatcgacgactccaacttcgacctgagcgagatggtgcagtgggccttcgataacgagctgaccgacgagtccgacatggcctttgaatacgccctgctggcagacagcaactccaatgccgccgctttcctgaagtccaactgccaggccaagtacctgaaggactgcgccaccatgtgcaagcactaccgcagagcacagaaacggcagatgaacatgagccagtggatccggttccggtgcagcaagatcgatgaaggtggcgactggcggcctatcgtgcagttcctgagataccagcagatcgagttcatcacctttctgggcgccctgaagtcattcctgaagggaacccctaaaaagaactgcctggtgttctgcggccctgccaataccggcaagtcctacttcggcatgagctttatccacttcatccagggcgccgtgatcagcttcgtgaacagcaccagccacttttggctggaacccctgaccgataccaaggtggcaatgctggacgacgccaccaccacctgttggacctacttcgacacctacatgcggaacgccctggacggcaaccccatcagcatcgacagaaagcacaagcccctcatccagctgaaatgccctccaatcctgctgaccacaaacattcaccccgccaaggacaatcggtggccctacctggaaagccggatcaccgtgttcgagttccccaacgcctttccattcgacaagaacggcaatcccgtgtacgagatcaacgacaagaattggaagtgcttcttcgagcggacctggtccagactggatctgcacgaggaagaagaggacgccgacacagagggcaatcccttcggcacctttaagtgtgtggccgggcagaatcacagacccctgggatctatgcagaccccaaaagagacactgagcgagcggctgagcgccctccaggacaagatcatcgaccactacgagaacgacagcaaggacatcgacagccagattcagtactggcagctgatcagatgggagaacgccatcttcttcgccgctagagagcacggaattcagaccctgaaccaccaggtggtgcccgcctacaacatcagcaagagcaaggcccacaaggccattgaactccagatggccctgcaaggactggctcagagcgcctacaagaccgaggattggacactccaggatacctgcgaggaactgtggaacacagagcccacacactgcttcaagaaaggcggccagaccgtgcaggtctacttcgatggcaacaaggataactgcatgacctacgtggcctgggacagcgtgtactacatgacagatgccggcacctgggacaagaccgccacctgtgtttctcaccggggcctgtactacgtgaaagagggctacaacaccttctacatcgagtttaagagcgagtgcgagaagtatggcaacaccggcacatgggaagtgcatttcggcaacaacgtgatcgactgcaacgactccatgtgctccacctccgatgacaccgtgtctgccacacagctggtcaagcagctccagcacaccccatctccatacagctctacagtgtctgtgggcaccgccaagacctatggccaaacaagcgctgccacaagacctggccattgtggactggccgagaagcagcactgtggccctgttaatcctctgctgggagccgccacacctacaggcaacaacaagagaagaaagctgtgcagcggcaacaccacacctatcattcacctgaagggcgacagaaacagcctgaagtgcctgcggtacagactgaggaagcacagcgatcactaccgggacatcagctccacctggcactggacaggtgctggcaacgaaaagaccggcatcctgacagtgacctaccacagcgagactcagcggaccaagttcctgaacaccgtggctatccccgacagcgtgcagattctcgtgggctacatgaccatgggcagcggcgccaccaatttcagtctgcttaaacaggccggggacgtcgaagagaaccccggacctatgcatcggcgcagatctcggtcttgtagagaagaccaaaagcctgtcatggatgatcaaagagatttaatttctaataatgaacaattacccatgttgggacggcgccccggagcccccgaatctaagtgcagccggggagctttatatacaggattttctattttagtgaccttattattagctggccaagccacaacagcttatttcttataccaacaacaaggacggttagataaattaacagtcacatctcaaaatttacaattagagaatttaagaatgaaattacctaaaccacccaaacccgtcagtaaaatgagaatggctacccccttattaatgcaagccctccccatgggcgccttaccccaaggacccatgcaaaatgctacaaagtatggaaatatgacagaagatcatgtcatgcacttattacaaaacgctgaccccttaaaagtctatccccctttaaaaggatctttccctgaaaatttaagacatttaaaaaatacaatggagacaattgattggaaagtctttgaatcttggatgcatcattggttattatttgaaatgtctaggcattctttagagcaaaaacccaccgatgctccacccaaggaaagtttagagttagaggacccttctagcggattaggagtcaccaaacaggacttaggacccgtccccatgatggccagattcgaggaccctaccagacggccctacaagctgcctgatctgtgcaccgaactgaacaccagccttcaggatatcgagatcacctgtgtgtactgcaagaccgtgctcgagctgacagaagtgtttgagttcgccttcaaggacctgttcgtggtgtacagagacagcatccctcacgccgcctgtcacaagtgcatcgacttctacagcagaatcagagagctgcggcactacagcgactccgtgtacggcgataccctggaaaaactgaccaacaccggactgtacaacctgctgatccggtgcctgagatgccagaaacctcagaaccccgccgagaaactgagacatctgaacgagaagcggcggttccacaatatcgccggccactacagaggccagtgccacagctgttgcaaccgggccagacaagaaagactccagcggagaagaggcagcatgcacggacctaaagccacacttcaggacatcgtgctgcacctcgagcctcagaatgagatccccgtggatctgctcggccatgagcagctgagcgatagcgaggaagagaacgacgagatcgacggcgtcaaccaccagcatctgcctgccagaagggccgaacctcagagacacaccatgctgtgcatgtgctgcaagtgcgaggcccggatcgagagagtggtggaatctagcgccgacgacctgagagctttccagcagctgttcctgaatactctgagcttcgtggccccttggtgcgctagtcagcagtgagcggccgcggaagaatgtgcacagtgctaaccagaaaattcaggacaagactagtgactctagatcataatcagccataccacatttgtagaggttttacttgctttaaaaaacctcccacacctccccctgaacctgaaacataaaatgaatgcaattgttgttgttaacttgtttattgcagcttataatggttacaaataaagcaatagcatcacaaatttcacaaataaagcatttttttcactgc

SEQ ID NO: 67 - DNA, 4442 bp, synthetic construct
  misc_feature 1..4442: note = HPV18 E1E2E6E7 as carried by Adenoviral vector
  source 1..4442: mol_type = other DNA
gtgatgcggttttggcagtacatcaatgggcgtggatagcggtttgactcacggggatttccaagtctccaccccattgacgtcaatgggagtttgttttggaaccaaaatcaacgggactttccaaaatgtcgtaacaactccgccccattgacgcaaatgggcggtaggcgtgtacggtgggaggtctatataagcagagctctccctatcagtgatagagatctccctatcagtgatagagatcgtcgacgagctcgtttagtgaaccgtcagatcgcctggagacgccatccacgctgttttgacctctagcaccggtgccaccatggccgatcctgagggaacagatggcgaaggcacaggctgcaacggctggttttacgtgcaagccatcgtggacaagaagaccggcgacgtcatcagcgacgacgaggatgagaatgccaccgacaccggcagcgacatggtggactttatcgatacccagggcacgttctgcgagcaggccgaactggaaacagcccaggcactgtttcacgcccaagaggtgcacaacgatgcccaggtgctgcacgtgctgaagagaaagtttgccggcggaagcaccgagaacagcccactgggagaaagactggaagtggacaccgagctgagccccagactgcaagagatcagcctgaacagcggccagaagaaggccaagcggagactgttcaccatctccgacagcggctacggctgtagcgaagtggaagccacacagatccaagtgaccaccaatggcgagcacggcggcaatgtttgttctggcggatctaccgaggccatcgacaatggcggaaccgagggcaacaatagctccgtggatggcaccagcgacaacagcaacatcgagaacgtgaaccctcagtgcacaatcgcccagctgaaggacctgctgaaagtgaacaacaagcagggcgccatgctggccgtgtttaaggatacctacggcctgagcttcaccgacctcgtgcggaacttcaagagcgacaagaccacctgtaccgactgggtcaccgccatctttggcgtgaacccaacaatcgccgagggcttcaagaccctgatccagcctttcatcctgtacgcccacatccagtgtctggactgcaaatggggcgtgctgattctggccctgctgagatacaagtgcggcaagagcagactgaccgtggccaagggactgagcactctgctgcatgtgcccgagacatgcatgctgattcagcctccaaagctgcggtctagcgtggccgctctgtactggtacagaaccggcatcagcaatatcagcgaagtgatgggcgacacccctgagtggattcagcggctgacaatcatccagcacggcatcgacgactccaacttcgacctgagcgagatggtgcagtgggccttcgataacgagctgaccgacgagtccgacatggcctttgaatacgccctgctggcagacagcaactccaatgccgccgctttcctgaagtccaactgccaggccaagtacctgaaggactgcgccaccatgtgcaagcactaccgcagagcacagaaacggcagatgaacatgagccagtggatccggttccggtgcagcaagatcgatgaaggtggcgactggcggcctatcgtgcagttcctgagataccagcagatcgagttcatcacctttctgggcgccctgaagtcattcctgaagggaacccctaaaaagaactgcctggtgttctgcggccctgccaataccggcaagtcctacttcggcatgagctttatccacttcatccagggcgccgtgatcagcttcgtgaacagcaccagccacttttggctggaacccctgaccgataccaaggtggcaatgctggacgacgccaccaccacctgttggacctacttcgacacctacatgcggaacgccctggacggcaaccccatcagcatcgacagaaagcacaagcccctcatccagctgaaatgccctccaatcctgctgaccacaaacattcaccccgccaaggacaatcggtggccctacctggaaagccggatcaccgtgttcgagttccccaacgcctttccattcgacaagaacggcaatcccgtgtacgagatcaacgacaagaattggaagtgcttcttcgagcggacctggtccagactggatctgcacgaggaagaagaggacgccgacacagagggcaatcccttcggcacctttaagtgtgtggccgggcagaatcacagacccctgggatctatgcagaccccaaaagagacactgagcgagcggctgagcgccctccaggacaagatcatcgaccactacgagaacgacagcaaggacatcgacagccagattcagtactggcagctgatcagatgggagaacgccatcttcttcgccgctagagagcacggaattcagaccctgaaccaccaggtggtgcccgcctacaacatcagcaagagcaaggcccacaaggccattgaactccagatggccctgcaaggactggctcagagcgcctacaagaccgaggattggacactccaggatacctgcgaggaactgtggaacacagagcccacacactgcttcaagaaaggcggccagaccgtgcaggtctacttcgatggcaacaaggataactgcatgacctacgtggcctgggacagcgtgtactacatgacagatgccggcacctgggacaagaccgccacctgtgtttctcaccggggcctgtactacgtgaaagagggctacaacaccttctacatcgagtttaagagcgagtgcgagaagtatggcaacaccggcacatgggaagtgcatttcggcaacaacgtgatcgactgcaacgactccatgtgctccacctccgatgacaccgtgtctgccacacagctggtcaagcagctccagcacaccccatctccatacagctctacagtgtctgtgggcaccgccaagacctatggccaaacaagcgctgccacaagacctggccattgtggactggccgagaagcagcactgtggccctgttaatcctctgctgggagccgccacacctacaggcaacaacaagagaagaaagctgtgcagcggcaacaccacacctatcattcacctgaagggcgacagaaacagcctgaagtgcctgcggtacagactgaggaagcacagcgatcactaccgggacatcagctccacctggcactggacaggtgctggcaacgaaaagaccggcatcctgacagtgacctaccacagcgagactcagcggaccaagttcctgaacaccgtggctatccccgacagcgtgcagattctcgtgggctacatgaccatgggcagcatggccagattcgaggaccctaccagacggccctacaagctgcctgatctgtgcaccgaactgaacaccagccttcaggatatcgagatcacctgtgtgtactgcaagaccgtgctcgagctgacagaagtgtttgagttcgccttcaaggacctgttcgtggtgtacagagacagcatccctcacgccgcctgtcacaagtgcatcgacttctacagcagaatcagagagctgcggcactacagcgactccgtgtacggcgataccctggaaaaactgaccaacaccggactgtacaacctgctgatccggtgcctgagatgccagaaacctcagaaccccgccgagaaactgagacatctgaacgagaagcggcggttccacaatatcgccggccactacagaggccagtgccacagctgttgcaaccgggccagacaagaaagactccagcggagaagaggcagcatgcacggacctaaagccacacttcaggacatcgtgctgcacctcgagcctcagaatgagatccccgtggatctgctcggccatgagcagctgagcgatagcgaggaagagaacgacgagatcgacggcgtcaaccaccagcatctgcctgccagaagggccgaacctcagagacacaccatgctgtgcatgtgctgcaagtgcgaggcccggatcgagagagtggtggaatctagcgccgacgacctgagagctttccagcagctgttcctgaatactctgagcttcgtggccccttggtgcgctagtcagcagtgagcggccgcggaagaatgtgcacagtgctaaccagaaaattcaggacaagactagtgactctagatcataatcagccataccacatttgtagaggttttacttgctttaaaaaacctcccacacctccccctgaacctgaaacataaaatgaatgcaattgttgttgttaacttgtttattgcagcttataatggttacaaataaagcaatagcatcacaaatttcacaaataaagcatttttttcactgc

SEQ ID NO: 68 - DNA, 5894 bp, synthetic construct
  misc_feature 1..5894: note = HPV18 Ii-E1E6E7-p2a-Ii-E2 as carried by Adenoviral vector
  source 1..5894: mol_type = other DNA
gtgatgcggttttggcagtacatcaatgggcgtggatagcggtttgactcacggggatttccaagtctccaccccattgacgtcaatgggagtttgttttggaaccaaaatcaacgggactttccaaaatgtcgtaacaactccgccccattgacgcaaatgggcggtaggcgtgtacggtgggaggtctatataagcagagctctccctatcagtgatagagatctccctatcagtgatagagatcgtcgacgagctcgtttagtgaaccgtcagatcgcctggagacgccatccacgctgttttgacctctagcaccggtgccaccatgcacagaaggcggagcagaagctgccgcgaggatcagaaacccgtgatggacgaccagcgggacctgatcagcaacaacgagcagctgcctatgctcggcagaaggcctggcgctcctgagagcaaatgttctagaggcgccctgtacaccggcttcagcatcctggttacactgctgctggccggacaggctaccaccgcctactttctgtatcagcagcagggcagactggacaagctgaccgtgaccagccagaacctgcagctggaaaacctgcggatgaagctgcccaagcctcctaagcctgtgtccaagatgcggatggccacacctctgctgatgcaggctttgcctatgggagcactgcctcagggccctatgcagaacgccaccaaatacggcaacatgaccgaggaccacgtgatgcatctgctgcagaatgccgatcctctgaaggtgtaccctccactgaagggcagctttcccgagaacctgcggcacctgaagaacaccatggaaaccatcgactggaaggtgttcgagagctggatgcaccactggctgctgttcgagatgagccggcacagcctggaacagaagcctacagacgcccctcctaaagagtccctggaactggaagatcccagctctggcctgggcgtgacaaagcaagatctgggccctgtgcctatgatggccgatcctgagggaacagatggcgaaggcacaggctgcaacggctggttttacgtgcaagccatcgtggacaagaagaccggcgacgtcatcagcgacgacgaggatgagaatgccaccgacaccggcagcgacatggtggactttatcgatacccagggcacgttctgcgagcaggccgaactggaaacagcccaggcactgtttcacgcccaagaggtgcacaacgatgcccaggtgctgcacgtgctgaagagaaagtttgccggcggaagcaccgagaacagcccactgggagaaagactggaagtggacaccgagctgagccccagactgcaagagatcagcctgaacagcggccagaagaaggccaagcggagactgttcaccatctccgacagcggctacggctgtagcgaagtggaagccacacagatccaagtgaccaccaatggcgagcacggcggcaatgtttgttctggcggatctaccgaggccatcgacaatggcggaaccgagggcaacaatagctccgtggatggcaccagcgacaacagcaacatcgagaacgtgaaccctcagtgcacaatcgcccagctgaaggacctgctgaaagtgaacaacaagcagggcgccatgctggccgtgtttaaggatacctacggcctgagcttcaccgacctcgtgcggaacttcaagagcgacaagaccacctgtaccgactgggtcaccgccatctttggcgtgaacccaacaatcgccgagggcttcaagaccctgatccagcctttcatcctgtacgcccacatccagtgtctggactgcaaatggggcgtgctgattctggccctgctgagatacaagtgcggcaagagcagactgaccgtggccaagggactgagcactctgctgcatgtgcccgagacatgcatgctgattcagcctccaaagctgcggtctagcgtggccgctctgtactggtacagaaccggcatcagcaatatcagcgaagtgatgggcgacacccctgagtggattcagcggctgacaatcatccagcacggcatcgacgactccaacttcgacctgagcgagatggtgcagtgggccttcgataacgagctgaccgacgagtccgacatggcctttgaatacgccctgctggcagacagcaactccaatgccgccgctttcctgaagtccaactgccaggccaagtacctgaaggactgcgccaccatgtgcaagcactaccgcagagcacagaaacggcagatgaacatgagccagtggatccggttccggtgcagcaagatcgatgaaggtggcgactggcggcctatcgtgcagttcctgagataccagcagatcgagttcatcacctttctgggcgccctgaagtcattcctgaagggaacccctaaaaagaactgcctggtgttctgcggccctgccaataccggcaagtcctacttcggcatgagctttatccacttcatccagggcgccgtgatcagcttcgtgaacagcaccagccacttttggctggaacccctgaccgataccaaggtggcaatgctggacgacgccaccaccacctgttggacctacttcgacacctacatgcggaacgccctggacggcaaccccatcagcatcgacagaaagcacaagcccctcatccagctgaaatgccctccaatcctgctgaccacaaacattcaccccgccaaggacaatcggtggccctacctggaaagccggatcaccgtgttcgagttccccaacgcctttccattcgacaagaacggcaatcccgtgtacgagatcaacgacaagaattggaagtgcttcttcgagcggacctggtccagactggatctgcacgaggaagaagaggacgccgacacagagggcaatcccttcggcacctttaagtgtgtggccgggcagaatcacagacccctgggcagcatggccagattcgaggaccctaccagacggccctacaagctgcctgatctgtgcaccgaactgaacaccagccttcaggatatcgagatcacctgtgtgtactgcaagaccgtgctcgagctgacagaagtgtttgagttcgccttcaaggacctgttcgtggtgtacagagacagcatccctcacgccgcctgtcacaagtgcatcgacttctacagcagaatcagagagctgcggcactacagcgactccgtgtacggcgataccctggaaaaactgaccaacaccggactgtacaacctgctgatccggtgcctgagatgccagaaacctcagaaccccgccgagaaactgagacatctgaacgagaagcggcggttccacaatatcgccggccactacagaggccagtgccacagctgttgcaaccgggccagacaagaaagactccagcggagaagaggcagcatgcacggacctaaagccacacttcaggacatcgtgctgcacctcgagcctcagaatgagatccccgtggatctgctcggccatgagcagctgagcgatagcgaggaagagaacgacgagatcgacggcgtcaaccaccagcatctgcctgccagaagggccgaacctcagagacacaccatgctgtgcatgtgctgcaagtgcgaggcccggatcgagagagtggtggaatctagcgccgacgacctgagagctttccagcagctgttcctgaatactctgagcttcgtggccccttggtgcgctagtcagcagggcagcggcgccaccaatttcagtctgcttaaacaggccggggacgtcgaagagaaccccggacctatgcatcggcgcagatctcggtcttgtagagaagaccaaaagcctgtcatggatgatcaaagagatttaatttctaataatgaacaattacccatgttgggacggcgccccggagcccccgaatctaagtgcagccggggagctttatatacaggattttctattttagtgaccttattattagctggccaagccacaacagcttatttcttataccaacaacaaggacggttagataaattaacagtcacatctcaaaatttacaattagagaatttaagaatgaaattacctaaaccacccaaacccgtcagtaaaatgagaatggctacccccttattaatgcaagccctccccatgggcgccttaccccaaggacccatgcaaaatgctacaaagtatggaaatatgacagaagatcatgtcatgcacttattacaaaacgctgaccccttaaaagtctatccccctttaaaaggatctttccctgaaaatttaagacatttaaaaaatacaatggagacaattgattggaaagtctttgaatcttggatgcatcattggttattatttgaaatgtctaggcattctttagagcaaaaacccaccgatgctccacccaaggaaagtttagagttagaggacccttctagcggattaggagtcaccaaacaggacttaggacccgtccccatgatgcagaccccaaaagagacactgagcgagcggctgagcgccctccaggacaagatcatcgaccactacgagaacgacagcaaggacatcgacagccagattcagtactggcagctgatcagatgggagaacgccatcttcttcgccgctagagagcacggaattcagaccctgaaccaccaggtggtgcccgcctacaacatcagcaagagcaaggcccacaaggccattgaactccagatggccctgcaaggactggctcagagcgcctacaagaccgaggattggacactccaggatacctgcgaggaactgtggaacacagagcccacacactgcttcaagaaaggcggccagaccgtgcaggtctacttcgatggcaacaaggataactgcatgacctacgtggcctgggacagcgtgtactacatgacagatgccggcacctgggacaagaccgccacctgtgtttctcaccggggcctgtactacgtgaaagagggctacaacaccttctacatcgagtttaagagcgagtgcgagaagtatggcaacaccggcacatgggaagtgcatttcggcaacaacgtgatcgactgcaacgactccatgtgctccacctccgatgacaccgtgtctgccacacagctggtcaagcagctccagcacaccccatctccatacagctctacagtgtctgtgggcaccgccaagacctatggccaaacaagcgctgccacaagacctggccattgtggactggccgagaagcagcactgtggccctgttaatcctctgctgggagccgccacacctacaggcaacaacaagagaagaaagctgtgcagcggcaacaccacacctatcattcacctgaagggcgacagaaacagcctgaagtgcctgcggtacagactgaggaagcacagcgatcactaccgggacatcagctccacctggcactggacaggtgctggcaacgaaaagaccggcatcctgacagtgacctaccacagcgagactcagcggaccaagttcctgaacaccgtggctatccccgacagcgtgcagattctcgtgggctacatgaccatgtgagcggccgcggaagaatgtgcacagtgctaaccagaaaattcaggacaagactagtgactctagatcataatcagccataccacatttgtagaggttttacttgctttaaaaaacctcccacacctccccctgaacctgaaacataaaatgaatgcaattgttgttgttaacttgtttattgcagcttataatggttacaaataaagcaatagcatcacaaatttcacaaataaagcatttttttcactgc

SEQ ID NO: 69 - DNA, 4037 bp, synthetic construct
  misc_feature 1..4037: note = HPV18 Ii-E1E6E7 as carried by Adenoviral vector
  source 1..4037: mol_type = other DNA
gtgatgcggttttggcagtacatcaatgggcgtggatagcggtttgactcacggggatttccaagtctccaccccattgacgtcaatgggagtttgttttggaaccaaaatcaacgggactttccaaaatgtcgtaacaactccgccccattgacgcaaatgggcggtaggcgtgtacggtgggaggtctatataagcagagctctccctatcagtgatagagatctccctatcagtgatagagatcgtcgacgagctcgtttagtgaaccgtcagatcgcctggagacgccatccacgctgttttgacctctagcaccggtgccaccatgcacagaaggcggagcagaagctgccgcgaggatcagaaacccgtgatggacgaccagcgggacctgatcagcaacaacgagcagctgcctatgctcggcagaaggcctggcgctcctgagagcaaatgttctagaggcgccctgtacaccggcttcagcatcctggttacactgctgctggccggacaggctaccaccgcctactttctgtatcagcagcagggcagactggacaagctgaccgtgaccagccagaacctgcagctggaaaacctgcggatgaagctgcccaagcctcctaagcctgtgtccaagatgcggatggccacacctctgctgatgcaggctttgcctatgggagcactgcctcagggccctatgcagaacgccaccaaatacggcaacatgaccgaggaccacgtgatgcatctgctgcagaatgccgatcctctgaaggtgtaccctccactgaagggcagctttcccgagaacctgcggcacctgaagaacaccatggaaaccatcgactggaaggtgttcgagagctggatgcaccactggctgctgttcgagatgagccggcacagcctggaacagaagcctacagacgcccctcctaaagagtccctggaactggaagatcccagctctggcctgggcgtgacaaagcaagatctgggccctgtgcctatgatggccgatcctgagggaacagatggcgaaggcacaggctgcaacggctggttttacgtgcaagccatcgtggacaagaagaccggcgacgtcatcagcgacgacgaggatgagaatgccaccgacaccggcagcgacatggtggactttatcgatacccagggcacgttctgcgagcaggccgaactggaaacagcccaggcactgtttcacgcccaagaggtgcacaacgatgcccaggtgctgcacgtgctgaagagaaagtttgccggcggaagcaccgagaacagcccactgggagaaagactggaagtggacaccgagctgagccccagactgcaagagatcagcctgaacagcggccagaagaaggccaagcggagactgttcaccatctccgacagcggctacggctgtagcgaagtggaagccacacagatccaagtgaccaccaatggcgagcacggcggcaatgtttgttctggcggatctaccgaggccatcgacaatggcggaaccgagggcaacaatagctccgtggatggcaccagcgacaacagcaacatcgagaacgtgaaccctcagtgcacaatcgcccagctgaaggacctgctgaaagtgaacaacaagcagggcgccatgctggccgtgtttaaggatacctacggcctgagcttcaccgacctcgtgcggaacttcaagagcgacaagaccacctgtaccgactgggtcaccgccatctttggcgtgaacccaacaatcgccgagggcttcaagaccctgatccagcctttcatcctgtacgcccacatccagtgtctggactgcaaatggggcgtgctgattctggccctgctgagatacaagtgcggcaagagcagactgaccgtggccaagggactgagcactctgctgcatgtgcccgagacatgcatgctgattcagcctccaaagctgcggtctagcgtggccgctctgtactggtacagaaccggcatcagcaatatcagcgaagtgatgggcgacacccctgagtggattcagcggctgacaatcatccagcacggcatcgacgactccaacttcgacctgagcgagatggtgcagtgggccttcgataacgagctgaccgacgagtccgacatggcctttgaatacgccctgctggcagacagcaactccaatgccgccgctttcctgaagtccaactgccaggccaagtacctgaaggactgcgccaccatgtgcaagcactaccgcagagcacagaaacggcagatgaacatgagccagtggatccggttccggtgcagcaagatcgatgaaggtggcgactggcggcctatcgtgcagttcctgagataccagcagatcgagttcatcacctttctgggcgccctgaagtcattcctgaagggaacccctaaaaagaactgcctggtgttctgcggccctgccaataccggcaagtcctacttcggcatgagctttatccacttcatccagggcgccgtgatcagcttcgtgaacagcaccagccacttttggctggaacccctgaccgataccaaggtggcaatgctggacgacgccaccaccacctgttggacctacttcgacacctacatgcggaacgccctggacggcaaccccatcagcatcgacagaaagcacaagcccctcatccagctgaaatgccctccaatcctgctgaccacaaacattcaccccgccaaggacaatcggtggccctacctggaaagccggatcaccgtgttcgagttccccaacgcctttccattcgacaagaacggcaatcccgtgtacgagatcaacgacaagaattggaagtgcttcttcgagcggacctggtccagactggatctgcacgaggaagaagaggacgccgacacagagggcaatcccttcggcacctttaagtgtgtggccgggcagaatcacagacccctgggcagcatggccagattcgaggaccctaccagacggccctacaagctgcctgatctgtgcaccgaactgaacaccagccttcaggatatcgagatcacctgtgtgtactgcaagaccgtgctcgagctgacagaagtgtttgagttcgccttcaaggacctgttcgtggtgtacagagacagcatccctcacgccgcctgtcacaagtgcatcgacttctacagcagaatcagagagctgcggcactacagcgactccgtgtacggcgataccctggaaaaactgaccaacaccggactgtacaacctgctgatccggtgcctgagatgccagaaacctcagaaccccgccgagaaactgagacatctgaacgagaagcggcggttccacaatatcgccggccactacagaggccagtgccacagctgttgcaaccgggccagacaagaaagactccagcggagaagaggcagcatgcacggacctaaagccacacttcaggacatcgtgctgcacctcgagcctcagaatgagatccccgtggatctgctcggccatgagcagctgagcgatagcgaggaagagaacgacgagatcgacggcgtcaaccaccagcatctgcctgccagaagggccgaacctcagagacacaccatgctgtgcatgtgctgcaagtgcgaggcccggatcgagagagtggtggaatctagcgccgacgacctgagagctttccagcagctgttcctgaatactctgagcttcgtggccccttggtgcgctagtcagcagtgagcggccgcggaagaatgtgcacagtgctaaccagaaaattcaggacaagactagtgactctagatcataatcagccataccacatttgtagaggttttacttgctttaaaaaacctcccacacctccccctgaacctgaaacataaaatgaatgcaattgttgttgttaacttgtttattgcagcttataatggttacaaataaagcaatagcatcacaaatttcacaaataaagcatttttttcactgc

SEQ ID NO: 70 - DNA, 4748 bp, synthetic construct
  misc_feature 1..4748: note = MfPV3 Ii-E1E2E6E7 integrated into DelIII locus of the poxviral vector
  source 1..4748: mol_type = other DNA
aaaaattaaaattttattttttttttttggaatataaataagatctatccctatcagtgatagagatctccctatcagtgatagagatcgtcgacgagctcgtttagtgaaccgtcagatcgcctggagacgccatccacgctgttttgacctctagcaccggtgccaccatgcacagaaggcggagcagaagctgccgcgaggatcagaaacccgtgatggacgaccagcgggacctgatcagcaacaacgagcagctgcctatgctcggcagaaggcctggcgctcctgagagcaaatgttctagaggcgccctgtacaccggcttcagcatcctggttacactgctgctggccggacaggctaccaccgcctactttctgtatcagcagcagggcagactggacaagctgaccgtgaccagccagaacctgcagctggaaaacctgcggatgaagctgcccaagcctcctaagcctgtgtccaagatgcggatggccacacctctgctgatgcaggctttgcctatgggagcactgcctcagggccctatgcagaacgccaccaaatacggcaacatgaccgaggaccacgtgatgcatctgctgcagaatgccgatcctctgaaggtgtaccctccactgaagggcagctttcccgagaacctgcggcacctgaagaacaccatggaaaccatcgactggaaggtgttcgagagctggatgcaccactggctgctgttcgagatgagccggcacagcctggaacagaagcctacagacgcccctcctaaagagtccctggaactggaagatcccagctctggcctgggcgtgacaaagcaagatctgggccctgtgcctatgatggacccttctggaattcacggcgagggcaccagatgcaccggatggttcgatgtggaagccatcgtggaaagacggaccggcgacgtggtgtccgaggacgaagatgagaacctggaagataccggcgtggacctggtggacttcatcgacgatacctgcgagtttgagcaggccggcgacgaagctcctagagcactgctgcatgcccaagagacacaggcccatgtgaacgccgtgcactacctgaagcggaagttcacatgcagccctaccgtgtctcccctgggcaatgtgcagtgttctatcgagggcgagctgagccctagactgcacgccatccaactgaatgaaggcagcggcaaggccaagcggagactgtttctgcctgatagcggctacggcaataccgaggtggaaacccagctgtctcaggtggcaggatccgatggcgaatctcaagcccctggcggcaaagaaaatgtgcagcctgctgaaggcgctggcggagatgttcgacagctgctgagaagcagcaacctgaaggccacactgctgggcaagttcaaggacgtgtacggcatcggcttcaacgaactcgtgcggtgcttcaagagcgacaagaccgtgtgcaccgattgggttgtgggagctgctggcgtgcaccattctgtggccgaaggactgaagcacctgatcgagcctttctgcacatacgcccacatgcagtgcctgacctgcaactggggagtgtttctgctgctgctcatccggttcaagtgcggcaagaacagactgaccgtggccaagtgcatctctaccctgctgaacgtgcaagagccctacctgctgatcgaacctccaaagctgagatccacagccgccgctctgtactggtacagaaccggcatcagcaacgtgtccgaagtgatcggagagcctcctgagtggatcgtgcggcagaccatgtttcagcacagcctcgaggacagcatcttcgacctgtctcagatggtgcagtgggcctacgaccacgatctggtggacgatagcgccattgcctaccagtatgcccagctggccgacgtggactctaatgccgccgctttcctgaagtctaacgctcaggccaaatacgtgaaggactgcgccaccatgtgcagacactacaagagagccgagaagcaggccatgaccatgagccagtggatcagagccagatgcgataagaccgacgacggcggagattggaggcctatcgtgcagttcctgagataccagaacgtcgagttcatcacctttctgagcgcctttaaggccttcctgaagggaatccccaagagaagctgcatcgtgctgtacggccctccaaacaccggcaagagctacttcggaatgagcctgatgcagtttctgcagggcagcatcatcagctacgtgaacagcaacagccacttttggctggcccctctggccgaagctaaagtggccatgctggacgatgccacaccacagtgctggtcctacatcgacacctacatgcggagcgtgctggacggcaaccccatgagcattgaccggaagcacaagaacctgatccagatgaagtgtccgcctctgatcatcaccagcaacaccaacgtgggcgaagatgacagatggcgctacctgcactccagagtggtggtgttcaagttcgagcagacctttccattcgaccagaacggcaatcccgtgtacgagctgtccgacaagaattggaagtccttcttcagccggacctggtccagactcgagctgcaagaggatgaggacaccgagcaggatggctctacagtgcgggcctttaagtgtgtgcccggccagaacaccaatatcctgggctctatggaagccctgagcgagagactgaacgcctaccaggacaagatcctgaccttctacgaagaggactccaaggacctgagggaccagatcgagcattggagatgtgtgcggggagagtgcgccgtgttctacaaggccagagagatgggccacagccacatcaaccaccagcaggttccacctctgcacgtgtccagaggcaaggcccacaatgccattgagctgcagatggccctggaaagcctgaaccagagcgacttcaacaccgaggaatggaccctgcaggacacaagcctggaaatgtggcggaccgagcctcagtgctgcttcaaaaagaaaggcgagacagtgaccgtgctgtatgactgcgacaaggacaacagcatggactacaccctgtggggccacgtgtacgtgtggggagatacaggctgggacaaagtgtgcggaaaggtggactacggcggcctgtactatgtgctgcacggcctgaaagtgtactacgttgagttccagcaagaggccgtgaagtacagcaagaccaacaagtgggaagtgcatatcggcagaacagtgatcccctacagcgagtccgtgtcctctacagccctgtgcgaaagagtgcctatcgccgagatcgccaatggcctccagcagaccagcattccctctcacacacacgccaccaccaaagagaacgcttgggtttcctctcagcctcctaccaagcggtccagaagatctgatccaggcggcgatcctgtcagagccctggatgccaatagcagaaccctgctgtctggaaccgcctgcgagaataccaccggcaactcttacagcgactgcacccctgtggtgcacctgaaaggcgaccccaattgtctgaaggccctgcggttcagattcgtgaaacacaagaatctgttcgtgaacgtgtcctccacctggcggtggaccaacaacaccgaagagaaggccatcgtgactgtgacctttaaggacgagcagcagagacagcagttcctcggcagcgtgaagatccccaactccatctgtgtgtgcaagggcgtgatgacagttggcagcatggtggaaggcagccaagagagagcccggacactgcacgaactgtgcgagcagagagaacagacccagcacgacatcgagctggactgcgtgtactgcctgaatcctctgaccagaaccgaggtgtacgacttcgccagatgggagctgagaatcgtgtacagacgggataccgcctacggcgtgtgcaagctgtgcctgaggttccacagcaaagtgcggaagtacaggcggtacaactactccatctacggccccacactggaacggcggacaaagaagcagctcgaagaactcgtgatccggtgctactgctgccagaaacctcagtgccccgtggaaaagcagcgccacgttgaccagggccagagattccacaacattgccgagcagtggaccggcagatgcctgcaatgttggaggccaacagccgctgaaaccggctctatgatcggccctggacctacactgcaggatatcgtgcttgagctggaacccgagcctgtggacctgagaggatatgagcagctggactccagcgaggacgaggacgagattgaccaccaccaacagcaggacagacccgatgtgcaagaagatgacggcgagcggtttagaatcctgagcgactgtcacgtgtgccagaataccgtgtcaagagtggtgcacagcacccacgacgacctgcacattctcgaggacctgcttatgggcaccctcgagatcgtggcccctaattgtgccgtgggcagcgaacagaaactgatctccgaggaagatctctgaggaagaatgtgcacagtgctaaccagaaaattcaggacaagactagtgcggccgctttggaaagttttataggtagttgatagaacaaaatacataattttgtaaaaataaatcactttttat

SEQ ID NO: 71 - DNA, 5504 bp, synthetic construct
  misc_feature 1..5504: note = MfPV3 Ii-E1E2-p2a-Ii-E6E7 integrated into DelIII locus of the poxviral vector
  source 1..5504: mol_type = other DNA
aaaaattaaaattttattttttttttttggaatataaataagatctatccctatcagtgatagagatctccctatcagtgatagagatcgtcgacgagctcgtttagtgaaccgtcagatcgcctggagacgccatccacgctgttttgacctctagcaccggtgccaccatgcacagaaggcggagcagaagctgccgcgaggatcagaaacccgtgatggacgaccagcgggacctgatcagcaacaacgagcagctgcctatgctcggcagaaggcctggcgctcctgagagcaaatgttctagaggcgccctgtacaccggcttcagcatcctggttacactgctgctggccggacaggctaccaccgcctactttctgtatcagcagcagggcagactggacaagctgaccgtgaccagccagaacctgcagctggaaaacctgcggatgaagctgcccaagcctcctaagcctgtgtccaagatgcggatggccacacctctgctgatgcaggctttgcctatgggagcactgcctcagggccctatgcagaacgccaccaaatacggcaacatgaccgaggaccacgtgatgcatctgctgcagaatgccgatcctctgaaggtgtaccctccactgaagggcagctttcccgagaacctgcggcacctgaagaacaccatggaaaccatcgactggaaggtgttcgagagctggatgcaccactggctgctgttcgagatgagccggcacagcctggaacagaagcctacagacgcccctcctaaagagtccctggaactggaagatcccagctctggcctgggcgtgacaaagcaagatctgggccctgtgcctatgatggacccttctggaattcacggcgagggcaccagatgcaccggatggttcgatgtggaagccatcgtggaaagacggaccggcgacgtggtgtccgaggacgaagatgagaacctggaagataccggcgtggacctggtggacttcatcgacgatacctgcgagtttgagcaggccggcgacgaagctcctagagcactgctgcatgcccaagagacacaggcccatgtgaacgccgtgcactacctgaagcggaagttcacatgcagccctaccgtgtctcccctgggcaatgtgcagtgttctatcgagggcgagctgagccctagactgcacgccatccaactgaatgaaggcagcggcaaggccaagcggagactgtttctgcctgatagcggctacggcaataccgaggtggaaacccagctgtctcaggtggcaggatccgatggcgaatctcaagcccctggcggcaaagaaaatgtgcagcctgctgaaggcgctggcggagatgttcgacagctgctgagaagcagcaacctgaaggccacactgctgggcaagttcaaggacgtgtacggcatcggcttcaacgaactcgtgcggtgcttcaagagcgacaagaccgtgtgcaccgattgggttgtgggagctgctggcgtgcaccattctgtggccgaaggactgaagcacctgatcgagcctttctgcacatacgcccacatgcagtgcctgacctgcaactggggagtgtttctgctgctgctcatccggttcaagtgcggcaagaacagactgaccgtggccaagtgcatctctaccctgctgaacgtgcaagagccctacctgctgatcgaacctccaaagctgagatccacagccgccgctctgtactggtacagaaccggcatcagcaacgtgtccgaagtgatcggagagcctcctgagtggatcgtgcggcagaccatgtttcagcacagcctcgaggacagcatcttcgacctgtctcagatggtgcagtgggcctacgaccacgatctggtggacgatagcgccattgcctaccagtatgcccagctggccgacgtggactctaatgccgccgctttcctgaagtctaacgctcaggccaaatacgtgaaggactgcgccaccatgtgcagacactacaagagagccgagaagcaggccatgaccatgagccagtggatcagagccagatgcgataagaccgacgacggcggagattggaggcctatcgtgcagttcctgagataccagaacgtcgagttcatcacctttctgagcgcctttaaggccttcctgaagggaatccccaagagaagctgcatcgtgctgtacggccctccaaacaccggcaagagctacttcggaatgagcctgatgcagtttctgcagggcagcatcatcagctacgtgaacagcaacagccacttttggctggcccctctggccgaagctaaagtggccatgctggacgatgccacaccacagtgctggtcctacatcgacacctacatgcggagcgtgctggacggcaaccccatgagcattgaccggaagcacaagaacctgatccagatgaagtgtccgcctctgatcatcaccagcaacaccaacgtgggcgaagatgacagatggcgctacctgcactccagagtggtggtgttcaagttcgagcagacctttccattcgaccagaacggcaatcccgtgtacgagctgtccgacaagaattggaagtccttcttcagccggacctggtccagactcgagctgcaagaggatgaggacaccgagcaggatggctctacagtgcgggcctttaagtgtgtgcccggccagaacaccaatatcctgggctctatggaagccctgagcgagagactgaacgcctaccaggacaagatcctgaccttctacgaagaggactccaaggacctgagggaccagatcgagcattggagatgtgtgcggggagagtgcgccgtgttctacaaggccagagagatgggccacagccacatcaaccaccagcaggttccacctctgcacgtgtccagaggcaaggcccacaatgccattgagctgcagatggccctggaaagcctgaaccagagcgacttcaacaccgaggaatggaccctgcaggacacaagcctggaaatgtggcggaccgagcctcagtgctgcttcaaaaagaaaggcgagacagtgaccgtgctgtatgactgcgacaaggacaacagcatggactacaccctgtggggccacgtgtacgtgtggggagatacaggctgggacaaagtgtgcggaaaggtggactacggcggcctgtactatgtgctgcacggcctgaaagtgtactacgttgagttccagcaagaggccgtgaagtacagcaagaccaacaagtgggaagtgcatatcggcagaacagtgatcccctacagcgagtccgtgtcctctacagccctgtgcgaaagagtgcctatcgccgagatcgccaatggcctccagcagaccagcattccctctcacacacacgccaccaccaaagagaacgcttgggtttcctctcagcctcctaccaagcggtccagaagatctgatccaggcggcgatcctgtcagagccctggatgccaatagcagaaccctgctgtctggaaccgcctgcgagaataccaccggcaactcttacagcgactgcacccctgtggtgcacctgaaaggcgaccccaattgtctgaaggccctgcggttcagattcgtgaaacacaagaatctgttcgtgaacgtgtcctccacctggcggtggaccaacaacaccgaagagaaggccatcgtgactgtgacctttaaggacgagcagcagagacagcagttcctcggcagcgtgaagatccccaactccatctgtgtgtgcaagggcgtgatgacagttggcagcggcgccaccaatttcagtctgcttaaacaggccggggacgtcgaagagaaccccggacctatgcatcggcgcagatctcggtcttgtagagaagaccaaaagcctgtcatggatgatcaaagagatttaatttctaataatgaacaattacccatgttgggacggcgccccggagcccccgaatctaagtgcagccggggagctttatatacaggattttctattttagtgaccttattattagctggccaagccacaacagcttatttcttataccaacaacaaggacggttagataaattaacagtcacatctcaaaatttacaattagagaatttaagaatgaaattacctaaaccacccaaacccgtcagtaaaatgagaatggctacccccttattaatgcaagccctccccatgggcgccttaccccaaggacccatgcaaaatgctacaaagtatggaaatatgacagaagatcatgtcatgcacttattacaaaacgctgaccccttaaaagtctatccccctttaaaaggatctttccctgaaaatttaagacatttaaaaaatacaatggagacaattgattggaaagtctttgaatcttggatgcatcattggttattatttgaaatgtctaggcattctttagagcaaaaacccaccgatgctccacccaaggaaagtttagagttagaggacccttctagcggattaggagtcaccaaacaggacttaggacccgtccccatgatggtggaaggcagccaagagagagcccggacactgcacgaactgtgcgagcagagagaacagacccagcacgacatcgagctggactgcgtgtactgcctgaatcctctgaccagaaccgaggtgtacgacttcgccagatgggagctgagaatcgtgtacagacgggataccgcctacggcgtgtgcaagctgtgcctgaggttccacagcaaagtgcggaagtacaggcggtacaactactccatctacggccccacactggaacggcggacaaagaagcagctcgaagaactcgtgatccggtgctactgctgccagaaacctcagtgccccgtggaaaagcagcgccacgttgaccagggccagagattccacaacattgccgagcagtggaccggcagatgcctgcaatgttggaggccaacagccgctgaaaccggctctatgatcggccctggacctacactgcaggatatcgtgcttgagctggaacccgagcctgtggacctgagaggatatgagcagctggactccagcgaggacgaggacgagattgaccaccaccaacagcaggacagacccgatgtgcaagaagatgacggcgagcggtttagaatcctgagcgactgtcacgtgtgccagaataccgtgtcaagagtggtgcacagcacccacgacgacctgcacattctcgaggacctgcttatgggcaccctcgagatcgtggcccctaattgtgccgtgggcagcgaacagaaactgatctccgaggaagatctctgaggaagaatgtgcacagtgctaaccagaaaattcaggacaagactagtgcggccgctttggaaagttttataggtagttgatagaacaaaatacataattttgtaaaaataaatcactttttat

SEQ ID NO: 72 - DNA, 4052 bp, synthetic construct
  misc_feature 1..4052: note = MfPV3 E1E2E6E7 integrated into DelIII locus of the poxviral vector
  source 1..4052: mol_type = other DNA
aaaaattaaaattttattttttttttttggaatataaataagatctatccctatcagtgatagagatctccctatcagtgatagagatcgtcgacgagctcgtttagtgaaccgtcagatcgcctggagacgccatccacgctgttttgacctctagcaccggtgccaccatggacccttctggaattcacggcgagggcaccagatgcaccggatggttcgatgtggaagccatcgtggaaagacggaccggcgacgtggtgtccgaggacgaagatgagaacctggaagataccggcgtggacctggtggacttcatcgacgatacctgcgagtttgagcaggccggcgacgaagctcctagagcactgctgcatgcccaagagacacaggcccatgtgaacgccgtgcactacctgaagcggaagttcacatgcagccctaccgtgtctcccctgggcaatgtgcagtgttctatcgagggcgagctgagccctagactgcacgccatccaactgaatgaaggcagcggcaaggccaagcggagactgtttctgcctgatagcggctacggcaataccgaggtggaaacccagctgtctcaggtggcaggatccgatggcgaatctcaagcccctggcggcaaagaaaatgtgcagcctgctgaaggcgctggcggagatgttcgacagctgctgagaagcagcaacctgaaggccacactgctgggcaagttcaaggacgtgtacggcatcggcttcaacgaactcgtgcggtgcttcaagagcgacaagaccgtgtgcaccgattgggttgtgggagctgctggcgtgcaccattctgtggccgaaggactgaagcacctgatcgagcctttctgcacatacgcccacatgcagtgcctgacctgcaactggggagtgtttctgctgctgctcatccggttcaagtgcggcaagaacagactgaccgtggccaagtgcatctctaccctgctgaacgtgcaagagccctacctgctgatcgaacctccaaagctgagatccacagccgccgctctgtactggtacagaaccggcatcagcaacgtgtccgaagtgatcggagagcctcctgagtggatcgtgcggcagaccatgtttcagcacagcctcgaggacagcatcttcgacctgtctcagatggtgcagtgggcctacgaccacgatctggtggacgatagcgccattgcctaccagtatgcccagctggccgacgtggactctaatgccgccgctttcctgaagtctaacgctcaggccaaatacgtgaaggactgcgccaccatgtgcagacactacaagagagccgagaagcaggccatgaccatgagccagtggatcagagccagatgcgataagaccgacgacggcggagattggaggcctatcgtgcagttcctgagataccagaacgtcgagttcatcacctttctgagcgcctttaaggccttcctgaagggaatccccaagagaagctgcatcgtgctgtacggccctccaaacaccggcaagagctacttcggaatgagcctgatgcagtttctgcagggcagcatcatcagctacgtgaacagcaacagccacttttggctggcccctctggccgaagctaaagtggccatgctggacgatgccacaccacagtgctggtcctacatcgacacctacatgcggagcgtgctggacggcaaccccatgagcattgaccggaagcacaagaacctgatccagatgaagtgtccgcctctgatcatcaccagcaacaccaacgtgggcgaagatgacagatggcgctacctgcactccagagtggtggtgttcaagttcgagcagacctttccattcgaccagaacggcaatcccgtgtacgagctgtccgacaagaattggaagtccttcttcagccggacctggtccagactcgagctgcaagaggatgaggacaccgagcaggatggctctacagtgcgggcctttaagtgtgtgcccggccagaacaccaatatcctgggctctatggaagccctgagcgagagactgaacgcctaccaggacaagatcctgaccttctacgaagaggactccaaggacctgagggaccagatcgagcattggagatgtgtgcggggagagtgcgccgtgttctacaaggccagagagatgggccacagccacatcaaccaccagcaggttccacctctgcacgtgtccagaggcaaggcccacaatgccattgagctgcagatggccctggaaagcctgaaccagagcgacttcaacaccgaggaatggaccctgcaggacacaagcctggaaatgtggcggaccgagcctcagtgctgcttcaaaaagaaaggcgagacagtgaccgtgctgtatgactgcgacaaggacaacagcatggactacaccctgtggggccacgtgtacgtgtggggagatacaggctgggacaaagtgtgcggaaaggtggactacggcggcctgtactatgtgctgcacggcctgaaagtgtactacgttgagttccagcaagaggccgtgaagtacagcaagaccaacaagtgggaagtgcatatcggcagaacagtgatcccctacagcgagtccgtgtcctctacagccctgtgcgaaagagtgcctatcgccgagatcgccaatggcctccagcagaccagcattccctctcacacacacgccaccaccaaagagaacgcttgggtttcctctcagcctcctaccaagcggtccagaagatctgatccaggcggcgatcctgtcagagccctggatgccaatagcagaaccctgctgtctggaaccgcctgcgagaataccaccggcaactcttacagcgactgcacccctgtggtgcacctgaaaggcgaccccaattgtctgaaggccctgcggttcagattcgtgaaacacaagaatctgttcgtgaacgtgtcctccacctggcggtggaccaacaacaccgaagagaaggccatcgtgactgtgacctttaaggacgagcagcagagacagcagttcctcggcagcgtgaagatccccaactccatctgtgtgtgcaagggcgtgatgacagttggcagcatggtggaaggcagccaagagagagcccggacactgcacgaactgtgcgagcagagagaacagacccagcacgacatcgagctggactgcgtgtactgcctgaatcctctgaccagaaccgaggtgtacgacttcgccagatgggagctgagaatcgtgtacagacgggataccgcctacggcgtgtgcaagctgtgcctgaggttccacagcaaagtgcggaagtacaggcggtacaactactccatctacggccccacactggaacggcggacaaagaagcagctcgaagaactcgtgatccggtgctactgctgccagaaacctcagtgccccgtggaaaagcagcgccacgttgaccagggccagagattccacaacattgccgagcagtggaccggcagatgcctgcaatgttggaggccaacagccgctgaaaccggctctatgatcggccctggacctacactgcaggatatcgtgcttgagctggaacccgagcctgtggacctgagaggatatgagcagctggactccagcgaggacgaggacgagattgaccaccaccaacagcaggacagacccgatgtgcaagaagatgacggcgagcggtttagaatcctgagcgactgtcacgtgtgccagaataccgtgtcaagagtggtgcacagcacccacgacgacctgcacattctcgaggacctgcttatgggcaccctcgagatcgtggcccctaattgtgccgtgggcagcgaacagaaactgatctccgaggaagatctctgaggaagaatgtgcacagtgctaaccagaaaattcaggacaagactagtgcggccgctttggaaagttttataggtagttgatagaacaaaatacataattttgtaaaaataaatcactttttat

SEQ ID NO: 73 - DNA, 5504 bp, synthetic construct
  misc_feature 1..5504: note = MfPV3 Ii-E1E6E7-p2a-Ii-E2 integrated into DelIII locus of the poxviral vector
  source 1..5504: mol_type = other DNA
aaaaattaaaattttattttttttttttggaatataaataagatctatccctatcagtgatagagatctccctatcagtgatagagatcgtcgacgagctcgtttagtgaaccgtcagatcgcctggagacgccatccacgctgttttgacctctagcaccggtgccaccatgcacagaaggcggagcagaagctgccgcgaggatcagaaacccgtgatggacgaccagcgggacctgatcagcaacaacgagcagctgcctatgctcggcagaaggcctggcgctcctgagagcaaatgttctagaggcgccctgtacaccggcttcagcatcctggttacactgctgctggccggacaggctaccaccgcctactttctgtatcagcagcagggcagactggacaagctgaccgtgaccagccagaacctgcagctggaaaacctgcggatgaagctgcccaagcctcctaagcctgtgtccaagatgcggatggccacacctctgctgatgcaggctttgcctatgggagcactgcctcagggccctatgcagaacgccaccaaatacggcaacatgaccgaggaccacgtgatgcatctgctgcagaatgccgatcctctgaaggtgtaccctccactgaagggcagctttcccgagaacctgcggcacctgaagaacaccatggaaaccatcgactggaaggtgttcgagagctggatgcaccactggctgctgttcgagatgagccggcacagcctggaacagaagcctacagacgcccctcctaaagagtccctggaactggaagatcccagctctggcctgggcgtgacaaagcaagatctgggccctgtgcctatgatggacccttctggaattcacggcgagggcaccagatgcaccggatggttcgatgtggaagccatcgtggaaagacggaccggcgacgtggtgtccgaggacgaagatgagaacctggaagataccggcgtggacctggtggacttcatcgacgatacctgcgagtttgagcaggccggcgacgaagctcctagagcactgctgcatgcccaagagacacaggcccatgtgaacgccgtgcactacctgaagcggaagttcacatgcagccctaccgtgtctcccctgggcaatgtgcagtgttctatcgagggcgagctgagccctagactgcacgccatccaactgaatgaaggcagcggcaaggccaagcggagactgtttctgcctgatagcggctacggcaataccgaggtggaaacccagctgtctcaggtggcaggatccgatggcgaatctcaagcccctggcggcaaagaaaatgtgcagcctgctgaaggcgctggcggagatgttcgacagctgctgagaagcagcaacctgaaggccacactgctgggcaagttcaaggacgtgtacggcatcggcttcaacgaactcgtgcggtgcttcaagagcgacaagaccgtgtgcaccgattgggttgtgggagctgctggcgtgcaccattctgtggccgaaggactgaagcacctgatcgagcctttctgcacatacgcccacatgcagtgcctgacctgcaactggggagtgtttctgctgctgctcatccggttcaagtgcggcaagaacagactgaccgtggccaagtgcatctctaccctgctgaacgtgcaagagccctacctgctgatcgaacctccaaagctgagatccacagccgccgctctgtactggtacagaaccggcatcagcaacgtgtccgaagtgatcggagagcctcctgagtggatcgtgcggcagaccatgtttcagcacagcctcgaggacagcatcttcgacctgtctcagatggtgcagtgggcctacgaccacgatctggtggacgatagcgccattgcctaccagtatgcccagctggccgacgtggactctaatgccgccgctttcctgaagtctaacgctcaggccaaatacgtgaaggactgcgccaccatgtgcagacactacaagagagccgagaagcaggccatgaccatgagccagtggatcagagccagatgcgataagaccgacgacggcggagattggaggcctatcgtgcagttcctgagataccagaacgtcgagttcatcacctttctgagcgcctttaaggccttcctgaagggaatccccaagagaagctgcatcgtgctgtacggccctccaaacaccggcaagagctacttcggaatgagcctgatgcagtttctgcagggcagcatcatcagctacgtgaacagcaacagccacttttggctggcccctctggccgaagctaaagtggccatgctggacgatgccacaccacagtgctggtcctacatcgacacctacatgcggagcgtgctggacggcaaccccatgagcattgaccggaagcacaagaacctgatccagatgaagtgtccgcctctgatcatcaccagcaacaccaacgtgggcgaagatgacagatggcgctacctgcactccagagtggtggtgttcaagttcgagcagacctttccattcgaccagaacggcaatcccgtgtacgagctgtccgacaagaattggaagtccttcttcagccggacctggtccagactcgagctgcaagaggatgaggacaccgagcaggatggctctacagtgcgggcctttaagtgtgtgcccggccagaacaccaatatcctgggctctatggtggaaggcagccaagagagagcccggacactgcacgaactgtgcgagcagagagaacagacccagcacgacatcgagctggactgcgtgtactgcctgaatcctctgaccagaaccgaggtgtacgacttcgccagatgggagctgagaatcgtgtacagacgggataccgcctacggcgtgtgcaagctgtgcctgaggttccacagcaaagtgcggaagtacaggcggtacaactactccatctacggccccacactggaacggcggacaaagaagcagctcgaagaactcgtgatccggtgctactgctgccagaaacctcagtgccccgtggaaaagcagcgccacgttgaccagggccagagattccacaacattgccgagcagtggaccggcagatgcctgcaatgttggaggccaacagccgctgaaaccggctctatgatcggccctggacctacactgcaggatatcgtgcttgagctggaacccgagcctgtggacctgagaggatatgagcagctggactccagcgaggacgaggacgagattgaccaccaccaacagcaggacagacccgatgtgcaagaagatgacggcgagcggtttagaatcctgagcgactgtcacgtgtgccagaataccgtgtcaagagtggtgcacagcacccacgacgacctgcacattctcgaggacctgcttatgggcaccctcgagatcgtggcccctaattgtgccgtgggcagcggcgccaccaatttcagtctgcttaaacaggccggggacgtcgaagagaaccccggacctatgcatcggcgcagatctcggtcttgtagagaagaccaaaagcctgtcatggatgatcaaagagatttaatttctaataatgaacaattacccatgttgggacggcgccccggagcccccgaatctaagtgcagccggggagctttatatacaggattttctattttagtgaccttattattagctggccaagccacaacagcttatttcttataccaacaacaaggacggttagataaattaacagtcacatctcaaaatttacaattagagaatttaagaatgaaattacctaaaccacccaaacccgtcagtaaaatgagaatggctacccccttattaatgcaagccctccccatgggcgccttaccccaaggacccatgcaaaatgctacaaagtatggaaatatgacagaagatcatgtcatgcacttattacaaaacgctgaccccttaaaagtctatccccctttaaaaggatctttccctgaaaatttaagacatttaaaaaatacaatggagacaattgattggaaagtctttgaatcttggatgcatcattggttattatttgaaatgtctaggcattctttagagcaaaaacccaccgatgctccacccaaggaaagtttagagttagaggacccttctagcggattaggagtcaccaaacaggacttaggacccgtccccatgatggaagccctgagcgagagactgaacgcctaccaggacaagatcctgaccttctacgaagaggactccaaggacctgagggaccagatcgagcattggagatgtgtgcggggagagtgcgccgtgttctacaaggccagagagatgggccacagccacatcaaccaccagcaggttccacctctgcacgtgtccagaggcaaggcccacaatgccattgagctgcagatggccctggaaagcctgaaccagagcgacttcaacaccgaggaatggaccctgcaggacacaagcctggaaatgtggcggaccgagcctcagtgctgcttcaaaaagaaaggcgagacagtgaccgtgctgtatgactgcgacaaggacaacagcatggactacaccctgtggggccacgtgtacgtgtggggagatacaggctgggacaaagtgtgcggaaaggtggactacggcggcctgtactatgtgctgcacggcctgaaagtgtactacgttgagttccagcaagaggccgtgaagtacagcaagaccaacaagtgggaagtgcatatcggcagaacagtgatcccctacagcgagtccgtgtcctctacagccctgtgcgaaagagtgcctatcgccgagatcgccaatggcctccagcagaccagcattccctctcacacacacgccaccaccaaagagaacgcttgggtttcctctcagcctcctaccaagcggtccagaagatctgatccaggcggcgatcctgtcagagccctggatgccaatagcagaaccctgctgtctggaaccgcctgcgagaataccaccggcaactcttacagcgactgcacccctgtggtgcacctgaaaggcgaccccaattgtctgaaggccctgcggttcagattcgtgaaacacaagaatctgttcgtgaacgtgtcctccacctggcggtggaccaacaacaccgaagagaaggccatcgtgactgtgacctttaaggacgagcagcagagacagcagttcctcggcagcgtgaagatccccaactccatctgtgtgtgcaagggcgtgatgacagttggcagcgaacagaaactgatctccgaggaagatctctgaggaagaatgtgcacagtgctaaccagaaaattcaggacaagactagtgcggccgctttggaaagttttataggtagttgatagaacaaaatacataattttgtaaaaataaatcactttttat

SEQ ID NO: 74 - DNA, 3662 bp, synthetic construct
  misc_feature 1..3662: note = MfPV3 Ii-E1E6E7 integrated into DelIII locus of the poxviral vector
  source 1..3662: mol_type = other DNA
aaaaattaaaattttattttttttttttggaatataaataagatctatccctatcagtgatagagatctccctatcagtgatagagatcgtcgacgagctcgtttagtgaaccgtcagatcgcctggagacgccatccacgctgttttgacctctagcaccggtgccaccatgcacagaaggcggagcagaagctgccgcgaggatcagaaacccgtgatggacgaccagcgggacctgatcagcaacaacgagcagctgcctatgctcggcagaaggcctggcgctcctgagagcaaatgttctagaggcgccctgtacaccggcttcagcatcctggttacactgctgctggccggacaggctaccaccgcctactttctgtatcagcagcagggcagactggacaagctgaccgtgaccagccagaacctgcagctggaaaacctgcggatgaagctgcccaagcctcctaagcctgtgtccaagatgcggatggccacacctctgctgatgcaggctttgcctatgggagcactgcctcagggccctatgcagaacgccaccaaatacggcaacatgaccgaggaccacgtgatgcatctgctgcagaatgccgatcctctgaaggtgtaccctccactgaagggcagctttcccgagaacctgcggcacctgaagaacaccatggaaaccatcgactggaaggtgttcgagagctggatgcaccactggctgctgttcgagatgagccggcacagcctggaacagaagcctacagacgcccctcctaaagagtccctggaactggaagatcccagctctggcctgggcgtgacaaagcaagatctgggccctgtgcctatgatggacccttctggaattcacggcgagggcaccagatgcaccggatggttcgatgtggaagccatcgtggaaagacggaccggcgacgtggtgtccgaggacgaagatgagaacctggaagataccggcgtggacctggtggacttcatcgacgatacctgcgagtttgagcaggccggcgacgaagctcctagagcactgctgcatgcccaagagacacaggcccatgtgaacgccgtgcactacctgaagcggaagttcacatgcagccctaccgtgtctcccctgggcaatgtgcagtgttctatcgagggcgagctgagccctagactgcacgccatccaactgaatgaaggcagcggcaaggccaagcggagactgtttctgcctgatagcggctacggcaataccgaggtggaaacccagctgtctcaggtggcaggatccgatggcgaatctcaagcccctggcggcaaagaaaatgtgcagcctgctgaaggcgctggcggagatgttcgacagctgctgagaagcagcaacctgaaggccacactgctgggcaagttcaaggacgtgtacggcatcggcttcaacgaactcgtgcggtgcttcaagagcgacaagaccgtgtgcaccgattgggttgtgggagctgctggcgtgcaccattctgtggccgaaggactgaagcacctgatcgagcctttctgcacatacgcccacatgcagtgcctgacctgcaactggggagtgtttctgctgctgctcatccggttcaagtgcggcaagaacagactgaccgtggccaagtgcatctctaccctgctgaacgtgcaagagccctacctgctgatcgaacctccaaagctgagatccacagccgccgctctgtactggtacagaaccggcatcagcaacgtgtccgaagtgatcggagagcctcctgagtggatcgtgcggcagaccatgtttcagcacagcctcgaggacagcatcttcgacctgtctcagatggtgcagtgggcctacgaccacgatctggtggacgatagcgccattgcctaccagtatgcccagctggccgacgtggactctaatgccgccgctttcctgaagtctaacgctcaggccaaatacgtgaaggactgcgccaccatgtgcagacactacaagagagccgagaagcaggccatgaccatgagccagtggatcagagccagatgcgataagaccgacgacggcggagattggaggcctatcgtgcagttcctgagataccagaacgtcgagttcatcacctttctgagcgcctttaaggccttcctgaagggaatccccaagagaagctgcatcgtgctgtacggccctccaaacaccggcaagagctacttcggaatgagcctgatgcagtttctgcagggcagcatcatcagctacgtgaacagcaacagccacttttggctggcccctctggccgaagctaaagtggccatgctggacgatgccacaccacagtgctggtcctacatcgacacctacatgcggagcgtgctggacggcaaccccatgagcattgaccggaagcacaagaacctgatccagatgaagtgtccgcctctgatcatcaccagcaacaccaacgtgggcgaagatgacagatggcgctacctgcactccagagtggtggtgttcaagttcgagcagacctttccattcgaccagaacggcaatcccgtgtacgagctgtccgacaagaattggaagtccttcttcagccggacctggtccagactcgagctgcaagaggatgaggacaccgagcaggatggctctacagtgcgggcctttaagtgtgtgcccggccagaacaccaatatcctgggctctatggtggaaggcagccaagagagagcccggacactgcacgaactgtgcgagcagagagaacagacccagcacgacatcgagctggactgcgtgtactgcctgaatcctctgaccagaaccgaggtgtacgacttcgccagatgggagctgagaatcgtgtacagacgggataccgcctacggcgtgtgcaagctgtgcctgaggttccacagcaaagtgcggaagtacaggcggtacaactactccatctacggccccacactggaacggcggacaaagaagcagctcgaagaactcgtgatccggtgctactgctgccagaaacctcagtgccccgtggaaaagcagcgccacgttgaccagggccagagattccacaacattgccgagcagtggaccggcagatgcctgcaatgttggaggccaacagccgctgaaaccggctctatgatcggccctggacctacactgcaggatatcgtgcttgagctggaacccgagcctgtggacctgagaggatatgagcagctggactccagcgaggacgaggacgagattgaccaccaccaacagcaggacagacccgatgtgcaagaagatgacggcgagcggtttagaatcctgagcgactgtcacgtgtgccagaataccgtgtcaagagtggtgcacagcacccacgacgacctgcacattctcgaggacctgcttatgggcaccctcgagatcgtggcccctaattgtgccgtgggcagcgaacagaaactgatctccgaggaagatctctgaggaagaatgtgcacagtgctaaccagaaaattcaggacaagactagtgcggccgctttggaaagttttataggtagttgatagaacaaaatacataattttgtaaaaataaatcactttttat

SEQ ID NO: 75 - DNA, 4808 bp, synthetic construct
  misc_feature 1..4808: note = HPV16 Ii-E1E2E6E7 integrated into DelIII locus of the poxviral vector
  source 1..4808: mol_type = other DNA
aaaaattaaaattttattttttttttttggaatataaataagatctatccctatcagtgatagagatctccctatcagtgatagagatcgtcgacgagctcgtttagtgaaccgtcagatcgcctggagacgccatccacgctgttttgacctctagcaccggtgccaccatgcacagaaggcggagcagaagctgccgcgaggatcagaaacccgtgatggacgaccagcgggacctgatcagcaacaacgagcagctgcctatgctcggcagaaggcctggcgctcctgagagcaaatgttctagaggcgccctgtacaccggcttcagcatcctggttacactgctgctggccggacaggctaccaccgcctactttctgtatcagcagcagggcagactggacaagctgaccgtgaccagccagaacctgcagctggaaaacctgcggatgaagctgcccaagcctcctaagcctgtgtccaagatgcggatggccacacctctgctgatgcaggctttgcctatgggagcactgcctcagggccctatgcagaacgccaccaaatacggcaacatgaccgaggaccacgtgatgcatctgctgcagaatgccgatcctctgaaggtgtaccctccactgaagggcagctttcccgagaacctgcggcacctgaagaacaccatggaaaccatcgactggaaggtgttcgagagctggatgcaccactggctgctgttcgagatgagccggcacagcctggaacagaagcctacagacgcccctcctaaagagtccctggaactggaagatcccagctctggcctgggcgtgacaaagcaagatctgggccctgtgcctatgatggccgatcctgctggcacaaacggcgaagaaggcacaggctgcaacggctggttttacgtggaagccgtggtggaaaagaaaaccggcgacgccatcagcgacgacgagaacgagaatgacagcgacaccggcgaggacctggtggacttcatcgtgaacgacaacgactacctgacacaggccgagacagagacagcccacgctctgtttacagcccaagaggccaagcagcacagagatgccgttcaggtgctgaagagaaagtacctgggcagccctctgagcgacatcagcggctgtgtggacaacaacatcagccccagactgaaggccatctgcatcgagaagcagagcagagccgccaagcggagactgtttgagagcgaggatagcggctacggcaataccgaggtggaaacccagcaaatgctgcaggtcgagggcagacacgaaaccgagacaccttgcagccagtacagcggaggatctggcggaggctgtagccagtactcttctggaagtggcggagagggcgtgtccgagagacacaccatttgtcagacccctctgaccaacatcctgaacgtgctgaaaaccagcaacgccaaggccgccatgctggccaagttcaaagagctgtacggcgtcagcttcagcgaactcgtgcggcccttcaagagcaacaagtccacctgttgcgactggtgcattgccgcctttggcctgacaccttctatcgccgacagcatcaagaccctgctccagcagtactgtctgtacctgcacattcagagcctggcctgctcttggggaatggtggtgctgctgctcgtgcggtacaagtgcggcaagaacagagagacaattgagaagctgctgagcaaactgctgtgcgtgtcccctatgtgcatgatgatcgagcctccaaagctgcggtctacagccgccgctctgtactggtacaagaccggcatcagcaatatctccgaggtgtacggcgacacccctgagtggattcagagacagaccgtcctgcagcacagcttcaacgactgcaccttcgagctgtcccagatggtgcagtgggcctacgacaatgacatcgtggacgactccgagatcgcctataagtacgcccagctggccgacaccaacagcaatgccagcgccttcctgaagtccaacagccaggccaagatcgtgaaggactgcgccaccatgtgcagacactacaagcgggccgagaagaaacagatgagcatgagccagtggatcaagtacagatgcgacagagtggacgacggcggcgattggaagcagatcgtgatgttcctgagataccagggcgtcgagttcatgagctttctgacagccctgaagcggttcctgcagggcatccccaagaagaactgcatcctgctgtatggcgccgctaacaccggcaagagcctgttcggaatgagcctgatgaagtttctgcagggctccgtgatctgctttgtgaacagcaagagccacttctggctccagccactggccgatgccaagattggcatgctggacgatgccaccgtgccttgctggaactacatcgacgacaacctgagaaacgccctggacggcaacctggtttctatggacgtgaagcacagacccctggtgcagctgaagtgtcctccactgctgatcaccagcaacatcaacgccggcaccgattccagatggccctatctgcacaaccggctggtggtgtttacctttcctaacgagttccccttcgacgaaaacggcaaccccgtgtacgagctgaacgataagaattggaagtccttcttcagccggacctggtccagactgagcctgcacgaggatgaggacaaagagaacgacggcgacagcctgcctaccttcaaatgtgtcagcggccagaacaccaacacactgggcagcatggaaacactgtgccagcggctgaacgtgtgccaggacaagattctgacccactatgagaacgacagcaccgacctgagggaccacatcgactactggaagcacatgcggctggaatgcgccatctactacaaggctagagagatgggcttcaagcacatcaaccaccaggtggtgcctacactggccgtgtctaagaacaaggctctgcaggccatcgaactgcagctgaccctggaaacaatctacaactcccagtactccaacgagaagtggaccctccaggacgtgtcactggaagtgtacctgaccgctcctaccggctgcatcaagaaacacggctacaccgtggaagtgcagtttgacggcgatatctgcaataccatgcactacaccaactggacccacatctacatctgcgaagaggccagcgtgacagtggtggaaggccaggtcgactactacggcctgtactacgtgcacgagggcatcagaacctacttcgtgcagttcaaggatgacgccgagaagtacagcaagaacaaagtgtgggaagtgcacgctggcggccaagtgatcctgtgtcctacaagcgtgttcagcagcaacgaagtgtctagccccgagatcatcagacagcacctggccaatcatcctgccgccacacacacaaaagctgtggccctgggcaccgaggaaacacagaccacaattcagaggcccagaagcgagcccgataccggcaatccttgtcacaccaccaagctgctgcaccgggattccgtggattctgcccctatcctgaccgccttcaacagctcccacaaaggccggatcaactgcaacagcaacaccacacctatcgtgcacctgaaaggggacgccaacactctgaaggccctgcggtacagattcaagaagcactgtaccctgtataccgccgtgtccagcacatggcactggaccggacacaacgtgaaacacaagagcgccatcgtgaccctgacctacgatagcgagtggcagagggaccagttcctgagccaagtgaagatccctaagaccatcaccgtgtccaccggctttatgagcattggcagcatgcaccagaaacggacagccatgttccaggatcctcaagagcggccacggaagctgcctcagctgtgtacagaactgcagaccaccatccacgacatcatcctcgagtgcgtgtactgcaagcagcagctcctgcggagagaagtgtacgacttcgccttccgggatctgtgcatcgtgtacagagatggcaacccctacgccgtgtgcgacaagtgcctgaagttctactccaagatcagcgagtaccggcactactgctactccctgtacggcaccacacttgagcagcagtacaacaagcccctgtgcgatctgctgatccggtgcatcaactgccagaaacctcagtgccccgaggaaaagcagagacacctggacaagaagcagcggttccacaacattagaggcagatggacaggccggtgcatgagctgctgtagaagcagcagaacaagacggggctccatgcacggcgataccccaacactgcacgagtacatgctggatctgcagcccgagacaaccgatctgtacggctatgagcagctgaatgactccagcgaggaagaggacgagatcgacggacctgctggacaggctgaacctgatagagcccactacaatatcgtgaccttctgctgcaagtgcgatagcaccctgagaagatgcgtgcagagcacccacgtggacatcaggaccctcgaggatctgctcatgggcaccctgggaattgtggccccaatctgctctcagaagccttgaggaagaatgtgcacagtgctaaccagaaaattcaggacaagactagtgcggccgctttggaaagttttataggtagttgatagaacaaaatacataattttgtaaaaataaatcactttttat

SEQ ID NO: 76 - DNA, 5570 bp, synthetic construct
  misc_feature 1..5570: note = HPV16 Ii-E1E2-p2a-Ii-E6E7 integrated into DelIII locus of the poxviral vector
  source 1..5570: mol_type = other DNA
aaaaattaaaattttattttttttttttggaatataaataagatctatccctatcagtgatagagatctccctatcagtgatagagatcgtcgacgagctcgtttagtgaaccgtcagatcgcctggagacgccatccacgctgttttgacctctagcaccggtgccaccatgcacagaaggcggagcagaagctgccgcgaggatcagaaacccgtgatggacgaccagcgggacctgatcagcaacaacgagcagctgcctatgctcggcagaaggcctggcgctcctgagagcaaatgttctagaggcgccctgtacaccggcttcagcatcctggttacactgctgctggccggacaggctaccaccgcctactttctgtatcagcagcagggcagactggacaagctgaccgtgaccagccagaacctgcagctggaaaacctgcggatgaagctgcccaagcctcctaagcctgtgtccaagatgcggatggccacacctctgctgatgcaggctttgcctatgggagcactgcctcagggccctatgcagaacgccaccaaatacggcaacatgaccgaggaccacgtgatgcatctgctgcagaatgccgatcctctgaaggtgtaccctccactgaagggcagctttcccgagaacctgcggcacctgaagaacaccatggaaaccatcgactggaaggtgttcgagagctggatgcaccactggctgctgttcgagatgagccggcacagcctggaacagaagcctacagacgcccctcctaaagagtccctggaactggaagatcccagctctggcctgggcgtgacaaagcaagatctgggccctgtgcctatgatggccgatcctgctggcacaaacggcgaagaaggcacaggctgcaacggctggttttacgtggaagccgtggtggaaaagaaaaccggcgacgccatcagcgacgacgagaacgagaatgacagcgacaccggcgaggacctggtggacttcatcgtgaacgacaacgactacctgacacaggccgagacagagacagcccacgctctgtttacagcccaagaggccaagcagcacagagatgccgttcaggtgctgaagagaaagtacctgggcagccctctgagcgacatcagcggctgtgtggacaacaacatcagccccagactgaaggccatctgcatcgagaagcagagcagagccgccaagcggagactgtttgagagcgaggatagcggctacggcaataccgaggtggaaacccagcaaatgctgcaggtcgagggcagacacgaaaccgagacaccttgcagccagtacagcggaggatctggcggaggctgtagccagtactcttctggaagtggcggagagggcgtgtccgagagacacaccatttgtcagacccctctgaccaacatcctgaacgtgctgaaaaccagcaacgccaaggccgccatgctggccaagttcaaagagctgtacggcgtcagcttcagcgaactcgtgcggcccttcaagagcaacaagtccacctgttgcgactggtgcattgccgcctttggcctgacaccttctatcgccgacagcatcaagaccctgctccagcagtactgtctgtacctgcacattcagagcctggcctgctcttggggaatggtggtgctgctgctcgtgcggtacaagtgcggcaagaacagagagacaattgagaagctgctgagcaaactgctgtgcgtgtcccctatgtgcatgatgatcgagcctccaaagctgcggtctacagccgccgctctgtactggtacaagaccggcatcagcaatatctccgaggtgtacggcgacacccctgagtggattcagagacagaccgtcctgcagcacagcttcaacgactgcaccttcgagctgtcccagatggtgcagtgggcctacgacaatgacatcgtggacgactccgagatcgcctataagtacgcccagctggccgacaccaacagcaatgccagcgccttcctgaagtccaacagccaggccaagatcgtgaaggactgcgccaccatgtgcagacactacaagcgggccgagaagaaacagatgagcatgagccagtggatcaagtacagatgcgacagagtggacgacggcggcgattggaagcagatcgtgatgttcctgagataccagggcgtcgagttcatgagctttctgacagccctgaagcggttcctgcagggcatccccaagaagaactgcatcctgctgtatggcgccgctaacaccggcaagagcctgttcggaatgagcctgatgaagtttctgcagggctccgtgatctgctttgtgaacagcaagagccacttctggctccagccactggccgatgccaagattggcatgctggacgatgccaccgtgccttgctggaactacatcgacgacaacctgagaaacgccctggacggcaacctggtttctatggacgtgaagcacagacccctggtgcagctgaagtgtcctccactgctgatcaccagcaacatcaacgccggcaccgattccagatggccctatctgcacaaccggctggtggtgtttacctttcctaacgagttccccttcgacgaaaacggcaaccccgtgtacgagctgaacgataagaattggaagtccttcttcagccggacctggtccagactgagcctgcacgaggatgaggacaaagagaacgacggcgacagcctgcctaccttcaaatgtgtcagcggccagaacaccaacacactgggcagcatggaaacactgtgccagcggctgaacgtgtgccaggacaagattctgacccactatgagaacgacagcaccgacctgagggaccacatcgactactggaagcacatgcggctggaatgcgccatctactacaaggctagagagatgggcttcaagcacatcaaccaccaggtggtgcctacactggccgtgtctaagaacaaggctctgcaggccatcgaactgcagctgaccctggaaacaatctacaactcccagtactccaacgagaagtggaccctccaggacgtgtcactggaagtgtacctgaccgctcctaccggctgcatcaagaaacacggctacaccgtggaagtgcagtttgacggcgatatctgcaataccatgcactacaccaactggacccacatctacatctgcgaagaggccagcgtgacagtggtggaaggccaggtcgactactacggcctgtactacgtgcacgagggcatcagaacctacttcgtgcagttcaaggatgacgccgagaagtacagcaagaacaaagtgtgggaagtgcacgctggcggccaagtgatcctgtgtcctacaagcgtgttcagcagcaacgaagtgtctagccccgagatcatcagacagcacctggccaatcatcctgccgccacacacacaaaagctgtggccctgggcaccgaggaaacacagaccacaattcagaggcccagaagcgagcccgataccggcaatccttgtcacaccaccaagctgctgcaccgggattccgtggattctgcccctatcctgaccgccttcaacagctcccacaaaggccggatcaactgcaacagcaacaccacacctatcgtgcacctgaaaggggacgccaacactctgaaggccctgcggtacagattcaagaagcactgtaccctgtataccgccgtgtccagcacatggcactggaccggacacaacgtgaaacacaagagcgccatcgtgaccctgacctacgatagcgagtggcagagggaccagttcctgagccaagtgaagatccctaagaccatcaccgtgtccaccggctttatgagcattggcagcggcagcggcgccaccaatttcagtctgcttaaacaggccggggacgtcgaagagaaccccggacctatgcatcggcgcagatctcggtcttgtagagaagaccaaaagcctgtcatggatgatcaaagagatttaatttctaataatgaacaattacccatgttgggacggcgccccggagcccccgaatctaagtgcagccggggagctttatatacaggattttctattttagtgaccttattattagctggccaagccacaacagcttatttcttataccaacaacaaggacggttagataaattaacagtcacatctcaaaatttacaattagagaatttaagaatgaaattacctaaaccacccaaacccgtcagtaaaatgagaatggctacccccttattaatgcaagccctccccatgggcgccttaccccaaggacccatgcaaaatgctacaaagtatggaaatatgacagaagatcatgtcatgcacttattacaaaacgctgaccccttaaaagtctatccccctttaaaaggatctttccctgaaaatttaagacatttaaaaaatacaatggagacaattgattggaaagtctttgaatcttggatgcatcattggttattatttgaaatgtctaggcattctttagagcaaaaacccaccgatgctccacccaaggaaagtttagagttagaggacccttctagcggattaggagtcaccaaacaggacttaggacccgtccccatgatgcaccagaaacggacagccatgttccaggatcctcaagagcggccacggaagctgcctcagctgtgtacagaactgcagaccaccatccacgacatcatcctcgagtgcgtgtactgcaagcagcagctcctgcggagagaagtgtacgacttcgccttccgggatctgtgcatcgtgtacagagatggcaacccctacgccgtgtgcgacaagtgcctgaagttctactccaagatcagcgagtaccggcactactgctactccctgtacggcaccacacttgagcagcagtacaacaagcccctgtgcgatctgctgatccggtgcatcaactgccagaaacctcagtgccccgaggaaaagcagagacacctggacaagaagcagcggttccacaacattagaggcagatggacaggccggtgcatgagctgctgtagaagcagcagaacaagacggggctccatgcacggcgataccccaacactgcacgagtacatgctggatctgcagcccgagacaaccgatctgtacggctatgagcagctgaatgactccagcgaggaagaggacgagatcgacggacctgctggacaggctgaacctgatagagcccactacaatatcgtgaccttctgctgcaagtgcgatagcaccctgagaagatgcgtgcagagcacccacgtggacatcaggaccctcgaggatctgctcatgggcaccctgggaattgtggccccaatctgctctcagaagccttgaggaagaatgtgcacagtgctaaccagaaaattcaggacaagactagtgcggccgctttggaaagttttataggtagttgatagaacaaaatacataattttgtaaaaataaatcactttttat

SEQ ID NO: 77 - DNA, 4112 bp, synthetic construct
  misc_feature 1..4112: note = HPV16 E1E2E6E7 integrated into DelIII locus of the poxviral vector
  source 1..4112: mol_type = other DNA
aaaaattaaaattttattttttttttttggaatataaataagatctatccctatcagtgatagagatctccctatcagtgatagagatcgtcgacgagctcgtttagtgaaccgtcagatcgcctggagacgccatccacgctgttttgacctctagcaccggtgccaccatggccgatcctgctggcacaaacggcgaagaaggcacaggctgcaacggctggttttacgtggaagccgtggtggaaaagaaaaccggcgacgccatcagcgacgacgagaacgagaatgacagcgacaccggcgaggacctggtggacttcatcgtgaacgacaacgactacctgacacaggccgagacagagacagcccacgctctgtttacagcccaagaggccaagcagcacagagatgccgttcaggtgctgaagagaaagtacctgggcagccctctgagcgacatcagcggctgtgtggacaacaacatcagccccagactgaaggccatctgcatcgagaagcagagcagagccgccaagcggagactgtttgagagcgaggatagcggctacggcaataccgaggtggaaacccagcaaatgctgcaggtcgagggcagacacgaaaccgagacaccttgcagccagtacagcggaggatctggcggaggctgtagccagtactcttctggaagtggcggagagggcgtgtccgagagacacaccatttgtcagacccctctgaccaacatcctgaacgtgctgaaaaccagcaacgccaaggccgccatgctggccaagttcaaagagctgtacggcgtcagcttcagcgaactcgtgcggcccttcaagagcaacaagtccacctgttgcgactggtgcattgccgcctttggcctgacaccttctatcgccgacagcatcaagaccctgctccagcagtactgtctgtacctgcacattcagagcctggcctgctcttggggaatggtggtgctgctgctcgtgcggtacaagtgcggcaagaacagagagacaattgagaagctgctgagcaaactgctgtgcgtgtcccctatgtgcatgatgatcgagcctccaaagctgcggtctacagccgccgctctgtactggtacaagaccggcatcagcaatatctccgaggtgtacggcgacacccctgagtggattcagagacagaccgtcctgcagcacagcttcaacgactgcaccttcgagctgtcccagatggtgcagtgggcctacgacaatgacatcgtggacgactccgagatcgcctataagtacgcccagctggccgacaccaacagcaatgccagcgccttcctgaagtccaacagccaggccaagatcgtgaaggactgcgccaccatgtgcagacactacaagcgggccgagaagaaacagatgagcatgagccagtggatcaagtacagatgcgacagagtggacgacggcggcgattggaagcagatcgtgatgttcctgagataccagggcgtcgagttcatgagctttctgacagccctgaagcggttcctgcagggcatccccaagaagaactgcatcctgctgtatggcgccgctaacaccggcaagagcctgttcggaatgagcctgatgaagtttctgcagggctccgtgatctgctttgtgaacagcaagagccacttctggctccagccactggccgatgccaagattggcatgctggacgatgccaccgtgccttgctggaactacatcgacgacaacctgagaaacgccctggacggcaacctggtttctatggacgtgaagcacagacccctggtgcagctgaagtgtcctccactgctgatcaccagcaacatcaacgccggcaccgattccagatggccctatctgcacaaccggctggtggtgtttacctttcctaacgagttccccttcgacgaaaacggcaaccccgtgtacgagctgaacgataagaattggaagtccttcttcagccggacctggtccagactgagcctgcacgaggatgaggacaaagagaacgacggcgacagcctgcctaccttcaaatgtgtcagcggccagaacaccaacacactgggcagcatggaaacactgtgccagcggctgaacgtgtgccaggacaagattctgacccactatgagaacgacagcaccgacctgagggaccacatcgactactggaagcacatgcggctggaatgcgccatctactacaaggctagagagatgggcttcaagcacatcaaccaccaggtggtgcctacactggccgtgtctaagaacaaggctctgcaggccatcgaactgcagctgaccctggaaacaatctacaactcccagtactccaacgagaagtggaccctccaggacgtgtcactggaagtgtacctgaccgctcctaccggctgcatcaagaaacacggctacaccgtggaagtgcagtttgacggcgatatctgcaataccatgcactacaccaactggacccacatctacatctgcgaagaggccagcgtgacagtggtggaaggccaggtcgactactacggcctgtactacgtgcacgagggcatcagaacctacttcgtgcagttcaaggatgacgccgagaagtacagcaagaacaaagtgtgggaagtgcacgctggcggccaagtgatcctgtgtcctacaagcgtgttcagcagcaacgaagtgtctagccccgagatcatcagacagcacctggccaatcatcctgccgccacacacacaaaagctgtggccctgggcaccgaggaaacacagaccacaattcagaggcccagaagcgagcccgataccggcaatccttgtcacaccaccaagctgctgcaccgggattccgtggattctgcccctatcctgaccgccttcaacagctcccacaaaggccggatcaactgcaacagcaacaccacacctatcgtgcacctgaaaggggacgccaacactctgaaggccctgcggtacagattcaagaagcactgtaccctgtataccgccgtgtccagcacatggcactggaccggacacaacgtgaaacacaagagcgccatcgtgaccctgacctacgatagcgagtggcagagggaccagttcctgagccaagtgaagatccctaagaccatcaccgtgtccaccggctttatgagcattggcagcatgcaccagaaacggacagccatgttccaggatcctcaagagcggccacggaagctgcctcagctgtgtacagaactgcagaccaccatccacgacatcatcctcgagtgcgtgtactgcaagcagcagctcctgcggagagaagtgtacgacttcgccttccgggatctgtgcatcgtgtacagagatggcaacccctacgccgtgtgcgacaagtgcctgaagttctactccaagatcagcgagtaccggcactactgctactccctgtacggcaccacacttgagcagcagtacaacaagcccctgtgcgatctgctgatccggtgcatcaactgccagaaacctcagtgccccgaggaaaagcagagacacctggacaagaagcagcggttccacaacattagaggcagatggacaggccggtgcatgagctgctgtagaagcagcagaacaagacggggctccatgcacggcgataccccaacactgcacgagtacatgctggatctgcagcccgagacaaccgatctgtacggctatgagcagctgaatgactccagcgaggaagaggacgagatcgacggacctgctggacaggctgaacctgatagagcccactacaatatcgtgaccttctgctgcaagtgcgatagcaccctgagaagatgcgtgcagagcacccacgtggacatcaggaccctcgaggatctgctcatgggcaccctgggaattgtggccccaatctgctctcagaagccttgaggaagaatgtgcacagtgctaaccagaaaattcaggacaagactagtgcggccgctttggaaagttttataggtagttgatagaacaaaatacataattttgtaaaaataaatcactttttat

SEQ ID NO: 78 - DNA, 5564 bp, synthetic construct
  misc_feature 1..5564: note = HPV16 Ii-E1E6E7-p2a-Ii-E2 integrated into DelIII locus of the poxviral vector
  source 1..5564: mol_type = other DNA
aaaaattaaaattttattttttttttttggaatataaataagatctatccctatcagtgatagagatctccctatcagtgatagagatcgtcgacgagctcgtttagtgaaccgtcagatcgcctggagacgccatccacgctgttttgacctctagcaccggtgccaccatgcacagaaggcggagcagaagctgccgcgaggatcagaaacccgtgatggacgaccagcgggacctgatcagcaacaacgagcagctgcctatgctcggcagaaggcctggcgctcctgagagcaaatgttctagaggcgccctgtacaccggcttcagcatcctggttacactgctgctggccggacaggctaccaccgcctactttctgtatcagcagcagggcagactggacaagctgaccgtgaccagccagaacctgcagctggaaaacctgcggatgaagctgcccaagcctcctaagcctgtgtccaagatgcggatggccacacctctgctgatgcaggctttgcctatgggagcactgcctcagggccctatgcagaacgccaccaaatacggcaacatgaccgaggaccacgtgatgcatctgctgcagaatgccgatcctctgaaggtgtaccctccactgaagggcagctttcccgagaacctgcggcacctgaagaacaccatggaaaccatcgactggaaggtgttcgagagctggatgcaccactggctgctgttcgagatgagccggcacagcctggaacagaagcctacagacgcccctcctaaagagtccctggaactggaagatcccagctctggcctgggcgtgacaaagcaagatctgggccctgtgcctatgatggccgatcctgctggcacaaacggcgaagaaggcacaggctgcaacggctggttttacgtggaagccgtggtggaaaagaaaaccggcgacgccatcagcgacgacgagaacgagaatgacagcgacaccggcgaggacctggtggacttcatcgtgaacgacaacgactacctgacacaggccgagacagagacagcccacgctctgtttacagcccaagaggccaagcagcacagagatgccgttcaggtgctgaagagaaagtacctgggcagccctctgagcgacatcagcggctgtgtggacaacaacatcagccccagactgaaggccatctgcatcgagaagcagagcagagccgccaagcggagactgtttgagagcgaggatagcggctacggcaataccgaggtggaaacccagcaaatgctgcaggtcgagggcagacacgaaaccgagacaccttgcagccagtacagcggaggatctggcggaggctgtagccagtactcttctggaagtggcggagagggcgtgtccgagagacacaccatttgtcagacccctctgaccaacatcctgaacgtgctgaaaaccagcaacgccaaggccgccatgctggccaagttcaaagagctgtacggcgtcagcttcagcgaactcgtgcggcccttcaagagcaacaagtccacctgttgcgactggtgcattgccgcctttggcctgacaccttctatcgccgacagcatcaagaccctgctccagcagtactgtctgtacctgcacattcagagcctggcctgctcttggggaatggtggtgctgctgctcgtgcggtacaagtgcggcaagaacagagagacaattgagaagctgctgagcaaactgctgtgcgtgtcccctatgtgcatgatgatcgagcctccaaagctgcggtctacagccgccgctctgtactggtacaagaccggcatcagcaatatctccgaggtgtacggcgacacccctgagtggattcagagacagaccgtcctgcagcacagcttcaacgactgcaccttcgagctgtcccagatggtgcagtgggcctacgacaatgacatcgtggacgactccgagatcgcctataagtacgcccagctggccgacaccaacagcaatgccagcgccttcctgaagtccaacagccaggccaagatcgtgaaggactgcgccaccatgtgcagacactacaagcgggccgagaagaaacagatgagcatgagccagtggatcaagtacagatgcgacagagtggacgacggcggcgattggaagcagatcgtgatgttcctgagataccagggcgtcgagttcatgagctttctgacagccctgaagcggttcctgcagggcatccccaagaagaactgcatcctgctgtatggcgccgctaacaccggcaagagcctgttcggaatgagcctgatgaagtttctgcagggctccgtgatctgctttgtgaacagcaagagccacttctggctccagccactggccgatgccaagattggcatgctggacgatgccaccgtgccttgctggaactacatcgacgacaacctgagaaacgccctggacggcaacctggtttctatggacgtgaagcacagacccctggtgcagctgaagtgtcctccactgctgatcaccagcaacatcaacgccggcaccgattccagatggccctatctgcacaaccggctggtggtgtttacctttcctaacgagttccccttcgacgaaaacggcaaccccgtgtacgagctgaacgataagaattggaagtccttcttcagccggacctggtccagactgagcctgcacgaggatgaggacaaagagaacgacggcgacagcctgcctaccttcaaatgtgtcagcggccagaacaccaacacactgggcagcatgcaccagaaacggacagccatgttccaggatcctcaagagcggccacggaagctgcctcagctgtgtacagaactgcagaccaccatccacgacatcatcctcgagtgcgtgtactgcaagcagcagctcctgcggagagaagtgtacgacttcgccttccgggatctgtgcatcgtgtacagagatggcaacccctacgccgtgtgcgacaagtgcctgaagttctactccaagatcagcgagtaccggcactactgctactccctgtacggcaccacacttgagcagcagtacaacaagcccctgtgcgatctgctgatccggtgcatcaactgccagaaacctcagtgccccgaggaaaagcagagacacctggacaagaagcagcggttccacaacattagaggcagatggacaggccggtgcatgagctgctgtagaagcagcagaacaagacggggctccatgcacggcgataccccaacactgcacgagtacatgctggatctgcagcccgagacaaccgatctgtacggctatgagcagctgaatgactccagcgaggaagaggacgagatcgacggacctgctggacaggctgaacctgatagagcccactacaatatcgtgaccttctgctgcaagtgcgatagcaccctgagaagatgcgtgcagagcacccacgtggacatcaggaccctcgaggatctgctcatgggcaccctgggaattgtggccccaatctgctctcagaagcctggcagcggcgccaccaatttcagtctgcttaaacaggccggggacgtcgaagagaaccccggacctatgcatcggcgcagatctcggtcttgtagagaagaccaaaagcctgtcatggatgatcaaagagatttaatttctaataatgaacaattacccatgttgggacggcgccccggagcccccgaatctaagtgcagccggggagctttatatacaggattttctattttagtgaccttattattagctggccaagccacaacagcttatttcttataccaacaacaaggacggttagataaattaacagtcacatctcaaaatttacaattagagaatttaagaatgaaattacctaaaccacccaaacccgtcagtaaaatgagaatggctacccccttattaatgcaagccctccccatgggcgccttaccccaaggacccatgcaaaatgctacaaagtatggaaatatgacagaagatcatgtcatgcacttattacaaaacgctgaccccttaaaagtctatccccctttaaaaggatctttccctgaaaatttaagacatttaaaaaatacaatggagacaattgattggaaagtctttgaatcttggatgcatcattggttattatttgaaatgtctaggcattctttagagcaaaaacccaccgatgctccacccaaggaaagtttagagttagaggacccttctagcggattaggagtcaccaaacaggacttaggacccgtccccatgatggaaacactgtgccagcggctgaacgtgtgccaggacaagattctgacccactatgagaacgacagcaccgacctgagggaccacatcgactactggaagcacatgcggctggaatgcgccatctactacaaggctagagagatgggcttcaagcacatcaaccaccaggtggtgcctacactggccgtgtctaagaacaaggctctgcaggccatcgaactgcagctgaccctggaaacaatctacaactcccagtactccaacgagaagtggaccctccaggacgtgtcactggaagtgtacctgaccgctcctaccggctgcatcaagaaacacggctacaccgtggaagtgcagtttgacggcgatatctgcaataccatgcactacaccaactggacccacatctacatctgcgaagaggccagcgtgacagtggtggaaggccaggtcgactactacggcctgtactacgtgcacgagggcatcagaacctacttcgtgcagttcaaggatgacgccgagaagtacagcaagaacaaagtgtgggaagtgcacgctggcggccaagtgatcctgtgtcctacaagcgtgttcagcagcaacgaagtgtctagccccgagatcatcagacagcacctggccaatcatcctgccgccacacacacaaaagctgtggccctgggcaccgaggaaacacagaccacaattcagaggcccagaagcgagcccgataccggcaatccttgtcacaccaccaagctgctgcaccgggattccgtggattctgcccctatcctgaccgccttcaacagctcccacaaaggccggatcaactgcaacagcaacaccacacctatcgtgcacctgaaaggggacgccaacactctgaaggccctgcggtacagattcaagaagcactgtaccctgtataccgccgtgtccagcacatggcactggaccggacacaacgtgaaacacaagagcgccatcgtgaccctgacctacgatagcgagtggcagagggaccagttcctgagccaagtgaagatccctaagaccatcaccgtgtccaccggctttatgagcatttgaggaagaatgtgcacagtgctaaccagaaaattcaggacaagactagtgcggccgctttggaaagttttataggtagttgatagaacaaaatacataattttgtaaaaataaatcactttttat

SEQ ID NO: 79 - DNA, 3707 bp, synthetic construct
  misc_feature 1..3707: note = HPV16 Ii-E1E6E7 integrated into DelIII locus of the poxviral vector
  source 1..3707: mol_type = other DNA
aaaaattaaaattttattttttttttttggaatataaataagatctatccctatcagtgatagagatctccctatcagtgatagagatcgtcgacgagctcgtttagtgaaccgtcagatcgcctggagacgccatccacgctgttttgacctctagcaccggtgccaccatgcacagaaggcggagcagaagctgccgcgaggatcagaaacccgtgatggacgaccagcgggacctgatcagcaacaacgagcagctgcctatgctcggcagaaggcctggcgctcctgagagcaaatgttctagaggcgccctgtacaccggcttcagcatcctggttacactgctgctggccggacaggctaccaccgcctactttctgtatcagcagcagggcagactggacaagctgaccgtgaccagccagaacctgcagctggaaaacctgcggatgaagctgcccaagcctcctaagcctgtgtccaagatgcggatggccacacctctgctgatgcaggctttgcctatgggagcactgcctcagggccctatgcagaacgccaccaaatacggcaacatgaccgaggaccacgtgatgcatctgctgcagaatgccgatcctctgaaggtgtaccctccactgaagggcagctttcccgagaacctgcggcacctgaagaacaccatggaaaccatcgactggaaggtgttcgagagctggatgcaccactggctgctgttcgagatgagccggcacagcctggaacagaagcctacagacgcccctcctaaagagtccctggaactggaagatcccagctctggcctgggcgtgacaaagcaagatctgggccctgtgcctatgatggccgatcctgctggcacaaacggcgaagaaggcacaggctgcaacggctggttttacgtggaagccgtggtggaaaagaaaaccggcgacgccatcagcgacgacgagaacgagaatgacagcgacaccggcgaggacctggtggacttcatcgtgaacgacaacgactacctgacacaggccgagacagagacagcccacgctctgtttacagcccaagaggccaagcagcacagagatgccgttcaggtgctgaagagaaagtacctgggcagccctctgagcgacatcagcggctgtgtggacaacaacatcagccccagactgaaggccatctgcatcgagaagcagagcagagccgccaagcggagactgtttgagagcgaggatagcggctacggcaataccgaggtggaaacccagcaaatgctgcaggtcgagggcagacacgaaaccgagacaccttgcagccagtacagcggaggatctggcggaggctgtagccagtactcttctggaagtggcggagagggcgtgtccgagagacacaccatttgtcagacccctctgaccaacatcctgaacgtgctgaaaaccagcaacgccaaggccgccatgctggccaagttcaaagagctgtacggcgtcagcttcagcgaactcgtgcggcccttcaagagcaacaagtccacctgttgcgactggtgcattgccgcctttggcctgacaccttctatcgccgacagcatcaagaccctgctccagcagtactgtctgtacctgcacattcagagcctggcctgctcttggggaatggtggtgctgctgctcgtgcggtacaagtgcggcaagaacagagagacaattgagaagctgctgagcaaactgctgtgcgtgtcccctatgtgcatgatgatcgagcctccaaagctgcggtctacagccgccgctctgtactggtacaagaccggcatcagcaatatctccgaggtgtacggcgacacccctgagtggattcagagacagaccgtcctgcagcacagcttcaacgactgcaccttcgagctgtcccagatggtgcagtgggcctacgacaatgacatcgtggacgactccgagatcgcctataagtacgcccagctggccgacaccaacagcaatgccagcgccttcctgaagtccaacagccaggccaagatcgtgaaggactgcgccaccatgtgcagacactacaagcgggccgagaagaaacagatgagcatgagccagtggatcaagtacagatgcgacagagtggacgacggcggcgattggaagcagatcgtgatgttcctgagataccagggcgtcgagttcatgagctttctgacagccctgaagcggttcctgcagggcatccccaagaagaactgcatcctgctgtatggcgccgctaacaccggcaagagcctgttcggaatgagcctgatgaagtttctgcagggctccgtgatctgctttgtgaacagcaagagccacttctggctccagccactggccgatgccaagattggcatgctggacgatgccaccgtgccttgctggaactacatcgacgacaacctgagaaacgccctggacggcaacctggtttctatggacgtgaagcacagacccctggtgcagctgaagtgtcctccactgctgatcaccagcaacatcaacgccggcaccgattccagatggccctatctgcacaaccggctggtggtgtttacctttcctaacgagttccccttcgacgaaaacggcaaccccgtgtacgagctgaacgataagaattggaagtccttcttcagccggacctggtccagactgagcctgcacgaggatgaggacaaagagaacgacggcgacagcctgcctaccttcaaatgtgtcagcggccagaacaccaacacactgggcagcatgcaccagaaacggacagccatgttccaggatcctcaagagcggccacggaagctgcctcagctgtgtacagaactgcagaccaccatccacgacatcatcctcgagtgcgtgtactgcaagcagcagctcctgcggagagaagtgtacgacttcgccttccgggatctgtgcatcgtgtacagagatggcaacccctacgccgtgtgcgacaagtgcctgaagttctactccaagatcagcgagtaccggcactactgctactccctgtacggcaccacacttgagcagcagtacaacaagcccctgtgcgatctgctgatccggtgcatcaactgccagaaacctcagtgccccgaggaaaagcagagacacctggacaagaagcagcggttccacaacattagaggcagatggacaggccggtgcatgagctgctgtagaagcagcagaacaagacggggctccatgcacggcgataccccaacactgcacgagtacatgctggatctgcagcccgagacaaccgatctgtacggctatgagcagctgaatgactccagcgaggaagaggacgagatcgacggacctgctggacaggctgaacctgatagagcccactacaatatcgtgaccttctgctgcaagtgcgatagcaccctgagaagatgcgtgcagagcacccacgtggacatcaggaccctcgaggatctgctcatgggcaccctgggaattgtggccccaatctgctctcagaagccttgaggaagaatgtgcacagtgctaaccagaaaattcaggacaagactagtgcggccgctttggaaagttttataggtagttgatagaacaaaatacataattttgtaaaaataaatcactttttat

SEQ ID NO: 80 - DNA, 4853 bp, synthetic construct
  misc_feature 1..4853: note = HPV18 Ii-E1E2E6E7 integrated into DelIII locus of the poxviral vector
  source 1..4853: mol_type = other DNA
aaaaattaaaattttattttttttttttggaatataaataagatctatccctatcagtgatagagatctccctatcagtgatagagatcgtcgacgagctcgtttagtgaaccgtcagatcgcctggagacgccatccacgctgttttgacctctagcaccggtgccaccatgcacagaaggcggagcagaagctgccgcgaggatcagaaacccgtgatggacgaccagcgggacctgatcagcaacaacgagcagctgcctatgctcggcagaaggcctggcgctcctgagagcaaatgttctagaggcgccctgtacaccggcttcagcatcctggttacactgctgctggccggacaggctaccaccgcctactttctgtatcagcagcagggcagactggacaagctgaccgtgaccagccagaacctgcagctggaaaacctgcggatgaagctgcccaagcctcctaagcctgtgtccaagatgcggatggccacacctctgctgatgcaggctttgcctatgggagcactgcctcagggccctatgcagaacgccaccaaatacggcaacatgaccgaggaccacgtgatgcatctgctgcagaatgccgatcctctgaaggtgtaccctccactgaagggcagctttcccgagaacctgcggcacctgaagaacaccatggaaaccatcgactggaaggtgttcgagagctggatgcaccactggctgctgttcgagatgagccggcacagcctggaacagaagcctacagacgcccctcctaaagagtccctggaactggaagatcccagctctggcctgggcgtgacaaagcaagatctgggccctgtgcctatgatggccgatcctgagggaacagatggcgaaggcacaggctgcaacggctggttttacgtgcaagccatcgtggacaagaagaccggcgacgtcatcagcgacgacgaggatgagaatgccaccgacaccggcagcgacatggtggactttatcgatacccagggcacgttctgcgagcaggccgaactggaaacagcccaggcactgtttcacgcccaagaggtgcacaacgatgcccaggtgctgcacgtgctgaagagaaagtttgccggcggaagcaccgagaacagcccactgggagaaagactggaagtggacaccgagctgagccccagactgcaagagatcagcctgaacagcggccagaagaaggccaagcggagactgttcaccatctccgacagcggctacggctgtagcgaagtggaagccacacagatccaagtgaccaccaatggcgagcacggcggcaatgtttgttctggcggatctaccgaggccatcgacaatggcggaaccgagggcaacaatagctccgtggatggcaccagcgacaacagcaacatcgagaacgtgaaccctcagtgcacaatcgcccagctgaaggacctgctgaaagtgaacaacaagcagggcgccatgctggccgtgtttaaggatacctacggcctgagcttcaccgacctcgtgcggaacttcaagagcgacaagaccacctgtaccgactgggtcaccgccatctttggcgtgaacccaacaatcgccgagggcttcaagaccctgatccagcctttcatcctgtacgcccacatccagtgtctggactgcaaatggggcgtgctgattctggccctgctgagatacaagtgcggcaagagcagactgaccgtggccaagggactgagcactctgctgcatgtgcccgagacatgcatgctgattcagcctccaaagctgcggtctagcgtggccgctctgtactggtacagaaccggcatcagcaatatcagcgaagtgatgggcgacacccctgagtggattcagcggctgacaatcatccagcacggcatcgacgactccaacttcgacctgagcgagatggtgcagtgggccttcgataacgagctgaccgacgagtccgacatggcctttgaatacgccctgctggcagacagcaactccaatgccgccgctttcctgaagtccaactgccaggccaagtacctgaaggactgcgccaccatgtgcaagcactaccgcagagcacagaaacggcagatgaacatgagccagtggatccggttccggtgcagcaagatcgatgaaggtggcgactggcggcctatcgtgcagttcctgagataccagcagatcgagttcatcacctttctgggcgccctgaagtcattcctgaagggaacccctaaaaagaactgcctggtgttctgcggccctgccaataccggcaagtcctacttcggcatgagctttatccacttcatccagggcgccgtgatcagcttcgtgaacagcaccagccacttttggctggaacccctgaccgataccaaggtggcaatgctggacgacgccaccaccacctgttggacctacttcgacacctacatgcggaacgccctggacggcaaccccatcagcatcgacagaaagcacaagcccctcatccagctgaaatgccctccaatcctgctgaccacaaacattcaccccgccaaggacaatcggtggccctacctggaaagccggatcaccgtgttcgagttccccaacgcctttccattcgacaagaacggcaatcccgtgtacgagatcaacgacaagaattggaagtgcttcttcgagcggacctggtccagactggatctgcacgaggaagaagaggacgccgacacagagggcaatcccttcggcacctttaagtgtgtggccgggcagaatcacagacccctgggatctatgcagaccccaaaagagacactgagcgagcggctgagcgccctccaggacaagatcatcgaccactacgagaacgacagcaaggacatcgacagccagattcagtactggcagctgatcagatgggagaacgccatcttcttcgccgctagagagcacggaattcagaccctgaaccaccaggtggtgcccgcctacaacatcagcaagagcaaggcccacaaggccattgaactccagatggccctgcaaggactggctcagagcgcctacaagaccgaggattggacactccaggatacctgcgaggaactgtggaacacagagcccacacactgcttcaagaaaggcggccagaccgtgcaggtctacttcgatggcaacaaggataactgcatgacctacgtggcctgggacagcgtgtactacatgacagatgccggcacctgggacaagaccgccacctgtgtttctcaccggggcctgtactacgtgaaagagggctacaacaccttctacatcgagtttaagagcgagtgcgagaagtatggcaacaccggcacatgggaagtgcatttcggcaacaacgtgatcgactgcaacgactccatgtgctccacctccgatgacaccgtgtctgccacacagctggtcaagcagctccagcacaccccatctccatacagctctacagtgtctgtgggcaccgccaagacctatggccaaacaagcgctgccacaagacctggccattgtggactggccgagaagcagcactgtggccctgttaatcctctgctgggagccgccacacctacaggcaacaacaagagaagaaagctgtgcagcggcaacaccacacctatcattcacctgaagggcgacagaaacagcctgaagtgcctgcggtacagactgaggaagcacagcgatcactaccgggacatcagctccacctggcactggacaggtgctggcaacgaaaagaccggcatcctgacagtgacctaccacagcgagactcagcggaccaagttcctgaacaccgtggctatccccgacagcgtgcagattctcgtgggctacatgaccatgggcagcatggccagattcgaggaccctaccagacggccctacaagctgcctgatctgtgcaccgaactgaacaccagccttcaggatatcgagatcacctgtgtgtactgcaagaccgtgctcgagctgacagaagtgtttgagttcgccttcaaggacctgttcgtggtgtacagagacagcatccctcacgccgcctgtcacaagtgcatcgacttctacagcagaatcagagagctgcggcactacagcgactccgtgtacggcgataccctggaaaaactgaccaacaccggactgtacaacctgctgatccggtgcctgagatgccagaaacctcagaaccccgccgagaaactgagacatctgaacgagaagcggcggttccacaatatcgccggccactacagaggccagtgccacagctgttgcaaccgggccagacaagaaagactccagcggagaagaggcagcatgcacggacctaaagccacacttcaggacatcgtgctgcacctcgagcctcagaatgagatccccgtggatctgctcggccatgagcagctgagcgatagcgaggaagagaacgacgagatcgacggcgtcaaccaccagcatctgcctgccagaagggccgaacctcagagacacaccatgctgtgcatgtgctgcaagtgcgaggcccggatcgagagagtggtggaatctagcgccgacgacctgagagctttccagcagctgttcctgaatactctgagcttcgtggccccttggtgcgctagtcagcagtgaggaagaatgtgcacagtgctaaccagaaaattcaggacaagactagtgcggccgctttggaaagttttataggtagttgatagaacaaaatacataattttgtaaaaataaatcactttttat

SEQ ID NO: 81 - DNA, 5609 bp, synthetic construct
  misc_feature 1..5609: note = HPV18 Ii-E1E2-p2a-Ii-E6E7 integrated into DelIII locus of the poxviral vector
  source 1..5609: mol_type = other DNA
aaaaattaaaattttattttttttttttggaatataaataagatctatccctatcagtgatagagatctccctatcagtgatagagatcgtcgacgagctcgtttagtgaaccgtcagatcgcctggagacgccatccacgctgttttgacctctagcaccggtgccaccatgcacagaaggcggagcagaagctgccgcgaggatcagaaacccgtgatggacgaccagcgggacctgatcagcaacaacgagcagctgcctatgctcggcagaaggcctggcgctcctgagagcaaatgttctagaggcgccctgtacaccggcttcagcatcctggttacactgctgctggccggacaggctaccaccgcctactttctgtatcagcagcagggcagactggacaagctgaccgtgaccagccagaacctgcagctggaaaacctgcggatgaagctgcccaagcctcctaagcctgtgtccaagatgcggatggccacacctctgctgatgcaggctttgcctatgggagcactgcctcagggccctatgcagaacgccaccaaatacggcaacatgaccgaggaccacgtgatgcatctgctgcagaatgccgatcctctgaaggtgtaccctccactgaagggcagctttcccgagaacctgcggcacctgaagaacaccatggaaaccatcgactggaaggtgttcgagagctggatgcaccactggctgctgttcgagatgagccggcacagcctggaacagaagcctacagacgcccctcctaaagagtccctggaactggaagatcccagctctggcctgggcgtgacaaagcaagatctgggccctgtgcctatgatggccgatcctgagggaacagatggcgaaggcacaggctgcaacggctggttttacgtgcaagccatcgtggacaagaagaccggcgacgtcatcagcgacgacgaggatgagaatgccaccgacaccggcagcgacatggtggactttatcgatacccagggcacgttctgcgagcaggccgaactggaaacagcccaggcactgtttcacgcccaagaggtgcacaacgatgcccaggtgctgcacgtgctgaagagaaagtttgccggcggaagcaccgagaacagcccactgggagaaagactggaagtggacaccgagctgagccccagactgcaagagatcagcctgaacagcggccagaagaaggccaagcggagactgttcaccatctccgacagcggctacggctgtagcgaagtggaagccacacagatccaagtgaccaccaatggcgagcacggcggcaatgtttgttctggcggatctaccgaggccatcgacaatggcggaaccgagggcaacaatagctccgtggatggcaccagcgacaacagcaacatcgagaacgtgaaccctcagtgcacaatcgcccagctgaaggacctgctgaaagtgaacaacaagcagggcgccatgctggccgtgtttaaggatacctacggcctgagcttcaccgacctcgtgcggaacttcaagagcgacaagaccacctgtaccgactgggtcaccgccatctttggcgtgaacccaacaatcgccgagggcttcaagaccctgatccagcctttcatcctgtacgcccacatccagtgtctggactgcaaatggggcgtgctgattctggccctgctgagatacaagtgcggcaagagcagactgaccgtggccaagggactgagcactctgctgcatgtgcccgagacatgcatgctgattcagcctccaaagctgcggtctagcgtggccgctctgtactggtacagaaccggcatcagcaatatcagcgaagtgatgggcgacacccctgagtggattcagcggctgacaatcatccagcacggcatcgacgactccaacttcgacctgagcgagatggtgcagtgggccttcgataacgagctgaccgacgagtccgacatggcctttgaatacgccctgctggcagacagcaactccaatgccgccgctttcctgaagtccaactgccaggccaagtacctgaaggactgcgccaccatgtgcaagcactaccgcagagcacagaaacggcagatgaacatgagccagtggatccggttccggtgcagcaagatcgatgaaggtggcgactggcggcctatcgtgcagttcctgagataccagcagatcgagttcatcacctttctgggcgccctgaagtcattcctgaagggaacccctaaaaagaactgcctggtgttctgcggccctgccaataccggcaagtcctacttcggcatgagctttatccacttcatccagggcgccgtgatcagcttcgtgaacagcaccagccacttttggctggaacccctgaccgataccaaggtggcaatgctggacgacgccaccaccacctgttggacctacttcgacacctacatgcggaacgccctggacggcaaccccatcagcatcgacagaaagcacaagcccctcatccagctgaaatgccctccaatcctgctgaccacaaacattcaccccgccaaggacaatcggtggccctacctggaaagccggatcaccgtgttcgagttccccaacgcctttccattcgacaagaacggcaatcccgtgtacgagatcaacgacaagaattggaagtgcttcttcgagcggacctggtccagactggatctgcacgaggaagaagaggacgccgacacagagggcaatcccttcggcacctttaagtgtgtggccgggcagaatcacagacccctgggatctatgcagaccccaaaagagacactgagcgagcggctgagcgccctccaggacaagatcatcgaccactacgagaacgacagcaaggacatcgacagccagattcagtactggcagctgatcagatgggagaacgccatcttcttcgccgctagagagcacggaattcagaccctgaaccaccaggtggtgcccgcctacaacatcagcaagagcaaggcccacaaggccattgaactccagatggccctgcaaggactggctcagagcgcctacaagaccgaggattggacactccaggatacctgcgaggaactgtggaacacagagcccacacactgcttcaagaaaggcggccagaccgtgcaggtctacttcgatggcaacaaggataactgcatgacctacgtggcctgggacagcgtgtactacatgacagatgccggcacctgggacaagaccgccacctgtgtttctcaccggggcctgtactacgtgaaagagggctacaacaccttctacatcgagtttaagagcgagtgcgagaagtatggcaacaccggcacatgggaagtgcatttcggcaacaacgtgatcgactgcaacgactccatgtgctccacctccgatgacaccgtgtctgccacacagctggtcaagcagctccagcacaccccatctccatacagctctacagtgtctgtgggcaccgccaagacctatggccaaacaagcgctgccacaagacctggccattgtggactggccgagaagcagcactgtggccctgttaatcctctgctgggagccgccacacctacaggcaacaacaagagaagaaagctgtgcagcggcaacaccacacctatcattcacctgaagggcgacagaaacagcctgaagtgcctgcggtacagactgaggaagcacagcgatcactaccgggacatcagctccacctggcactggacaggtgctggcaacgaaaagaccggcatcctgacagtgacctaccacagcgagactcagcggaccaagttcctgaacaccgtggctatccccgacagcgtgcagattctcgtgggctacatgaccatgggcagcggcgccaccaatttcagtctgcttaaacaggccggggacgtcgaagagaaccccggacctatgcatcggcgcagatctcggtcttgtagagaagaccaaaagcctgtcatggatgatcaaagagatttaatttctaataatgaacaattacccatgttgggacggcgccccggagcccccgaatctaagtgcagccggggagctttatatacaggattttctattttagtgaccttattattagctggccaagccacaacagcttatttcttataccaacaacaaggacggttagataaattaacagtcacatctcaaaatttacaattagagaatttaagaatgaaattacctaaaccacccaaacccgtcagtaaaatgagaatggctacccccttattaatgcaagccctccccatgggcgccttaccccaaggacccatgcaaaatgctacaaagtatggaaatatgacagaagatcatgtcatgcacttattacaaaacgctgaccccttaaaagtctatccccctttaaaaggatctttccctgaaaatttaagacatttaaaaaatacaatggagacaattgattggaaagtctttgaatcttggatgcatcattggttattatttgaaatgtctaggcattctttagagcaaaaacccaccgatgctccacccaaggaaagtttagagttagaggacccttctagcggattaggagtcaccaaacaggacttaggacccgtccccatgatggccagattcgaggaccctaccagacggccctacaagctgcctgatctgtgcaccgaactgaacaccagccttcaggatatcgagatcacctgtgtgtactgcaagaccgtgctcgagctgacagaagtgtttgagttcgccttcaaggacctgttcgtggtgtacagagacagcatccctcacgccgcctgtcacaagtgcatcgacttctacagcagaatcagagagctgcggcactacagcgactccgtgtacggcgataccctggaaaaactgaccaacaccggactgtacaacctgctgatccggtgcctgagatgccagaaacctcagaaccccgccgagaaactgagacatctgaacgagaagcggcggttccacaatatcgccggccactacagaggccagtgccacagctgttgcaaccgggccagacaagaaagactccagcggagaagaggcagcatgcacggacctaaagccacacttcaggacatcgtgctgcacctcgagcctcagaatgagatccccgtggatctgctcggccatgagcagctgagcgatagcgaggaagagaacgacgagatcgacggcgtcaaccaccagcatctgcctgccagaagggccgaacctcagagacacaccatgctgtgcatgtgctgcaagtgcgaggcccggatcgagagagtggtggaatctagcgccgacgacctgagagctttccagcagctgttcctgaatactctgagcttcgtggccccttggtgcgctagtcagcagtgaggaagaatgtgcacagtgctaaccagaaaattcaggacaagactagtgcggccgctttggaaagttttataggtagttgatagaacaaaatacataattttgtaaaaataaatcactttttat

SEQ ID NO: 82 - DNA, 4157 bp, synthetic construct
  misc_feature 1..4157: note = HPV18 E1E2E6E7 integrated into DelIII locus of the poxviral vector
  source 1..4157: mol_type = other DNA
aaaaattaaaattttattttttttttttggaatataaataagatctatccctatcagtgatagagatctccctatcagtgatagagatcgtcgacgagctcgtttagtgaaccgtcagatcgcctggagacgccatccacgctgttttgacctctagcaccggtgccaccatggccgatcctgagggaacagatggcgaaggcacaggctgcaacggctggttttacgtgcaagccatcgtggacaagaagaccggcgacgtcatcagcgacgacgaggatgagaatgccaccgacaccggcagcgacatggtggactttatcgatacccagggcacgttctgcgagcaggccgaactggaaacagcccaggcactgtttcacgcccaagaggtgcacaacgatgcccaggtgctgcacgtgctgaagagaaagtttgccggcggaagcaccgagaacagcccactgggagaaagactggaagtggacaccgagctgagccccagactgcaagagatcagcctgaacagcggccagaagaaggccaagcggagactgttcaccatctccgacagcggctacggctgtagcgaagtggaagccacacagatccaagtgaccaccaatggcgagcacggcggcaatgtttgttctggcggatctaccgaggccatcgacaatggcggaaccgagggcaacaatagctccgtggatggcaccagcgacaacagcaacatcgagaacgtgaaccctcagtgcacaatcgcccagctgaaggacctgctgaaagtgaacaacaagcagggcgccatgctggccgtgtttaaggatacctacggcctgagcttcaccgacctcgtgcggaacttcaagagcgacaagaccacctgtaccgactgggtcaccgccatctttggcgtgaacccaacaatcgccgagggcttcaagaccctgatccagcctttcatcctgtacgcccacatccagtgtctggactgcaaatggggcgtgctgattctggccctgctgagatacaagtgcggcaagagcagactgaccgtggccaagggactgagcactctgctgcatgtgcccgagacatgcatgctgattcagcctccaaagctgcggtctagcgtggccgctctgtactggtacagaaccggcatcagcaatatcagcgaagtgatgggcgacacccctgagtggattcagcggctgacaatcatccagcacggcatcgacgactccaacttcgacctgagcgagatggtgcagtgggccttcgataacgagctgaccgacgagtccgacatggcctttgaatacgccctgctggcagacagcaactccaatgccgccgctttcctgaagtccaactgccaggccaagtacctgaaggactgcgccaccatgtgcaagcactaccgcagagcacagaaacggcagatgaacatgagccagtggatccggttccggtgcagcaagatcgatgaaggtggcgactggcggcctatcgtgcagttcctgagataccagcagatcgagttcatcacctttctgggcgccctgaagtcattcctgaagggaacccctaaaaagaactgcctggtgttctgcggccctgccaataccggcaagtcctacttcggcatgagctttatccacttcatccagggcgccgtgatcagcttcgtgaacagcaccagccacttttggctggaacccctgaccgataccaaggtggcaatgctggacgacgccaccaccacctgttggacctacttcgacacctacatgcggaacgccctggacggcaaccccatcagcatcgacagaaagcacaagcccctcatccagctgaaatgccctccaatcctgctgaccacaaacattcaccccgccaaggacaatcggtggccctacctggaaagccggatcaccgtgttcgagttccccaacgcctttccattcgacaagaacggcaatcccgtgtacgagatcaacgacaagaattggaagtgcttcttcgagcggacctggtccagactggatctgcacgaggaagaagaggacgccgacacagagggcaatcccttcggcacctttaagtgtgtggccgggcagaatcacagacccctgggatctatgcagaccccaaaagagacactgagcgagcggctgagcgccctccaggacaagatcatcgaccactacgagaacgacagcaaggacatcgacagccagattcagtactggcagctgatcagatgggagaacgccatcttcttcgccgctagagagcacggaattcagaccctgaaccaccaggtggtgcccgcctacaacatcagcaagagcaaggcccacaaggccattgaactccagatggccctgcaaggactggctcagagcgcctacaagaccgaggattggacactccaggatacctgcgaggaactgtggaacacagagcccacacactgcttcaagaaaggcggccagaccgtgcaggtctacttcgatggcaacaaggataactgcatgacctacgtggcctgggacagcgtgtactacatgacagatgccggcacctgggacaagaccgccacctgtgtttctcaccggggcctgtactacgtgaaagagggctacaacaccttctacatcgagtttaagagcgagtgcgagaagtatggcaacaccggcacatgggaagtgcatttcggcaacaacgtgatcgactgcaacgactccatgtgctccacctccgatgacaccgtgtctgccacacagctggtcaagcagctccagcacaccccatctccatacagctctacagtgtctgtgggcaccgccaagacctatggccaaacaagcgctgccacaagacctggccattgtggactggccgagaagcagcactgtggccctgttaatcctctgctgggagccgccacacctacaggcaacaacaagagaagaaagctgtgcagcggcaacaccacacctatcattcacctgaagggcgacagaaacagcctgaagtgcctgcggtacagactgaggaagcacagcgatcactaccgggacatcagctccacctggcactggacaggtgctggcaacgaaaagaccggcatcctgacagtgacctaccacagcgagactcagcggaccaagttcctgaacaccgtggctatccccgacagcgtgcagattctcgtgggctacatgaccatgggcagcatggccagattcgaggaccctaccagacggccctacaagctgcctgatctgtgcaccgaactgaacaccagccttcaggatatcgagatcacctgtgtgtactgcaagaccgtgctcgagctgacagaagtgtttgagttcgccttcaaggacctgttcgtggtgtacagagacagcatccctcacgccgcctgtcacaagtgcatcgacttctacagcagaatcagagagctgcggcactacagcgactccgtgtacggcgataccctggaaaaactgaccaacaccggactgtacaacctgctgatccggtgcctgagatgccagaaacctcagaaccccgccgagaaactgagacatctgaacgagaagcggcggttccacaatatcgccggccactacagaggccagtgccacagctgttgcaaccgggccagacaagaaagactccagcggagaagaggcagcatgcacggacctaaagccacacttcaggacatcgtgctgcacctcgagcctcagaatgagatccccgtggatctgctcggccatgagcagctgagcgatagcgaggaagagaacgacgagatcgacggcgtcaaccaccagcatctgcctgccagaagggccgaacctcagagacacaccatgctgtgcatgtgctgcaagtgcgaggcccggatcgagagagtggtggaatctagcgccgacgacctgagagctttccagcagctgttcctgaatactctgagcttcgtggccccttggtgcgctagtcagcagtgaggaagaatgtgcacagtgctaaccagaaaattcaggacaagactagtgcggccgctttggaaagttttataggtagttgatagaacaaaatacataattttgtaaaaataaatcactttttat

SEQ ID NO: 83 - DNA, 5609 bp, synthetic construct
  misc_feature 1..5609: note = HPV18 Ii-E1E6E7-p2a-Ii-E2 integrated into DelIII locus of the poxviral vector
  source 1..5609: mol_type = other DNA
aaaaattaaaattttattttttttttttggaatataaataagatctatccctatcagtgatagagatctccctatcagtgatagagatcgtcgacgagctcgtttagtgaaccgtcagatcgcctggagacgccatccacgctgttttgacctctagcaccggtgccaccatgcacagaaggcggagcagaagctgccgcgaggatcagaaacccgtgatggacgaccagcgggacctgatcagcaacaacgagcagctgcctatgctcggcagaaggcctggcgctcctgagagcaaatgttctagaggcgccctgtacaccggcttcagcatcctggttacactgctgctggccggacaggctaccaccgcctactttctgtatcagcagcagggcagactggacaagctgaccgtgaccagccagaacctgcagctggaaaacctgcggatgaagctgcccaagcctcctaagcctgtgtccaagatgcggatggccacacctctgctgatgcaggctttgcctatgggagcactgcctcagggccctatgcagaacgccaccaaatacggcaacatgaccgaggaccacgtgatgcatctgctgcagaatgccgatcctctgaaggtgtaccctccactgaagggcagctttcccgagaacctgcggcacctgaagaacaccatggaaaccatcgactggaaggtgttcgagagctggatgcaccactggctgctgttcgagatgagccggcacagcctggaacagaagcctacagacgcccctcctaaagagtccctggaactggaagatcccagctctggcctgggcgtgacaaagcaagatctgggccctgtgcctatgatggccgatcctgagggaacagatggcgaaggcacaggctgcaacggctggttttacgtgcaagccatcgtggacaagaagaccggcgacgtcatcagcgacgacgaggatgagaatgccaccgacaccggcagcgacatggtggactttatcgatacccagggcacgttctgcgagcaggccgaactggaaacagcccaggcactgtttcacgcccaagaggtgcacaacgatgcccaggtgctgcacgtgctgaagagaaagtttgccggcggaagcaccgagaacagcccactgggagaaagactggaagtggacaccgagctgagccccagactgcaagagatcagcctgaacagcggccagaagaaggccaagcggagactgttcaccatctccgacagcggctacggctgtagcgaagtggaagccacacagatccaagtgaccaccaatggcgagcacggcggcaatgtttgttctggcggatctaccgaggccatcgacaatggcggaaccgagggcaacaatagctccgtggatggcaccagcgacaacagcaacatcgagaacgtgaaccctcagtgcacaatcgcccagctgaaggacctgctgaaagtgaacaacaagcagggcgccatgctggccgtgtttaaggatacctacggcctgagcttcaccgacctcgtgcggaacttcaagagcgacaagaccacctgtaccgactgggtcaccgccatctttggcgtgaacccaacaatcgccgagggcttcaagaccctgatccagcctttcatcctgtacgcccacatccagtgtctggactgcaaatggggcgtgctgattctggccctgctgagatacaagtgcggcaagagcagactgaccgtggccaagggactgagcactctgctgcatgtgcccgagacatgcatgctgattcagcctccaaagctgcggtctagcgtggccgctctgtactggtacagaaccggcatcagcaatatcagcgaagtgatgggcgacacccctgagtggattcagcggctgacaatcatccagcacggcatcgacgactccaacttcgacctgagcgagatggtgcagtgggccttcgataacgagctgaccgacgagtccgacatggcctttgaatacgccctgctggcagacagcaactccaatgccgccgctttcctgaagtccaactgccaggccaagtacctgaaggactgcgccaccatgtgcaagcactaccgcagagcacagaaacggcagatgaacatgagccagtggatccggttccggtgcagcaagatcgatgaaggtggcgactggcggcctatcgtgcagttcctgagataccagcagatcgagttcatcacctttctgggcgccctgaagtcattcctgaagggaacccctaaaaagaactgcctggtgttctgcggccctgccaataccggcaagtcctacttcggcatgagctttatccacttcatccagggcgccgtgatcagcttcgtgaacagcaccagccacttttggctggaacccctgaccgataccaaggtggcaatgctggacgacgccaccaccacctgttggacctacttcgacacctacatgcggaacgccctggacggcaaccccatcagcatcgacagaaagcacaagcccctcatccagctgaaatgccctccaatcctgctgaccacaaacattcaccccgccaaggacaatcggtggccctacctggaaagccggatcaccgtgttcgagttccccaacgcctttccattcgacaagaacggcaatcccgtgtacgagatcaacgacaagaattggaagtgcttcttcgagcggacctggtccagactggatctgcacgaggaagaagaggacgccgacacagagggcaatcccttcggcacctttaagtgtgtggccgggcagaatcacagacccctgggcagcatggccagattcgaggaccctaccagacggccctacaagctgcctgatctgtgcaccgaactgaacaccagccttcaggatatcgagatcacctgtgtgtactgcaagaccgtgctcgagctgacagaagtgtttgagttcgccttcaaggacctgttcgtggtgtacagagacagcatccctcacgccgcctgtcacaagtgcatcgacttctacagcagaatcagagagctgcggcactacagcgactccgtgtacggcgataccctggaaaaactgaccaacaccggactgtacaacctgctgatccggtgcctgagatgccagaaacctcagaaccccgccgagaaactgagacatctgaacgagaagcggcggttccacaatatcgccggccactacagaggccagtgccacagctgttgcaaccgggccagacaagaaagactccagcggagaagaggcagcatgcacggacctaaagccacacttcaggacatcgtgctgcacctcgagcctcagaatgagatccccgtggatctgctcggccatgagcagctgagcgatagcgaggaagagaacgacgagatcgacggcgtcaaccaccagcatctgcctgccagaagggccgaacctcagagacacaccatgctgtgcatgtgctgcaagtgcgaggcccggatcgagagagtggtggaatctagcgccgacgacctgagagctttccagcagctgttcctgaatactctgagcttcgtggccccttggtgcgctagtcagcagggcagcggcgccaccaatttcagtctgcttaaacaggccggggacgtcgaagagaaccccggacctatgcatcggcgcagatctcggtcttgtagagaagaccaaaagcctgtcatggatgatcaaagagatttaatttctaataatgaacaattacccatgttgggacggcgccccggagcccccgaatctaagtgcagccggggagctttatatacaggattttctattttagtgaccttattattagctggccaagccacaacagcttatttcttataccaacaacaaggacggttagataaattaacagtcacatctcaaaatttacaattagagaatttaagaatgaaattacctaaaccacccaaacccgtcagtaaaatgagaatggctacccccttattaatgcaagccctccccatgggcgccttaccccaaggacccatgcaaaatgctacaaagtatggaaatatgacagaagatcatgtcatgcacttattacaaaacgctgaccccttaaaagtctatccccctttaaaaggatctttccctgaaaatttaagacatttaaaaaatacaatggagacaattgattggaaagtctttgaatcttggatgcatcattggttattatttgaaatgtctaggcattctttagagcaaaaacccaccgatgctccacccaaggaaagtttagagttagaggacccttctagcggattaggagtcaccaaacaggacttaggacccgtccccatgatgcagaccccaaaagagacactgagcgagcggctgagcgccctccaggacaagatcatcgaccactacgagaacgacagcaaggacatcgacagccagattcagtactggcagctgatcagatgggagaacgccatcttcttcgccgctagagagcacggaattcagaccctgaaccaccaggtggtgcccgcctacaacatcagcaagagcaaggcccacaaggccattgaactccagatggccctgcaaggactggctcagagcgcctacaagaccgaggattggacactccaggatacctgcgaggaactgtggaacacagagcccacacactgcttcaagaaaggcggccagaccgtgcaggtctacttcgatggcaacaaggataactgcatgacctacgtggcctgggacagcgtgtactacatgacagatgccggcacctgggacaagaccgccacctgtgtttctcaccggggcctgtactacgtgaaagagggctacaacaccttctacatcgagtttaagagcgagtgcgagaagtatggcaacaccggcacatgggaagtgcatttcggcaacaacgtgatcgactgcaacgactccatgtgctccacctccgatgacaccgtgtctgccacacagctggtcaagcagctccagcacaccccatctccatacagctctacagtgtctgtgggcaccgccaagacctatggccaaacaagcgctgccacaagacctggccattgtggactggccgagaagcagcactgtggccctgttaatcctctgctgggagccgccacacctacaggcaacaacaagagaagaaagctgtgcagcggcaacaccacacctatcattcacctgaagggcgacagaaacagcctgaagtgcctgcggtacagactgaggaagcacagcgatcactaccgggacatcagctccacctggcactggacaggtgctggcaacgaaaagaccggcatcctgacagtgacctaccacagcgagactcagcggaccaagttcctgaacaccgtggctatccccgacagcgtgcagattctcgtgggctacatgaccatgtgaggaagaatgtgcacagtgctaaccagaaaattcaggacaagactagtgcggccgctttggaaagttttataggtagttgatagaacaaaatacataattttgtaaaaataaatcactttttat

SEQ ID NO: 84 - DNA, 3752 bp, synthetic construct
  misc_feature 1..3752: note = HPV18 Ii-E1E6E7 integrated into DelIII locus of the poxviral vector
  source 1..3752: mol_type = other DNA
aaaaattaaaattttattttttttttttggaatataaataagatctatccctatcagtgatagagatctccctatcagtgatagagatcgtcgacgagctcgtttagtgaaccgtcagatcgcctggagacgccatccacgctgttttgacctctagcaccggtgccaccatgcacagaaggcggagcagaagctgccgcgaggatcagaaacccgtgatggacgaccagcgggacctgatcagcaacaacgagcagctgcctatgctcggcagaaggcctggcgctcctgagagcaaatgttctagaggcgccctgtacaccggcttcagcatcctggttacactgctgctggccggacaggctaccaccgcctactttctgtatcagcagcagggcagactggacaagctgaccgtgaccagccagaacctgcagctggaaaacctgcggatgaagctgcccaagcctcctaagcctgtgtccaagatgcggatggccacacctctgctgatgcaggctttgcctatgggagcactgcctcagggccctatgcagaacgccaccaaatacggcaacatgaccgaggaccacgtgatgcatctgctgcagaatgccgatcctctgaaggtgtaccctccactgaagggcagctttcccgagaacctgcggcacctgaagaacaccatggaaaccatcgactggaaggtgttcgagagctggatgcaccactggctgctgttcgagatgagccggcacagcctggaacagaagcctacagacgcccctcctaaagagtccctggaactggaagatcccagctctggcctgggcgtgacaaagcaagatctgggccctgtgcctatgatggccgatcctgagggaacagatggcgaaggcacaggctgcaacggctggttttacgtgcaagccatcgtggacaagaagaccggcgacgtcatcagcgacgacgaggatgagaatgccaccgacaccggcagcgacatggtggactttatcgatacccagggcacgttctgcgagcaggccgaactggaaacagcccaggcactgtttcacgcccaagaggtgcacaacgatgcccaggtgctgcacgtgctgaagagaaagtttgccggcggaagcaccgagaacagcccactgggagaaagactggaagtggacaccgagctgagccccagactgcaagagatcagcctgaacagcggccagaagaaggccaagcggagactgttcaccatctccgacagcggctacggctgtagcgaagtggaagccacacagatccaagtgaccaccaatggcgagcacggcggcaatgtttgttctggcggatctaccgaggccatcgacaatggcggaaccgagggcaacaatagctccgtggatggcaccagcgacaacagcaacatcgagaacgtgaaccctcagtgcacaatcgcccagctgaaggacctgctgaaagtgaacaacaagcagggcgccatgctggccgtgtttaaggatacctacggcctgagcttcaccgacctcgtgcggaacttcaagagcgacaagaccacctgtaccgactgggtcaccgccatctttggcgtgaacccaacaatcgccgagggcttcaagaccctgatccagcctttcatcctgtacgcccacatccagtgtctggactgcaaatggggcgtgctgattctggccctgctgagatacaagtgcggcaagagcagactgaccgtggccaagggactgagcactctgctgcatgtgcccgagacatgcatgctgattcagcctccaaagctgcggtctagcgtggccgctctgtactggtacagaaccggcatcagcaatatcagcgaagtgatgggcgacacccctgagtggattcagcggctgacaatcatccagcacggcatcgacgactccaacttcgacctgagcgagatggtgcagtgggccttcgataacgagctgaccgacgagtccgacatggcctttgaatacgccctgctggcagacagcaactccaatgccgccgctttcctgaagtccaactgccaggccaagtacctgaaggactgcgccaccatgtgcaagcactaccgcagagcacagaaacggcagatgaacatgagccagtggatccggttccggtgcagcaagatcgatgaaggtggcgactggcggcctatcgtgcagttcctgagataccagcagatcgagttcatcacctttctgggcgccctgaagtcattcctgaagggaacccctaaaaagaactgcctggtgttctgcggccctgccaataccggcaagtcctacttcggcatgagctttatccacttcatccagggcgccgtgatcagcttcgtgaacagcaccagccacttttggctggaacccctgaccgataccaaggtggcaatgctggacgacgccaccaccacctgttggacctacttcgacacctacatgcggaacgccctggacggcaaccccatcagcatcgacagaaagcacaagcccctcatccagctgaaatgccctccaatcctgctgaccacaaacattcaccccgccaaggacaatcggtggccctacctggaaagccggatcaccgtgttcgagttccccaacgcctttccattcgacaagaacggcaatcccgtgtacgagatcaacgacaagaattggaagtgcttcttcgagcggacctggtccagactggatctgcacgaggaagaagaggacgccgacacagagggcaatcccttcggcacctttaagtgtgtggccgggcagaatcacagacccctgggcagcatggccagattcgaggaccctaccagacggccctacaagctgcctgatctgtgcaccgaactgaacaccagccttcaggatatcgagatcacctgtgtgtactgcaagaccgtgctcgagctgacagaagtgtttgagttcgccttcaaggacctgttcgtggtgtacagagacagcatccctcacgccgcctgtcacaagtgcatcgacttctacagcagaatcagagagctgcggcactacagcgactccgtgtacggcgataccctggaaaaactgaccaacaccggactgtacaacctgctgatccggtgcctgagatgccagaaacctcagaaccccgccgagaaactgagacatctgaacgagaagcggcggttccacaatatcgccggccactacagaggccagtgccacagctgttgcaaccgggccagacaagaaagactccagcggagaagaggcagcatgcacggacctaaagccacacttcaggacatcgtgctgcacctcgagcctcagaatgagatccccgtggatctgctcggccatgagcagctgagcgatagcgaggaagagaacgacgagatcgacggcgtcaaccaccagcatctgcctgccagaagggccgaacctcagagacacaccatgctgtgcatgtgctgcaagtgcgaggcccggatcgagagagtggtggaatctagcgccgacgacctgagagctttccagcagctgttcctgaatactctgagcttcgtggccccttggtgcgctagtcagcagtgaggaagaatgtgcacagtgctaaccagaaaattcaggacaagactagtgcggccgctttggaaagttttataggtagttgatagaacaaaatacataattttgtaaaaataaatcactttttat

SEQ ID NO: 85 - DNA, 4684 bp, synthetic construct
  misc_feature 1..4684: note = MfPV3 Ii-E1E2E6E7 integrated into TK locus of the poxviral vector
  source 1..4684: mol_type = other DNA
ggccaaaaattgaaattttattttttttttttggaatataaataatccctatcagtgatagagatctccctatcagtgatagagatcgtcgacgagctcgtttagtgaaccgtcagatcgcctggagacgccatccacgctgttttgacctctagcaccggtgccaccatgcacagaaggcggagcagaagctgccgcgaggatcagaaacccgtgatggacgaccagcgggacctgatcagcaacaacgagcagctgcctatgctcggcagaaggcctggcgctcctgagagcaaatgttctagaggcgccctgtacaccggcttcagcatcctggttacactgctgctggccggacaggctaccaccgcctactttctgtatcagcagcagggcagactggacaagctgaccgtgaccagccagaacctgcagctggaaaacctgcggatgaagctgcccaagcctcctaagcctgtgtccaagatgcggatggccacacctctgctgatgcaggctttgcctatgggagcactgcctcagggccctatgcagaacgccaccaaatacggcaacatgaccgaggaccacgtgatgcatctgctgcagaatgccgatcctctgaaggtgtaccctccactgaagggcagctttcccgagaacctgcggcacctgaagaacaccatggaaaccatcgactggaaggtgttcgagagctggatgcaccactggctgctgttcgagatgagccggcacagcctggaacagaagcctacagacgcccctcctaaagagtccctggaactggaagatcccagctctggcctgggcgtgacaaagcaagatctgggccctgtgcctatgatggacccttctggaattcacggcgagggcaccagatgcaccggatggttcgatgtggaagccatcgtggaaagacggaccggcgacgtggtgtccgaggacgaagatgagaacctggaagataccggcgtggacctggtggacttcatcgacgatacctgcgagtttgagcaggccggcgacgaagctcctagagcactgctgcatgcccaagagacacaggcccatgtgaacgccgtgcactacctgaagcggaagttcacatgcagccctaccgtgtctcccctgggcaatgtgcagtgttctatcgagggcgagctgagccctagactgcacgccatccaactgaatgaaggcagcggcaaggccaagcggagactgtttctgcctgatagcggctacggcaataccgaggtggaaacccagctgtctcaggtggcaggatccgatggcgaatctcaagcccctggcggcaaagaaaatgtgcagcctgctgaaggcgctggcggagatgttcgacagctgctgagaagcagcaacctgaaggccacactgctgggcaagttcaaggacgtgtacggcatcggcttcaacgaactcgtgcggtgcttcaagagcgacaagaccgtgtgcaccgattgggttgtgggagctgctggcgtgcaccattctgtggccgaaggactgaagcacctgatcgagcctttctgcacatacgcccacatgcagtgcctgacctgcaactggggagtgtttctgctgctgctcatccggttcaagtgcggcaagaacagactgaccgtggccaagtgcatctctaccctgctgaacgtgcaagagccctacctgctgatcgaacctccaaagctgagatccacagccgccgctctgtactggtacagaaccggcatcagcaacgtgtccgaagtgatcggagagcctcctgagtggatcgtgcggcagaccatgtttcagcacagcctcgaggacagcatcttcgacctgtctcagatggtgcagtgggcctacgaccacgatctggtggacgatagcgccattgcctaccagtatgcccagctggccgacgtggactctaatgccgccgctttcctgaagtctaacgctcaggccaaatacgtgaaggactgcgccaccatgtgcagacactacaagagagccgagaagcaggccatgaccatgagccagtggatcagagccagatgcgataagaccgacgacggcggagattggaggcctatcgtgcagttcctgagataccagaacgtcgagttcatcacctttctgagcgcctttaaggccttcctgaagggaatccccaagagaagctgcatcgtgctgtacggccctccaaacaccggcaagagctacttcggaatgagcctgatgcagtttctgcagggcagcatcatcagctacgtgaacagcaacagccacttttggctggcccctctggccgaagctaaagtggccatgctggacgatgccacaccacagtgctggtcctacatcgacacctacatgcggagcgtgctggacggcaaccccatgagcattgaccggaagcacaagaacctgatccagatgaagtgtccgcctctgatcatcaccagcaacaccaacgtgggcgaagatgacagatggcgctacctgcactccagagtggtggtgttcaagttcgagcagacctttccattcgaccagaacggcaatcccgtgtacgagctgtccgacaagaattggaagtccttcttcagccggacctggtccagactcgagctgcaagaggatgaggacaccgagcaggatggctctacagtgcgggcctttaagtgtgtgcccggccagaacaccaatatcctgggctctatggaagccctgagcgagagactgaacgcctaccaggacaagatcctgaccttctacgaagaggactccaaggacctgagggaccagatcgagcattggagatgtgtgcggggagagtgcgccgtgttctacaaggccagagagatgggccacagccacatcaaccaccagcaggttccacctctgcacgtgtccagaggcaaggcccacaatgccattgagctgcagatggccctggaaagcctgaaccagagcgacttcaacaccgaggaatggaccctgcaggacacaagcctggaaatgtggcggaccgagcctcagtgctgcttcaaaaagaaaggcgagacagtgaccgtgctgtatgactgcgacaaggacaacagcatggactacaccctgtggggccacgtgtacgtgtggggagatacaggctgggacaaagtgtgcggaaaggtggactacggcggcctgtactatgtgctgcacggcctgaaagtgtactacgttgagttccagcaagaggccgtgaagtacagcaagaccaacaagtgggaagtgcatatcggcagaacagtgatcccctacagcgagtccgtgtcctctacagccctgtgcgaaagagtgcctatcgccgagatcgccaatggcctccagcagaccagcattccctctcacacacacgccaccaccaaagagaacgcttgggtttcctctcagcctcctaccaagcggtccagaagatctgatccaggcggcgatcctgtcagagccctggatgccaatagcagaaccctgctgtctggaaccgcctgcgagaataccaccggcaactcttacagcgactgcacccctgtggtgcacctgaaaggcgaccccaattgtctgaaggccctgcggttcagattcgtgaaacacaagaatctgttcgtgaacgtgtcctccacctggcggtggaccaacaacaccgaagagaaggccatcgtgactgtgacctttaaggacgagcagcagagacagcagttcctcggcagcgtgaagatccccaactccatctgtgtgtgcaagggcgtgatgacagttggcagcatggtggaaggcagccaagagagagcccggacactgcacgaactgtgcgagcagagagaacagacccagcacgacatcgagctggactgcgtgtactgcctgaatcctctgaccagaaccgaggtgtacgacttcgccagatgggagctgagaatcgtgtacagacgggataccgcctacggcgtgtgcaagctgtgcctgaggttccacagcaaagtgcggaagtacaggcggtacaactactccatctacggccccacactggaacggcggacaaagaagcagctcgaagaactcgtgatccggtgctactgctgccagaaacctcagtgccccgtggaaaagcagcgccacgttgaccagggccagagattccacaacattgccgagcagtggaccggcagatgcctgcaatgttggaggccaacagccgctgaaaccggctctatgatcggccctggacctacactgcaggatatcgtgcttgagctggaacccgagcctgtggacctgagaggatatgagcagctggactccagcgaggacgaggacgagattgaccaccaccaacagcaggacagacccgatgtgcaagaagatgacggcgagcggtttagaatcctgagcgactgtcacgtgtgccagaataccgtgtcaagagtggtgcacagcacccacgacgacctgcacattctcgaggacctgcttatgggcaccctcgagatcgtggcccctaattgtgccgtgggcagcgaacagaaactgatctccgaggaagatctctgaggaagaatgtgcacagtgctaaccagaaaattcaggacaagactagtgcggccgcttttat

SEQ ID NO: 86 - DNA, 5440 bp, synthetic construct
  misc_feature 1..5440: note = MfPV3 Ii-E1E2-p2a-Ii-E6E7 integrated into TK locus of the poxviral vector
  source 1..5440: mol_type = other DNA
ggccaaaaattgaaattttattttttttttttggaatataaataatccctatcagtgatagagatctccctatcagtgatagagatcgtcgacgagctcgtttagtgaaccgtcagatcgcctggagacgccatccacgctgttttgacctctagcaccggtgccaccatgcacagaaggcggagcagaagctgccgcgaggatcagaaacccgtgatggacgaccagcgggacctgatcagcaacaacgagcagctgcctatgctcggcagaaggcctggcgctcctgagagcaaatgttctagaggcgccctgtacaccggcttcagcatcctggttacactgctgctggccggacaggctaccaccgcctactttctgtatcagcagcagggcagactggacaagctgaccgtgaccagccagaacctgcagctggaaaacctgcggatgaagctgcccaagcctcctaagcctgtgtccaagatgcggatggccacacctctgctgatgcaggctttgcctatgggagcactgcctcagggccctatgcagaacgccaccaaatacggcaacatgaccgaggaccacgtgatgcatctgctgcagaatgccgatcctctgaaggtgtaccctccactgaagggcagctttcccgagaacctgcggcacctgaagaacaccatggaaaccatcgactggaaggtgttcgagagctggatgcaccactggctgctgttcgagatgagccggcacagcctggaacagaagcctacagacgcccctcctaaagagtccctggaactggaagatcccagctctggcctgggcgtgacaaagcaagatctgggccctgtgcctatgatggacccttctggaattcacggcgagggcaccagatgcaccggatggttcgatgtggaagccatcgtggaaagacggaccggcgacgtggtgtccgaggacgaagatgagaacctggaagataccggcgtggacctggtggacttcatcgacgatacctgcgagtttgagcaggccggcgacgaagctcctagagcactgctgcatgcccaagagacacaggcccatgtgaacgccgtgcactacctgaagcggaagttcacatgcagccctaccgtgtctcccctgggcaatgtgcagtgttctatcgagggcgagctgagccctagactgcacgccatccaactgaatgaaggcagcggcaaggccaagcggagactgtttctgcctgatagcggctacggcaataccgaggtggaaacccagctgtctcaggtggcaggatccgatggcgaatctcaagcccctggcggcaaagaaaatgtgcagcctgctgaaggcgctggcggagatgttcgacagctgctgagaagcagcaacctgaaggccacactgctgggcaagttcaaggacgtgtacggcatcggcttcaacgaactcgtgcggtgcttcaagagcgacaagaccgtgtgcaccgattgggttgtgggagctgctggcgtgcaccattctgtggccgaaggactgaagcacctgatcgagcctttctgcacatacgcccacatgcagtgcctgacctgcaactggggagtgtttctgctgctgctcatccggttcaagtgcggcaagaacagactgaccgtggccaagtgcatctctaccctgctgaacgtgcaagagccctacctgctgatcgaacctccaaagctgagatccacagccgccgctctgtactggtacagaaccggcatcagcaacgtgtccgaagtgatcggagagcctcctgagtggatcgtgcggcagaccatgtttcagcacagcctcgaggacagcatcttcgacctgtctcagatggtgcagtgggcctacgaccacgatctggtggacgatagcgccattgcctaccagtatgcccagctggccgacgtggactctaatgccgccgctttcctgaagtctaacgctcaggccaaatacgtgaaggactgcgccaccatgtgcagacactacaagagagccgagaagcaggccatgaccatgagccagtggatcagagccagatgcgataagaccgacgacggcggagattggaggcctatcgtgcagttcctgagataccagaacgtcgagttcatcacctttctgagcgcctttaaggccttcctgaagggaatccccaagagaagctgcatcgtgctgtacggccctccaaacaccggcaagagctacttcggaatgagcctgatgcagtttctgcagggcagcatcatcagctacgtgaacagcaacagccacttttggctggcccctctggccgaagctaaagtggccatgctggacgatgccacaccacagtgctggtcctacatcgacacctacatgcggagcgtgctggacggcaaccccatgagcattgaccggaagcacaagaacctgatccagatgaagtgtccgcctctgatcatcaccagcaacaccaacgtgggcgaagatgacagatggcgctacctgcactccagagtggtggtgttcaagttcgagcagacctttccattcgaccagaacggcaatcccgtgtacgagctgtccgacaagaattggaagtccttcttcagccggacctggtccagactcgagctgcaagaggatgaggacaccgagcaggatggctctacagtgcgggcctttaagtgtgtgcccggccagaacaccaatatcctgggctctatggaagccctgagcgagagactgaacgcctaccaggacaagatcctgaccttctacgaagaggactccaaggacctgagggaccagatcgagcattggagatgtgtgcggggagagtgcgccgtgttctacaaggccagagagatgggccacagccacatcaaccaccagcaggttccacctctgcacgtgtccagaggcaaggcccacaatgccattgagctgcagatggccctggaaagcctgaaccagagcgacttcaacaccgaggaatggaccctgcaggacacaagcctggaaatgtggcggaccgagcctcagtgctgcttcaaaaagaaaggcgagacagtgaccgtgctgtatgactgcgacaaggacaacagcatggactacaccctgtggggccacgtgtacgtgtggggagatacaggctgggacaaagtgtgcggaaaggtggactacggcggcctgtactatgtgctgcacggcctgaaagtgtactacgttgagttccagcaagaggccgtgaagtacagcaagaccaacaagtgggaagtgcatatcggcagaacagtgatcccctacagcgagtccgtgtcctctacagccctgtgcgaaagagtgcctatcgccgagatcgccaatggcctccagcagaccagcattccctctcacacacacgccaccaccaaagagaacgcttgggtttcctctcagcctcctaccaagcggtccagaagatctgatccaggcggcgatcctgtcagagccctggatgccaatagcagaaccctgctgtctggaaccgcctgcgagaataccaccggcaactcttacagcgactgcacccctgtggtgcacctgaaaggcgaccccaattgtctgaaggccctgcggttcagattcgtgaaacacaagaatctgttcgtgaacgtgtcctccacctggcggtggaccaacaacaccgaagagaaggccatcgtgactgtgacctttaaggacgagcagcagagacagcagttcctcggcagcgtgaagatccccaactccatctgtgtgtgcaagggcgtgatgacagttggcagcggcgccaccaatttcagtctgcttaaacaggccggggacgtcgaagagaaccccggacctatgcatcggcgcagatctcggtcttgtagagaagaccaaaagcctgtcatggatgatcaaagagatttaatttctaataatgaacaattacccatgttgggacggcgccccggagcccccgaatctaagtgcagccggggagctttatatacaggattttctattttagtgaccttattattagctggccaagccacaacagcttatttcttataccaacaacaaggacggttagataaattaacagtcacatctcaaaatttacaattagagaatttaagaatgaaattacctaaaccacccaaacccgtcagtaaaatgagaatggctacccccttattaatgcaagccctccccatgggcgccttaccccaaggacccatgcaaaatgctacaaagtatggaaatatgacagaagatcatgtcatgcacttattacaaaacgctgaccccttaaaagtctatccccctttaaaaggatctttccctgaaaatttaagacatttaaaaaatacaatggagacaattgattggaaagtctttgaatcttggatgcatcattggttattatttgaaatgtctaggcattctttagagcaaaaacccaccgatgctccacccaaggaaagtttagagttagaggacccttctagcggattaggagtcaccaaacaggacttaggacccgtccccatgatggtggaaggcagccaagagagagcccggacactgcacgaactgtgcgagcagagagaacagacccagcacgacatcgagctggactgcgtgtactgcctgaatcctctgaccagaaccgaggtgtacgacttcgccagatgggagctgagaatcgtgtacagacgggataccgcctacggcgtgtgcaagctgtgcctgaggttccacagcaaagtgcggaagtacaggcggtacaactactccatctacggccccacactggaacggcggacaaagaagcagctcgaagaactcgtgatccggtgctactgctgccagaaacctcagtgccccgtggaaaagcagcgccacgttgaccagggccagagattccacaacattgccgagcagtggaccggcagatgcctgcaatgttggaggccaacagccgctgaaaccggctctatgatcggccctggacctacactgcaggatatcgtgcttgagctggaacccgagcctgtggacctgagaggatatgagcagctggactccagcgaggacgaggacgagattgaccaccaccaacagcaggacagacccgatgtgcaagaagatgacggcgagcggtttagaatcctgagcgactgtcacgtgtgccagaataccgtgtcaagagtggtgcacagcacccacgacgacctgcacattctcgaggacctgcttatgggcaccctcgagatcgtggcccctaattgtgccgtgggcagcgaacagaaactgatctccgaggaagatctctgaggaagaatgtgcacagtgctaaccagaaaattcaggacaagactagtgcggccgcttttat

SEQ ID NO: 87 - DNA, 3988 bp, synthetic construct
  misc_feature 1..3988: note = MfPV3 E1E2E6E7 integrated into TK locus of the poxviral vector
  source 1..3988: mol_type = other DNA
ggccaaaaattgaaattttattttttttttttggaatataaataatccctatcagtgatagagatctccctatcagtgatagagatcgtcgacgagctcgtttagtgaaccgtcagatcgcctggagacgccatccacgctgttttgacctctagcaccggtgccaccatggacccttctggaattcacggcgagggcaccagatgcaccggatggttcgatgtggaagccatcgtggaaagacggaccggcgacgtggtgtccgaggacgaagatgagaacctggaagataccggcgtggacctggtggacttcatcgacgatacctgcgagtttgagcaggccggcgacgaagctcctagagcactgctgcatgcccaagagacacaggcccatgtgaacgccgtgcactacctgaagcggaagttcacatgcagccctaccgtgtctcccctgggcaatgtgcagtgttctatcgagggcgagctgagccctagactgcacgccatccaactgaatgaaggcagcggcaaggccaagcggagactgtttctgcctgatagcggctacggcaataccgaggtggaaacccagctgtctcaggtggcaggatccgatggcgaatctcaagcccctggcggcaaagaaaatgtgcagcctgctgaaggcgctggcggagatgttcgacagctgctgagaagcagcaacctgaaggccacactgctgggcaagttcaaggacgtgtacggcatcggcttcaacgaactcgtgcggtgcttcaagagcgacaagaccgtgtgcaccgattgggttgtgggagctgctggcgtgcaccattctgtggccgaaggactgaagcacctgatcgagcctttctgcacatacgcccacatgcagtgcctgacctgcaactggggagtgtttctgctgctgctcatccggttcaagtgcggcaagaacagactgaccgtggccaagtgcatctctaccctgctgaacgtgcaagagccctacctgctgatcgaacctccaaagctgagatccacagccgccgctctgtactggtacagaaccggcatcagcaacgtgtccgaagtgatcggagagcctcctgagtggatcgtgcggcagaccatgtttcagcacagcctcgaggacagcatcttcgacctgtctcagatggtgcagtgggcctacgaccacgatctggtggacgatagcgccattgcctaccagtatgcccagctggccgacgtggactctaatgccgccgctttcctgaagtctaacgctcaggccaaatacgtgaaggactgcgccaccatgtgcagacactacaagagagccgagaagcaggccatgaccatgagccagtggatcagagccagatgcgataagaccgacgacggcggagattggaggcctatcgtgcagttcctgagataccagaacgtcgagttcatcacctttctgagcgcctttaaggccttcctgaagggaatccccaagagaagctgcatcgtgctgtacggccctccaaacaccggcaagagctacttcggaatgagcctgatgcagtttctgcagggcagcatcatcagctacgtgaacagcaacagccacttttggctggcccctctggccgaagctaaagtggccatgctggacgatgccacaccacagtgctggtcctacatcgacacctacatgcggagcgtgctggacggcaaccccatgagcattgaccggaagcacaagaacctgatccagatgaagtgtccgcctctgatcatcaccagcaacaccaacgtgggcgaagatgacagatggcgctacctgcactccagagtggtggtgttcaagttcgagcagacctttccattcgaccagaacggcaatcccgtgtacgagctgtccgacaagaattggaagtccttcttcagccggacctggtccagactcgagctgcaagaggatgaggacaccgagcaggatggctctacagtgcgggcctttaagtgtgtgcccggccagaacaccaatatcctgggctctatggaagccctgagcgagagactgaacgcctaccaggacaagatcctgaccttctacgaagaggactccaaggacctgagggaccagatcgagcattggagatgtgtgcggggagagtgcgccgtgttctacaaggccagagagatgggccacagccacatcaaccaccagcaggttccacctctgcacgtgtccagaggcaaggcccacaatgccattgagctgcagatggccctggaaagcctgaaccagagcgacttcaacaccgaggaatggaccctgcaggacacaagcctggaaatgtggcggaccgagcctcagtgctgcttcaaaaagaaaggcgagacagtgaccgtgctgtatgactgcgacaaggacaacagcatggactacaccctgtggggccacgtgtacgtgtggggagatacaggctgggacaaagtgtgcggaaaggtggactacggcggcctgtactatgtgctgcacggcctgaaagtgtactacgttgagttccagcaagaggccgtgaagtacagcaagaccaacaagtgggaagtgcatatcggcagaacagtgatcccctacagcgagtccgtgtcctctacagccctgtgcgaaagagtgcctatcgccgagatcgccaatggcctccagcagaccagcattccctctcacacacacgccaccaccaaagagaacgcttgggtttcctctcagcctcctaccaagcggtccagaagatctgatccaggcggcgatcctgtcagagccctggatgccaatagcagaaccctgctgtctggaaccgcctgcgagaataccaccggcaactcttacagcgactgcacccctgtggtgcacctgaaaggcgaccccaattgtctgaaggccctgcggttcagattcgtgaaacacaagaatctgttcgtgaacgtgtcctccacctggcggtggaccaacaacaccgaagagaaggccatcgtgactgtgacctttaaggacgagcagcagagacagcagttcctcggcagcgtgaagatccccaactccatctgtgtgtgcaagggcgtgatgacagttggcagcatggtggaaggcagccaagagagagcccggacactgcacgaactgtgcgagcagagagaacagacccagcacgacatcgagctggactgcgtgtactgcctgaatcctctgaccagaaccgaggtgtacgacttcgccagatgggagctgagaatcgtgtacagacgggataccgcctacggcgtgtgcaagctgtgcctgaggttccacagcaaagtgcggaagtacaggcggtacaactactccatctacggccccacactggaacggcggacaaagaagcagctcgaagaactcgtgatccggtgctactgctgccagaaacctcagtgccccgtggaaaagcagcgccacgttgaccagggccagagattccacaacattgccgagcagtggaccggcagatgcctgcaatgttggaggccaacagccgctgaaaccggctctatgatcggccctggacctacactgcaggatatcgtgcttgagctggaacccgagcctgtggacctgagaggatatgagcagctggactccagcgaggacgaggacgagattgaccaccaccaacagcaggacagacccgatgtgcaagaagatgacggcgagcggtttagaatcctgagcgactgtcacgtgtgccagaataccgtgtcaagagtggtgcacagcacccacgacgacctgcacattctcgaggacctgcttatgggcaccctcgagatcgtggcccctaattgtgccgtgggcagcgaacagaaactgatctccgaggaagatctctgaggaagaatgtgcacagtgctaaccagaaaattcaggacaagactagtgcggccgcttttat

SEQ ID NO: 88 - DNA, 5440 bp, synthetic construct
  misc_feature 1..5440: note = MfPV3 Ii-E1E6E7-p2a-Ii-E2 integrated into TK locus of the poxviral vector
  source 1..5440: mol_type = other DNA
ggccaaaaattgaaattttattttttttttttggaatataaataatccctatcagtgatagagatctccctatcagtgatagagatcgtcgacgagctcgtttagtgaaccgtcagatcgcctggagacgccatccacgctgttttgacctctagcaccggtgccaccatgcacagaaggcggagcagaagctgccgcgaggatcagaaacccgtgatggacgaccagcgggacctgatcagcaacaacgagcagctgcctatgctcggcagaaggcctggcgctcctgagagcaaatgttctagaggcgccctgtacaccggcttcagcatcctggttacactgctgctggccggacaggctaccaccgcctactttctgtatcagcagcagggcagactggacaagctgaccgtgaccagccagaacctgcagctggaaaacctgcggatgaagctgcccaagcctcctaagcctgtgtccaagatgcggatggccacacctctgctgatgcaggctttgcctatgggagcactgcctcagggccctatgcagaacgccaccaaatacggcaacatgaccgaggaccacgtgatgcatctgctgcagaatgccgatcctctgaaggtgtaccctccactgaagggcagctttcccgagaacctgcggcacctgaagaacaccatggaaaccatcgactggaaggtgttcgagagctggatgcaccactggctgctgttcgagatgagccggcacagcctggaacagaagcctacagacgcccctcctaaagagtccctggaactggaagatcccagctctggcctgggcgtgacaaagcaagatctgggccctgtgcctatgatggacccttctggaattcacggcgagggcaccagatgcaccggatggttcgatgtggaagccatcgtggaaagacggaccggcgacgtggtgtccgaggacgaagatgagaacctggaagataccggcgtggacctggtggacttcatcgacgatacctgcgagtttgagcaggccggcgacgaagctcctagagcactgctgcatgcccaagagacacaggcccatgtgaacgccgtgcactacctgaagcggaagttcacatgcagccctaccgtgtctcccctgggcaatgtgcagtgttctatcgagggcgagctgagccctagactgcacgccatccaactgaatgaaggcagcggcaaggccaagcggagactgtttctgcctgatagcggctacggcaataccgaggtggaaacccagctgtctcaggtggcaggatccgatggcgaatctcaagcccctggcggcaaagaaaatgtgcagcctgctgaaggcgctggcggagatgttcgacagctgctgagaagcagcaacctgaaggccacactgctgggcaagttcaaggacgtgtacggcatcggcttcaacgaactcgtgcggtgcttcaagagcgacaagaccgtgtgcaccgattgggttgtgggagctgctggcgtgcaccattctgtggccgaaggactgaagcacctgatcgagcctttctgcacatacgcccacatgcagtgcctgacctgcaactggggagtgtttctgctgctgctcatccggttcaagtgcggcaagaacagactgaccgtggccaagtgcatctctaccctgctgaacgtgcaagagccctacctgctgatcgaacctccaaagctgagatccacagccgccgctctgtactggtacagaaccggcatcagcaacgtgtccgaagtgatcggagagcctcctgagtggatcgtgcggcagaccatgtttcagcacagcctcgaggacagcatcttcgacctgtctcagatggtgcagtgggcctacgaccacgatctggtggacgatagcgccattgcctaccagtatgcccagctggccgacgtggactctaatgccgccgctttcctgaagtctaacgctcaggccaaatacgtgaaggactgcgccaccatgtgcagacactacaagagagccgagaagcaggccatgaccatgagccagtggatcagagccagatgcgataagaccgacgacggcggagattggaggcctatcgtgcagttcctgagataccagaacgtcgagttcatcacctttctgagcgcctttaaggccttcctgaagggaatccccaagagaagctgcatcgtgctgtacggccctccaaacaccggcaagagctacttcggaatgagcctgatgcagtttctgcagggcagcatcatcagctacgtgaacagcaacagccacttttggctggcccctctggccgaagctaaagtggccatgctggacgatgccacaccacagtgctggtcctacatcgacacctacatgcggagcgtgctggacggcaaccccatgagcattgaccggaagcacaagaacctgatccagatgaagtgtccgcctctgatcatcaccagcaacaccaacgtgggcgaagatgacagatggcgctacctgcactccagagtggtggtgttcaagttcgagcagacctttccattcgaccagaacggcaatcccgtgtacgagctgtccgacaagaattggaagtccttcttcagccggacctggtccagactcgagctgcaagaggatgaggacaccgagcaggatggctctacagtgcgggcctttaagtgtgtgcccggccagaacaccaatatcctgggctctatggtggaaggcagccaagagagagcccggacactgcacgaactgtgcgagcagagagaacagacccagcacgacatcgagctggactgcgtgtactgcctgaatcctctgaccagaaccgaggtgtacgacttcgccagatgggagctgagaatcgtgtacagacgggataccgcctacggcgtgtgcaagctgtgcctgaggttccacagcaaagtgcggaagtacaggcggtacaactactccatctacggccccacactggaacggcggacaaagaagcagctcgaagaactcgtgatccggtgctactgctgccagaaacctcagtgccccgtggaaaagcagcgccacgttgaccagggccagagattccacaacattgccgagcagtggaccggcagatgcctgcaatgttggaggccaacagccgctgaaaccggctctatgatcggccctggacctacactgcaggatatcgtgcttgagctggaacccgagcctgtggacctgagaggatatgagcagctggactccagcgaggacgaggacgagattgaccaccaccaacagcaggacagacccgatgtgcaagaagatgacggcgagcggtttagaatcctgagcgactgtcacgtgtgccagaataccgtgtcaagagtggtgcacagcacccacgacgacctgcacattctcgaggacctgcttatgggcaccctcgagatcgtggcccctaattgtgccgtgggcagcggcgccaccaatttcagtctgcttaaacaggccggggacgtcgaagagaaccccggacctatgcatcggcgcagatctcggtcttgtagagaagaccaaaagcctgtcatggatgatcaaagagatttaatttctaataatgaacaattacccatgttgggacggcgccccggagcccccgaatctaagtgcagccggggagctttatatacaggattttctattttagtgaccttattattagctggccaagccacaacagcttatttcttataccaacaacaaggacggttagataaattaacagtcacatctcaaaatttacaattagagaatttaagaatgaaattacctaaaccacccaaacccgtcagtaaaatgagaatggctacccccttattaatgcaagccctccccatgggcgccttaccccaaggacccatgcaaaatgctacaaagtatggaaatatgacagaagatcatgtcatgcacttattacaaaacgctgaccccttaaaagtctatccccctttaaaaggatctttccctgaaaatttaagacatttaaaaaatacaatggagacaattgattggaaagtctttgaatcttggatgcatcattggttattatttgaaatgtctaggcattctttagagcaaaaacccaccgatgctccacccaaggaaagtttagagttagaggacccttctagcggattaggagtcaccaaacaggacttaggacccgtccccatgatggaagccctgagcgagagactgaacgcctaccaggacaagatcctgaccttctacgaagaggactccaaggacctgagggaccagatcgagcattggagatgtgtgcggggagagtgcgccgtgttctacaaggccagagagatgggccacagccacatcaaccaccagcaggttccacctctgcacgtgtccagaggcaaggcccacaatgccattgagctgcagatggccctggaaagcctgaaccagagcgacttcaacaccgaggaatggaccctgcaggacacaagcctggaaatgtggcggaccgagcctcagtgctgcttcaaaaagaaaggcgagacagtgaccgtgctgtatgactgcgacaaggacaacagcatggactacaccctgtggggccacgtgtacgtgtggggagatacaggctgggacaaagtgtgcggaaaggtggactacggcggcctgtactatgtgctgcacggcctgaaagtgtactacgttgagttccagcaagaggccgtgaagtacagcaagaccaacaagtgggaagtgcatatcggcagaacagtgatcccctacagcgagtccgtgtcctctacagccctgtgcgaaagagtgcctatcgccgagatcgccaatggcctccagcagaccagcattccctctcacacacacgccaccaccaaagagaacgcttgggtttcctctcagcctcctaccaagcggtccagaagatctgatccaggcggcgatcctgtcagagccctggatgccaatagcagaaccctgctgtctggaaccgcctgcgagaataccaccggcaactcttacagcgactgcacccctgtggtgcacctgaaaggcgaccccaattgtctgaaggccctgcggttcagattcgtgaaacacaagaatctgttcgtgaacgtgtcctccacctggcggtggaccaacaacaccgaagagaaggccatcgtgactgtgacctttaaggacgagcagcagagacagcagttcctcggcagcgtgaagatccccaactccatctgtgtgtgcaagggcgtgatgacagttggcagcgaacagaaactgatctccgaggaagatctctgaggaagaatgtgcacagtgctaaccagaaaattcaggacaagactagtgcggccgcttttat

SEQ ID NO: 89 - DNA, 3598 bp, synthetic construct
  misc_feature 1..3598: note = MfPV3 Ii-E1E6E7 integrated into TK locus of the poxviral vector
  source 1..3598: mol_type = other DNA
ggccaaaaattgaaattttattttttttttttggaatataaataatccctatcagtgatagagatctccctatcagtgatagagatcgtcgacgagctcgtttagtgaaccgtcagatcgcctggagacgccatccacgctgttttgacctctagcaccggtgccaccatgcacagaaggcggagcagaagctgccgcgaggatcagaaacccgtgatggacgaccagcgggacctgatcagcaacaacgagcagctgcctatgctcggcagaaggcctggcgctcctgagagcaaatgttctagaggcgccctgtacaccggcttcagcatcctggttacactgctgctggccggacaggctaccaccgcctactttctgtatcagcagcagggcagactggacaagctgaccgtgaccagccagaacctgcagctggaaaacctgcggatgaagctgcccaagcctcctaagcctgtgtccaagatgcggatggccacacctctgctgatgcaggctttgcctatgggagcactgcctcagggccctatgcagaacgccaccaaatacggcaacatgaccgaggaccacgtgatgcatctgctgcagaatgccgatcctctgaaggtgtaccctccactgaagggcagctttcccgagaacctgcggcacctgaagaacaccatggaaaccatcgactggaaggtgttcgagagctggatgcaccactggctgctgttcgagatgagccggcacagcctggaacagaagcctacagacgcccctcctaaagagtccctggaactggaagatcccagctctggcctgggcgtgacaaagcaagatctgggccctgtgcctatgatggacccttctggaattcacggcgagggcaccagatgcaccggatggttcgatgtggaagccatcgtggaaagacggaccggcgacgtggtgtccgaggacgaagatgagaacctggaagataccggcgtggacctggtggacttcatcgacgatacctgcgagtttgagcaggccggcgacgaagctcctagagcactgctgcatgcccaagagacacaggcccatgtgaacgccgtgcactacctgaagcggaagttcacatgcagccctaccgtgtctcccctgggcaatgtgcagtgttctatcgagggcgagctgagccctagactgcacgccatccaactgaatgaaggcagcggcaaggccaagcggagactgtttctgcctgatagcggctacggcaataccgaggtggaaacccagctgtctcaggtggcaggatccgatggcgaatctcaagcccctggcggcaaagaaaatgtgcagcctgctgaaggcgctggcggagatgttcgacagctgctgagaagcagcaacctgaaggccacactgctgggcaagttcaaggacgtgtacggcatcggcttcaacgaactcgtgcggtgcttcaagagcgacaagaccgtgtgcaccgattgggttgtgggagctgctggcgtgcaccattctgtggccgaaggactgaagcacctgatcgagcctttctgcacatacgcccacatgcagtgcctgacctgcaactggggagtgtttctgctgctgctcatccggttcaagtgcggcaagaacagactgaccgtggccaagtgcatctctaccctgctgaacgtgcaagagccctacctgctgatcgaacctccaaagctgagatccacagccgccgctctgtactggtacagaaccggcatcagcaacgtgtccgaagtgatcggagagcctcctgagtggatcgtgcggcagaccatgtttcagcacagcctcgaggacagcatcttcgacctgtctcagatggtgcagtgggcctacgaccacgatctggtggacgatagcgccattgcctaccagtatgcccagctggccgacgtggactctaatgccgccgctttcctgaagtctaacgctcaggccaaatacgtgaaggactgcgccaccatgtgcagacactacaagagagccgagaagcaggccatgaccatgagccagtggatcagagccagatgcgataagaccgacgacggcggagattggaggcctatcgtgcagttcctgagataccagaacgtcgagttcatcacctttctgagcgcctttaaggccttcctgaagggaatccccaagagaagctgcatcgtgctgtacggccctccaaacaccggcaagagctacttcggaatgagcctgatgcagtttctgcagggcagcatcatcagctacgtgaacagcaacagccacttttggctggcccctctggccgaagctaaagtggccatgctggacgatgccacaccacagtgctggtcctacatcgacacctacatgcggagcgtgctggacggcaaccccatgagcattgaccggaagcacaagaacctgatccagatgaagtgtccgcctctgatcatcaccagcaacaccaacgtgggcgaagatgacagatggcgctacctgcactccagagtggtggtgttcaagttcgagcagacctttccattcgaccagaacggcaatcccgtgtacgagctgtccgacaagaattggaagtccttcttcagccggacctggtccagactcgagctgcaagaggatgaggacaccgagcaggatggctctacagtgcgggcctttaagtgtgtgcccggccagaacaccaatatcctgggctctatggtggaaggcagccaagagagagcccggacactgcacgaactgtgcgagcagagagaacagacccagcacgacatcgagctggactgcgtgtactgcctgaatcctctgaccagaaccgaggtgtacgacttcgccagatgggagctgagaatcgtgtacagacgggataccgcctacggcgtgtgcaagctgtgcctgaggttccacagcaaagtgcggaagtacaggcggtacaactactccatctacggccccacactggaacggcggacaaagaagcagctcgaagaactcgtgatccggtgctactgctgccagaaacctcagtgccccgtggaaaagcagcgccacgttgaccagggccagagattccacaacattgccgagcagtggaccggcagatgcctgcaatgttggaggccaacagccgctgaaaccggctctatgatcggccctggacctacactgcaggatatcgtgcttgagctggaacccgagcctgtggacctgagaggatatgagcagctggactccagcgaggacgaggacgagattgaccaccaccaacagcaggacagacccgatgtgcaagaagatgacggcgagcggtttagaatcctgagcgactgtcacgtgtgccagaataccgtgtcaagagtggtgcacagcacccacgacgacctgcacattctcgaggacctgcttatgggcaccctcgagatcgtggcccctaattgtgccgtgggcagcgaacagaaactgatctccgaggaagatctctgaggaagaatgtgcacagtgctaaccagaaaattcaggacaagactagtgcggccgcttttat

SEQ ID NO: 90 - DNA, 4744 bp, synthetic construct
  misc_feature 1..4744: note = HPV16 Ii-E1E2E6E7 integrated into TK locus of the poxviral vector
  source 1..4744: mol_type = other DNA
ggccaaaaattgaaattttattttttttttttggaatataaataatccctatcagtgatagagatctccctatcagtgatagagatcgtcgacgagctcgtttagtgaaccgtcagatcgcctggagacgccatccacgctgttttgacctctagcaccggtgccaccatgcacagaaggcggagcagaagctgccgcgaggatcagaaacccgtgatggacgaccagcgggacctgatcagcaacaacgagcagctgcctatgctcggcagaaggcctggcgctcctgagagcaaatgttctagaggcgccctgtacaccggcttcagcatcctggttacactgctgctggccggacaggctaccaccgcctactttctgtatcagcagcagggcagactggacaagctgaccgtgaccagccagaacctgcagctggaaaacctgcggatgaagctgcccaagcctcctaagcctgtgtccaagatgcggatggccacacctctgctgatgcaggctttgcctatgggagcactgcctcagggccctatgcagaacgccaccaaatacggcaacatgaccgaggaccacgtgatgcatctgctgcagaatgccgatcctctgaaggtgtaccctccactgaagggcagctttcccgagaacctgcggcacctgaagaacaccatggaaaccatcgactggaaggtgttcgagagctggatgcaccactggctgctgttcgagatgagccggcacagcctggaacagaagcctacagacgcccctcctaaagagtccctggaactggaagatcccagctctggcctgggcgtgacaaagcaagatctgggccctgtgcctatgatggccgatcctgctggcacaaacggcgaagaaggcacaggctgcaacggctggttttacgtggaagccgtggtggaaaagaaaaccggcgacgccatcagcgacgacgagaacgagaatgacagcgacaccggcgaggacctggtggacttcatcgtgaacgacaacgactacctgacacaggccgagacagagacagcccacgctctgtttacagcccaagaggccaagcagcacagagatgccgttcaggtgctgaagagaaagtacctgggcagccctctgagcgacatcagcggctgtgtggacaacaacatcagccccagactgaaggccatctgcatcgagaagcagagcagagccgccaagcggagactgtttgagagcgaggatagcggctacggcaataccgaggtggaaacccagcaaatgctgcaggtcgagggcagacacgaaaccgagacaccttgcagccagtacagcggaggatctggcggaggctgtagccagtactcttctggaagtggcggagagggcgtgtccgagagacacaccatttgtcagacccctctgaccaacatcctgaacgtgctgaaaaccagcaacgccaaggccgccatgctggccaagttcaaagagctgtacggcgtcagcttcagcgaactcgtgcggcccttcaagagcaacaagtccacctgttgcgactggtgcattgccgcctttggcctgacaccttctatcgccgacagcatcaagaccctgctccagcagtactgtctgtacctgcacattcagagcctggcctgctcttggggaatggtggtgctgctgctcgtgcggtacaagtgcggcaagaacagagagacaattgagaagctgctgagcaaactgctgtgcgtgtcccctatgtgcatgatgatcgagcctccaaagctgcggtctacagccgccgctctgtactggtacaagaccggcatcagcaatatctccgaggtgtacggcgacacccctgagtggattcagagacagaccgtcctgcagcacagcttcaacgactgcaccttcgagctgtcccagatggtgcagtgggcctacgacaatgacatcgtggacgactccgagatcgcctataagtacgcccagctggccgacaccaacagcaatgccagcgccttcctgaagtccaacagccaggccaagatcgtgaaggactgcgccaccatgtgcagacactacaagcgggccgagaagaaacagatgagcatgagccagtggatcaagtacagatgcgacagagtggacgacggcggcgattggaagcagatcgtgatgttcctgagataccagggcgtcgagttcatgagctttctgacagccctgaagcggttcctgcagggcatccccaagaagaactgcatcctgctgtatggcgccgctaacaccggcaagagcctgttcggaatgagcctgatgaagtttctgcagggctccgtgatctgctttgtgaacagcaagagccacttctggctccagccactggccgatgccaagattggcatgctggacgatgccaccgtgccttgctggaactacatcgacgacaacctgagaaacgccctggacggcaacctggtttctatggacgtgaagcacagacccctggtgcagctgaagtgtcctccactgctgatcaccagcaacatcaacgccggcaccgattccagatggccctatctgcacaaccggctggtggtgtttacctttcctaacgagttccccttcgacgaaaacggcaaccccgtgtacgagctgaacgataagaattggaagtccttcttcagccggacctggtccagactgagcctgcacgaggatgaggacaaagagaacgacggcgacagcctgcctaccttcaaatgtgtcagcggccagaacaccaacacactgggcagcatggaaacactgtgccagcggctgaacgtgtgccaggacaagattctgacccactatgagaacgacagcaccgacctgagggaccacatcgactactggaagcacatgcggctggaatgcgccatctactacaaggctagagagatgggcttcaagcacatcaaccaccaggtggtgcctacactggccgtgtctaagaacaaggctctgcaggccatcgaactgcagctgaccctggaaacaatctacaactcccagtactccaacgagaagtggaccctccaggacgtgtcactggaagtgtacctgaccgctcctaccggctgcatcaagaaacacggctacaccgtggaagtgcagtttgacggcgatatctgcaataccatgcactacaccaactggacccacatctacatctgcgaagaggccagcgtgacagtggtggaaggccaggtcgactactacggcctgtactacgtgcacgagggcatcagaacctacttcgtgcagttcaaggatgacgccgagaagtacagcaagaacaaagtgtgggaagtgcacgctggcggccaagtgatcctgtgtcctacaagcgtgttcagcagcaacgaagtgtctagccccgagatcatcagacagcacctggccaatcatcctgccgccacacacacaaaagctgtggccctgggcaccgaggaaacacagaccacaattcagaggcccagaagcgagcccgataccggcaatccttgtcacaccaccaagctgctgcaccgggattccgtggattctgcccctatcctgaccgccttcaacagctcccacaaaggccggatcaactgcaacagcaacaccacacctatcgtgcacctgaaaggggacgccaacactctgaaggccctgcggtacagattcaagaagcactgtaccctgtataccgccgtgtccagcacatggcactggaccggacacaacgtgaaacacaagagcgccatcgtgaccctgacctacgatagcgagtggcagagggaccagttcctgagccaagtgaagatccctaagaccatcaccgtgtccaccggctttatgagcattggcagcatgcaccagaaacggacagccatgttccaggatcctcaagagcggccacggaagctgcctcagctgtgtacagaactgcagaccaccatccacgacatcatcctcgagtgcgtgtactgcaagcagcagctcctgcggagagaagtgtacgacttcgccttccgggatctgtgcatcgtgtacagagatggcaacccctacgccgtgtgcgacaagtgcctgaagttctactccaagatcagcgagtaccggcactactgctactccctgtacggcaccacacttgagcagcagtacaacaagcccctgtgcgatctgctgatccggtgcatcaactgccagaaacctcagtgccccgaggaaaagcagagacacctggacaagaagcagcggttccacaacattagaggcagatggacaggccggtgcatgagctgctgtagaagcagcagaacaagacggggctccatgcacggcgataccccaacactgcacgagtacatgctggatctgcagcccgagacaaccgatctgtacggctatgagcagctgaatgactccagcgaggaagaggacgagatcgacggacctgctggacaggctgaacctgatagagcccactacaatatcgtgaccttctgctgcaagtgcgatagcaccctgagaagatgcgtgcagagcacccacgtggacatcaggaccctcgaggatctgctcatgggcaccctgggaattgtggccccaatctgctctcagaagccttgaggaagaatgtgcacagtgctaaccagaaaattcaggacaagactagtgcggccgcttttat

SEQ ID NO: 91 - DNA, 5506 bp, synthetic construct
  misc_feature 1..5506: note = HPV16 Ii-E1E2-p2a-Ii-E6E7 integrated into TK locus of the poxviral vector
  source 1..5506: mol_type = other DNA
ggccaaaaattgaaattttattttttttttttggaatataaataatccctatcagtgatagagatctccctatcagtgatagagatcgtcgacgagctcgtttagtgaaccgtcagatcgcctggagacgccatccacgctgttttgacctctagcaccggtgccaccatgcacagaaggcggagcagaagctgccgcgaggatcagaaacccgtgatggacgaccagcgggacctgatcagcaacaacgagcagctgcctatgctcggcagaaggcctggcgctcctgagagcaaatgttctagaggcgccctgtacaccggcttcagcatcctggttacactgctgctggccggacaggctaccaccgcctactttctgtatcagcagcagggcagactggacaagctgaccgtgaccagccagaacctgcagctggaaaacctgcggatgaagctgcccaagcctcctaagcctgtgtccaagatgcggatggccacacctctgctgatgcaggctttgcctatgggagcactgcctcagggccctatgcagaacgccaccaaatacggcaacatgaccgaggaccacgtgatgcatctgctgcagaatgccgatcctctgaaggtgtaccctccactgaagggcagctttcccgagaacctgcggcacctgaagaacaccatggaaaccatcgactggaaggtgttcgagagctggatgcaccactggctgctgttcgagatgagccggcacagcctggaacagaagcctacagacgcccctcctaaagagtccctggaactggaagatcccagctctggcctgggcgtgacaaagcaagatctgggccctgtgcctatgatggccgatcctgctggcacaaacggcgaagaaggcacaggctgcaacggctggttttacgtggaagccgtggtggaaaagaaaaccggcgacgccatcagcgacgacgagaacgagaatgacagcgacaccggcgaggacctggtggacttcatcgtgaacgacaacgactacctgacacaggccgagacagagacagcccacgctctgtttacagcccaagaggccaagcagcacagagatgccgttcaggtgctgaagagaaagtacctgggcagccctctgagcgacatcagcggctgtgtggacaacaacatcagccccagactgaaggccatctgcatcgagaagcagagcagagccgccaagcggagactgtttgagagcgaggatagcggctacggcaataccgaggtggaaacccagcaaatgctgcaggtcgagggcagacacgaaaccgagacaccttgcagccagtacagcggaggatctggcggaggctgtagccagtactcttctggaagtggcggagagggcgtgtccgagagacacaccatttgtcagacccctctgaccaacatcctgaacgtgctgaaaaccagcaacgccaaggccgccatgctggccaagttcaaagagctgtacggcgtcagcttcagcgaactcgtgcggcccttcaagagcaacaagtccacctgttgcgactggtgcattgccgcctttggcctgacaccttctatcgccgacagcatcaagaccctgctccagcagtactgtctgtacctgcacattcagagcctggcctgctcttggggaatggtggtgctgctgctcgtgcggtacaagtgcggcaagaacagagagacaattgagaagctgctgagcaaactgctgtgcgtgtcccctatgtgcatgatgatcgagcctccaaagctgcggtctacagccgccgctctgtactggtacaagaccggcatcagcaatatctccgaggtgtacggcgacacccctgagtggattcagagacagaccgtcctgcagcacagcttcaacgactgcaccttcgagctgtcccagatggtgcagtgggcctacgacaatgacatcgtggacgactccgagatcgcctataagtacgcccagctggccgacaccaacagcaatgccagcgccttcctgaagtccaacagccaggccaagatcgtgaaggactgcgccaccatgtgcagacactacaagcgggccgagaagaaacagatgagcatgagccagtggatcaagtacagatgcgacagagtggacgacggcggcgattggaagcagatcgtgatgttcctgagataccagggcgtcgagttcatgagctttctgacagccctgaagcggttcctgcagggcatccccaagaagaactgcatcctgctgtatggcgccgctaacaccggcaagagcctgttcggaatgagcctgatgaagtttctgcagggctccgtgatctgctttgtgaacagcaagagccacttctggctccagccactggccgatgccaagattggcatgctggacgatgccaccgtgccttgctggaactacatcgacgacaacctgagaaacgccctggacggcaacctggtttctatggacgtgaagcacagacccctggtgcagctgaagtgtcctccactgctgatcaccagcaacatcaacgccggcaccgattccagatggccctatctgcacaaccggctggtggtgtttacctttcctaacgagttccccttcgacgaaaacggcaaccccgtgtacgagctgaacgataagaattggaagtccttcttcagccggacctggtccagactgagcctgcacgaggatgaggacaaagagaacgacggcgacagcctgcctaccttcaaatgtgtcagcggccagaacaccaacacactgggcagcatggaaacactgtgccagcggctgaacgtgtgccaggacaagattctgacccactatgagaacgacagcaccgacctgagggaccacatcgactactggaagcacatgcggctggaatgcgccatctactacaaggctagagagatgggcttcaagcacatcaaccaccaggtggtgcctacactggccgtgtctaagaacaaggctctgcaggccatcgaactgcagctgaccctggaaacaatctacaactcccagtactccaacgagaagtggaccctccaggacgtgtcactggaagtgtacctgaccgctcctaccggctgcatcaagaaacacggctacaccgtggaagtgcagtttgacggcgatatctgcaataccatgcactacaccaactggacccacatctacatctgcgaagaggccagcgtgacagtggtggaaggccaggtcgactactacggcctgtactacgtgcacgagggcatcagaacctacttcgtgcagttcaaggatgacgccgagaagtacagcaagaacaaagtgtgggaagtgcacgctggcggccaagtgatcctgtgtcctacaagcgtgttcagcagcaacgaagtgtctagccccgagatcatcagacagcacctggccaatcatcctgccgccacacacacaaaagctgtggccctgggcaccgaggaaacacagaccacaattcagaggcccagaagcgagcccgataccggcaatccttgtcacaccaccaagctgctgcaccgggattccgtggattctgcccctatcctgaccgccttcaacagctcccacaaaggccggatcaactgcaacagcaacaccacacctatcgtgcacctgaaaggggacgccaacactctgaaggccctgcggtacagattcaagaagcactgtaccctgtataccgccgtgtccagcacatggcactggaccggacacaacgtgaaacacaagagcgccatcgtgaccctgacctacgatagcgagtggcagagggaccagttcctgagccaagtgaagatccctaagaccatcaccgtgtccaccggctttatgagcattggcagcggcagcggcgccaccaatttcagtctgcttaaacaggccggggacgtcgaagagaaccccggacctatgcatcggcgcagatctcggtcttgtagagaagaccaaaagcctgtcatggatgatcaaagagatttaatttctaataatgaacaattacccatgttgggacggcgccccggagcccccgaatctaagtgcagccggggagctttatatacaggattttctattttagtgaccttattattagctggccaagccacaacagcttatttcttataccaacaacaaggacggttagataaattaacagtcacatctcaaaatttacaattagagaatttaagaatgaaattacctaaaccacccaaacccgtcagtaaaatgagaatggctacccccttattaatgcaagccctccccatgggcgccttaccccaaggacccatgcaaaatgctacaaagtatggaaatatgacagaagatcatgtcatgcacttattacaaaacgctgaccccttaaaagtctatccccctttaaaaggatctttccctgaaaatttaagacatttaaaaaatacaatggagacaattgattggaaagtctttgaatcttggatgcatcattggttattatttgaaatgtctaggcattctttagagcaaaaacccaccgatgctccacccaaggaaagtttagagttagaggacccttctagcggattaggagtcaccaaacaggacttaggacccgtccccatgatgcaccagaaacggacagccatgttccaggatcctcaagagcggccacggaagctgcctcagctgtgtacagaactgcagaccaccatccacgacatcatcctcgagtgcgtgtactgcaagcagcagctcctgcggagagaagtgtacgacttcgccttccgggatctgtgcatcgtgtacagagatggcaacccctacgccgtgtgcgacaagtgcctgaagttctactccaagatcagcgagtaccggcactactgctactccctgtacggcaccacacttgagcagcagtacaacaagcccctgtgcgatctgctgatccggtgcatcaactgccagaaacctcagtgccccgaggaaaagcagagacacctggacaagaagcagcggttccacaacattagaggcagatggacaggccggtgcatgagctgctgtagaagcagcagaacaagacggggctccatgcacggcgataccccaacactgcacgagtacatgctggatctgcagcccgagacaaccgatctgtacggctatgagcagctgaatgactccagcgaggaagaggacgagatcgacggacctgctggacaggctgaacctgatagagcccactacaatatcgtgaccttctgctgcaagtgcgatagcaccctgagaagatgcgtgcagagcacccacgtggacatcaggaccctcgaggatctgctcatgggcaccctgggaattgtggccccaatctgctctcagaagccttgaggaagaatgtgcacagtgctaaccagaaaattcaggacaagactagtgcggccgcttttat

SEQ ID NO: 92 - DNA, 4048 bp, synthetic construct
  misc_feature 1..4048: note = HPV16 E1E2E6E7 integrated into TK locus of the poxviral vector
  source 1..4048: mol_type = other DNA
ggccaaaaattgaaattttattttttttttttggaatataaataatccctatcagtgatagagatctccctatcagtgatagagatcgtcgacgagctcgtttagtgaaccgtcagatcgcctggagacgccatccacgctgttttgacctctagcaccggtgccaccatggccgatcctgctggcacaaacggcgaagaaggcacaggctgcaacggctggttttacgtggaagccgtggtggaaaagaaaaccggcgacgccatcagcgacgacgagaacgagaatgacagcgacaccggcgaggacctggtggacttcatcgtgaacgacaacgactacctgacacaggccgagacagagacagcccacgctctgtttacagcccaagaggccaagcagcacagagatgccgttcaggtgctgaagagaaagtacctgggcagccctctgagcgacatcagcggctgtgtggacaacaacatcagccccagactgaaggccatctgcatcgagaagcagagcagagccgccaagcggagactgtttgagagcgaggatagcggctacggcaataccgaggtggaaacccagcaaatgctgcaggtcgagggcagacacgaaaccgagacaccttgcagccagtacagcggaggatctggcggaggctgtagccagtactcttctggaagtggcggagagggcgtgtccgagagacacaccatttgtcagacccctctgaccaacatcctgaacgtgctgaaaaccagcaacgccaaggccgccatgctggccaagttcaaagagctgtacggcgtcagcttcagcgaactcgtgcggcccttcaagagcaacaagtccacctgttgcgactggtgcattgccgcctttggcctgacaccttctatcgccgacagcatcaagaccctgctccagcagtactgtctgtacctgcacattcagagcctggcctgctcttggggaatggtggtgctgctgctcgtgcggtacaagtgcggcaagaacagagagacaattgagaagctgctgagcaaactgctgtgcgtgtcccctatgtgcatgatgatcgagcctccaaagctgcggtctacagccgccgctctgtactggtacaagaccggcatcagcaatatctccgaggtgtacggcgacacccctgagtggattcagagacagaccgtcctgcagcacagcttcaacgactgcaccttcgagctgtcccagatggtgcagtgggcctacgacaatgacatcgtggacgactccgagatcgcctataagtacgcccagctggccgacaccaacagcaatgccagcgccttcctgaagtccaacagccaggccaagatcgtgaaggactgcgccaccatgtgcagacactacaagcgggccgagaagaaacagatgagcatgagccagtggatcaagtacagatgcgacagagtggacgacggcggcgattggaagcagatcgtgatgttcctgagataccagggcgtcgagttcatgagctttctgacagccctgaagcggttcctgcagggcatccccaagaagaactgcatcctgctgtatggcgccgctaacaccggcaagagcctgttcggaatgagcctgatgaagtttctgcagggctccgtgatctgctttgtgaacagcaagagccacttctggctccagccactggccgatgccaagattggcatgctggacgatgccaccgtgccttgctggaactacatcgacgacaacctgagaaacgccctggacggcaacctggtttctatggacgtgaagcacagacccctggtgcagctgaagtgtcctccactgctgatcaccagcaacatcaacgccggcaccgattccagatggccctatctgcacaaccggctggtggtgtttacctttcctaacgagttccccttcgacgaaaacggcaaccccgtgtacgagctgaacgataagaattggaagtccttcttcagccggacctggtccagactgagcctgcacgaggatgaggacaaagagaacgacggcgacagcctgcctaccttcaaatgtgtcagcggccagaacaccaacacactgggcagcatggaaacactgtgccagcggctgaacgtgtgccaggacaagattctgacccactatgagaacgacagcaccgacctgagggaccacatcgactactggaagcacatgcggctggaatgcgccatctactacaaggctagagagatgggcttcaagcacatcaaccaccaggtggtgcctacactggccgtgtctaagaacaaggctctgcaggccatcgaactgcagctgaccctggaaacaatctacaactcccagtactccaacgagaagtggaccctccaggacgtgtcactggaagtgtacctgaccgctcctaccggctgcatcaagaaacacggctacaccgtggaagtgcagtttgacggcgatatctgcaataccatgcactacaccaactggacccacatctacatctgcgaagaggccagcgtgacagtggtggaaggccaggtcgactactacggcctgtactacgtgcacgagggcatcagaacctacttcgtgcagttcaaggatgacgccgagaagtacagcaagaacaaagtgtgggaagtgcacgctggcggccaagtgatcctgtgtcctacaagcgtgttcagcagcaacgaagtgtctagccccgagatcatcagacagcacctggccaatcatcctgccgccacacacacaaaagctgtggccctgggcaccgaggaaacacagaccacaattcagaggcccagaagcgagcccgataccggcaatccttgtcacaccaccaagctgctgcaccgggattccgtggattctgcccctatcctgaccgccttcaacagctcccacaaaggccggatcaactgcaacagcaacaccacacctatcgtgcacctgaaaggggacgccaacactctgaaggccctgcggtacagattcaagaagcactgtaccctgtataccgccgtgtccagcacatggcactggaccggacacaacgtgaaacacaagagcgccatcgtgaccctgacctacgatagcgagtggcagagggaccagttcctgagccaagtgaagatccctaagaccatcaccgtgtccaccggctttatgagcattggcagcatgcaccagaaacggacagccatgttccaggatcctcaagagcggccacggaagctgcctcagctgtgtacagaactgcagaccaccatccacgacatcatcctcgagtgcgtgtactgcaagcagcagctcctgcggagagaagtgtacgacttcgccttccgggatctgtgcatcgtgtacagagatggcaacccctacgccgtgtgcgacaagtgcctgaagttctactccaagatcagcgagtaccggcactactgctactccctgtacggcaccacacttgagcagcagtacaacaagcccctgtgcgatctgctgatccggtgcatcaactgccagaaacctcagtgccccgaggaaaagcagagacacctggacaagaagcagcggttccacaacattagaggcagatggacaggccggtgcatgagctgctgtagaagcagcagaacaagacggggctccatgcacggcgataccccaacactgcacgagtacatgctggatctgcagcccgagacaaccgatctgtacggctatgagcagctgaatgactccagcgaggaagaggacgagatcgacggacctgctggacaggctgaacctgatagagcccactacaatatcgtgaccttctgctgcaagtgcgatagcaccctgagaagatgcgtgcagagcacccacgtggacatcaggaccctcgaggatctgctcatgggcaccctgggaattgtggccccaatctgctctcagaagccttgaggaagaatgtgcacagtgctaaccagaaaattcaggacaagactagtgcggccgcttttat

SEQ ID NO: 93 - DNA, 5500 bp, synthetic construct
  misc_feature 1..5500: note = HPV16 Ii-E1E6E7-p2a-Ii-E2 integrated into TK locus of the poxviral vector
  source 1..5500: mol_type = other DNA
ggccaaaaattgaaattttattttttttttttggaatataaataatccctatcagtgatagagatctccctatcagtgatagagatcgtcgacgagctcgtttagtgaaccgtcagatcgcctggagacgccatccacgctgttttgacctctagcaccggtgccaccatgcacagaaggcggagcagaagctgccgcgaggatcagaaacccgtgatggacgaccagcgggacctgatcagcaacaacgagcagctgcctatgctcggcagaaggcctggcgctcctgagagcaaatgttctagaggcgccctgtacaccggcttcagcatcctggttacactgctgctggccggacaggctaccaccgcctactttctgtatcagcagcagggcagactggacaagctgaccgtgaccagccagaacctgcagctggaaaacctgcggatgaagctgcccaagcctcctaagcctgtgtccaagatgcggatggccacacctctgctgatgcaggctttgcctatgggagcactgcctcagggccctatgcagaacgccaccaaatacggcaacatgaccgaggaccacgtgatgcatctgctgcagaatgccgatcctctgaaggtgtaccctccactgaagggcagctttcccgagaacctgcggcacctgaagaacaccatggaaaccatcgactggaaggtgttcgagagctggatgcaccactggctgctgttcgagatgagccggcacagcctggaacagaagcctacagacgcccctcctaaagagtccctggaactggaagatcccagctctggcctgggcgtgacaaagcaagatctgggccctgtgcctatgatggccgatcctgctggcacaaacggcgaagaaggcacaggctgcaacggctggttttacgtggaagccgtggtggaaaagaaaaccggcgacgccatcagcgacgacgagaacgagaatgacagcgacaccggcgaggacctggtggacttcatcgtgaacgacaacgactacctgacacaggccgagacagagacagcccacgctctgtttacagcccaagaggccaagcagcacagagatgccgttcaggtgctgaagagaaagtacctgggcagccctctgagcgacatcagcggctgtgtggacaacaacatcagccccagactgaaggccatctgcatcgagaagcagagcagagccgccaagcggagactgtttgagagcgaggatagcggctacggcaataccgaggtggaaacccagcaaatgctgcaggtcgagggcagacacgaaaccgagacaccttgcagccagtacagcggaggatctggcggaggctgtagccagtactcttctggaagtggcggagagggcgtgtccgagagacacaccatttgtcagacccctctgaccaacatcctgaacgtgctgaaaaccagcaacgccaaggccgccatgctggccaagttcaaagagctgtacggcgtcagcttcagcgaactcgtgcggcccttcaagagcaacaagtccacctgttgcgactggtgcattgccgcctttggcctgacaccttctatcgccgacagcatcaagaccctgctccagcagtactgtctgtacctgcacattcagagcctggcctgctcttggggaatggtggtgctgctgctcgtgcggtacaagtgcggcaagaacagagagacaattgagaagctgctgagcaaactgctgtgcgtgtcccctatgtgcatgatgatcgagcctccaaagctgcggtctacagccgccgctctgtactggtacaagaccggcatcagcaatatctccgaggtgtacggcgacacccctgagtggattcagagacagaccgtcctgcagcacagcttcaacgactgcaccttcgagctgtcccagatggtgcagtgggcctacgacaatgacatcgtggacgactccgagatcgcctataagtacgcccagctggccgacaccaacagcaatgccagcgccttcctgaagtccaacagccaggccaagatcgtgaaggactgcgccaccatgtgcagacactacaagcgggccgagaagaaacagatgagcatgagccagtggatcaagtacagatgcgacagagtggacgacggcggcgattggaagcagatcgtgatgttcctgagataccagggcgtcgagttcatgagctttctgacagccctgaagcggttcctgcagggcatccccaagaagaactgcatcctgctgtatggcgccgctaacaccggcaagagcctgttcggaatgagcctgatgaagtttctgcagggctccgtgatctgctttgtgaacagcaagagccacttctggctccagccactggccgatgccaagattggcatgctggacgatgccaccgtgccttgctggaactacatcgacgacaacctgagaaacgccctggacggcaacctggtttctatggacgtgaagcacagacccctggtgcagctgaagtgtcctccactgctgatcaccagcaacatcaacgccggcaccgattccagatggccctatctgcacaaccggctggtggtgtttacctttcctaacgagttccccttcgacgaaaacggcaaccccgtgtacgagctgaacgataagaattggaagtccttcttcagccggacctggtccagactgagcctgcacgaggatgaggacaaagagaacgacggcgacagcctgcctaccttcaaatgtgtcagcggccagaacaccaacacactgggcagcatgcaccagaaacggacagccatgttccaggatcctcaagagcggccacggaagctgcctcagctgtgtacagaactgcagaccaccatccacgacatcatcctcgagtgcgtgtactgcaagcagcagctcctgcggagagaagtgtacgacttcgccttccgggatctgtgcatcgtgtacagagatggcaacccctacgccgtgtgcgacaagtgcctgaagttctactccaagatcagcgagtaccggcactactgctactccctgtacggcaccacacttgagcagcagtacaacaagcccctgtgcgatctgctgatccggtgcatcaactgccagaaacctcagtgccccgaggaaaagcagagacacctggacaagaagcagcggttccacaacattagaggcagatggacaggccggtgcatgagctgctgtagaagcagcagaacaagacggggctccatgcacggcgataccccaacactgcacgagtacatgctggatctgcagcccgagacaaccgatctgtacggctatgagcagctgaatgactccagcgaggaagaggacgagatcgacggacctgctggacaggctgaacctgatagagcccactacaatatcgtgaccttctgctgcaagtgcgatagcaccctgagaagatgcgtgcagagcacccacgtggacatcaggaccctcgaggatctgctcatgggcaccctgggaattgtggccccaatctgctctcagaagcctggcagcggcgccaccaatttcagtctgcttaaacaggccggggacgtcgaagagaaccccggacctatgcatcggcgcagatctcggtcttgtagagaagaccaaaagcctgtcatggatgatcaaagagatttaatttctaataatgaacaattacccatgttgggacggcgccccggagcccccgaatctaagtgcagccggggagctttatatacaggattttctattttagtgaccttattattagctggccaagccacaacagcttatttcttataccaacaacaaggacggttagataaattaacagtcacatctcaaaatttacaattagagaatttaagaatgaaattacctaaaccacccaaacccgtcagtaaaatgagaatggctacccccttattaatgcaagccctccccatgggcgccttaccccaaggacccatgcaaaatgctacaaagtatggaaatatgacagaagatcatgtcatgcacttattacaaaacgctgaccccttaaaagtctatccccctttaaaaggatctttccctgaaaatttaagacatttaaaaaatacaatggagacaattgattggaaagtctttgaatcttggatgcatcattggttattatttgaaatgtctaggcattctttagagcaaaaacccaccgatgctccacccaaggaaagtttagagttagaggacccttctagcggattaggagtcaccaaacaggacttaggacccgtccccatgatggaaacactgtgccagcggctgaacgtgtgccaggacaagattctgacccactatgagaacgacagcaccgacctgagggaccacatcgactactggaagcacatgcggctggaatgcgccatctactacaaggctagagagatgggcttcaagcacatcaaccaccaggtggtgcctacactggccgtgtctaagaacaaggctctgcaggccatcgaactgcagctgaccctggaaacaatctacaactcccagtactccaacgagaagtggaccctccaggacgtgtcactggaagtgtacctgaccgctcctaccggctgcatcaagaaacacggctacaccgtggaagtgcagtttgacggcgatatctgcaataccatgcactacaccaactggacccacatctacatctgcgaagaggccagcgtgacagtggtggaaggccaggtcgactactacggcctgtactacgtgcacgagggcatcagaacctacttcgtgcagttcaaggatgacgccgagaagtacagcaagaacaaagtgtgggaagtgcacgctggcggccaagtgatcctgtgtcctacaagcgtgttcagcagcaacgaagtgtctagccccgagatcatcagacagcacctggccaatcatcctgccgccacacacacaaaagctgtggccctgggcaccgaggaaacacagaccacaattcagaggcccagaagcgagcccgataccggcaatccttgtcacaccaccaagctgctgcaccgggattccgtggattctgcccctatcctgaccgccttcaacagctcccacaaaggccggatcaactgcaacagcaacaccacacctatcgtgcacctgaaaggggacgccaacactctgaaggccctgcggtacagattcaagaagcactgtaccctgtataccgccgtgtccagcacatggcactggaccggacacaacgtgaaacacaagagcgccatcgtgaccctgacctacgatagcgagtggcagagggaccagttcctgagccaagtgaagatccctaagaccatcaccgtgtccaccggctttatgagcatttgaggaagaatgtgcacagtgctaaccagaaaattcaggacaagactagtgcggccgcttttat

SEQ ID NO: 94 - DNA, 3643 bp, synthetic construct
  misc_feature 1..3643: note = HPV16 Ii-E1E6E7 integrated into TK locus of the poxviral vector
  source 1..3643: mol_type = other DNA
ggccaaaaattgaaattttattttttttttttggaatataaataatccctatcagtgatagagatctccctatcagtgatagagatcgtcgacgagctcgtttagtgaaccgtcagatcgcctggagacgccatccacgctgttttgacctctagcaccggtgccaccatgcacagaaggcggagcagaagctgccgcgaggatcagaaacccgtgatggacgaccagcgggacctgatcagcaacaacgagcagctgcctatgctcggcagaaggcctggcgctcctgagagcaaatgttctagaggcgccctgtacaccggcttcagcatcctggttacactgctgctggccggacaggctaccaccgcctactttctgtatcagcagcagggcagactggacaagctgaccgtgaccagccagaacctgcagctggaaaacctgcggatgaagctgcccaagcctcctaagcctgtgtccaagatgcggatggccacacctctgctgatgcaggctttgcctatgggagcactgcctcagggccctatgcagaacgccaccaaatacggcaacatgaccgaggaccacgtgatgcatctgctgcagaatgccgatcctctgaaggtgtaccctccactgaagggcagctttcccgagaacctgcggcacctgaagaacaccatggaaaccatcgactggaaggtgttcgagagctggatgcaccactggctgctgttcgagatgagccggcacagcctggaacagaagcctacagacgcccctcctaaagagtccctggaactggaagatcccagctctggcctgggcgtgacaaagcaagatctgggccctgtgcctatgatggccgatcctgctggcacaaacggcgaagaaggcacaggctgcaacggctggttttacgtggaagccgtggtggaaaagaaaaccggcgacgccatcagcgacgacgagaacgagaatgacagcgacaccggcgaggacctggtggacttcatcgtgaacgacaacgactacctgacacaggccgagacagagacagcccacgctctgtttacagcccaagaggccaagcagcacagagatgccgttcaggtgctgaagagaaagtacctgggcagccctctgagcgacatcagcggctgtgtggacaacaacatcagccccagactgaaggccatctgcatcgagaagcagagcagagccgccaagcggagactgtttgagagcgaggatagcggctacggcaataccgaggtggaaacccagcaaatgctgcaggtcgagggcagacacgaaaccgagacaccttgcagccagtacagcggaggatctggcggaggctgtagccagtactcttctggaagtggcggagagggcgtgtccgagagacacaccatttgtcagacccctctgaccaacatcctgaacgtgctgaaaaccagcaacgccaaggccgccatgctggccaagttcaaagagctgtacggcgtcagcttcagcgaactcgtgcggcccttcaagagcaacaagtccacctgttgcgactggtgcattgccgcctttggcctgacaccttctatcgccgacagcatcaagaccctgctccagcagtactgtctgtacctgcacattcagagcctggcctgctcttggggaatggtggtgctgctgctcgtgcggtacaagtgcggcaagaacagagagacaattgagaagctgctgagcaaactgctgtgcgtgtcccctatgtgcatgatgatcgagcctccaaagctgcggtctacagccgccgctctgtactggtacaagaccggcatcagcaatatctccgaggtgtacggcgacacccctgagtggattcagagacagaccgtcctgcagcacagcttcaacgactgcaccttcgagctgtcccagatggtgcagtgggcctacgacaatgacatcgtggacgactccgagatcgcctataagtacgcccagctggccgacaccaacagcaatgccagcgccttcctgaagtccaacagccaggccaagatcgtgaaggactgcgccaccatgtgcagacactacaagcgggccgagaagaaacagatgagcatgagccagtggatcaagtacagatgcgacagagtggacgacggcggcgattggaagcagatcgtgatgttcctgagataccagggcgtcgagttcatgagctttctgacagccctgaagcggttcctgcagggcatccccaagaagaactgcatcctgctgtatggcgccgctaacaccggcaagagcctgttcggaatgagcctgatgaagtttctgcagggctccgtgatctgctttgtgaacagcaagagccacttctggctccagccactggccgatgccaagattggcatgctggacgatgccaccgtgccttgctggaactacatcgacgacaacctgagaaacgccctggacggcaacctggtttctatggacgtgaagcacagacccctggtgcagctgaagtgtcctccactgctgatcaccagcaacatcaacgccggcaccgattccagatggccctatctgcacaaccggctggtggtgtttacctttcctaacgagttccccttcgacgaaaacggcaaccccgtgtacgagctgaacgataagaattggaagtccttcttcagccggacctggtccagactgagcctgcacgaggatgaggacaaagagaacgacggcgacagcctgcctaccttcaaatgtgtcagcggccagaacaccaacacactgggcagcatgcaccagaaacggacagccatgttccaggatcctcaagagcggccacggaagctgcctcagctgtgtacagaactgcagaccaccatccacgacatcatcctcgagtgcgtgtactgcaagcagcagctcctgcggagagaagtgtacgacttcgccttccgggatctgtgcatcgtgtacagagatggcaacccctacgccgtgtgcgacaagtgcctgaagttctactccaagatcagcgagtaccggcactactgctactccctgtacggcaccacacttgagcagcagtacaacaagcccctgtgcgatctgctgatccggtgcatcaactgccagaaacctcagtgccccgaggaaaagcagagacacctggacaagaagcagcggttccacaacattagaggcagatggacaggccggtgcatgagctgctgtagaagcagcagaacaagacggggctccatgcacggcgataccccaacactgcacgagtacatgctggatctgcagcccgagacaaccgatctgtacggctatgagcagctgaatgactccagcgaggaagaggacgagatcgacggacctgctggacaggctgaacctgatagagcccactacaatatcgtgaccttctgctgcaagtgcgatagcaccctgagaagatgcgtgcagagcacccacgtggacatcaggaccctcgaggatctgctcatgggcaccctgggaattgtggccccaatctgctctcagaagccttgaggaagaatgtgcacagtgctaaccagaaaattcaggacaagactagtgcggccgcttttat

SEQ ID NO: 95 - DNA, 4789 bp, synthetic construct
  misc_feature 1..4789: note = HPV18 Ii-E1E2E6E7 integrated into TK locus of the poxviral vector
  source 1..4789: mol_type = other DNA
ggccaaaaattgaaattttattttttttttttggaatataaataatccctatcagtgatagagatctccctatcagtgatagagatcgtcgacgagctcgtttagtgaaccgtcagatcgcctggagacgccatccacgctgttttgacctctagcaccggtgccaccatgcacagaaggcggagcagaagctgccgcgaggatcagaaacccgtgatggacgaccagcgggacctgatcagcaacaacgagcagctgcctatgctcggcagaaggcctggcgctcctgagagcaaatgttctagaggcgccctgtacaccggcttcagcatcctggttacactgctgctggccggacaggctaccaccgcctactttctgtatcagcagcagggcagactggacaagctgaccgtgaccagccagaacctgcagctggaaaacctgcggatgaagctgcccaagcctcctaagcctgtgtccaagatgcggatggccacacctctgctgatgcaggctttgcctatgggagcactgcctcagggccctatgcagaacgccaccaaatacggcaacatgaccgaggaccacgtgatgcatctgctgcagaatgccgatcctctgaaggtgtaccctccactgaagggcagctttcccgagaacctgcggcacctgaagaacaccatggaaaccatcgactggaaggtgttcgagagctggatgcaccactggctgctgttcgagatgagccggcacagcctggaacagaagcctacagacgcccctcctaaagagtccctggaactggaagatcccagctctggcctgggcgtgacaaagcaagatctgggccctgtgcctatgatggccgatcctgagggaacagatggcgaaggcacaggctgcaacggctggttttacgtgcaagccatcgtggacaagaagaccggcgacgtcatcagcgacgacgaggatgagaatgccaccgacaccggcagcgacatggtggactttatcgatacccagggcacgttctgcgagcaggccgaactggaaacagcccaggcactgtttcacgcccaagaggtgcacaacgatgcccaggtgctgcacgtgctgaagagaaagtttgccggcggaagcaccgagaacagcccactgggagaaagactggaagtggacaccgagctgagccccagactgcaagagatcagcctgaacagcggccagaagaaggccaagcggagactgttcaccatctccgacagcggctacggctgtagcgaagtggaagccacacagatccaagtgaccaccaatggcgagcacggcggcaatgtttgttctggcggatctaccgaggccatcgacaatggcggaaccgagggcaacaatagctccgtggatggcaccagcgacaacagcaacatcgagaacgtgaaccctcagtgcacaatcgcccagctgaaggacctgctgaaagtgaacaacaagcagggcgccatgctggccgtgtttaaggatacctacggcctgagcttcaccgacctcgtgcggaacttcaagagcgacaagaccacctgtaccgactgggtcaccgccatctttggcgtgaacccaacaatcgccgagggcttcaagaccctgatccagcctttcatcctgtacgcccacatccagtgtctggactgcaaatggggcgtgctgattctggccctgctgagatacaagtgcggcaagagcagactgaccgtggccaagggactgagcactctgctgcatgtgcccgagacatgcatgctgattcagcctccaaagctgcggtctagcgtggccgctctgtactggtacagaaccggcatcagcaatatcagcgaagtgatgggcgacacccctgagtggattcagcggctgacaatcatccagcacggcatcgacgactccaacttcgacctgagcgagatggtgcagtgggccttcgataacgagctgaccgacgagtccgacatggcctttgaatacgccctgctggcagacagcaactccaatgccgccgctttcctgaagtccaactgccaggccaagtacctgaaggactgcgccaccatgtgcaagcactaccgcagagcacagaaacggcagatgaacatgagccagtggatccggttccggtgcagcaagatcgatgaaggtggcgactggcggcctatcgtgcagttcctgagataccagcagatcgagttcatcacctttctgggcgccctgaagtcattcctgaagggaacccctaaaaagaactgcctggtgttctgcggccctgccaataccggcaagtcctacttcggcatgagctttatccacttcatccagggcgccgtgatcagcttcgtgaacagcaccagccacttttggctggaacccctgaccgataccaaggtggcaatgctggacgacgccaccaccacctgttggacctacttcgacacctacatgcggaacgccctggacggcaaccccatcagcatcgacagaaagcacaagcccctcatccagctgaaatgccctccaatcctgctgaccacaaacattcaccccgccaaggacaatcggtggccctacctggaaagccggatcaccgtgttcgagttccccaacgcctttccattcgacaagaacggcaatcccgtgtacgagatcaacgacaagaattggaagtgcttcttcgagcggacctggtccagactggatctgcacgaggaagaagaggacgccgacacagagggcaatcccttcggcacctttaagtgtgtggccgggcagaatcacagacccctgggatctatgcagaccccaaaagagacactgagcgagcggctgagcgccctccaggacaagatcatcgaccactacgagaacgacagcaaggacatcgacagccagattcagtactggcagctgatcagatgggagaacgccatcttcttcgccgctagagagcacggaattcagaccctgaaccaccaggtggtgcccgcctacaacatcagcaagagcaaggcccacaaggccattgaactccagatggccctgcaaggactggctcagagcgcctacaagaccgaggattggacactccaggatacctgcgaggaactgtggaacacagagcccacacactgcttcaagaaaggcggccagaccgtgcaggtctacttcgatggcaacaaggataactgcatgacctacgtggcctgggacagcgtgtactacatgacagatgccggcacctgggacaagaccgccacctgtgtttctcaccggggcctgtactacgtgaaagagggctacaacaccttctacatcgagtttaagagcgagtgcgagaagtatggcaacaccggcacatgggaagtgcatttcggcaacaacgtgatcgactgcaacgactccatgtgctccacctccgatgacaccgtgtctgccacacagctggtcaagcagctccagcacaccccatctccatacagctctacagtgtctgtgggcaccgccaagacctatggccaaacaagcgctgccacaagacctggccattgtggactggccgagaagcagcactgtggccctgttaatcctctgctgggagccgccacacctacaggcaacaacaagagaagaaagctgtgcagcggcaacaccacacctatcattcacctgaagggcgacagaaacagcctgaagtgcctgcggtacagactgaggaagcacagcgatcactaccgggacatcagctccacctggcactggacaggtgctggcaacgaaaagaccggcatcctgacagtgacctaccacagcgagactcagcggaccaagttcctgaacaccgtggctatccccgacagcgtgcagattctcgtgggctacatgaccatgggcagcatggccagattcgaggaccctaccagacggccctacaagctgcctgatctgtgcaccgaactgaacaccagccttcaggatatcgagatcacctgtgtgtactgcaagaccgtgctcgagctgacagaagtgtttgagttcgccttcaaggacctgttcgtggtgtacagagacagcatccctcacgccgcctgtcacaagtgcatcgacttctacagcagaatcagagagctgcggcactacagcgactccgtgtacggcgataccctggaaaaactgaccaacaccggactgtacaacctgctgatccggtgcctgagatgccagaaacctcagaaccccgccgagaaactgagacatctgaacgagaagcggcggttccacaatatcgccggccactacagaggccagtgccacagctgttgcaaccgggccagacaagaaagactccagcggagaagaggcagcatgcacggacctaaagccacacttcaggacatcgtgctgcacctcgagcctcagaatgagatccccgtggatctgctcggccatgagcagctgagcgatagcgaggaagagaacgacgagatcgacggcgtcaaccaccagcatctgcctgccagaagggccgaacctcagagacacaccatgctgtgcatgtgctgcaagtgcgaggcccggatcgagagagtggtggaatctagcgccgacgacctgagagctttccagcagctgttcctgaatactctgagcttcgtggccccttggtgcgctagtcagcagtgaggaagaatgtgcacagtgctaaccagaaaattcaggacaagactagtgcggccgcttttat

SEQ ID NO: 96 - DNA, 5545 bp, synthetic construct
  misc_feature 1..5545: note = HPV18 Ii-E1E2-p2a-Ii-E6E7 integrated into TK locus of the poxviral vector
  source 1..5545: mol_type = other DNA
ggccaaaaattgaaattttattttttttttttggaatataaataatccctatcagtgatagagatctccctatcagtgatagagatcgtcgacgagctcgtttagtgaaccgtcagatcgcctggagacgccatccacgctgttttgacctctagcaccggtgccaccatgcacagaaggcggagcagaagctgccgcgaggatcagaaacccgtgatggacgaccagcgggacctgatcagcaacaacgagcagctgcctatgctcggcagaaggcctggcgctcctgagagcaaatgttctagaggcgccctgtacaccggcttcagcatcctggttacactgctgctggccggacaggctaccaccgcctactttctgtatcagcagcagggcagactggacaagctgaccgtgaccagccagaacctgcagctggaaaacctgcggatgaagctgcccaagcctcctaagcctgtgtccaagatgcggatggccacacctctgctgatgcaggctttgcctatgggagcactgcctcagggccctatgcagaacgccaccaaatacggcaacatgaccgaggaccacgtgatgcatctgctgcagaatgccgatcctctgaaggtgtaccctccactgaagggcagctttcccgagaacctgcggcacctgaagaacaccatggaaaccatcgactggaaggtgttcgagagctggatgcaccactggctgctgttcgagatgagccggcacagcctggaacagaagcctacagacgcccctcctaaagagtccctggaactggaagatcccagctctggcctgggcgtgacaaagcaagatctgggccctgtgcctatgatggccgatcctgagggaacagatggcgaaggcacaggctgcaacggctggttttacgtgcaagccatcgtggacaagaagaccggcgacgtcatcagcgacgacgaggatgagaatgccaccgacaccggcagcgacatggtggactttatcgatacccagggcacgttctgcgagcaggccgaactggaaacagcccaggcactgtttcacgcccaagaggtgcacaacgatgcccaggtgctgcacgtgctgaagagaaagtttgccggcggaagcaccgagaacagcccactgggagaaagactggaagtggacaccgagctgagccccagactgcaagagatcagcctgaacagcggccagaagaaggccaagcggagactgttcaccatctccgacagcggctacggctgtagcgaagtggaagccacacagatccaagtgaccaccaatggcgagcacggcggcaatgtttgttctggcggatctaccgaggccatcgacaatggcggaaccgagggcaacaatagctccgtggatggcaccagcgacaacagcaacatcgagaacgtgaaccctcagtgcacaatcgcccagctgaaggacctgctgaaagtgaacaacaagcagggcgccatgctggccgtgtttaaggatacctacggcctgagcttcaccgacctcgtgcggaacttcaagagcgacaagaccacctgtaccgactgggtcaccgccatctttggcgtgaacccaacaatcgccgagggcttcaagaccctgatccagcctttcatcctgtacgcccacatccagtgtctggactgcaaatggggcgtgctgattctggccctgctgagatacaagtgcggcaagagcagactgaccgtggccaagggactgagcactctgctgcatgtgcccgagacatgcatgctgattcagcctccaaagctgcggtctagcgtggccgctctgtactggtacagaaccggcatcagcaatatcagcgaagtgatgggcgacacccctgagtggattcagcggctgacaatcatccagcacggcatcgacgactccaacttcgacctgagcgagatggtgcagtgggccttcgataacgagctgaccgacgagtccgacatggcctttgaatacgccctgctggcagacagcaactccaatgccgccgctttcctgaagtccaactgccaggccaagtacctgaaggactgcgccaccatgtgcaagcactaccgcagagcacagaaacggcagatgaacatgagccagtggatccggttccggtgcagcaagatcgatgaaggtggcgactggcggcctatcgtgcagttcctgagataccagcagatcgagttcatcacctttctgggcgccctgaagtcattcctgaagggaacccctaaaaagaactgcctggtgttctgcggccctgccaataccggcaagtcctacttcggcatgagctttatccacttcatccagggcgccgtgatcagcttcgtgaacagcaccagccacttttggctggaacccctgaccgataccaaggtggcaatgctggacgacgccaccaccacctgttggacctacttcgacacctacatgcggaacgccctggacggcaaccccatcagcatcgacagaaagcacaagcccctcatccagctgaaatgccctccaatcctgctgaccacaaacattcaccccgccaaggacaatcggtggccctacctggaaagccggatcaccgtgttcgagttccccaacgcctttccattcgacaagaacggcaatcccgtgtacgagatcaacgacaagaattggaagtgcttcttcgagcggacctggtccagactggatctgcacgaggaagaagaggacgccgacacagagggcaatcccttcggcacctttaagtgtgtggccgggcagaatcacagacccctgggatctatgcagaccccaaaagagacactgagcgagcggctgagcgccctccaggacaagatcatcgaccactacgagaacgacagcaaggacatcgacagccagattcagtactggcagctgatcagatgggagaacgccatcttcttcgccgctagagagcacggaattcagaccctgaaccaccaggtggtgcccgcctacaacatcagcaagagcaaggcccacaaggccattgaactccagatggccctgcaaggactggctcagagcgcctacaagaccgaggattggacactccaggatacctgcgaggaactgtggaacacagagcccacacactgcttcaagaaaggcggccagaccgtgcaggtctacttcgatggcaacaaggataactgcatgacctacgtggcctgggacagcgtgtactacatgacagatgccggcacctgggacaagaccgccacctgtgtttctcaccggggcctgtactacgtgaaagagggctacaacaccttctacatcgagtttaagagcgagtgcgagaagtatggcaacaccggcacatgggaagtgcatttcggcaacaacgtgatcgactgcaacgactccatgtgctccacctccgatgacaccgtgtctgccacacagctggtcaagcagctccagcacaccccatctccatacagctctacagtgtctgtgggcaccgccaagacctatggccaaacaagcgctgccacaagacctggccattgtggactggccgagaagcagcactgtggccctgttaatcctctgctgggagccgccacacctacaggcaacaacaagagaagaaagctgtgcagcggcaacaccacacctatcattcacctgaagggcgacagaaacagcctgaagtgcctgcggtacagactgaggaagcacagcgatcactaccgggacatcagctccacctggcactggacaggtgctggcaacgaaaagaccggcatcctgacagtgacctaccacagcgagactcagcggaccaagttcctgaacaccgtggctatccccgacagcgtgcagattctcgtgggctacatgaccatgggcagcggcgccaccaatttcagtctgcttaaacaggccggggacgtcgaagagaaccccggacctatgcatcggcgcagatctcggtcttgtagagaagaccaaaagcctgtcatggatgatcaaagagatttaatttctaataatgaacaattacccatgttgggacggcgccccggagcccccgaatctaagtgcagccggggagctttatatacaggattttctattttagtgaccttattattagctggccaagccacaacagcttatttcttataccaacaacaaggacggttagataaattaacagtcacatctcaaaatttacaattagagaatttaagaatgaaattacctaaaccacccaaacccgtcagtaaaatgagaatggctacccccttattaatgcaagccctccccatgggcgccttaccccaaggacccatgcaaaatgctacaaagtatggaaatatgacagaagatcatgtcatgcacttattacaaaacgctgaccccttaaaagtctatccccctttaaaaggatctttccctgaaaatttaagacatttaaaaaatacaatggagacaattgattggaaagtctttgaatcttggatgcatcattggttattatttgaaatgtctaggcattctttagagcaaaaacccaccgatgctccacccaaggaaagtttagagttagaggacccttctagcggattaggagtcaccaaacaggacttaggacccgtccccatgatggccagattcgaggaccctaccagacggccctacaagctgcctgatctgtgcaccgaactgaacaccagccttcaggatatcgagatcacctgtgtgtactgcaagaccgtgctcgagctgacagaagtgtttgagttcgccttcaaggacctgttcgtggtgtacagagacagcatccctcacgccgcctgtcacaagtgcatcgacttctacagcagaatcagagagctgcggcactacagcgactccgtgtacggcgataccctggaaaaactgaccaacaccggactgtacaacctgctgatccggtgcctgagatgccagaaacctcagaaccccgccgagaaactgagacatctgaacgagaagcggcggttccacaatatcgccggccactacagaggccagtgccacagctgttgcaaccgggccagacaagaaagactccagcggagaagaggcagcatgcacggacctaaagccacacttcaggacatcgtgctgcacctcgagcctcagaatgagatccccgtggatctgctcggccatgagcagctgagcgatagcgaggaagagaacgacgagatcgacggcgtcaaccaccagcatctgcctgccagaagggccgaacctcagagacacaccatgctgtgcatgtgctgcaagtgcgaggcccggatcgagagagtggtggaatctagcgccgacgacctgagagctttccagcagctgttcctgaatactctgagcttcgtggccccttggtgcgctagtcagcagtgaggaagaatgtgcacagtgctaaccagaaaattcaggacaagactagtgcggccgcttttat

SEQ ID NO: 97 - DNA, 4093 bp, synthetic construct
  misc_feature 1..4093: note = HPV18 E1E2E6E7 integrated into TK locus of the poxviral vector
  source 1..4093: mol_type = other DNA
ggccaaaaattgaaattttattttttttttttggaatataaataatccctatcagtgatagagatctccctatcagtgatagagatcgtcgacgagctcgtttagtgaaccgtcagatcgcctggagacgccatccacgctgttttgacctctagcaccggtgccaccatggccgatcctgagggaacagatggcgaaggcacaggctgcaacggctggttttacgtgcaagccatcgtggacaagaagaccggcgacgtcatcagcgacgacgaggatgagaatgccaccgacaccggcagcgacatggtggactttatcgatacccagggcacgttctgcgagcaggccgaactggaaacagcccaggcactgtttcacgcccaagaggtgcacaacgatgcccaggtgctgcacgtgctgaagagaaagtttgccggcggaagcaccgagaacagcccactgggagaaagactggaagtggacaccgagctgagccccagactgcaagagatcagcctgaacagcggccagaagaaggccaagcggagactgttcaccatctccgacagcggctacggctgtagcgaagtggaagccacacagatccaagtgaccaccaatggcgagcacggcggcaatgtttgttctggcggatctaccgaggccatcgacaatggcggaaccgagggcaacaatagctccgtggatggcaccagcgacaacagcaacatcgagaacgtgaaccctcagtgcacaatcgcccagctgaaggacctgctgaaagtgaacaacaagcagggcgccatgctggccgtgtttaaggatacctacggcctgagcttcaccgacctcgtgcggaacttcaagagcgacaagaccacctgtaccgactgggtcaccgccatctttggcgtgaacccaacaatcgccgagggcttcaagaccctgatccagcctttcatcctgtacgcccacatccagtgtctggactgcaaatggggcgtgctgattctggccctgctgagatacaagtgcggcaagagcagactgaccgtggccaagggactgagcactctgctgcatgtgcccgagacatgcatgctgattcagcctccaaagctgcggtctagcgtggccgctctgtactggtacagaaccggcatcagcaatatcagcgaagtgatgggcgacacccctgagtggattcagcggctgacaatcatccagcacggcatcgacgactccaacttcgacctgagcgagatggtgcagtgggccttcgataacgagctgaccgacgagtccgacatggcctttgaatacgccctgctggcagacagcaactccaatgccgccgctttcctgaagtccaactgccaggccaagtacctgaaggactgcgccaccatgtgcaagcactaccgcagagcacagaaacggcagatgaacatgagccagtggatccggttccggtgcagcaagatcgatgaaggtggcgactggcggcctatcgtgcagttcctgagataccagcagatcgagttcatcacctttctgggcgccctgaagtcattcctgaagggaacccctaaaaagaactgcctggtgttctgcggccctgccaataccggcaagtcctacttcggcatgagctttatccacttcatccagggcgccgtgatcagcttcgtgaacagcaccagccacttttggctggaacccctgaccgataccaaggtggcaatgctggacgacgccaccaccacctgttggacctacttcgacacctacatgcggaacgccctggacggcaaccccatcagcatcgacagaaagcacaagcccctcatccagctgaaatgccctccaatcctgctgaccacaaacattcaccccgccaaggacaatcggtggccctacctggaaagccggatcaccgtgttcgagttccccaacgcctttccattcgacaagaacggcaatcccgtgtacgagatcaacgacaagaattggaagtgcttcttcgagcggacctggtccagactggatctgcacgaggaagaagaggacgccgacacagagggcaatcccttcggcacctttaagtgtgtggccgggcagaatcacagacccctgggatctatgcagaccccaaaagagacactgagcgagcggctgagcgccctccaggacaagatcatcgaccactacgagaacgacagcaaggacatcgacagccagattcagtactggcagctgatcagatgggagaacgccatcttcttcgccgctagagagcacggaattcagaccctgaaccaccaggtggtgcccgcctacaacatcagcaagagcaaggcccacaaggccattgaactccagatggccctgcaaggactggctcagagcgcctacaagaccgaggattggacactccaggatacctgcgaggaactgtggaacacagagcccacacactgcttcaagaaaggcggccagaccgtgcaggtctacttcgatggcaacaaggataactgcatgacctacgtggcctgggacagcgtgtactacatgacagatgccggcacctgggacaagaccgccacctgtgtttctcaccggggcctgtactacgtgaaagagggctacaacaccttctacatcgagtttaagagcgagtgcgagaagtatggcaacaccggcacatgggaagtgcatttcggcaacaacgtgatcgactgcaacgactccatgtgctccacctccgatgacaccgtgtctgccacacagctggtcaagcagctccagcacaccccatctccatacagctctacagtgtctgtgggcaccgccaagacctatggccaaacaagcgctgccacaagacctggccattgtggactggccgagaagcagcactgtggccctgttaatcctctgctgggagccgccacacctacaggcaacaacaagagaagaaagctgtgcagcggcaacaccacacctatcattcacctgaagggcgacagaaacagcctgaagtgcctgcggtacagactgaggaagcacagcgatcactaccgggacatcagctccacctggcactggacaggtgctggcaacgaaaagaccggcatcctgacagtgacctaccacagcgagactcagcggaccaagttcctgaacaccgtggctatccccgacagcgtgcagattctcgtgggctacatgaccatgggcagcatggccagattcgaggaccctaccagacggccctacaagctgcctgatctgtgcaccgaactgaacaccagccttcaggatatcgagatcacctgtgtgtactgcaagaccgtgctcgagctgacagaagtgtttgagttcgccttcaaggacctgttcgtggtgtacagagacagcatccctcacgccgcctgtcacaagtgcatcgacttctacagcagaatcagagagctgcggcactacagcgactccgtgtacggcgataccctggaaaaactgaccaacaccggactgtacaacctgctgatccggtgcctgagatgccagaaacctcagaaccccgccgagaaactgagacatctgaacgagaagcggcggttccacaatatcgccggccactacagaggccagtgccacagctgttgcaaccgggccagacaagaaagactccagcggagaagaggcagcatgcacggacctaaagccacacttcaggacatcgtgctgcacctcgagcctcagaatgagatccccgtggatctgctcggccatgagcagctgagcgatagcgaggaagagaacgacgagatcgacggcgtcaaccaccagcatctgcctgccagaagggccgaacctcagagacacaccatgctgtgcatgtgctgcaagtgcgaggcccggatcgagagagtggtggaatctagcgccgacgacctgagagctttccagcagctgttcctgaatactctgagcttcgtggccccttggtgcgctagtcagcagtgaggaagaatgtgcacagtgctaaccagaaaattcaggacaagactagtgcggccgcttttat

SEQ ID NO: 98 - DNA, 5545 bp, synthetic construct
  misc_feature 1..5545: note = HPV18 Ii-E1E6E7-p2a-Ii-E2 integrated into TK locus of the poxviral vector
  source 1..5545: mol_type = other DNA
ggccaaaaattgaaattttattttttttttttggaatataaataatccctatcagtgatagagatctccctatcagtgatagagatcgtcgacgagctcgtttagtgaaccgtcagatcgcctggagacgccatccacgctgttttgacctctagcaccggtgccaccatgcacagaaggcggagcagaagctgccgcgaggatcagaaacccgtgatggacgaccagcgggacctgatcagcaacaacgagcagctgcctatgctcggcagaaggcctggcgctcctgagagcaaatgttctagaggcgccctgtacaccggcttcagcatcctggttacactgctgctggccggacaggctaccaccgcctactttctgtatcagcagcagggcagactggacaagctgaccgtgaccagccagaacctgcagctggaaaacctgcggatgaagctgcccaagcctcctaagcctgtgtccaagatgcggatggccacacctctgctgatgcaggctttgcctatgggagcactgcctcagggccctatgcagaacgccaccaaatacggcaacatgaccgaggaccacgtgatgcatctgctgcagaatgccgatcctctgaaggtgtaccctccactgaagggcagctttcccgagaacctgcggcacctgaagaacaccatggaaaccatcgactggaaggtgttcgagagctggatgcaccactggctgctgttcgagatgagccggcacagcctggaacagaagcctacagacgcccctcctaaagagtccctggaactggaagatcccagctctggcctgggcgtgacaaagcaagatctgggccctgtgcctatgatggccgatcctgagggaacagatggcgaaggcacaggctgcaacggctggttttacgtgcaagccatcgtggacaagaagaccggcgacgtcatcagcgacgacgaggatgagaatgccaccgacaccggcagcgacatggtggactttatcgatacccagggcacgttctgcgagcaggccgaactggaaacagcccaggcactgtttcacgcccaagaggtgcacaacgatgcccaggtgctgcacgtgctgaagagaaagtttgccggcggaagcaccgagaacagcccactgggagaaagactggaagtggacaccgagctgagccccagactgcaagagatcagcctgaacagcggccagaagaaggccaagcggagactgttcaccatctccgacagcggctacggctgtagcgaagtggaagccacacagatccaagtgaccaccaatggcgagcacggcggcaatgtttgttctggcggatctaccgaggccatcgacaatggcggaaccgagggcaacaatagctccgtggatggcaccagcgacaacagcaacatcgagaacgtgaaccctcagtgcacaatcgcccagctgaaggacctgctgaaagtgaacaacaagcagggcgccatgctggccgtgtttaaggatacctacggcctgagcttcaccgacctcgtgcggaacttcaagagcgacaagaccacctgtaccgactgggtcaccgccatctttggcgtgaacccaacaatcgccgagggcttcaagaccctgatccagcctttcatcctgtacgcccacatccagtgtctggactgcaaatggggcgtgctgattctggccctgctgagatacaagtgcggcaagagcagactgaccgtggccaagggactgagcactctgctgcatgtgcccgagacatgcatgctgattcagcctccaaagctgcggtctagcgtggccgctctgtactggtacagaaccggcatcagcaatatcagcgaagtgatgggcgacacccctgagtggattcagcggctgacaatcatccagcacggcatcgacgactccaacttcgacctgagcgagatggtgcagtgggccttcgataacgagctgaccgacgagtccgacatggcctttgaatacgccctgctggcagacagcaactccaatgccgccgctttcctgaagtccaactgccaggccaagtacctgaaggactgcgccaccatgtgcaagcactaccgcagagcacagaaacggcagatgaacatgagccagtggatccggttccggtgcagcaagatcgatgaaggtggcgactggcggcctatcgtgcagttcctgagataccagcagatcgagttcatcacctttctgggcgccctgaagtcattcctgaagggaacccctaaaaagaactgcctggtgttctgcggccctgccaataccggcaagtcctacttcggcatgagctttatccacttcatccagggcgccgtgatcagcttcgtgaacagcaccagccacttttggctggaacccctgaccgataccaaggtggcaatgctggacgacgccaccaccacctgttggacctacttcgacacctacatgcggaacgccctggacggcaaccccatcagcatcgacagaaagcacaagcccctcatccagctgaaatgccctccaatcctgctgaccacaaacattcaccccgccaaggacaatcggtggccctacctggaaagccggatcaccgtgttcgagttccccaacgcctttccattcgacaagaacggcaatcccgtgtacgagatcaacgacaagaattggaagtgcttcttcgagcggacctggtccagactggatctgcacgaggaagaagaggacgccgacacagagggcaatcccttcggcacctttaagtgtgtggccgggcagaatcacagacccctgggcagcatggccagattcgaggaccctaccagacggccctacaagctgcctgatctgtgcaccgaactgaacaccagccttcaggatatcgagatcacctgtgtgtactgcaagaccgtgctcgagctgacagaagtgtttgagttcgccttcaaggacctgttcgtggtgtacagagacagcatccctcacgccgcctgtcacaagtgcatcgacttctacagcagaatcagagagctgcggcactacagcgactccgtgtacggcgataccctggaaaaactgaccaacaccggactgtacaacctgctgatccggtgcctgagatgccagaaacctcagaaccccgccgagaaactgagacatctgaacgagaagcggcggttccacaatatcgccggccactacagaggccagtgccacagctgttgcaaccgggccagacaagaaagactccagcggagaagaggcagcatgcacggacctaaagccacacttcaggacatcgtgctgcacctcgagcctcagaatgagatccccgtggatctgctcggccatgagcagctgagcgatagcgaggaagagaacgacgagatcgacggcgtcaaccaccagcatctgcctgccagaagggccgaacctcagagacacaccatgctgtgcatgtgctgcaagtgcgaggcccggatcgagagagtggtggaatctagcgccgacgacctgagagctttccagcagctgttcctgaatactctgagcttcgtggccccttggtgcgctagtcagcagggcagcggcgccaccaatttcagtctgcttaaacaggccggggacgtcgaagagaaccccggacctatgcatcggcgcagatctcggtcttgtagagaagaccaaaagcctgtcatggatgatcaaagagatttaatttctaataatgaacaattacccatgttgggacggcgccccggagcccccgaatctaagtgcagccggggagctttatatacaggattttctattttagtgaccttattattagctggccaagccacaacagcttatttcttataccaacaacaaggacggttagataaattaacagtcacatctcaaaatttacaattagagaatttaagaatgaaattacctaaaccacccaaacccgtcagtaaaatgagaatggctacccccttattaatgcaagccctccccatgggcgccttaccccaaggacccatgcaaaatgctacaaagtatggaaatatgacagaagatcatgtcatgcacttattacaaaacgctgaccccttaaaagtctatccccctttaaaaggatctttccctgaaaatttaagacatttaaaaaatacaatggagacaattgattggaaagtctttgaatcttggatgcatcattggttattatttgaaatgtctaggcattctttagagcaaaaacccaccgatgctccacccaaggaaagtttagagttagaggacccttctagcggattaggagtcaccaaacaggacttaggacccgtccccatgatgcagaccccaaaagagacactgagcgagcggctgagcgccctccaggacaagatcatcgaccactacgagaacgacagcaaggacatcgacagccagattcagtactggcagctgatcagatgggagaacgccatcttcttcgccgctagagagcacggaattcagaccctgaaccaccaggtggtgcccgcctacaacatcagcaagagcaaggcccacaaggccattgaactccagatggccctgcaaggactggctcagagcgcctacaagaccgaggattggacactccaggatacctgcgaggaactgtggaacacagagcccacacactgcttcaagaaaggcggccagaccgtgcaggtctacttcgatggcaacaaggataactgcatgacctacgtggcctgggacagcgtgtactacatgacagatgccggcacctgggacaagaccgccacctgtgtttctcaccggggcctgtactacgtgaaagagggctacaacaccttctacatcgagtttaagagcgagtgcgagaagtatggcaacaccggcacatgggaagtgcatttcggcaacaacgtgatcgactgcaacgactccatgtgctccacctccgatgacaccgtgtctgccacacagctggtcaagcagctccagcacaccccatctccatacagctctacagtgtctgtgggcaccgccaagacctatggccaaacaagcgctgccacaagacctggccattgtggactggccgagaagcagcactgtggccctgttaatcctctgctgggagccgccacacctacaggcaacaacaagagaagaaagctgtgcagcggcaacaccacacctatcattcacctgaagggcgacagaaacagcctgaagtgcctgcggtacagactgaggaagcacagcgatcactaccgggacatcagctccacctggcactggacaggtgctggcaacgaaaagaccggcatcctgacagtgacctaccacagcgagactcagcggaccaagttcctgaacaccgtggctatccccgacagcgtgcagattctcgtgggctacatgaccatgtgaggaagaatgtgcacagtgctaaccagaaaattcaggacaagactagtgcggccgcttttat

SEQ ID NO: 99 - DNA, 3688 bp, synthetic construct
  misc_feature 1..3688: note = HPV18 Ii-E1E6E7 integrated into TK locus of the poxviral vector
  source 1..3688: mol_type = other DNA
ggccaaaaattgaaattttattttttttttttggaatataaataatccctatcagtgatagagatctccctatcagtgatagagatcgtcgacgagctcgtttagtgaaccgtcagatcgcctggagacgccatccacgctgttttgacctctagcaccggtgccaccatgcacagaaggcggagcagaagctgccgcgaggatcagaaacccgtgatggacgaccagcgggacctgatcagcaacaacgagcagctgcctatgctcggcagaaggcctggcgctcctgagagcaaatgttctagaggcgccctgtacaccggcttcagcatcctggttacactgctgctggccggacaggctaccaccgcctactttctgtatcagcagcagggcagactggacaagctgaccgtgaccagccagaacctgcagctggaaaacctgcggatgaagctgcccaagcctcctaagcctgtgtccaagatgcggatggccacacctctgctgatgcaggctttgcctatgggagcactgcctcagggccctatgcagaacgccaccaaatacggcaacatgaccgaggaccacgtgatgcatctgctgcagaatgccgatcctctgaaggtgtaccctccactgaagggcagctttcccgagaacctgcggcacctgaagaacaccatggaaaccatcgactggaaggtgttcgagagctggatgcaccactggctgctgttcgagatgagccggcacagcctggaacagaagcctacagacgcccctcctaaagagtccctggaactggaagatcccagctctggcctgggcgtgacaaagcaagatctgggccctgtgcctatgatggccgatcctgagggaacagatggcgaaggcacaggctgcaacggctggttttacgtgcaagccatcgtggacaagaagaccggcgacgtcatcagcgacgacgaggatgagaatgccaccgacaccggcagcgacatggtggactttatcgatacccagggcacgttctgcgagcaggccgaactggaaacagcccaggcactgtttcacgcccaagaggtgcacaacgatgcccaggtgctgcacgtgctgaagagaaagtttgccggcggaagcaccgagaacagcccactgggagaaagactggaagtggacaccgagctgagccccagactgcaagagatcagcctgaacagcggccagaagaaggccaagcggagactgttcaccatctccgacagcggctacggctgtagcgaagtggaagccacacagatccaagtgaccaccaatggcgagcacggcggcaatgtttgttctggcggatctaccgaggccatcgacaatggcggaaccgagggcaacaatagctccgtggatggcaccagcgacaacagcaacatcgagaacgtgaaccctcagtgcacaatcgcccagctgaaggacctgctgaaagtgaacaacaagcagggcgccatgctggccgtgtttaaggatacctacggcctgagcttcaccgacctcgtgcggaacttcaagagcgacaagaccacctgtaccgactgggtcaccgccatctttggcgtgaacccaacaatcgccgagggcttcaagaccctgatccagcctttcatcctgtacgcccacatccagtgtctggactgcaaatggggcgtgctgattctggccctgctgagatacaagtgcggcaagagcagactgaccgtggccaagggactgagcactctgctgcatgtgcccgagacatgcatgctgattcagcctccaaagctgcggtctagcgtggccgctctgtactggtacagaaccggcatcagcaatatcagcgaagtgatgggcgacacccctgagtggattcagcggctgacaatcatccagcacggcatcgacgactccaacttcgacctgagcgagatggtgcagtgggccttcgataacgagctgaccgacgagtccgacatggcctttgaatacgccctgctggcagacagcaactccaatgccgccgctttcctgaagtccaactgccaggccaagtacctgaaggactgcgccaccatgtgcaagcactaccgcagagcacagaaacggcagatgaacatgagccagtggatccggttccggtgcagcaagatcgatgaaggtggcgactggcggcctatcgtgcagttcctgagataccagcagatcgagttcatcacctttctgggcgccctgaagtcattcctgaagggaacccctaaaaagaactgcctggtgttctgcggccctgccaataccggcaagtcctacttcggcatgagctttatccacttcatccagggcgccgtgatcagcttcgtgaacagcaccagccacttttggctggaacccctgaccgataccaaggtggcaatgctggacgacgccaccaccacctgttggacctacttcgacacctacatgcggaacgccctggacggcaaccccatcagcatcgacagaaagcacaagcccctcatccagctgaaatgccctccaatcctgctgaccacaaacattcaccccgccaaggacaatcggtggccctacctggaaagccggatcaccgtgttcgagttccccaacgcctttccattcgacaagaacggcaatcccgtgtacgagatcaacgacaagaattggaagtgcttcttcgagcggacctggtccagactggatctgcacgaggaagaagaggacgccgacacagagggcaatcccttcggcacctttaagtgtgtggccgggcagaatcacagacccctgggcagcatggccagattcgaggaccctaccagacggccctacaagctgcctgatctgtgcaccgaactgaacaccagccttcaggatatcgagatcacctgtgtgtactgcaagaccgtgctcgagctgacagaagtgtttgagttcgccttcaaggacctgttcgtggtgtacagagacagcatccctcacgccgcctgtcacaagtgcatcgacttctacagcagaatcagagagctgcggcactacagcgactccgtgtacggcgataccctggaaaaactgaccaacaccggactgtacaacctgctgatccggtgcctgagatgccagaaacctcagaaccccgccgagaaactgagacatctgaacgagaagcggcggttccacaatatcgccggccactacagaggccagtgccacagctgttgcaaccgggccagacaagaaagactccagcggagaagaggcagcatgcacggacctaaagccacacttcaggacatcgtgctgcacctcgagcctcagaatgagatccccgtggatctgctcggccatgagcagctgagcgatagcgaggaagagaacgacgagatcgacggcgtcaaccaccagcatctgcctgccagaagggccgaacctcagagacacaccatgctgtgcatgtgctgcaagtgcgaggcccggatcgagagagtggtggaatctagcgccgacgacctgagagctttccagcagctgttcctgaatactctgagcttcgtggccccttggtgcgctagtcagcagtgaggaagaatgtgcacagtgctaaccagaaaattcaggacaagactagtgcggccgcttttat

SEQ ID NO: 100 - DNA, 3462 bp, Human papillomavirus type 18
  misc_feature 1..3462: note = HPV18 Ii-E1E6E7 different linker
  source 1..3462: mol_type = other DNA
atgcacagaaggcggagcagaagctgccgcgaggatcagaaacccgtgatggacgaccagcgggacctgatcagcaacaacgagcagctgcctatgctcggcagaaggcctggcgctcctgagagcaaatgttctagaggcgccctgtacaccggcttcagcatcctggttacactgctgctggccggacaggctaccaccgcctactttctgtatcagcagcagggcagactggacaagctgaccgtgaccagccagaacctgcagctggaaaacctgcggatgaagctgcccaagcctcctaagcctgtgtccaagatgcggatggccacacctctgctgatgcaggctttgcctatgggagcactgcctcagggccctatgcagaacgccaccaaatacggcaacatgaccgaggaccacgtgatgcatctgctgcagaatgccgatcctctgaaggtgtaccctccactgaagggcagctttcccgagaacctgcggcacctgaagaacaccatggaaaccatcgactggaaggtgttcgagagctggatgcaccactggctgctgttcgagatgagccggcacagcctggaacagaagcctacagacgcccctcctaaagagtccctggaactggaagatcccagctctggcctgggcgtgacaaagcaagatctgggccctgtgcctatgatggccgatcctgagggaacagatggcgaaggcacaggctgcaacggctggttttacgtgcaagccatcgtggacaagaagaccggcgacgtcatcagcgacgacgaggatgagaatgccaccgacaccggcagcgacatggtggactttatcgatacccagggcacgttctgcgagcaggccgaactggaaacagcccaggcactgtttcacgcccaagaggtgcacaacgatgcccaggtgctgcacgtgctgaagagaaagtttgccggcggaagcaccgagaacagcccactgggagaaagactggaagtggacaccgagctgagccccagactgcaagagatcagcctgaacagcggccagaagaaggccaagcggagactgttcaccatctccgacagcggctacggctgtagcgaagtggaagccacacagatccaagtgaccaccaatggcgagcacggcggcaatgtttgttctggcggatctaccgaggccatcgacaatggcggaaccgagggcaacaatagctccgtggatggcaccagcgacaacagcaacatcgagaacgtgaaccctcagtgcacaatcgcccagctgaaggacctgctgaaagtgaacaacaagcagggcgccatgctggccgtgtttaaggatacctacggcctgagcttcaccgacctcgtgcggaacttcaagagcgacaagaccacctgtaccgactgggtcaccgccatctttggcgtgaacccaacaatcgccgagggcttcaagaccctgatccagcctttcatcctgtacgcccacatccagtgtctggactgcaaatggggcgtgctgattctggccctgctgagatacaagtgcggcaagagcagactgaccgtggccaagggactgagcactctgctgcatgtgcccgagacatgcatgctgattcagcctccaaagctgcggtctagcgtggccgctctgtactggtacagaaccggcatcagcaatatcagcgaagtgatgggcgacacccctgagtggattcagcggctgacaatcatccagcacggcatcgacgactccaacttcgacctgagcgagatggtgcagtgggccttcgataacgagctgaccgacgagtccgacatggcctttgaatacgccctgctggcagacagcaactccaatgccgccgctttcctgaagtccaactgccaggccaagtacctgaaggactgcgccaccatgtgcaagcactaccgcagagcacagaaacggcagatgaacatgagccagtggatccggttccggtgcagcaagatcgatgaaggtggcgactggcggcctatcgtgcagttcctgagataccagcagatcgagttcatcacctttctgggcgccctgaagtcattcctgaagggaacccctaaaaagaactgcctggtgttctgcggccctgccaataccggcaagtcctacttcggcatgagctttatccacttcatccagggcgccgtgatcagcttcgtgaacagcaccagccacttttggctggaacccctgaccgataccaaggtggcaatgctggacgacgccaccaccacctgttggacctacttcgacacctacatgcggaacgccctggacggcaaccccatcagcatcgacagaaagcacaagcccctcatccagctgaaatgccctccaatcctgctgaccacaaacattcaccccgccaaggacaatcggtggccctacctggaaagccggatcaccgtgttcgagttccccaacgcctttccattcgacaagaacggcaatcccgtgtacgagatcaacgacaagaattggaagtgcttcttcgagcggacctggtccagactggatctgcacgaggaagaagaggacgccgacacagagggcaatcccttcggcacctttaagtgtgtggccgggcagaatcacagacccctgggctgcagcatggccagattcgaggaccctaccagacggccctacaagctgcctgatctgtgcaccgaactgaacaccagccttcaggatatcgagatcacctgtgtgtactgcaagaccgtgctcgagctgacagaagtgtttgagttcgccttcaaggacctgttcgtggtgtacagagacagcatccctcacgccgcctgtcacaagtgcatcgacttctacagcagaatcagagagctgcggcactacagcgactccgtgtacggcgataccctggaaaaactgaccaacaccggactgtacaacctgctgatccggtgcctgagatgccagaaacctcagaaccccgccgagaaactgagacatctgaacgagaagcggcggttccacaatatcgccggccactacagaggccagtgccacagctgttgcaaccgggccagacaagaaagactccagcggagaagaggcagcatgcacggacctaaagccacacttcaggacatcgtgctgcacctcgagcctcagaatgagatccccgtggatctgctcggccatgagcagctgagcgatagcgaggaagagaacgacgagatcgacggcgtcaaccaccagcatctgcctgccagaagggccgaacctcagagacacaccatgctgtgcatgtgctgcaagtgcgaggcccggatcgagagagtggtggaatctagcgccgacgacctgagagctttccagcagctgttcctgaatactctgagcttcgtggccccttggtgcgctagtcagcagtga

SEQ ID NO: 101 - protein, 1153 aa, Human papillomavirus type 18
  REGION 1..1153: note = HPV18 Ii-E1E6E7 translated different linker
MHRRRSRSCREDQKPVMDDQRDLISNNEQLPMLGRRPGAPESKCSRGALYTGFSILVTLLLAGQATTAYFLYQQQGRLDKLTVTSQNLQLENLRMKLPKPPKPVSKMRMATPLLMQALPMGALPQGPMQNATKYGNMTEDHVMHLLQNADPLKVYPPLKGSFPENLRHLKNTMETIDWKVFESWMHHWLLFEMSRHSLEQKPTDAPPKESLELEDPSSGLGVTKQDLGPVPMMADPEGTDGEGTGCNGWFYVQAIVDKKTGDVISDDEDENATDTGSDMVDFIDTQGTFCEQAELETAQALFHAQEVHNDAQVLHVLKRKFAGGSTENSPLGERLEVDTELSPRLQEISLNSGQKKAKRRLFTISDSGYGCSEVEATQIQVTTNGEHGGNVCSGGSTEAIDNGGTEGNNSSVDGTSDNSNIENVNPQCTIAQLKDLLKVNNKQGAMLAVFKDTYGLSFTDLVRNFKSDKTTCTDWVTAIFGVNPTIAEGFKTLIQPFILYAHIQCLDCKWGVLILALLRYKCGKSRLTVAKGLSTLLHVPETCMLIQPPKLRSSVAALYWYRTGISNISEVMGDTPEWIQRLTIIQHGIDDSNFDLSEMVQWAFDNELTDESDMAFEYALLADSNSNAAAFLKSNCQAKYLKDCATMCKHYRRAQKRQMNMSQWIRFRCSKIDEGGDWRPIVQFLRYQQIEFITFLGALKSFLKGTPKKNCLVFCGPANTGKSYFGMSFIHFIQGAVISFVNSTSHFWLEPLTDTKVAMLDDATTTCWTYFDTYMRNALDGNPISIDRKHKPLIQLKCPPILLTTNIHPAKDNRWPYLESRITVFEFPNAFPFDKNGNPVYEINDKNWKCFFERTWSRLDLHEEEEDADTEGNPFGTFKCVAGQNHRPLGCSMARFEDPTRRPYKLPDLCTELNTSLQDIEITCVYCKTVLELTEVFEFAFKDLFVVYRDSIPHAACHKCIDFYSRIRELRHYSDSVYGDTLEKLTNTGLYNLLIRCLRCQKPQNPAEKLRHLNEKRRFHNIAGHYRGQCHSCCNRARQERLQRRRGSMHGPKATLQDIVLHLEPQNEIPVDLLGHEQLSDSEEENDEIDGVNHQHLPARRAEPQRHTMLCMCCKCEARIERVVESSADDLRAFQQLFLNTLSFVAPWCASQQ

SEQ ID NO: 102 - DNA, 4040 bp, synthetic construct
  misc_feature 1..4040: note = HPV18 Ii-E1E6E7 different linker as carried by Adenoviral vector
  source 1..4040: mol_type = other DNA
gtgatgcggttttggcagtacatcaatgggcgtggatagcggtttgactcacggggatttccaagtctccaccccattgacgtcaatgggagtttgttttggaaccaaaatcaacgggactttccaaaatgtcgtaacaactccgccccattgacgcaaatgggcggtaggcgtgtacggtgggaggtctatataagcagagctctccctatcagtgatagagatctccctatcagtgatagagatcgtcgacgagctcgtttagtgaaccgtcagatcgcctggagacgccatccacgctgttttgacctctagcaccggtgccaccatgcacagaaggcggagcagaagctgccgcgaggatcagaaacccgtgatggacgaccagcgggacctgatcagcaacaacgagcagctgcctatgctcggcagaaggcctggcgctcctgagagcaaatgttctagaggcgccctgtacaccggcttcagcatcctggttacactgctgctggccggacaggctaccaccgcctactttctgtatcagcagcagggcagactggacaagctgaccgtgaccagccagaacctgcagctggaaaacctgcggatgaagctgcccaagcctcctaagcctgtgtccaagatgcggatggccacacctctgctgatgcaggctttgcctatgggagcactgcctcagggccctatgcagaacgccaccaaatacggcaacatgaccgaggaccacgtgatgcatctgctgcagaatgccgatcctctgaaggtgtaccctccactgaagggcagctttcccgagaacctgcggcacctgaagaacaccatggaaaccatcgactggaaggtgttcgagagctggatgcaccactggctgctgttcgagatgagccggcacagcctggaacagaagcctacagacgcccctcctaaagagtccctggaactggaagatcccagctctggcctgggcgtgacaaagcaagatctgggccctgtgcctatgatggccgatcctgagggaacagatggcgaaggcacaggctgcaacggctggttttacgtgcaagccatcgtggacaagaagaccggcgacgtcatcagcgacgacgaggatgagaatgccaccgacaccggcagcgacatggtggactttatcgatacccagggcacgttctgcgagcaggccgaactggaaacagcccaggcactgtttcacgcccaagaggtgcacaacgatgcccaggtgctgcacgtgctgaagagaaagtttgccggcggaagcaccgagaacagcccactgggagaaagactggaagtggacaccgagctgagccccagactgcaagagatcagcctgaacagcggccagaagaaggccaagcggagactgttcaccatctccgacagcggctacggctgtagcgaagtggaagccacacagatccaagtgaccaccaatggcgagcacggcggcaatgtttgttctggcggatctaccgaggccatcgacaatggcggaaccgagggcaacaatagctccgtggatggcaccagcgacaacagcaacatcgagaacgtgaaccctcagtgcacaatcgcccagctgaaggacctgctgaaagtgaacaacaagcagggcgccatgctggccgtgtttaaggatacctacggcctgagcttcaccgacctcgtgcggaacttcaagagcgacaagaccacctgtaccgactgggtcaccgccatctttggcgtgaacccaacaatcgccgagggcttcaagaccctgatccagcctttcatcctgtacgcccacatccagtgtctggactgcaaatggggcgtgctgattctggccctgctgagatacaagtgcggcaagagcagactgaccgtggccaagggactgagcactctgctgcatgtgcccgagacatgcatgctgattcagcctccaaagctgcggtctagcgtggccgctctgtactggtacagaaccggcatcagcaatatcagcgaagtgatgggcgacacccctgagtggattcagcggctgacaatcatccagcacggcatcgacgactccaacttcgacctgagcgagatggtgcagtgggccttcgataacgagctgaccgacgagtccgacatggcctttgaatacgccctgctggcagacagcaactccaatgccgccgctttcctgaagtccaactgccaggccaagtacctgaaggactgcgccaccatgtgcaagcactaccgcagagcacagaaacggcagatgaacatgagccagtggatccggttccggtgcagcaagatcgatgaaggtggcgactggcggcctatcgtgcagttcctgagataccagcagatcgagttcatcacctttctgggcgccctgaagtcattcctgaagggaacccctaaaaagaactgcctggtgttctgcggccctgccaataccggcaagtcctacttcggcatgagctttatccacttcatccagggcgccgtgatcagcttcgtgaacagcaccagccacttttggctggaacccctgaccgataccaaggtggcaatgctggacgacgccaccaccacctgttggacctacttcgacacctacatgcggaacgccctggacggcaaccccatcagcatcgacagaaagcacaagcccctcatccagctgaaatgccctccaatcctgctgaccacaaacattcaccccgccaaggacaatcggtggccctacctggaaagccggatcaccgtgttcgagttccccaacgcctttccattcgacaagaacggcaatcccgtgtacgagatcaacgacaagaattggaagtgcttcttcgagcggacctggtccagactggatctgcacgaggaagaagaggacgccgacacagagggcaatcccttcggcacctttaagtgtgtggccgggcagaatcacagacccctgggctgcagcatggccagattcgaggaccctaccagacggccctacaagctgcctgatctgtgcaccgaactgaacaccagccttcaggatatcgagatcacctgtgtgtactgcaagaccgtgctcgagctgacagaagtgtttgagttcgccttcaaggacctgttcgtggtgtacagagacagcatccctcacgccgcctgtcacaagtgcatcgacttctacagcagaatcagagagctgcggcactacagcgactccgtgtacggcgataccctggaaaaactgaccaacaccggactgtacaacctgctgatccggtgcctgagatgccagaaacctcagaaccccgccgagaaactgagacatctgaacgagaagcggcggttccacaatatcgccggccactacagaggccagtgccacagctgttgcaaccgggccagacaagaaagactccagcggagaagaggcagcatgcacggacctaaagccacacttcaggacatcgtgctgcacctcgagcctcagaatgagatccccgtggatctgctcggccatgagcagctgagcgatagcgaggaagagaacgacgagatcgacggcgtcaaccaccagcatctgcctgccagaagggccgaacctcagagacacaccatgctgtgcatgtgctgcaagtgcgaggcccggatcgagagagtggtggaatctagcgccgacgacctgagagctttccagcagctgttcctgaatactctgagcttcgtggccccttggtgcgctagtcagcagtgagcggccgcggaagaatgtgcacagtgctaaccagaaaattcaggacaagactagtgactctagatcataatcagccataccacatttgtagaggttttacttgctttaaaaaacctcccacacctccccctgaacctgaaacataaaatgaatgcaattgttgttgttaacttgtttattgcagcttataatggttacaaataaagcaatagcatcacaaatttcacaaataaagcatttttttcactgc

SEQ ID NO: 103 - DNA, 3755 bp, synthetic construct
  misc_feature 1..3755: note = HPV18 Ii-E1E6E7 different linker integrated into DelIII locus of the poxviral vector
  source 1..3755: mol_type = other DNA
aaaaattaaaattttattttttttttttggaatataaataagatctatccctatcagtgatagagatctccctatcagtgatagagatcgtcgacgagctcgtttagtgaaccgtcagatcgcctggagacgccatccacgctgttttgacctctagcaccggtgccaccatgcacagaaggcggagcagaagctgccgcgaggatcagaaacccgtgatggacgaccagcgggacctgatcagcaacaacgagcagctgcctatgctcggcagaaggcctggcgctcctgagagcaaatgttctagaggcgccctgtacaccggcttcagcatcctggttacactgctgctggccggacaggctaccaccgcctactttctgtatcagcagcagggcagactggacaagctgaccgtgaccagccagaacctgcagctggaaaacctgcggatgaagctgcccaagcctcctaagcctgtgtccaagatgcggatggccacacctctgctgatgcaggctttgcctatgggagcactgcctcagggccctatgcagaacgccaccaaatacggcaacatgaccgaggaccacgtgatgcatctgctgcagaatgccgatcctctgaaggtgtaccctccactgaagggcagctttcccgagaacctgcggcacctgaagaacaccatggaaaccatcgactggaaggtgttcgagagctggatgcaccactggctgctgttcgagatgagccggcacagcctggaacagaagcctacagacgcccctcctaaagagtccctggaactggaagatcccagctctggcctgggcgtgacaaagcaagatctgggccctgtgcctatgatggccgatcctgagggaacagatggcgaaggcacaggctgcaacggctggttttacgtgcaagccatcgtggacaagaagaccggcgacgtcatcagcgacgacgaggatgagaatgccaccgacaccggcagcgacatggtggactttatcgatacccagggcacgttctgcgagcaggccgaactggaaacagcccaggcactgtttcacgcccaagaggtgcacaacgatgcccaggtgctgcacgtgctgaagagaaagtttgccggcggaagcaccgagaacagcccactgggagaaagactggaagtggacaccgagctgagccccagactgcaagagatcagcctgaacagcggccagaagaaggccaagcggagactgttcaccatctccgacagcggctacggctgtagcgaagtggaagccacacagatccaagtgaccaccaatggcgagcacggcggcaatgtttgttctggcggatctaccgaggccatcgacaatggcggaaccgagggcaacaatagctccgtggatggcaccagcgacaacagcaacatcgagaacgtgaaccctcagtgcacaatcgcccagctgaaggacctgctgaaagtgaacaacaagcagggcgccatgctggccgtgtttaaggatacctacggcctgagcttcaccgacctcgtgcggaacttcaagagcgacaagaccacctgtaccgactgggtcaccgccatctttggcgtgaacccaacaatcgccgagggcttcaagaccctgatccagcctttcatcctgtacgcccacatccagtgtctggactgcaaatggggcgtgctgattctggccctgctgagatacaagtgcggcaagagcagactgaccgtggccaagggactgagcactctgctgcatgtgcccgagacatgcatgctgattcagcctccaaagctgcggtctagcgtggccgctctgtactggtacagaaccggcatcagcaatatcagcgaagtgatgggcgacacccctgagtggattcagcggctgacaatcatccagcacggcatcgacgactccaacttcgacctgagcgagatggtgcagtgggccttcgataacgagctgaccgacgagtccgacatggcctttgaatacgccctgctggcagacagcaactccaatgccgccgctttcctgaagtccaactgccaggccaagtacctgaaggactgcgccaccatgtgcaagcactaccgcagagcacagaaacggcagatgaacatgagccagtggatccggttccggtgcagcaagatcgatgaaggtggcgactggcggcctatcgtgcagttcctgagataccagcagatcgagttcatcacctttctgggcgccctgaagtcattcctgaagggaacccctaaaaagaactgcctggtgttctgcggccctgccaataccggcaagtcctacttcggcatgagctttatccacttcatccagggcgccgtgatcagcttcgtgaacagcaccagccacttttggctggaacccctgaccgataccaaggtggcaatgctggacgacgccaccaccacctgttggacctacttcgacacctacatgcggaacgccctggacggcaaccccatcagcatcgacagaaagcacaagcccctcatccagctgaaatgccctccaatcctgctgaccacaaacattcaccccgccaaggacaatcggtggccctacctggaaagccggatcaccgtgttcgagttccccaacgcctttccattcgacaagaacggcaatcccgtgtacgagatcaacgacaagaattggaagtgcttcttcgagcggacctggtccagactggatctgcacgaggaagaagaggacgccgacacagagggcaatcccttcggcacctttaagtgtgtggccgggcagaatcacagacccctgggctgcagcatggccagattcgaggaccctaccagacggccctacaagctgcctgatctgtgcaccgaactgaacaccagccttcaggatatcgagatcacctgtgtgtactgcaagaccgtgctcgagctgacagaagtgtttgagttcgccttcaaggacctgttcgtggtgtacagagacagcatccctcacgccgcctgtcacaagtgcatcgacttctacagcagaatcagagagctgcggcactacagcgactccgtgtacggcgataccctggaaaaactgaccaacaccggactgtacaacctgctgatccggtgcctgagatgccagaaacctcagaaccccgccgagaaactgagacatctgaacgagaagcggcggttccacaatatcgccggccactacagaggccagtgccacagctgttgcaaccgggccagacaagaaagactccagcggagaagaggcagcatgcacggacctaaagccacacttcaggacatcgtgctgcacctcgagcctcagaatgagatccccgtggatctgctcggccatgagcagctgagcgatagcgaggaagagaacgacgagatcgacggcgtcaaccaccagcatctgcctgccagaagggccgaacctcagagacacaccatgctgtgcatgtgctgcaagtgcgaggcccggatcgagagagtggtggaatctagcgccgacgacctgagagctttccagcagctgttcctgaatactctgagcttcgtggccccttggtgcgctagtcagcagtgaggaagaatgtgcacagtgctaaccagaaaattcaggacaagactagtgcggccgctttggaaagttttataggtagttgatagaacaaaatacataattttgtaaaaataaatcactttttat

SEQ ID NO: 104 - DNA, 3691 bp, synthetic construct
  misc_feature 1..3691: note = HPV18 Ii-E1E6E7 different linker integrated into TK locus of the poxviral vector
  source 1..3691: mol_type = other DNA
ggccaaaaattgaaattttattttttttttttggaatataaataatccctatcagtgatagagatctccctatcagtgatagagatcgtcgacgagctcgtttagtgaaccgtcagatcgcctggagacgccatccacgctgttttgacctctagcaccggtgccaccatgcacagaaggcggagcagaagctgccgcgaggatcagaaacccgtgatggacgaccagcgggacctgatcagcaacaacgagcagctgcctatgctcggcagaaggcctggcgctcctgagagcaaatgttctagaggcgccctgtacaccggcttcagcatcctggttacactgctgctggccggacaggctaccaccgcctactttctgtatcagcagcagggcagactggacaagctgaccgtgaccagccagaacctgcagctggaaaacctgcggatgaagctgcccaagcctcctaagcctgtgtccaagatgcggatggccacacctctgctgatgcaggctttgcctatgggagcactgcctcagggccctatgcagaacgccaccaaatacggcaacatgaccgaggaccacgtgatgcatctgctgcagaatgccgatcctctgaaggtgtaccctccactgaagggcagctttcccgagaacctgcggcacctgaagaacaccatggaaaccatcgactggaaggtgttcgagagctggatgcaccactggctgctgttcgagatgagccggcacagcctggaacagaagcctacagacgcccctcctaaagagtccctggaactggaagatcccagctctggcctgggcgtgacaaagcaagatctgggccctgtgcctatgatggccgatcctgagggaacagatggcgaaggcacaggctgcaacggctggttttacgtgcaagccatcgtggacaagaagaccggcgacgtcatcagcgacgacgaggatgagaatgccaccgacaccggcagcgacatggtggactttatcgatacccagggcacgttctgcgagcaggccgaactggaaacagcccaggcactgtttcacgcccaagaggtgcacaacgatgcccaggtgctgcacgtgctgaagagaaagtttgccggcggaagcaccgagaacagcccactgggagaaagactggaagtggacaccgagctgagccccagactgcaagagatcagcctgaacagcggccagaagaaggccaagcggagactgttcaccatctccgacagcggctacggctgtagcgaagtggaagccacacagatccaagtgaccaccaatggcgagcacggcggcaatgtttgttctggcggatctaccgaggccatcgacaatggcggaaccgagggcaacaatagctccgtggatggcaccagcgacaacagcaacatcgagaacgtgaaccctcagtgcacaatcgcccagctgaaggacctgctgaaagtgaacaacaagcagggcgccatgctggccgtgtttaaggatacctacggcctgagcttcaccgacctcgtgcggaacttcaagagcgacaagaccacctgtaccgactgggtcaccgccatctttggcgtgaacccaacaatcgccgagggcttcaagaccctgatccagcctttcatcctgtacgcccacatccagtgtctggactgcaaatggggcgtgctgattctggccctgctgagatacaagtgcggcaagagcagactgaccgtggccaagggactgagcactctgctgcatgtgcccgagacatgcatgctgattcagcctccaaagctgcggtctagcgtggccgctctgtactggtacagaaccggcatcagcaatatcagcgaagtgatgggcgacacccctgagtggattcagcggctgacaatcatccagcacggcatcgacgactccaacttcgacctgagcgagatggtgcagtgggccttcgataacgagctgaccgacgagtccgacatggcctttgaatacgccctgctggcagacagcaactccaatgccgccgctttcctgaagtccaactgccaggccaagtacctgaaggactgcgccaccatgtgcaagcactaccgcagagcacagaaacggcagatgaacatgagccagtggatccggttccggtgcagcaagatcgatgaaggtggcgactggcggcctatcgtgcagttcctgagataccagcagatcgagttcatcacctttctgggcgccctgaagtcattcctgaagggaacccctaaaaagaactgcctggtgttctgcggccctgccaataccggcaagtcctacttcggcatgagctttatccacttcatccagggcgccgtgatcagcttcgtgaacagcaccagccacttttggctggaacccctgaccgataccaaggtggcaatgctggacgacgccaccaccacctgttggacctacttcgacacctacatgcggaacgccctggacggcaaccccatcagcatcgacagaaagcacaagcccctcatccagctgaaatgccctccaatcctgctgaccacaaacattcaccccgccaaggacaatcggtggccctacctggaaagccggatcaccgtgttcgagttccccaacgcctttccattcgacaagaacggcaatcccgtgtacgagatcaacgacaagaattggaagtgcttcttcgagcggacctggtccagactggatctgcacgaggaagaagaggacgccgacacagagggcaatcccttcggcacctttaagtgtgtggccgggcagaatcacagacccctgggctgcagcatggccagattcgaggaccctaccagacggccctacaagctgcctgatctgtgcaccgaactgaacaccagccttcaggatatcgagatcacctgtgtgtactgcaagaccgtgctcgagctgacagaagtgtttgagttcgccttcaaggacctgttcgtggtgtacagagacagcatccctcacgccgcctgtcacaagtgcatcgacttctacagcagaatcagagagctgcggcactacagcgactccgtgtacggcgataccctggaaaaactgaccaacaccggactgtacaacctgctgatccggtgcctgagatgccagaaacctcagaaccccgccgagaaactgagacatctgaacgagaagcggcggttccacaatatcgccggccactacagaggccagtgccacagctgttgcaaccgggccagacaagaaagactccagcggagaagaggcagcatgcacggacctaaagccacacttcaggacatcgtgctgcacctcgagcctcagaatgagatccccgtggatctgctcggccatgagcagctgagcgatagcgaggaagagaacgacgagatcgacggcgtcaaccaccagcatctgcctgccagaagggccgaacctcagagacacaccatgctgtgcatgtgctgcaagtgcgaggcccggatcgagagagtggtggaatctagcgccgacgacctgagagctttccagcagctgttcctgaatactctgagcttcgtggccccttggtgcgctagtcagcagtgaggaagaatgtgcacagtgctaaccagaaaattcaggacaagactagtgcggccgcttttat

SEQ ID NO: 105 - DNA, 18 bp, synthetic construct
  misc_feature 1..18: note = Tet operator site
  source 1..18: mol_type = other DNA
ccctatcagtgatagaga

(19) United States
(12) Patent Application Publication
(10) Pub. No.: US 2025/0066427 A1
(43) Pub. Date: Feb. 27, 2025

(54) THERAPEUTIC PAPILLOMA VIRUS VACCINES

(71) Applicants: SIRION BIOTECH GMBH, Gräfelfing (DE); UNIVERSITÄT REGENSBURG, Regensburg (DE); INPROTHER APS, Copenhagen (DK); PROBIOGEN AG, Berlin (DE)

(72) Inventors: Christian THIRION, München (DE); Cordula PERTL, München (DE); Alexander KARLAS, Oberkrämer (DE); Volker SANDIG, Berlin (DE); Ingo JORDAN, Berlin (DE); Peter Johannes HOLST, Søborg (DK); Ditte Rahbæk BOILESEN, Copenhagen (DK); Ralf WAGNER, Dietramszell (DE); Patrick NECKERMANN, Regensburg (DE); Benedikt ASBACH, Regensburg (DE)

(21) Appl. No.: 18/684,381

(22) PCT Filed: Aug. 17, 2022
(86) PCT No.: PCT/EP2022/073016

§ 371 Date: Feb. 16, 2024

(30) Foreign Application Priority Data
Aug. 18, 2021 (EP) 21191940.2

(51) Int. Cl. C07K 14/005; A61K 39/00; A61K 39/295; A61P 35/00; A61P 37/04

(52) U.S. Cl. CPC C07K 14/005; A61K 39/295; A61P 35/00; A61P 37/04; A61K 2039/5256; A61K 2039/53; A61K 2039/585; A61K 2039/70; C12N 2710/10343; C12N 2710/20022; C12N 2710/20034

(57) ABSTRACT

The present invention relates to a nucleic acid comprising or consisting of nucleic acid sequences encoding papilloma virus proteins E1, E6, and E7; wherein said nucleic acid molecule encodes a single polyprotein.

The present invention relates to a nucleic acid comprising or consisting of nucleic acid sequences encoding papilloma virus proteins E1, E6, and E7; wherein said nucleic acid molecule encodes a single polyprotein.

In this specification, a number of documents including patent applications and manufacturer's manuals are cited. The disclosure of these documents, while not considered relevant for the patentability of this invention, is herewith incorporated by reference in its entirety. More specifically, all referenced documents are incorporated by reference to the same extent as if each individual document was specifically and individually indicated to be incorporated by reference.

Despite efficient prophylactic vaccines and the possibility to screen for cervical lesions, infection with human papillomavirus (HPV) was still responsible for more than 340.000 cervical cancer-related deaths worldwide in 2020 (Sung, H. et al., Global Cancer Statistics 2020: GLOBOCAN Estimates of Incidence and Mortality Worldwide for 36 Cancers in 185 Countries, CA: A Cancer Journal for Clinicians, 71 (3), 209-249, 2001).

More than 85% of these cases occurred in middle- and low-income countries (Haghshenas, M. et al. Prevalence and type distribution of high-risk human papillomavirus in patients with cervical cancer: a population-based study. Infect. Agent. Cancer 8, 20, 2013). Currently, there are three approved prophylactic vaccines providing near complete protection against vaccine-targeted HPV types, yet vaccine uptake is incomplete (Brotherton, J. M. L., Zuber, P. L. F. & Bloem, P. J. N. Primary Prevention of HPV through Vaccination: Update on the Current Global Status. Curr. Obstet. Gynecol. Rep. 5, 210-224, 2016). Moreover, these vaccines do not lead to the eradication of pre-infected cells, since they target the major capsid protein L1, which is not expressed in infected basal layer- and cervical cancer cells (Lehtinen, M. et al. Overall efficacy of HPV-16/18 AS04-adjuvanted vaccine against grade 3 or greater cervical intraepithelial neoplasia: 4-year end-of-study analysis of the randomised, double-blind PATRICIA trial. Lancet. Oncol. 13, 89-99, 2012; Hildesheim, A. et al. Effect of human papillomavirus 16/18 L1 viruslike particle vaccine among young women with preexisting infection: a randomized trial. JAMA 298, 743-753, 2007; Schiller, J. T., Castellsagué, X. & Garland, S. M. A review of clinical trials of human papillomavirus prophylactic vaccines. Vaccine 30 Suppl 5, F123-38; 2012; Hung, C.-F., Ma, B., Monie, A., Tsen, S.-W. & Wu, T.-C. Therapeutic human papillomavirus vaccines: current clinical trials and future directions. Expert Opin. Biol. Ther. 8, 421-439, 2008). Thus, novel interventions such as therapeutic vaccines are desirable.

Whereas most HPV infections are spontaneously cleared within months, some persist for years (Ho, G. Y., Bierman, R., Beardsley, L., Chang, C. J. & Burk, R. D. Natural history of cervicovaginal papillomavirus infection in young women. N. Engl. J. Med. 338, 423-428, 1998). These can progress towards low-grade squamous intraepithelial lesions (LSIL), which can further progress to high-grade squamous intraepithelial lesions (HSIL) and cervical cancer. The expression pattern of viral proteins changes during progression of SILs: in LSIL, mainly the early proteins E1/E2 are expressed, whereas in HSIL and transformed cells, E6/E7 are highly expressed and E2 expression is low or absent (Xue, Y. et al. HPV16 E2 is an immediate early marker of viral infection, preceding E7 expression in precursor structures of cervical carcinoma. Cancer Res. 70, 5316-5325, 2010; Chang, L., He, X., Yu, G. & Wu, Y. Effectiveness of HPV 16 viral load and the E2/E6 ratio for the prediction of cervical cancer risk among Chinese women. J. Med. Virol. 85, 646-654, 2013).

In many cases of SIL, however, natural regression occurs as a result of CD4+ and CD8+ T cell infiltration (Hibma, M. H. The Immune Response to Papillomavirus During Infection Persistence and Regression. The Open Virology Journal vol. 6, 2012). It has been shown that CD4+ T cells play a major role in lesion regression with increased CD4: CD8 ratios being found in the stroma of LSIL (Monnier-Benoit, S. et al. Immunohistochemical analysis of CD4+ and CD8+ T-cell subsets in high risk human papillomavirus-associated pre-malignant and malignant lesions of the uterine cervix. Gynecol. Oncol. 102, 22-31, 2006). Genetic studies have shown that resistance and susceptibility loci for chronic infections and cancer cluster in the MHC II region. Furthermore, HPV16⁺cervical cancer patients have impaired memory CD4⁺ T-helper responses against E2 and E6, which emphasizes the important role of T cell responses in preventing progression and clearing lesions (Brotherton et al, 2016; Lehtinen et al, 2012; Hildesheim et al, 2007; Schiller et al, 2012; Hung et al, 2008; Ho et al, 1998; Xue et al, 2010; Chang et al, 2013; Hibma, 2012; Monnier-Benoit et al, 2006; de Jong, A. et al. Human Papillomavirus Type 16-Positive Cervical Cancer Is Associated with Impaired CD4+ T-Cell Immunity against Early Antigens E2 and E6. Cancer Res. 64, 5449 LP-5455; 2004; Woo, Y. L. et al. A prospective study on the natural course of low-grade squamous intraepithelial lesions and the presence of HPV16 E2-, E6- and E7-specific T-cell responses. Int. J. cancer 126, 133-141, 2010; Ragonnaud, E., Pedersen, A. G. & Holst, P. J. Breadth of T Cell Responses After Immunization with Adenovirus Vectors Encoding Ancestral Antigens or Polyvalent Papillomavirus Antigens. Scand. J. Immunol. 85, 182-190, 2017; Dillon, S. et al. Resolution of cervical dysplasia is associated with T-cell proliferative responses to human papillomavirus type 16 E2. J. Gen. Virol. 88, 803-813, 2007). It was also reported that HPV-exposed children have E2-specific T cell responses after clearing the infection (Koskimaa, H. M. et al. Human papillomavirus 16-specific cell-mediated immunity in children born to mothers with incident cervical intraepithelial neoplasia (CIN) and to those constantly HPV negative. J. Transl. Med. 13, 1-11, 2015). Yet, current research on therapeutic HPV vaccines is primarily focused on the HSIL and cancer-stage of the disease, and directed toward the oncogenic viral proteins E6 and E7 (Hung et al, 2008). However, targeting the infection prior to carcinogenesis could be advantageous in terms of reducing morbidity and suffering related to cancer treatment, and might be easier to achieve. Thus, to target pre-malignant infection, other early proteins should be included as antigens.

There is no suitable animal model to study persistent HPV infections in a preclinical setting, but Macaca fascicularis papillomavirus type 3 (MfPV3) has a close phylogenetic and phenotypic relationship to HPV16 (Ragonnaud et al, 2017; Chen, Z. et al. Non-human Primate Papillomaviruses Share Similar Evolutionary Histories and Niche Adaptation as the Human Counterparts. Front. Microbiol. 10, 2093, 2019). Naturally occurring infections with this virus are associated with long-term persistence and at least LSIL-like lesions in the cervix of breeding female cynomolgus macaques (Macaca fascicularis), making them an ideal non-human primate (NHP) animal model (Chen, Z. et al. Genomic diversity and interspecies host infection of alpha12 Macaca fascicularis papillomaviruses (MfPVs). Virology 393, 304-310, 2009; Wood, C. E., Chen, Z., Cline, J. M., Miller, B. E. & Burk, R. D. Characterization and Experimental Transmission of an Oncogenic Papillomavirus in Female Macaques. J. Virol. 81, 6339-6345 (2007).

In view of the deficiencies of the prior art, the technical problem underlying the present invention can be seen in the provision of improved means and methods for the eradication of papillomavirus as well as the prevention and treatment of papilloma virus-associated disorders. This technical problem is solved by the subject-matter of the enclosed claims.

In the first aspect, the present invention provides a nucleic acid comprising or consisting of nucleic acid sequences encoding papilloma virus proteins E1, E6, and E7; wherein said nucleic acid molecule encodes a single polyprotein.

The nucleic acid in accordance with the first aspect requires presence of sequences encoding three papilloma virus proteins. Preferred is that either exactly three or exactly four papilloma virus proteins are encoded. To the extent a fourth protein is encoded, preference is given to a further protein of the E family of papilloma virus proteins, in particular to E2. Preferred papillomavirus species and strains are disclosed further below. Particularly preferred is human papillomavirus 16 (HPV16).

In accordance with the invention, coding sequences encoding the corresponding full-length or substantially full-length proteins are employed. The concomitant presence of three or four full-length protein coding sequences provides for the presentation of a multitude of immunogenic epitopes in an organism vaccinated with the nucleic acid of the first aspect or a vector or plasmid comprising said nucleic acid. The term “substantially full-length” as used herein refers to the optional absence of up to 20, up to 15, up to 10, or up to 9, 8, 7, 6, 5, 4, 3, or up to 2, or 1 amino acid. Preferably these deletions are at the C-terminus, N-terminus and/or in regions where deletions do not remove immunogenic epitopes or do not affect the immunogenicity of epitopes present in other parts of the amino acid sequence of the encoded protein. Particularly preferred is that said deletions reduce the oncogenic potential of a papillomavirus protein. A preferred embodiment of the latter is detailed further below (C-terminal modification of E6). It is furthermore preferred that “substantially full-length” applies to E6 only, whereas E1, E7, and, to the extent present E2, are full-length.

Immune responses such as T cells directed against three or more, preferably four different full-length proteins will unfold synergistic activity against papilloma virus infections and disorders associated therewith. In particular, successful stimulation of E1/E2-specific cellular immunity primarily clears infections in the LSIL-stage, whereas E6/E7-specific responses mainly targets HSIL and cancer stages.

Further advantages of the invention include the surprising capability to completely eradicate a papillomavirus infection, and the usefulness of said nucleic acid for not only prophylactic, but also therapeutic purposes. This is particularly advantageous in view of the oncogenic potential of papillomavirus.

Of note, it is not just the concomitant presence of three, preferably four full length antigens in the vaccine of the present invention which provides superior performance, but in particular the particular order of them on the vector. Surprisingly, orders deviant from those of the invention fail to ensure proper epitope presentation of all four antigens. In other words, the invention not only relates to expanding the number of antigens, but also, and in particular to the order of them as defined herein.

The approach of using full-length coding sequences outperforms prior art vaccines which employed sequences encoding only fragments as opposed to full-length proteins. In particular, the synergistic effect arising from the concomitant expression of three or four full-length viral proteins is superior to the concomitant use of only fragments of a plurality of proteins inter alia owing to the larger number of immunogenic epitopes being provided. On the other hand, the use of full-length proteins for immunization in many instances will require further measures to the extent a protein with oncogenic potential is among said proteins. Such measures are subject of certain embodiments disclosed further below.

Unexpected and superior performance is evidenced by the Examples enclosed herewith. As shown in FIG. 17 and discussed in Example 4, constructs in accordance with the invention (which are characterized by both the number of antigens and their order) significantly outperform constructs comprising only two antigens. FIG. 17A shows a dramatic improve in the reduction of tumor volume, and FIG. 17B shows a big improvement in terms of survival.

Yet further, it is known that in case of persistent HPV infection, exhaustion of T cells may occur. Under such circumstances, T cell response is reduced. As shown in Example 3, a construct of the invention is capable of triggering immune response in non-human primates which are suffering from persistent HPV infection and T cell exhaustion. This is further surprising finding which is indicative of the constructs of the invention being useful and superior as vaccines and therapeutic vaccines.

The papillomavirus antigens of the invention, i.e., E1, E6, E7, and, to the extent present, E2, are encoded by the nucleic acid of the invention as a single polyprotein. In other words, the coding sequences therefor are part of a single open reading frame. The term “polyprotein” includes fusions of said antigens, more specifically direct fusions with no intervening amino acids between the sequences of the respective proteins. The term “polyprotein” also embraces proteins wherein one or more amino acids are inserted between the sequences of two neighboring proteins or placed N-terminal of the most N-Terminal E protein, or placed C-terminal of the most C-terminal E protein. Preferred implementations of such inserted sequences are given below and may serve to provide specific functions such as adjuvant function or self-cleaving function, and/or avoid structural interference between adjacent proteins. The latter issue may be addressed by sequences encoding short flexible linkers being located between the nucleic acid sequences encoding the antigens.

The notion of a single polyprotein being encoded by the nucleic acid of the invention does not exclude that such polyprotein, either during translation or once translation is completed, gives rise to more than one protein molecule. The latter applies in particular to those embodiments of the nucleic acid of the invention where, in addition to said papillomavirus proteins, a self-cleaving peptide is encoded.

The term “nucleic acid” refers to a polycondensate of nucleotides. Preference is given to nucleic acids which are recognized and processed by polymerases and/or a ribosome. Preferred implementations are DNA and RNA; for details see further below. Albeit less preferred, the term “nucleic acid” also extends to polycondensates comprising one or more modified nucleotides. Sites of modification in a nucleotide are those well known in the art, i.e., the sugar, the phosphate and the base. Preferred modification in that respect are 2′ modifications such 2′ O-alkyl including 2′ OMe and 2′ halogen substitutions such as 2′ F. Preferred phosphate modifications include thiophosphate. A preferred modified base is pseudouridine; see, for example, Nance and Meier, ACS Cent Sci. 2021 May 26; 7 (5): 748-756. The mentioned base, sugar and/or phosphate modifications are especially preferred in the context of an RNA vaccine, i.e., where the nucleic acid of the invention is implemented as RNA.

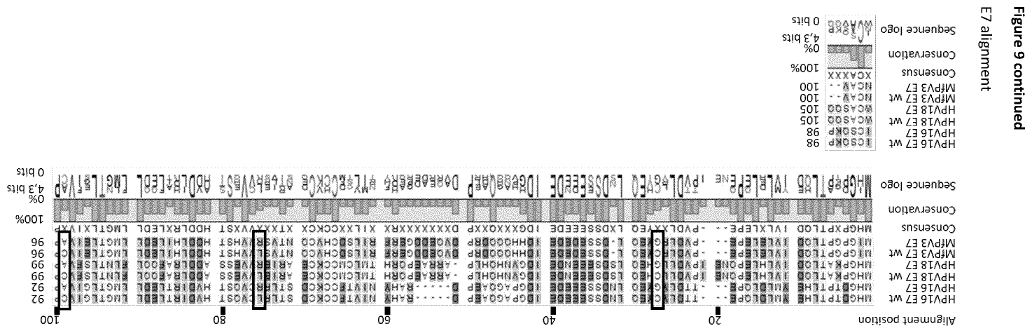

The E family of papillomavirus proteins are proteins referred to as “early” in the lifecycle of the virus. In functional terms, E1 has enzymatic function; it is an ATP-dependent helicase; see, e.g., Bergvall et al., Virology 2013 October; 445 (1-2): 35-56. E6 is capable of inducing cell division in resting cells. It inhibits apoptosis and the immune response; see, e.g. Vande Pol and Klingelhutz, Virology 2013 October; 445 (1-2): 115-37. Together with E7 it has transforming effect. E7 is involved in replication of the viral genome (see, e.g., Roman and Munger, Virology 2013 445:138-68) and, together with E6, transforms cells. FIG. 9 displays a multiple sequence alignment of wild-type E proteins with preferred E proteins of the invention.

In a preferred embodiment, said nucleic acid furthermore comprises or further consists of a nucleic acid sequence encoding papilloma virus protein E2.

E2 is multifunctional and mainly involved in genome replication and transcription; see, e.g. McBride, Virology. 2013 October; 445 (1-2): 57-79.

The term “further consists of” refers to a closed listing of a preceding aspect or embodiment and provides for an expansion of said closed list by addition of an explicitly recited feature, wherein the listing remains closed and confined to the features of said preceding aspect or embodiment, augmented by said explicitly recited additional feature.

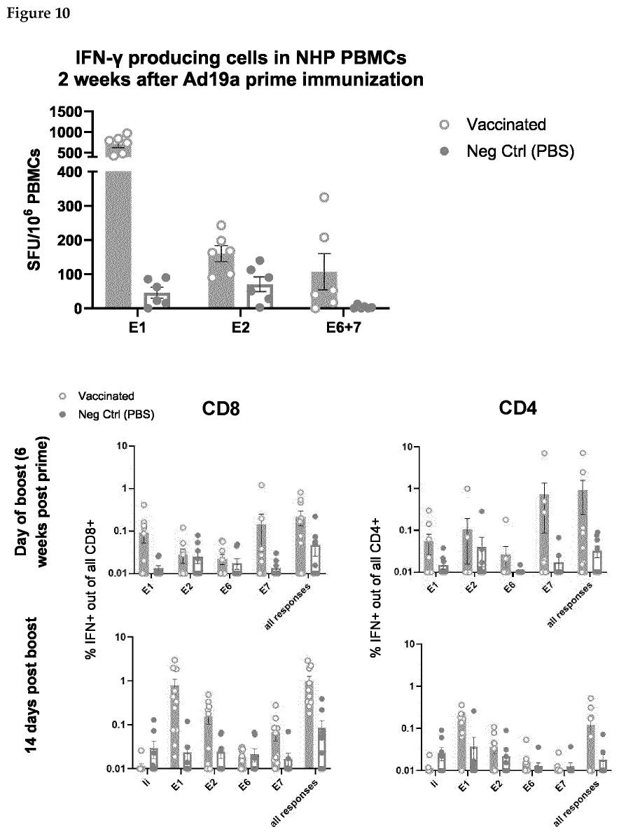

Of note, adding E2 as a further antigen does not deteriorate immune response; see, e.g. Example 4 and FIG. 10.

Antigen sequences in accordance with the invention are given in the sequence listing as follows: Amino acid sequences of E1, E2, E6 and E7 of MfPV3 are shown in SEQ ID NOs: 2, 4, 6, and 8, respectively.

Amino acid sequences of E1, E2, E6 and E7 of HPV16 are shown in SEQ ID NOs: 10, 12, 14, and 16, respectively.

Amino acid sequences of E1, E2, E6 and E7 of HPV18 are shown in SEQ ID NOs: 18, 20, 22, and 24, respectively.

The encoding nucleic acids for these proteins are shown, in the same order, in SEQ ID NOs: 1, 3, 5, 7, 9, 11, 13, 15, 17, 19, 21, and 23, respectively.

In accordance with said preferred embodiment, the nucleic acid of the invention encodes the four proteins E1, E2, E6 and E7 as a polyprotein, wherein preferably no further proteins of papillomavirus origin are encoded by said nucleic acid.

In a further preferred embodiment, the order of the nucleic acids encoding said proteins is, from 5′ to 3′, (a) to the extent E2 is present (i) E1, E2, E6, E7 or (ii) E1, E6, E7, E2; or (b) E1, E6, E7. Preferably, any of these listings is closed, i.e., no further sequence encoding a further protein of papillomavirus origin is present. The embodiment on the other hand extends to implementations where further amino acid sequences, preferably functional amino acid sequences, are present (for details see further below).

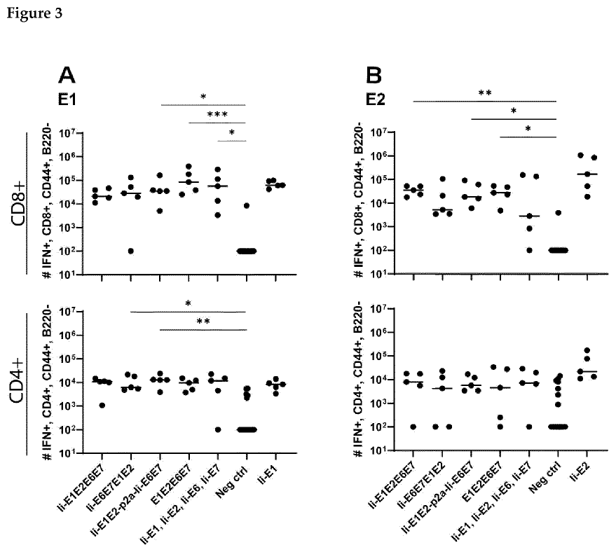

Most preferred is that sequences encoding the four proteins E1, E2, E6 and E7 are present and in the indicated order (from 5′ to 3′). While also constructs of the invention with a different order perform well, it can be seen e.g. in FIG. 3B that the order in the most preferred construct secures even better performance in terms of the E2 response.

In a further preferred embodiment, said nucleic acid of the first aspect comprises nucleic acid sequences encoding papilloma virus proteins E6 and/or E7, wherein said nucleic acid sequences are modified as compared to the wild-type counterparts by point mutations and/or deletions, wherein said point mutations and/or deletions (i) abolish colony forming in a soft agar assay, at least to a background level; and (ii) said modified proteins E6 and E7 maintain at least 70% of predicted high-affinity epitopes (IC₅₀≤50 nM) and at least 50%, at least 55% or at least 58% of predicted low-affinity epitopes (50 nM<IC₅₀≤5 μM) of the predicted epitopes of the respective wild-type protein, preferably in terms of (predicted) binding to the most frequent HLA-A, -B, and -C alleles which preferably together comprise 75% of the human HLA diversity for each HLA, respectively. This preferred embodiment allows for the presence of further papillomavirus proteins, E1 and optionally E2 being preferred in this context.

Said most frequent alleles are as follows:
HLA-A*01:01
HLA-A*02:01
HLA-A*02:02
HLA-A*02:03
HLA-A*02:05
HLA-A*02:06
HLA-A*02:07
HLA-A*02:11
HLA-A*02:12
HLA-A*02:16
HLA-A*02:17
HLA-A*02:19
HLA-A*02:50
HLA-A*03:01
HLA-A*11:01
HLA-A*24:02
HLA-A*24:03
HLA-A*30:01
HLA-A*30:02
HLA-A*33:01
HLA-B*07:02
HLA-B*08:01
HLA-B*08:02
HLA-B*08:03
HLA-B*15:01
HLA-B*15:02
HLA-B*15:03
HLA-B*15:09
HLA-B*15:17
HLA-B*18:01
HLA-B*27:05
HLA-B*27:20
HLA-B*35:01
HLA-B*35:03
HLA-B*39:01
HLA-B*40:01
HLA-B*40:02
HLA-B*40:13
HLA-B*44:02
HLA-B*44:03
HLA-B*51:01
HLA-B*58:01
HLA-B*58:02
HLA-C*03:03
HLA-C*04:01
HLA-C*06:02
HLA-C*07:01
HLA-C*07:02
HLA-C*08:02

In a particularly preferred embodiment, said modified E6 protein maintains at least 80%, at least 90%, at least 95%, or at least 98% of predicted high-affinity epitopes (IC₅₀≤50 nM) and at least 60%, at least 70%, at least 80%, at least 90%, or at least 93% of predicted low-affinity epitopes (50 nM<IC₅₀≤5 μM) of the predicted epitopes of wild-type E6, preferably determined as specified above.

Related thereto, and in a separate aspect, the present invention provides a nucleic acid comprising or consisting of nucleic acid sequences encoding papilloma virus proteins E6 and/or E7, wherein said nucleic acid sequences are modified as compared to the wild-type counterparts by point mutations and/or deletions, wherein said point mutations and/or deletions (i) abolish colony forming in a soft agar assay; and (ii) said modified proteins E6 and E7 maintain at least 70% of predicted high-affinity epitopes (IC₅₀≤50 nM) and at least 50%, at least 55% or at least 58% of predicted low-affinity epitopes (50 nM<IC₅₀≤5 μM) of the predicted epitopes of the respective wild-type protein, preferably in terms of binding to the most frequent HLA-A, -B, and -C alleles which preferably together comprise 75% of the human HLA diversity for each HLA, respectively. In a preferred embodiment of said separate aspect, said modified E6 protein maintains at least 80%, at least 90%, at least 95%, or at least 98% of predicted high-affinity epitopes (IC₅₀≤50 nM) and at least 60%, at least 70%, at least 80%, at least 90%, or at least 93% of predicted low-affinity epitopes (50 nM<IC₅₀≤5 μM) of the predicted epitopes of wild-type E6, preferably determined as specified above. This aspect does not require any further nucleic acid sequences encoding papilloma virus proteins to be present.

As noted above, proteins E6 and E7 play a role in controlling and modifying host cell behaviour and can unfold oncogenic or transforming potential. As a consequence, using these proteins as immunogens is desirable and attractive, especially when it comes to control, eradicate, prevent, mitigate or treat papillomavirus-associated malignant disorders. On the other hand, their oncogenic potential entails corresponding risks associated with their use as a vaccine or components of a vaccine. In line with these considerations, the above preferred embodiment provides measures to mitigate or abolish said oncogenic potential. In this context, a balance is struck by said embodiment in that oncogenicity is controlled while immunogenicity is retained. Preferably, said point mutations and deletions are minimal in number.

As a measure of oncogenic potential, the art-established standard which is a soft agar assay is used; see also the enclosed Examples.

As a measure of immunogenicity, in silico methods predicting immunogenicity and/or the number of immunogenic epitopes are used. Such methods include computer algorithms that predict T cell epitopes binding to MHC I molecules. A preferred in silico method in this respect is the IEDB analysis resource ANN (also known as NetMHC), ver 4.0 (Nielsen et al., Protein Sci 12:1007 (2003); Lundegaard et al., Nucl Acids Res 36, W509 (2008); Andreatta et al., Bioinformatics 32, 511 (2016)). Preferred is also that at least 50%, at least 60%, at least 70%, at least 75%, at least 80%, at least 90%, at least 95%, at least 98% or all immunogenic epitopes as they are present in the corresponding wild-type proteins are retained in modified E6 and/or E7.

As will become apparent in the following, the present invention provides, in a preferred embodiment, a minimum of modifications as compared to the corresponding wild-type sequences, wherein said minimum achieves both the required performance in a soft agar assay as well as in terms of retaining a high percentage of the immunogenic epitopes of the corresponding wild-type sequences.

Accordingly, in a particularly preferred embodiment, only the following modifications as compared to the wild-type are present in the encoded proteins:
(i) in E6: a modification which reduces or abolishes interactions mediated by the
   PDZ domain comprised in wild-type E6; and
   a. in HPV16 E6: L117X₁;
   b. in HPV18 E6: L112X₁;
   c. in MfPV3 E6: L110X₁;
   and
(ii) in E7:
   a. in HPV16 E7: C24X₂, L67X₃, and C91X₄;
   b. in HPV18 E7: C27X₂, L74X₃, and C98X₄;
   c. in MfPV3 E7: C24X₂, L71X₃, and C95X₄;
wherein X₁ to X₄ are independently a proteinogenic amino acid different from the respective amino acid they substitute.

Generally speaking, a position in an E2, E6 or E7 protein from a given first papillomavirus species corresponding to a position in an E2, E6 or E7 protein, respectively, from a given second papillomavirus species can be determined using the multiple sequence alignment shown in FIG. 9. Corresponding positions are one on top of the other. In other words, the determination of corresponding positions is straightforward in view of the high degree of conservation across different HPV strains, in particular using said multiple sequence alignment to which sequences from any further papillomavirus strain can be added without further ado. Such alignments can be prepared with computer programs well known to the skilled person, preferably with CLC Main Workbench version 8.0.1 (QIAGEN, Aarhus, Denmark) using the default settings (very accurate; gap open cost 10.0; gap extension cost 1.0, end gap cost: as any other).

Preferably, but not necessarily, said nucleic acid furthermore comprises one or both mutations encoding D21X₅ and C58X₆ in HPV 16 E7, D24X₅ and C65X₆ in HPV 18 E7, and D21X₅ and C62X₆ in MfPV3 E7, wherein X₅ and X₆ are independently a proteinogenic amino acid different from the respective amino acid they substitute.

In terms of specific modifications, it is preferred that (i) X₁ is selected from Q, Y, and N, and preferably is Q; (ii) X₂ is selected from G, A, V, L, and I, and preferably is G; (iii) X₃ is selected from R, K, and H, and preferably is R; (iv) X₄ is selected from A, G, V, L, and I, and preferably is A; (v) X₅ is selected from G, A, V, L, and I, and preferably is G; (vi) X₆ is selected from G, A, V, L, and I, and preferably is G; and/or (vii) said modification which reduces or abolishes interactions mediated by the PDZ domain is a deletion of 1 to 10 C-terminal amino acids, preferably counted from the C-terminus, more preferably ΔETQL in HPV16 E6, ΔETQV in HPV18 E6, and ΔETEV in MfPV3 E6; or said modification are one, two, three or four point mutation with the 10, preferably the four C-terminal amino acids.

The notation “Δ” is art-established and, in the context of this embodiment, refers to the deletion of the four indicated C-terminal residues.

In a further preferred embodiment, the total number of Cys residues in said polyprotein is an even number. How this is achieved is not particularly limited. Preference is given to implementations which do not entail loss of one or more immunogenic epitopes. Without wishing to be bound by a specific theory, it is envisaged that an even number of Cys residues increases antigen yield. To explain further, antigens, when fused to invariant chain (for details regarding the invariant chain see further below) are directed to the secretory pathway. In the endoplasmatic reticulum (ER), where chaperones (such as protein-disulfide-isomerase that binds to free cysteines) could detect free Cys which in turn might lead to an ER-stress-response that then would lead to a decrease or stop in protein-synthesis thus limiting expression of the antigen.

In a particularly preferred embodiment, if said total number of Cys residues is otherwise not even,
(i) a sequence encoding
   a. HPV16 E2 with a C300X₇ mutation;
   b. HPV18 E2 with a C301X₇ mutation; or
   c. MfPV3 E2 with a C297X₇ mutation
is comprised in said nucleic acid as sequence encoding E2, wherein X₇ is a proteinogenic amino acid other than C, preferably selected from A, S, G, M, T, Y, Q, N, V, L, I, F, W, H, K, R, Q, N, and P, more preferably A;
(ii) or a codon encoding Cys is inserted in said nucleic acid.

In a further preferred embodiment, said nucleic acid furthermore comprises at least one nucleic acid sequence encoding a genetic adjuvant, preferably T cell adjuvant MHC-II-associated invariant chain. Said invariant chain is also abbreviated as “li” in the following. The T cell adjuvant MHC-II-associated invariant chain has successfully been used as a genetic adjuvant (i.e., an adjuvant encoded by a nucleic acid, said nucleic acid optionally comprising also nucleic acid sequences encoding the antigen(s) against which an immune response is to be raised) in several contexts, see, for example, published international patent applications WO 2007/062656, WO 2010/057501 and WO 2018/172259. In terms of the species said invariant chain originates from, preference is given to vertebrates, including teleost invariant chain (see, e.g. WO 2020/079234), shark invariant chain (see, e.g., WO 2015/082922) and mammalian, preferably human invariant chain; see the above cited three WO documents. Particularly preferred is human invariant chain.

Further genetic adjuvants which may be used in accordance with the invention are heat-shock proteins, calreticulin and C4b.

Presence of said invariant chain entails unexpected performance owing to a synergistic effect. In particular, the combination of the above disclosed modifications of the oncogenic proteins E6 and E7 with li reduces oncogenicity to an unexpected degree. Without wishing to be bound by a specific theory, this is considered to be a consequence of a technical effect provided by li: export of E6 and E7, as such and in the absence of a fused li predominantly localized in the nucleus, into the cytoplasm where the oncogenic potential cannot unfold or only to a lesser extent.

In a particularly preferred embodiment, said invariant chain (i) is present once and precedes the sequence encoding the first E protein; or (ii) is present twice, one copy preceding the sequence encoding the first E protein, and a second copy preceding the sequence encoding E6 or, to the extent present, the sequence encoding E2. This embodiment relates to exemplary or preferred constructs, where a specific placement of said invariant chain within a nucleic acid of the invention applies. While such exemplary embodiments have been reduced to practice (see the Examples enclosed herewith), the invention is not so limited. The term “first E protein” refers to the most N-terminal protein selected from E1, E6 and E7, and, if present, E2 in the fusion protein encoded by the nucleic acid of the invention. Preferred is that E1 is the first E protein.

As an alternative or in addition to said genetic adjuvant, adjuvants known in the art may be used. Such adjuvants include cytokines, adhesion molecules, chemokines, CCR1/5 agonists, MIP-1 alpha, danger signals, LPS and derivatives thereof such as lipid A and lipid A derivatives, and TLR agonists.

In a further preferred embodiment, said nucleic is (i) DNA; (ii) a plasmid; or (iii) RNA, preferably mRNA. mRNA vaccines are being developed and several have received market approval such as Comirnaty of Biontech/Pfizer and mRNA-1273 of Moderna Therapeutics. In view of its inherent instability, RNAs may be chemically modified in order to enhance their stability; see, e.g. the modifications described for siRNAs in Hu et al., Signal Transduction and Targeted Therapy 2020, 5:101 as well the modifications disclosed herein further above. mRNA vaccines generally comprise a Kozak sequence at the translation start point, a translation termination codon at the end of the open reading frame, and a poly-adenylation site in the 3′-untranslated region.

In a preferred embodiment, said DNA and said plasmid comprise a promoter, preferably a promoter active in eukaryotic cells. Such promoters include a CMV promoter, preferably with a HTLV-1 enhancer element, UbC promoter, EF1alpha promoter and SV40 promoter.

In a preferred embodiment, said plasmid comprises a bacterial origin of replication such as pUC ori, and more preferably also selection marker such as an antibiotic resistance gene such as a Kanamycin resistance gene.

In a further preferred embodiment, said nucleic acid, in particular said DNA, comprises an untranslated 5′ terminus comprising a Tet operator. Among the known Tet-Off and Tet-On systems, preference is given to Tet-Off, given that the off-switch would be a constituent of the producer cell and not of the virus product. A preferred Tet operator site is shown in SEQ ID NO: 105. The Tet system employed by the present invention is the system known as T-REX system.

In the T-REX system the gene of interest is flanked by an upstream CMV promoter and two copies of tetracycline operator 2 (TetO2) sites. Expression of the gene of interest is repressed by the high affinity binding of TetR homodimers to each TetO2 sequences in the absence of tetracycline. In the present invention the production cell lines were modified to express the Tet repressor TetR. Introduction of tetracycline results in binding of one tetracycline on each TetR homodimer followed by release of the TetR homodimer from the TetO2. Unbinding of TetR homodimers form the TetO2 results in de-repression of the gene of interest. See, for example, Hillen W. and Berens. Mechanisms underlying expression of Tn10 encoded tetracycline resistance, Annu Rev Microbiol. 1994; 48:345-69; W Hillen, C Gatz, L Altschmied, K Schollmeier, I Meier. Control of expression of the Tn10-encoded tetracycline resistance genes. Equilibrium and kinetic investigation of the regulatory reactions, J Mol Biol. 1983 Sep. 25;169 (3): 707-21; Kathleen Postle, Toai T. Nguyen, Kevin P. Bertrand. Nucleotide sequence of the repressor gene of the TN10 tetracycline resistance determinant. Nucleic Acids Research, Volume 12, Issue 12, 25 Jun. 1984, Pages 4849-4863; and Feng Yao, Tor Svensjö, Thomas Winkler, Michael Lu, Carl Eriksson, and Elof Eriksson. Tetracycline Repressor, tetR, rather than the tetR-Mammalian Cell Transcription Factor Fusion Derivatives, Regulates Inducible Gene Expression in Mammalian Cells, Human Gene Therapy. September 1998.1939-1950.

In a preferred embodiment, said nucleic acid comprises at least one nucleic acid sequence encoding a self-cleaving peptide or a peptide which causes a ribosome not to form a peptide bond while continuing translation, wherein said nucleic acid sequence encoding said peptide preferably (i) precedes at least one nucleic acid sequence encoding a genetic adjuvant; or (ii) is located between and adjacent to two nucleic acid sequences encoding a papilloma virus protein.

Given that a maximum number of immunogenic epitopes is to be presented, it is not preferred to insert said nucleic acid sequence encoding a self-cleaving peptide into a nucleic acid sequence encoding one of the antigens of the invention.

A preferred peptide sequence is referred to as “p2a” herein.

Especially preferred nucleic acids in accordance with the invention are those which encode the following polyproteins:
li-E1E2E6E7
li-E1E2-p2a-li-E6E7
E1E2E6E7
li-E1E6E7-p2a-li-E2
li-E1E6E7

The sequences of nucleic acids encoding these constructs, wherein the encoded proteins are or are based on MfPV3 proteins are, in the same order: SEQ ID NOs: 25, 27, 29, 31 and 33, and the corresponding polyprotein sequences are shown in SEQ ID NOs: 26, 28, 30, 32 and 34, respectively.

The sequences of nucleic acids encoding these constructs, wherein the encoded proteins are or are based on HPV16 proteins are, in the same order: SEQ ID NOs: 35, 37, 39, 41 and 43, and the corresponding polyprotein sequences are shown in SEQ ID NOs: 36, 38, 40, 42 and 44, respectively.

The sequences of nucleic acids encoding these constructs, wherein the encoded proteins are or are based on HPV18 proteins are, in the same order: SEQ ID NOs: 45, 47, 49, 51 and 53, and the corresponding polyprotein sequences are shown in SEQ ID NOs: 46, 48, 50, 52 and 54, respectively.

SEQ ID NOs: 100 to 104 all relate to HPV18 li-E1E6E7, more specifically as follows: HPV18 li-E1E6E7 nucleic acid sequence (SEQ ID NO: 100), HPV18 li-E1E6E7 protein sequence (SEQ ID NO: 101), HPV18 li-E1E6E7 payload as inserted into various viral vectors (SEQ ID NOs: 102 to 104).

Of note, to the extent E antigens are adjacent, they are preferably separated by a short flexible linker, preferably GS (see also below). A linker between li and an adjacent antigen is not required, but not excluded. A linker connecting p2a to any of the adjacent sequences, be it an antigen or li, is not required, but not excluded.

In a further preferred embodiment, said papilloma virus is a primate papilloma virus, preferably a human papilloma virus (HPV) such as HPV16, HPV18, HPV45, HPV31, HPV33, HPV35, HPV52, HPV58, HPV6 and HPV11, more preferably HPV16 or HPV18, or macaque papilloma virus, preferably MfPV3. Especially preferred is HPV16. Experimental evidence for HPV16 can be found in Example 4.

In a further preferred embodiment, neighbouring nucleic acid sequences are connected in one, more or all instances by sequences encoding a flexible linker which is 10 or less amino acids in length such as 2, 3, 4, or 5 amino acids, optionally comprising a Cys residue, wherein GS and GCS are preferred linkers. Preferably, such linkers are present only between neighbouring antigens; see also the discussion of the most preferred nucleic acids of the invention as given above.

A linker comprising a Cys residue such as GCS is preferably to be viewed in the context of the above disclosed preferred embodiment requiring an even number of Cys residues in the polyprotein of the invention. In other words, while a Cys-containing linker such as GCS is generally useful, it is considered to employ it in particular in those cases where the polyprotein encoded by the nucleic acid of the invention would otherwise have an uneven number of Cys residues. For example, use may be made of a GCS linker for connecting E1 and E6 such as in SEQ ID NOs: 100 to 104.

In a second aspect, the present invention provides a viral vector comprising the nucleic acid of any one of the preceding claims.

In a preferred embodiment, said vector is a DNA viral vector, preferably (i) an adenoviral vector, preferably being deficient with regard to adenoviral E1 and E3 encoding sequences, said vector more preferably belonging to class C, D or E and/or being Ad19a/64, Ad5, Ad5 with fiber replacements such as Ad5F35, Ad26, Chimp63, or chAdOx1; or (ii) a Poxvirus vector, preferably MVA, MVA CR19, SCV, Vaccinia, or Fowlpox.

Ad5 has been described in Schiedner G, Hertel S, Kochanek S. Hum Gene Ther. 2000 Oct. 10; 11 (15): 2105-16. doi: 10.1089/104303400750001417. PMID: 11044912.

Ad19a is also known as Ad64; see, for example, Zhou X., Robinson C. M., Rajaiya J., Dehghan S., Seto D., Jones M. S., Dyer D. W., Chodosh J. Analysis of human adenovirus type 19 associated with epidemic keratoconjunctivitis and its reclassification as adenovirus type 64, Invest. Ophthalmol. Vis. Sci, 53 (2012), pp. 2804-2811. Ad19a is also described in Ruzsics Z, Wagner M, Osterlehner A, Cook J, Koszinowski U, Burgert H G. J Virol. 2006 August; 80 (16): 8100-13. doi: 10.1128/JVI.00687-06. PMID: 16873266.

Adenoviruses with fiber replacements are described, for example, in Shayakhmetov, D. M.; Papayannopoulou, T.; Stamatoyannopoulos, G.; Lieber, A. Efficient Gene Transfer into Human CD34+ Cells by a Retargeted Adenovirus Vector. J. Virol. 2000.

Ad5F35 specifically has been described in Flickinger Jr J C, Singh J, Carlson R, et al. Chimeric Ad5.F35 vector evades anti-adenovirus serotype 5 neutralization opposing GUCY2C-targeted antitumor immunity, Journal for ImmunoTherapy of Cancer. 2020. Here it is shown that Ad5 and Ad5F35 induces comparable immune responses and tumour control in naïve mice, and that Ad5F35 induces better responses than Ad5 in mice pre-exposed to Ad5 infection. Further, it was shown that fewer patients have neutralising antibodies against Ad5F35 compared to Ad5. As such, and in view of its superior performance, Ad5F35 is particularly preferred.

Particularly preferred DNA payloads (inserts; sequences comprising antigen-encoding sequences) in accordance with the invention for various viral vectors are shown in SEQ ID NOs: 55 to 99 and 102 to 104. For further information regarding the specific features of the respective vector, see the respective annotations in the sequence listing.

The procedure for producing adenoviral vectors with inserts is known in the art and described, for example, in Ruzsics Z, Lemnitzer F, Thirion C. Methods Mol Biol. 2014; 1089:143-58. doi: 10.1007/978-1-62703-679-5_11. PMID: 24132484.

The sequences of MVA vectors can be found in sequence databases as follows:

MVA sequence: GenBank (release 244.0) accession number: U94848, version number: U94848.1, release date 14.04.2003, https://www.ncbi.nlm.nih.gov/nuccore/U94848.1;

MVA.CR19 sequence: GenBank (release 244.0) accession number: KY633487, version number KY633487.1, release date 28.03.2017, https://www.ncbi.nlm.nih.gov/nuccore/KY633487.1;

For the purpose of producing or multiplying said vector, a cell line, preferably a eukaryotic cell line may be employed, wherein said cell line comprises said vector. Suitable eukaryotic cell lines include HEK293, PerC6, AGE1.CR.pIX (Jordan I, Vos A, Beilfuss S, Neubert A, Breul S, & Sandig V, 2009. An avian cell line designed for production of highly attenuated viruses. Vaccine 27, 748-756. https://doi.org/10.1016/j.vaccine.2008.11.066. PMID 19071186), HEK293 T-REX and AGE1.CR.pIX-T-REx (derived from HEK293 and AGE1.CR.pIX, respectively, already expressing TetR), A549, and A549 with engineered adenovirus E1 overexpression. As noted above, to the extent not already present, said cell lines are preferably, but not necessarily modified to express TetR.

MVA is adapted to replication in avian cells. For production of MVA a host is therefore preferred such as primary chicken embryo fibroblasts (CEF) or AGE1.CR.pIX that is derived from duck retina cells. As opposed to primary cells, an immortalized (or continuous) cell line such as AGE1.CR.pIX has several advantages: the cell substrate can be retrieved out of locally stored cryocultures and thus is resilient to supply constraints. An immortal cell line can furthermore be characterized against adventitious agents at the level of the cell bank, well ahead of the actual production processes. The AGE1.CR.pIX cell line (as opposed to primary material) furthermore proliferates in suspension in media free of animal derived components. This property allows highly efficient and scalable fed-batch production processes for poxviruses of different genera (Jordan I, Northoff S, Thiele M, Hartmann S, Horn D, Höwing K, Bernhardt H, Oehmke S, von Horsten H, Rebeski D, Hinrichsen L, Zelnik V, Mueller W, & Sandig V, 2011. A chemically defined production process for highly attenuated poxviruses. Biologicals 39, 50-58. https://doi.org/10.1016/j.biologicals.2010.11.005. PMID 21237672).

Vaccinia viruses mature into infectious particles with one or three membranes without the requirement for budding. One consequence of this complex infectious cycle is that the majority of the wild-type virions remain associated with the producer cell and that only a small fraction of infectious activity can be measured in the culture supernatant. The development of the novel strain MVA-CR19 has been induced by this observation (Jordan I, Horn D, John K, & Sandig V, 2013. A genotype of modified vaccinia Ankara (MVA) that facilitates replication in suspension cultures in chemically defined medium. Viruses 5, 321-339. https://doi.org/10.3390/v5010321. PMID 23337383). MVA-CR19 is a strain of MVA with a unique genotype (Jordan I, Horn D, Thiele K, Haag L, Fiddeke K, & Sandig V, 2019. A Deleted Deletion Site in a New Vector Strain and Exceptional Genomic Stability of Plaque-Purified Modified Vaccinia Ankara (MVA). Virol Sin. https://doi.org/10.1007/s12250-019-00176-3. PMID 31833037). Point mutations in structural genes and recombination of a large portion of the inverted terminal repeat (ITR) at the left side of the linear genomic DNA have profound effects on the phenotype of MVA-CR19. For example, compared to wild-type, MVA-CR19 releases a larger number of infectious particles into the culture supernatant and replicates to higher infectious titers. Viral factors that impact immune responses of the host and the infectious cycle are encoded in the ITRs. The recombination event in MVA-CR19 has changed the expression pattern of these factors (some were deleted, for others the gene dose has been duplicated) with positive effects on efficacy and stability as a vaccine vector.

The potentially enhanced release of MVA-CR19 from host cells can also be seen in the CPE in adherent cells: whereas wild-type MVA tends to induce cell fusion and syncytia with well circumscribed plaques, infection with MVA-CR19 leads to a pattern consisting of large but loosely packed (unfused) plaques surrounded by isolated infected cells scattered at greater distances to the primary plaque or localized in comets.

Generation and isolation of recombinant MVA is complex due to the large size of the viral genome (178 kb). The most commonly used technique relies on homologous recombination in infected host cells with a shuttle plasmid that contains the gene of interest. The recombinant viruses must be isolated and purified from a vast background of contaminating parental viruses without the insert. While MVA-CR19 has advantages for production and vaccine efficacy it can be more complex to purify due to the less confined nature of replication. Furthermore, for both wild-type and MVA-CR19, selection against expression and maintenance of a transgene may occur if the novel sequence impairs the infectious cycle. In such case, a cell line constitutively expressing TetR and an expression unit containing copies of tetO can be utilized to minimize expression of the transgene during virus generation and production.

In a third aspect, the present invention provides a polyprotein encoded by the nucleic acid or the vector of any of the preceding claims or cleavage products derived therefrom.

To the extent reference is made to cleavage products, these are preferably cleavage products arising from the activity of a self-cleaving sequence which in accordance with a preferred embodiment disclosed above may be present in the polyprotein encoded by the nucleic acid of the invention. It is understood that cleavage does not affect the number of epitopes presented by the proteins encoded by the nucleic acid of the invention. As such, the cleavage site in the encoded polyprotein are preferably located between sequences of antigens.

In a fourth aspect, the present invention provides a pharmaceutical composition, preferably a vaccine, comprising or consisting of (i) a nucleic acid of the first aspect; (ii) a vector of the second aspect; (iii) a polyprotein or cleavage products thereof of the third aspect; and/or (iv) a cell transduced with the nucleic acid of (i) or the vector of (ii), wherein preferably said cell is an ex vivo or in vitro cell and/or a dendritic cell or a monocyte.

As regards item (iv), it is known in the art that cells may be transformed with an antigen-encoding construct, thereby obtaining a population of cells with prophylactic or therapeutic properties against a disorder associated with said antigen. An example in the field of cancer is Maeng et al., Journal of Clinical Oncology 37, no. 15_suppl (May 20, 2019) 2639-2639.

Preferably, said cells are autologous cells from the individual to be treated. Especially preferred for item (iv), to the extent a vector is used, is Ad19a/64.

In terms of routes of administration, intramuscular, subcutaneous, intradermal, vaginal, rectal, and/or mucosal administration, including to mucosae of the genital tract are envisaged.

Formulations include gels, in particular for mucosal, preferably vaginal administration.

Excipients, diluents and conservants are well known to the skilled person.

In terms of dosages, 10⁹ to 10¹¹ infectious units are preferred for adenoviral vectors, and 5×10⁶ to 5×10⁸ for poxviral vectors. Generally speaking, suitable dosages can be determined by a clinician or manufacturer without further ado in view of the present disclosure, possibly of clinical studies, and, where necessary, taking into account parameters such as weight, age, sex etc. of the individual to be vaccinated. Experience tells, though, that in the field of vaccines there is generally a one-fits-all dosage or dosage regimen.

In a preferred embodiment said vector, said nucleic acid, said polyprotein or said cell is the only pharmaceutically active agent.

In an alternative preferred embodiment, said pharmaceutical composition comprises two or more pharmaceutically active agents, wherein a second pharmaceutically active agent is selected from (i) a second nucleic acid of the first aspect; (ii) a second vector of the second aspect; (iii) a second polyprotein or cleavage products of the third aspect; (iv) a second cell as defined above; (v) a protein-based vaccine, preferably against papilloma virus; and (vi) a chemotherapeutic, preferably cisplatin. The Examples demonstrate a synergistic effect when the vaccine according to the invention is given together with cisplatin.

There are known papillomavirus vaccines which are proteins or protein-based. In accordance with item (v) above, such known vaccines may be combined with a vaccine in accordance with the present invention. Of note, also the present invention may be implemented, albeit less preferred, as a protein-based vaccine; see above item (iii).

Further agents for a combination therapy together with constructs (nucleic acids, vectors, polyproteins, cells) of the invention include antibodies, preferably monoclonal antibodies targeting an immune checkpoint, such checkpoints preferably being PD-1, PD-L1 and CTLA-4.

Generally speaking, the components of a combination therapy may be administered together or separate in any order, possibly following different dosage regimens.

In a particularly preferred embodiment, said pharmaceutical composition comprises two pharmaceutically active agents which are (i) an adenoviral vector as defined above; and a Poxvirus vector as defined above, wherein said two pharmaceutically active agents are preferably confectioned as two pharmaceutical compositions which may be administered in any order or concomitantly, preferably the pharmaceutical composition comprising said Poxvirus vector after said pharmaceutical composition comprising said adenoviral vector, preferably after at least 4 weeks have elapsed.

This preferred embodiment defines preferred prime/boost schemes in accordance with the invention. As such, it is preferred that said two distinct vectors are administered in a manner which allows time to elapse between the two administrations. Preferred time spans are at least 2 weeks, at least 3 weeks, at least 4 weeks, at least 5 weeks, at least 6 weeks, at least 7 weeks or at least 8 weeks. Preferred upper limits for said time span are 6 weeks, 7 weeks, 8 weeks, 3 months, 4 months, 5 months or 6 months.

In a fifth aspect, the present invention provides the nucleic acid, the vector, the polyprotein, or the cell of any one of the preceding claims for use in medicine.

Preferably, said vaccine is a therapeutic vaccine. Of note, the combination of full-length antigens in accordance with the invention provides for eradication of the virus. As such, said vaccine has not only prophylactic applications, but is also a therapeutic agent.

In a sixth aspect, the present invention provides the nucleic acid, the vector, the polyprotein, the cell, or the vaccine of any of the preceding claims for use in a method of treating, ameliorating or preventing papilloma virus infection or papilloma virus induced disorders such as warts, malignant cell changes or cancer. Infection and associated disorders can occur in a variety of sites, e.g. nasal-oral, skin, genitals (inner and outer). Some of the best known papillomavirus associates malignancies and cancers are low-grade squamous intraepithelial lesions (LSIL), high-grade squamous intraepithelial lesions (HSIL), nasal polyposis or papilloma virus-associated malignant conditions such as cervical cancer, head and neck squamous cell carcinoma (HNSCC), and oropharyngeal squamous cell carcinoma (OPSCC).

Generally speaking, it is understood that these disorders are papilloma-virus associated. Papillomavirus strains which are frequently involved as causative agents are known in the art and include HPV16, HPV18, HPV45, HPV31, HPV33, HPV35, HPV52, HPV58, HPV6 and HPV11. To give an example, nasal polyposis may arise from infections with HPV 6 or HPV 11.

Table 1 below provides an overview of HPV strains associated with several malignant and non-malignant disorders.

| | | HNSCC (DOI: | | |
|---|---|---|---|---|
| HPV | Cervical (DOI: | 10.1016/j.ejca.2020.04.027 | | |
| type | 10.1056/NEJMoa021641) | or) | Other cancers | genital warts |
| HPV16 | 54.6% | 86.0% | | |
| HPV18 | 11.0% | 1.0% | | |
| HPV45 | 4.4% | 0.3% | | |
| HPV31 | 3.4% | 0.3% | | |
| HPV33 | 2.0% | 7.4% | | |
| HPV35 | 2.0% | 3.4% (HPV35 is not in | | |
| | | gardasil-9) | | |
| HPV52 | 2.2% | | | |
| HPV58 | 2.0% | 0.10% | | |
| HPV6 | 0.2% | HPV11 0.1% (7.8% in doi: | | 90% |
| and 11 | | 10.3390/jcm7090241) | | (https://www.cancer.gov/) |
| | | | HPV (of any type) is | |
| | | | found in 40% vulva, | |
| | | | vaginal and penile | |
| | | | cancer (doi: | |
| | | | 10.3390/jcm7090241) | |

In a seventh aspect, the present invention provides an in vitro or ex vivo method of stimulating and/or expanding T cells, said method comprising bringing into contact in vitro or ex vivo T cells with a nucleic acid, a vector, or a polyprotein of any one of the preceding aspects of the invention.

In a preferred embodiment, said bringing into contact is effected in the presence of antigen-presenting cells. Such antigen-presenting cells are preferably dendritic cells, monocytes, macrophages or B lymphocytes. The presence of antigen-presenting cells provides for the expanded T cells to be MHC-restricted.

Preferred is that said T cells are ex vivo, i.e., obtained from an individual or patient. Envisaged is to put back said T cells, once they are expanded, into the same patient or individual. Such re-introduction of expanded T cells is also known in the art as adoptive immunotherapy.

Related thereto, the present invention provides in a further aspect T cells, preferably MHC-restricted T cells obtained by the method of the seventh aspect.

The Figures show:

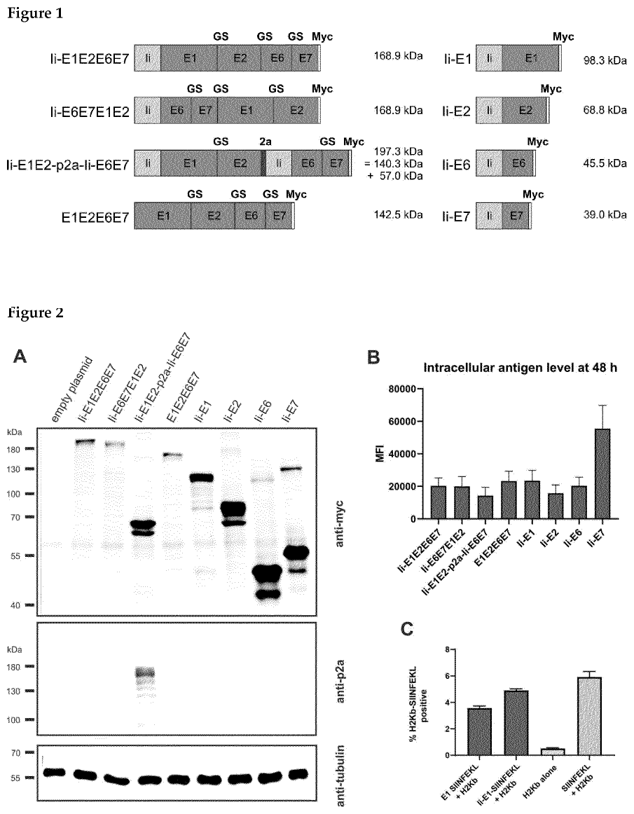

FIG. 1: Schematic representation of the conceived MfPV3 antigen variants.

MfPV3 antigens were designed as fusion proteins comprising either E1, E2, E6 and E7 altogether, or E1 plus E2 and E6 plus E7 fusion proteins linked by a p2a peptide, and fused to the MHC-II invariant chain (Ii), respectively. For reference, E1, E2, E6, and E7 were fused as single open reading frames without the li coding sequence. Theoretical molecular weight in kDa calculated from amino acid sequence. Abbreviations: Ii: MHC-II associated invariant chain, GS: glycine-serine-linker, 2a: p2a peptide for cotranslational separation, Myc: myc-tag sequence for antibody detection, kDa: kilo Dalton.

FIG. 2: Expression analysis of MfPV3 antigens.

A. Western blot analysis of HEK293T cell lysates 48 h following transfection with equimolar amounts of pURVac DNA vaccines expressing the various MfPV3 antigens. Antigens were detected with anti-myc (upper panel) and anti-p2a-peptide (middle panel) antibodies. Tubulin levels were monitored using an anti-tubulin antibody as loading control (lower panel). B. Flow cytometry analysis of HEK293T cells 48 hours following transfection with pURVac DNA vaccines. Intracellular staining was performed with anti-myc antibody. Depicted is the mean fluorescence intensity (MFI) of the average of 3 independent experiments. Error bars indicate standard error of the mean. C. Co-transfection of HEK293T cells with H2Kb alone or together with pURVac E1-SIINFEKL or Ii-E1-SIINFEKL, respectively, and analysis of SIINFEKL-H2Kb on cell surfaces 48 hours later by flow cytometry using an antibody recognizing the SIINFEKL-H2Kb-complex. Depicted is the percentage of H2Kb-SIINFEKL positive cells of the average of 6 independent experiments. Error bars indicate standard error of the mean. Light gray bars are negative and positive controls, respectively.

FIG. 3: T cell responses induced by the various pURVac DNA vaccines encoding E1, E2, E6 and E7 in outbred CD1 mice.

CD1 mice (5 per group) were immunized 4 times in 1 week intervals with 0.5 μg DNA of pURVac DNA encoding the indicated MfPV3 early antigens. Mice were sacrificed 7 days post last immunization, spleens were harvested and CD8 (top panels) and CD4 (bottom panels) T-cell immune responses against E1 (A), and E2 (B) were measured using ICS and flow cytometry. Negative control groups consist of all mice immunized with a pURVac DNA vaccine encoding antigens not covered by the peptide pools used for in vitro restimulation. Asterisks between groups indicate significant differences in response-levels after subtraction of background responses. Each symbol represents one mouse; the horizontal bar represents the median. Reference samples (mice vaccinated with pURVac DNA vaccine containing only the individual antigen linked to Ii, respectively) are not included in the statistical analysis (multiple comparison adjustment).

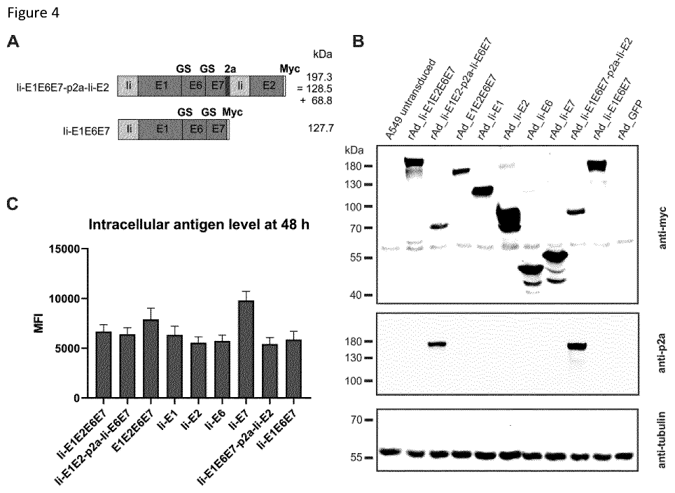

FIG. 4: Expression analysis of rAd-shuttled MfPV3 antigen constructs.

A. Schematic representation of the MfPV3 antigen variants Ii-E1E6E7-p2a-Ii-E2 and Ii-E1E6E7. MfPV3 antigens were designed as fusion proteins comprising both E1, E6 and E7 altogether, optionally fused to the MHC-II invariant chain (Ii) and E2, respectively. Theoretical molecular weight in kDa calculated from amino acid sequence. B. Western blot analysis of A549 cell lysates 48 h following transduction with rAds at an MOI of 30. Antigens were detected with anti-myc (upper panel) and anti-p2a-peptide (middle panel) antibodies. As loading control, tubulin levels were monitored using an anti-tubulin antibody (lower panel). C. Flow cytometry analysis of A549 cells 48 hours following transduction with rAds expressing the various MfPV3 antigens, at an MOI of 30. Intracellular staining was performed with anti-myc antibody. Cells were gated on non-infected cells. Depicted is the mean fluorescence intensity (MFI) of the average of 3 independent experiments. Error bars indicate standard error of the mean.

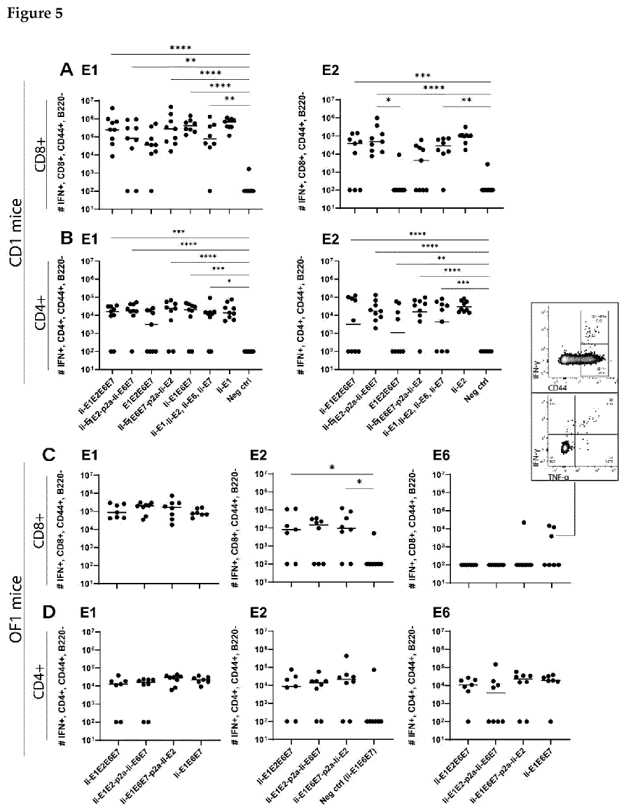

FIG. 5: Immunization of mice with adenovirus vectors induces potent cellular immune responses.

CD1 mice (A and B), or OF1 mice (C and D) were immunized with rAd vaccine (2×10⁷ IFU) encoding the various MfPV3 early antigens as indicated. Mice were sacrificed on day 14, spleens were harvested and CD8 and CD4 T-cell immune responses against E1, E2, E6, and E7 were measured using ICS and flow cytometry. Negative control groups consist of all mice immunized with rAd encoding antigens not covered by the peptide pools used for in vitro restimulation. Each symbol represents one mouse; the horizontal bar represents the median. Positive control samples (mice vaccinated with rAd containing only the relevant antigen linked to li) are not included in the statistical analysis (multiple comparison adjustment).

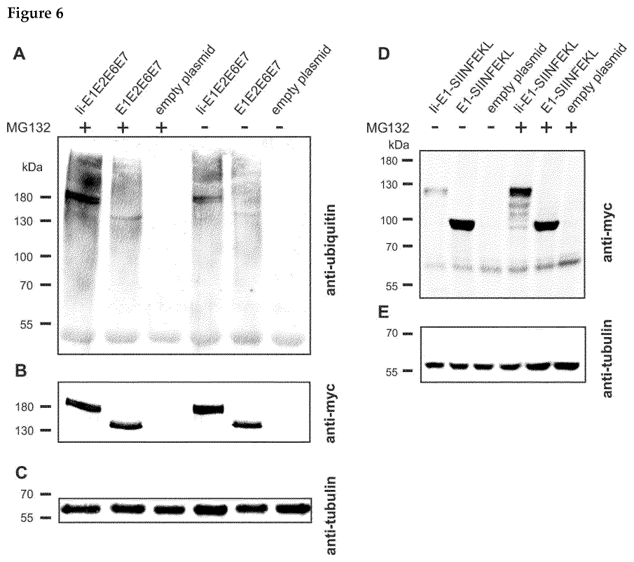

FIG. 6: Influence of li on ubiquitination and degradation.

A-C. pURVac Ii-E1E2E6E7, pURVac E1E2E6E7 or the empty plasmid were transfected into HEK293T cells. 24 h after transfection, cells were treated with MG132 or DMSO as control for 6 h. Myc-tagged proteins were immunoprecipitated and analyzed by western blot using an anti-ubiquitin antibody (A) and anti-myc antibody (B). Tubulin levels were monitored using an anti-tubulin antibody as loading control (C). D-E. pURVac li-E1-SIINFEKL, pURVac E1-SIINFEKL or empty plasmid were transfected into HEK293T cells. 24 h after transfection, cells were treated with MG132 or DMSO for 6 h. The samples were analysed by western blot using anti-myc antibody (D) and anti-tubulin antibody (E).

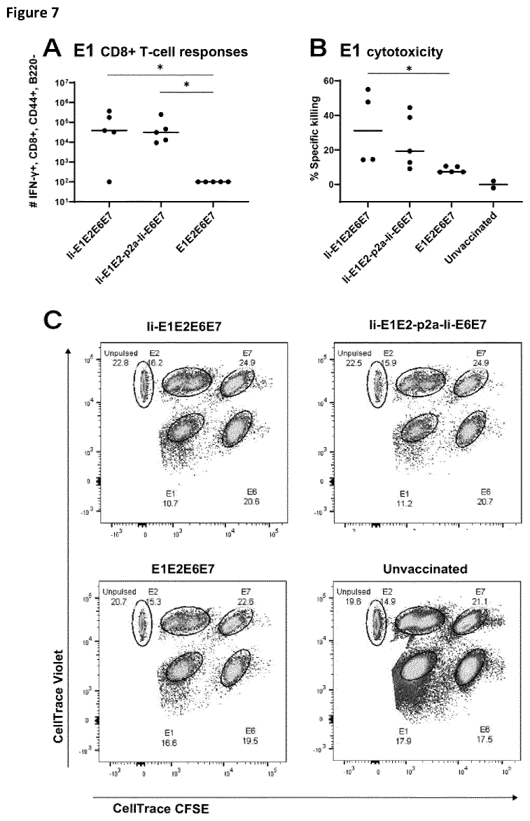

FIG. 7: Immune responses in inbred mice and in vivo cytotoxicity

Balb/C mice were immunized with rAd vectored vaccine encoding the indicated MfPV3 early antigens. 14 days post vaccination, immune responses were analyzed by ICS (A) or in vivo cytotoxicity regarding specific killing of E1-peptide pulsed cells (B). Each symbol represents one mouse. Two unvaccinated mice were included as negative controls. (C) Representative plot of in vivo cytotoxicity.

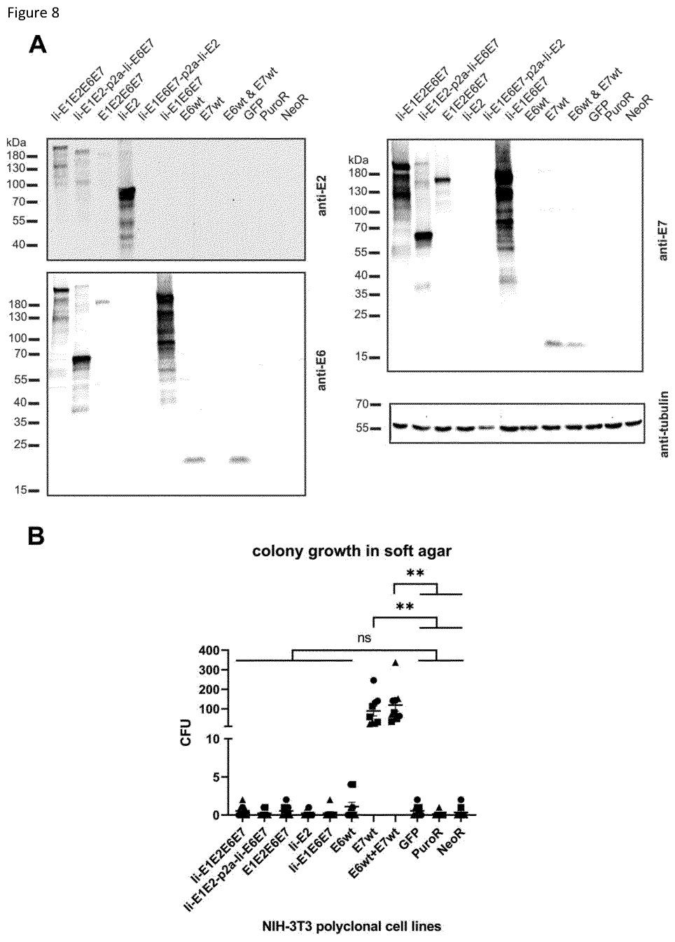

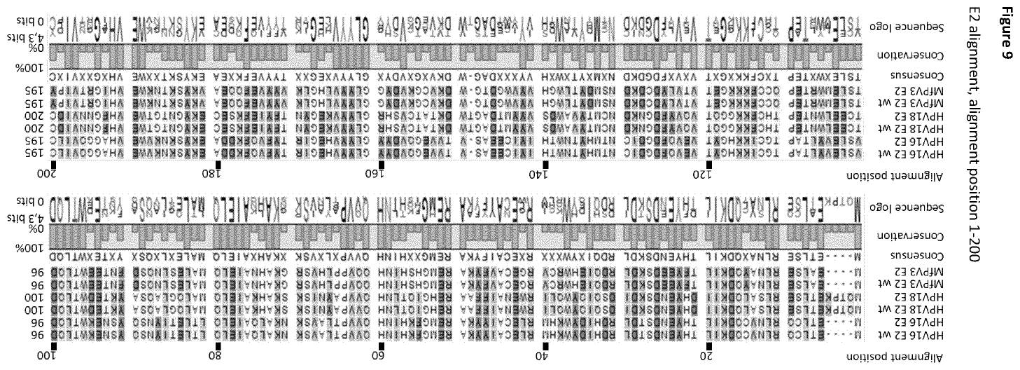

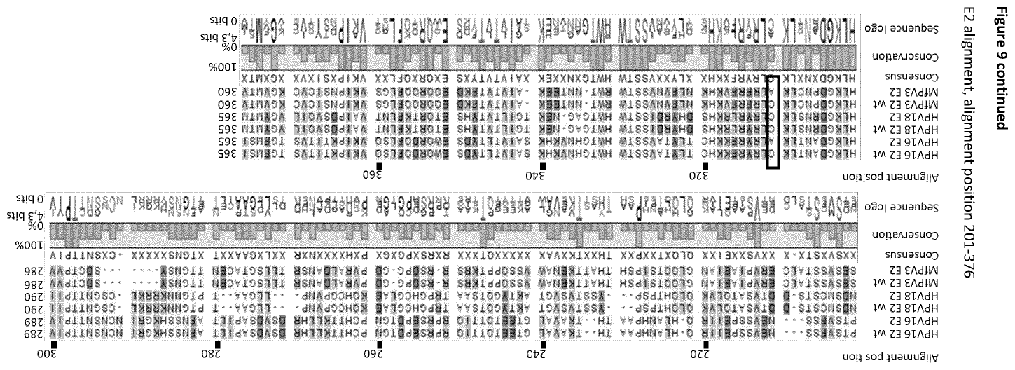

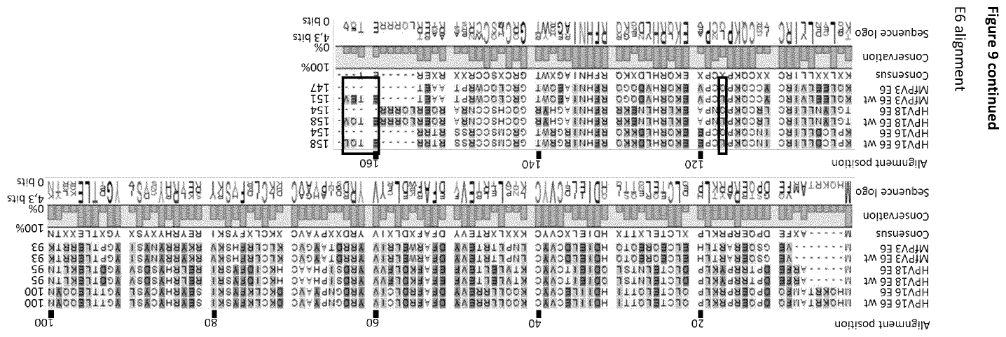

FIG. 8: Characterization of anchorage-independent growth. A. Analysis of transgene expression of polyclonal NIH-3T3 cell lines by western blot analysis with anti-E2, anti-E6, and anti-E7 antibodies. The anti-tubulin panel confirms uniform loading. B. Anchorage-independent colony growth in soft agar transformation assay. The number of colony-forming units (CFU) in the assay is shown for each NIH-3T3 cell line. Depicted is the mean with SEM of 3 technical replicates from 3 independent experiments. Individual groups were compared using two-sided, parameterized, unpaired t-test.

FIG. 9: Amino acid sequence alignments of E2, E6 and E7 proteins from preferred species. The alignment shows the wild-type (wt) amino acid sequence of the preferred papillomavirus species aligned with antigen sequences disclosed herein (without the “wt” qualifier), which possess the preferred modifications in accordance with the invention. The preferred modifications are highlighted with boxes.

FIG. 10: Top panel: Ad19a/64 prime with MfPV3 Ii-E1E2E6E7 induced IFN-producing MfPV3 specific T-cells in NHPs after 14 days after prime, measured by ELISpot. Bottom panels: Ad19a/64 prime, MVA boost vaccination with MfPV3 Ii-E1E2E6E7 induced IFN-producing MfPV3 specific T-cells in NHPs at the day of boost (6 weeks after prime) and 14 days after boost measured by ICS and flow cytometry

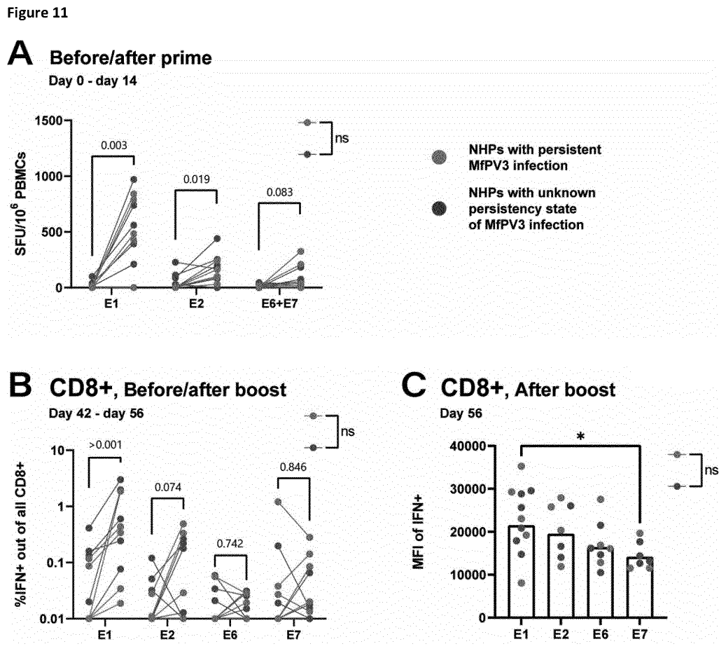

FIG. 11: The vaccinated MfPV3 infected female macaques were stratified by whether they had a known persistent infection (red) or not (blue). Persistent infection was defined by animals being MfPV3 positive at both screening, re-screening, and confirmatory test, as indicated in FIG. 1 and Table 1. Animals who were only tested for cervical MfPV3 presence once prior to enrolling in the vaccine trial are categorized as unknown persistency stage and were chosen as a comparator group, as it was not prioritized to include a non-infected control group in the vaccine trial. Animals were immunized as shown in FIG. 3A. Each dot represents an individual animal. “ns” on the right side of the plots refer to comparison of animals depending on their MfPV3 infection persistency status. Asterisks above the plot refers to a comparison of T cell responses of all vaccinated animals at the specific timepoint (A and B) or against the specific antigen (C), irrespective of their MfPV3 infection persistency status. A: induction of IFN-γ producing PBMCs, measured by ELISpot after 18-20 hours of peptide stimulation, before immunization and 14 days after prime immunization. B: IFN-γ producing CD8+ T cells measured by ICS and flow cytometry after 12-16 hours of peptide stimulation before boost-immunization and 14 days after boost immunization. C: Functionality of the CD8+ T cells was measured by the geometric mean fluorescence intensity (MFI) of the IFN-γ signal. Bars represent the geometric mean of all vaccinated animals, irrespective of their MfPV3 infection persistency status.

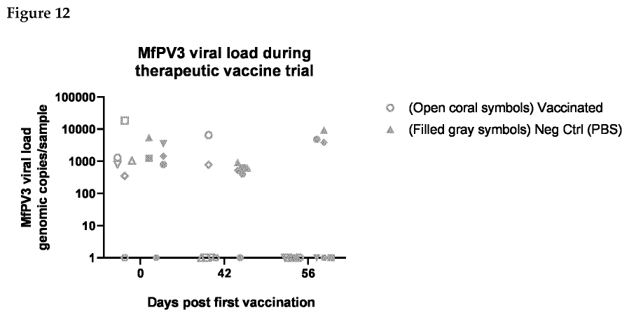

FIG. 12: MfPV3 viral load in the cervix of NHPs over time. Ad19a/64 prime, MVA boost vaccination with MfPV3 Ii-E1E2E6E7 cleared the infection in all vaccinated animals, compared to spontaneous clearance in 3 out of 6 of PBS injected negative ctrl NHPs.

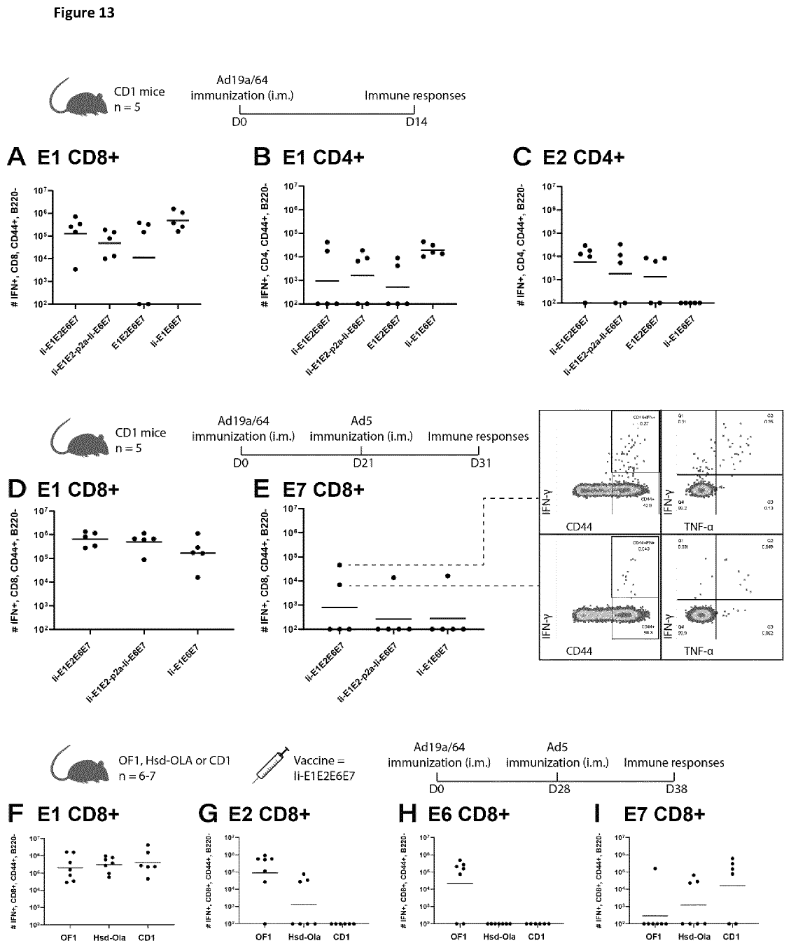

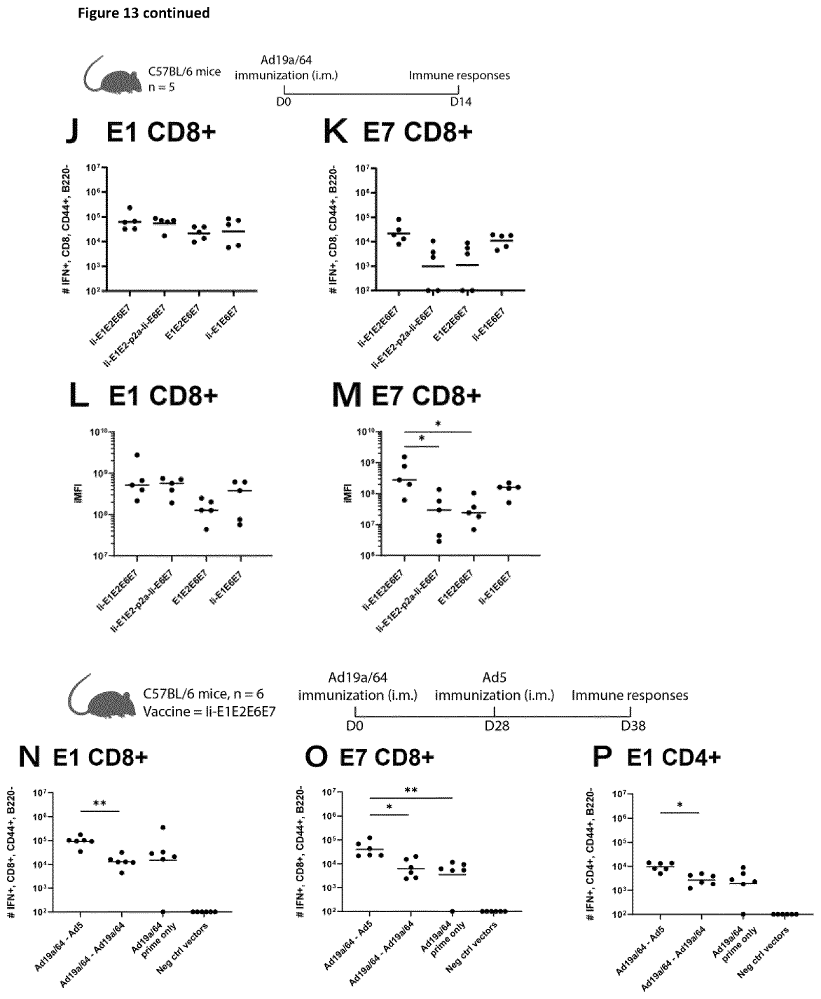

FIG. 13: CD1 mice (A-E), different outbred mice as indicated (F-I) or C57BL/6 mice (J-P) were immunized with Ad vaccine(s) (2×10⁷ IFU) encoding the various HPV16 early antigens as indicated. Mice were sacrificed at the timepoints indicated, spleens were harvested and CD8+ and CD4+ T-cell immune responses against E1, E2, E6, and E7 were measured using intracellular staining and flow cytometry. Each symbol represents one mouse, bars represent the geometric mean. For the two E7-responder mice (E), CD44+IFN-γ+ population and the populations of TNF-α+ and IFN-γ+CD8+CD44+ cells are shown.

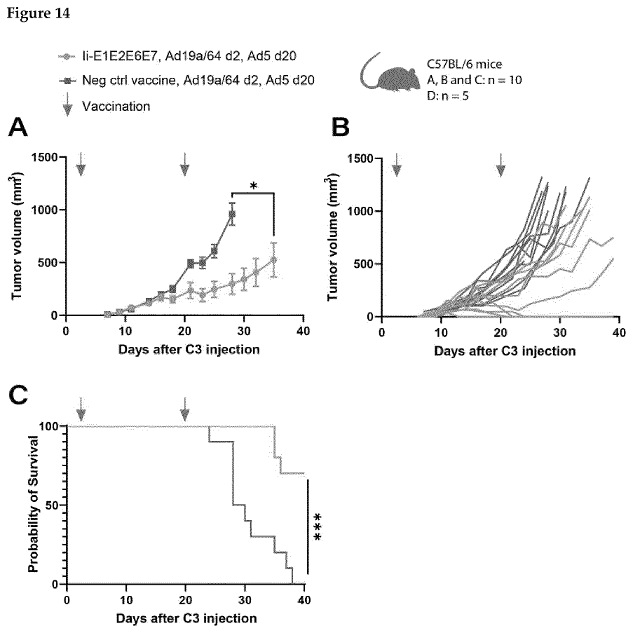

FIG. 14: 1×10⁶ C3 cells were injected s.c. into the flank of C57BL/6 mice, and the mice received Ad19a/64- and Ad5-vectored vaccines i.m. in the same side as the tumor on day 2 and day 20, respectively. Tumor growth (A and B) and survival (C). Pink curves with circles represent the therapeutic vaccine group. Gray lines with squares depict negative control vaccinated mice. Green arrows indicate time of vaccination.

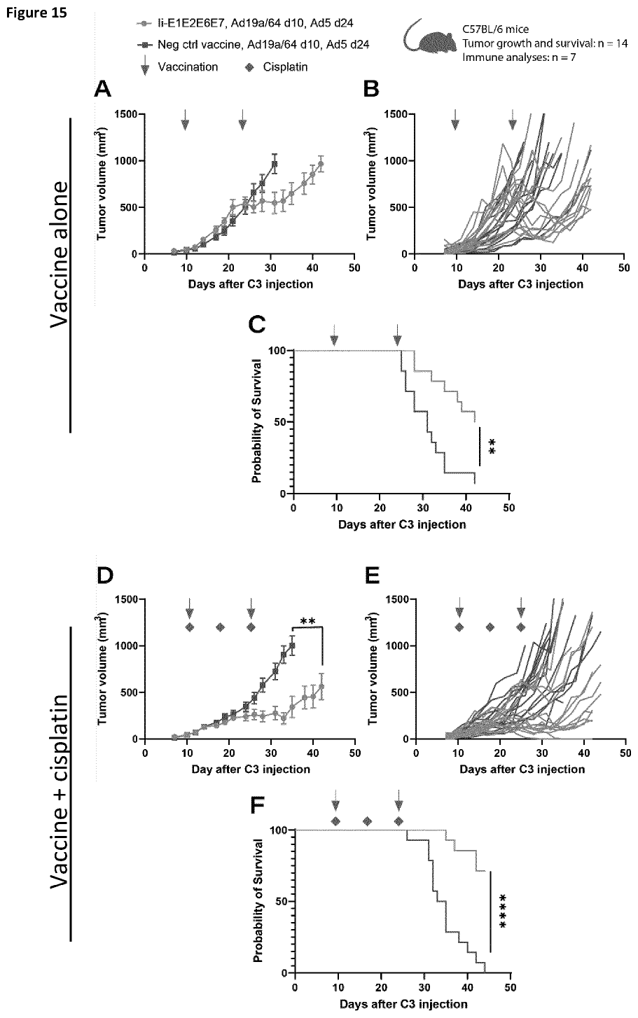

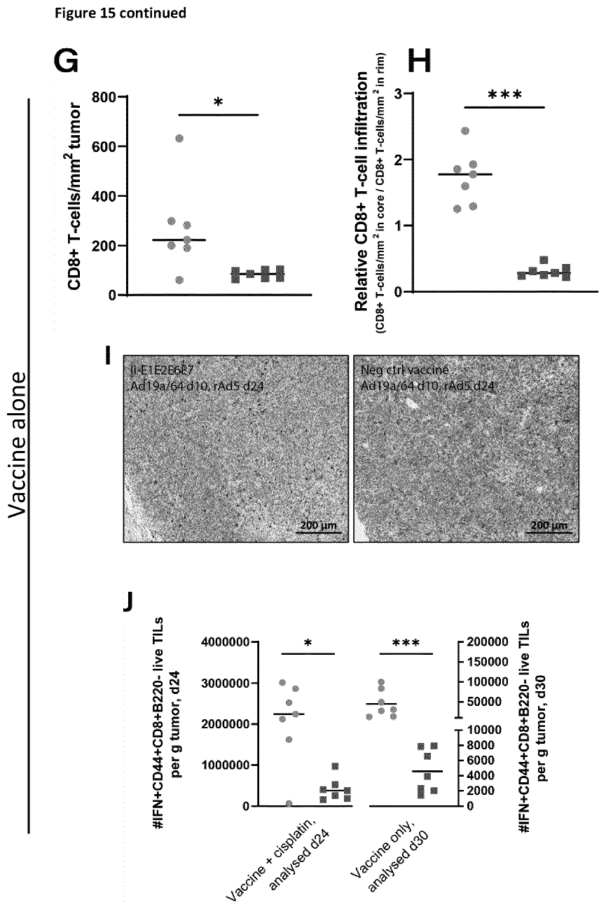

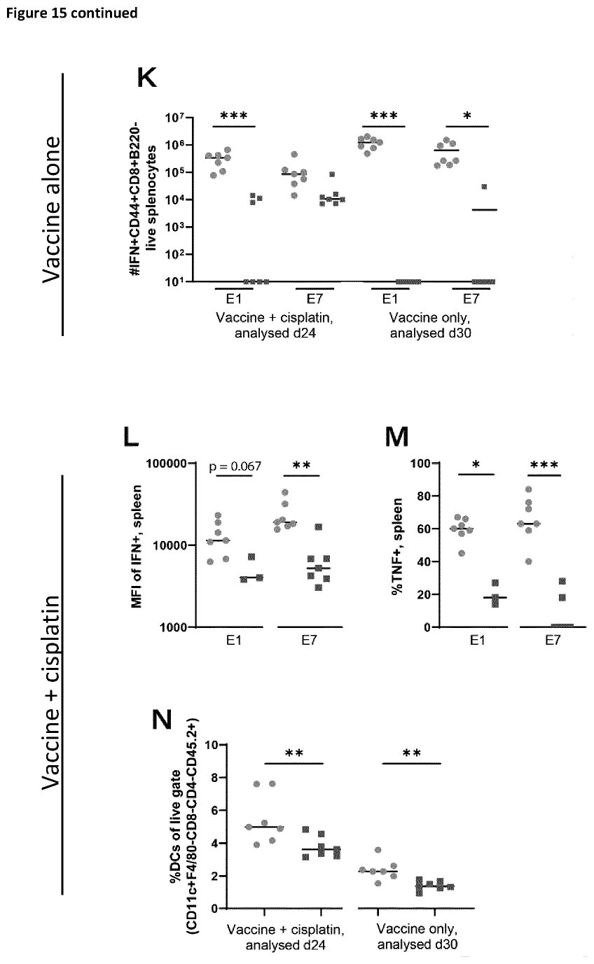

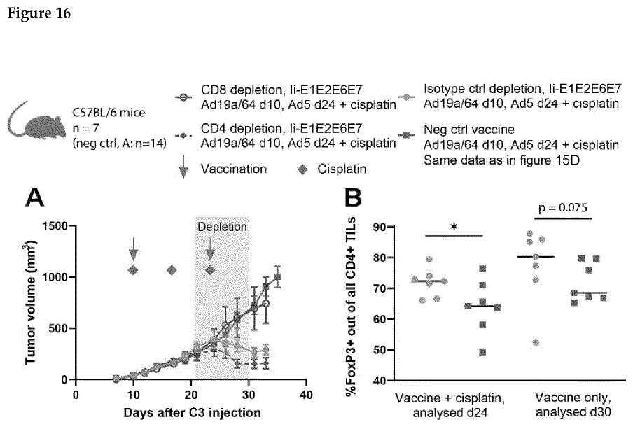

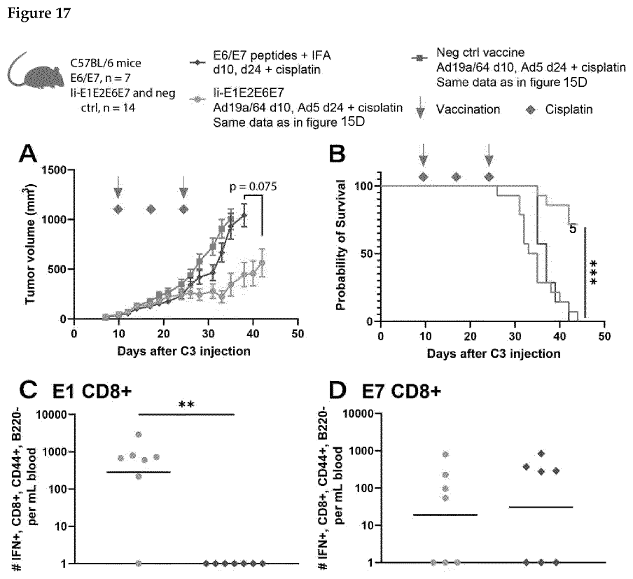

FIG. 15: 1×10⁶ C3 cells were injected s.c. into the flank of C57BL/6 mice, and the mice received Ad19a/64- and Ad5-vectored vaccines i.m. in the same side as the tumor on day 10 and day 24, respectively. Mean tumor growth with SEM (A), tumor growth for individual mice (B) and survival (C) was assessed. In Figure D-F, all mice were additionally treated with cisplatin on days 10, 17 and 24 (green diamonds).

Tumors of mice receiving therapeutic vaccine or negative control vaccine alone were harvested either once the tumor exceeded 1000 mm³ or at the end of the trial (day 42) and CD8+ cells were detected by immunohistochemistry. G: total number of CD8+spots per mm² tumor. H: ratio of CD8+spots per mm² tumor in the core vs the rim of the tumor. I: representative images of immunohistochemistry staining of a tumor from either a vaccinated (left) or a negative control vaccinated (right) mouse. The pictures show the rim (bottom left side of the pictures) and a bit of the core (top right side of the pictures). J: Mice were sacrificed at the time-points indicated, tumors were harvested and the resulting single cell suspension of a mix of tumor and immune cells were incubated for 5 hours without the presence of additional HPV16 peptides. IFN-y producing CD8+ T-cells were detected by intracellular staining and flow cytometry. K: Mice were sacrificed at the time-points indicated, spleens were harvested and CD8+ T-cell immune responses following E1 and E7 peptide stimulation were measured using intracellular staining and flow cytometry. L and M: Mean fluorescence intensity of IFN-γ (L), and the percentage of TNF-α+of all CD8+CD44+IFN-γ+splenocytes (M), for mice treated with cisplatin and vaccinated with therapeutic vaccine of negative control vaccine. N: tumors were harvested at the time indicated, single cell suspensions were prepared, and the fraction of DCs (CD11c+F4/80-CD8-CD4-CD45.2+TER119-live) was assessed by surface staining and flow cytometry.

Pink circles represent the therapeutic vaccine group. Gray squares depict negative control vaccinated mice. Green arrows indicate time of vaccination.

Green diamonds indicate time-point of cisplatin injections. G-N: Black bars indicate geometric mean. Figure A-F shows data pooled from two independent experiments.

FIG. 16: T1x106 C3 cells were injected s.c. into the flank of C57BL/6 mice, and the mice received Ad19a/64- and Ad5-vectored vaccines i.m. in the same side as the tumor on day 10 and day 24, respectively. Cisplatin treatment was given i.p. on days 10, 17 and 24. A: Mean tumor growth with SEM of mice treated i.p. with CD4-(green dashed, diamonds), or CD8-depleting (blue, open cicles) or isotype-control antibodies (pink, circles) on days 20, 23 and 26. The gray curve shows neg ctrl vaccinated mice for comparison (note that this is the same data as depicted in FIG. 4D, and pooled from two independent experiments). Green arrows indicate time of vaccination. Green diamonds indicate time-point of cisplatin injections. B: FoxP3 expression on tumor infiltrating CD4+CD8-CD45.2+TER119 live cells in mice li-E1E2E6E7 (pink circles) or neg ctrl (gray squares) vaccination with or without co-treatment with cisplatin. Tumor infiltrating cells were analysed on day 24 (combination treatment groups) or day 30 (vaccination alone) by intracellular staining and flow cytometry.

FIG. 17: 1×10⁶ C3 cells were injected s.c. into the flank of C57BL/6 mice, and the mice received Ad19a/64- and Ad5-vectored vaccines i.m. in the same side as the tumor, or peptide-based vaccine s.c. in the opposite flank on day 10 and day 24. Cisplatin treatment was given i.p. on days 10, 17 and 24. A and B: mean tumor growth with SEM (A) and survival curves (B) of peptide vaccine (blue, diamonds), Ii-E1E2E6E7*(pink, circles) or neg ctrl*(gray, squares). *Note, that this is the same data as depicted in FIG. 15D, and pooled from two independent experiments. Green arrows indicate time of vaccination. Green diamonds indicate time point of cisplatin injections. C and D: CD8+ T-cell responses (IFN-γ+CD44+CD8+CD4-B220-live cells) against E1 (C) and E7 (D) measured in the blood of Ii-E1E2E6E7 (pink circles) or peptide (blue diamonds) vaccinated mice on day 22 using intracellular staining and flow cytometry.

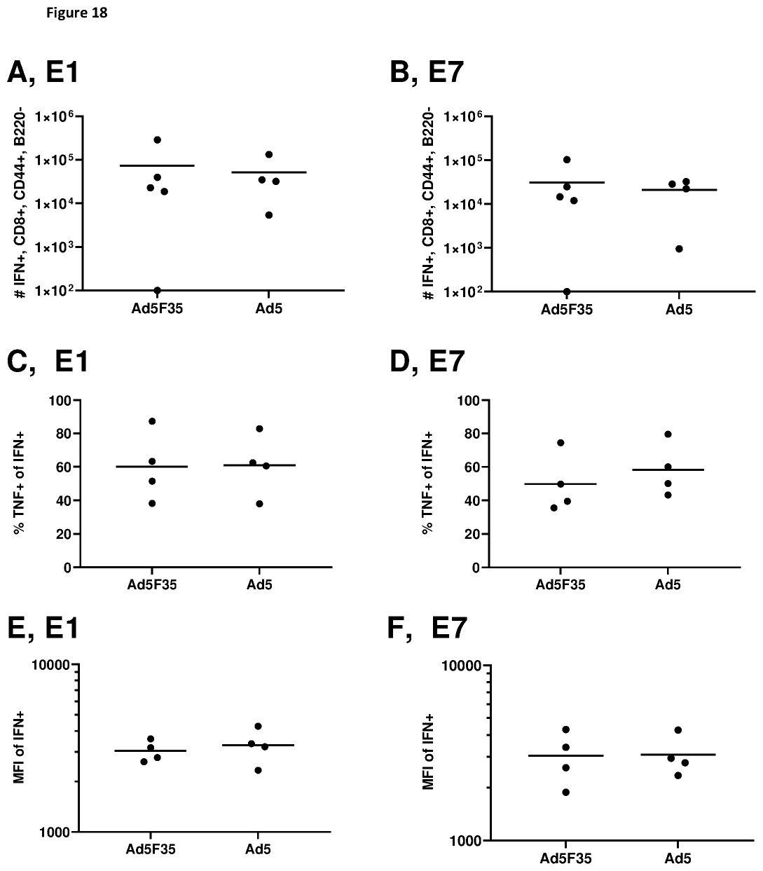

FIG. 18: C57BL/6 mice were immunized in the lower limb with rAd5 or Ad5F35 encoding HPV16 Ii-E1E2E6E7. Both vaccines induced CD8+ T-cell responses against E1 and E7 of similar magnitude and quality, indicating that Ad5F35 are equally efficient in induction of vaccine specific T-cell responses.

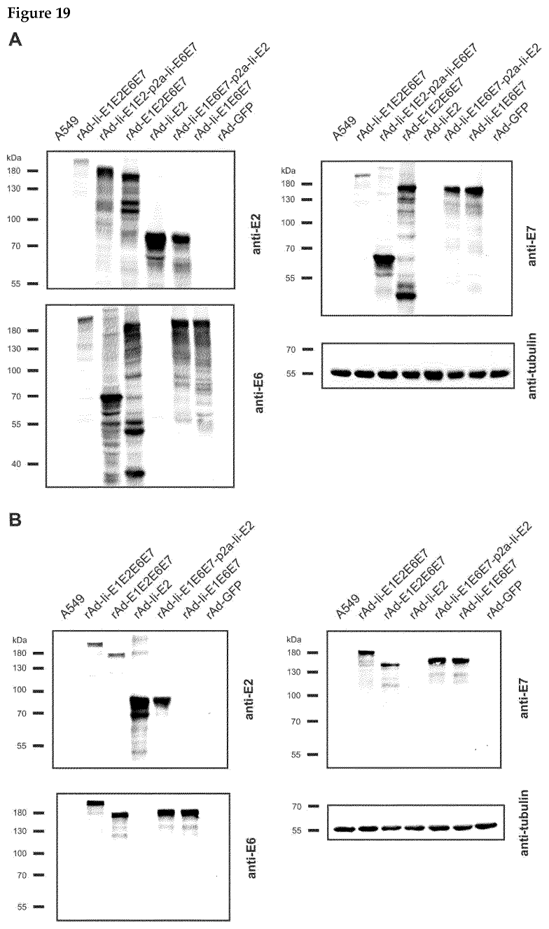

FIG. 19: Expression analysis of rAd-shuttled HPV16 and HPV18 antigen constructs. A. Western blot analysis of A549 cell lysates 48 h following transduction with rAds at an MOI of 100. Antigens were detected with anti-HPV16-E2 (upper left panel) and anti-HPV16-E6 (lower left panel), anti-HPV16-E7 (upper right panel) antibodies. As loading control, tubulin levels were monitored using an anti-tubulin antibody (lower right panel). B. Western blot analysis of A549 cell lysates 48 h following transduction with rAds at an MOI of 100. Antigens were detected with anti-HPV18-E2 (upper left panel) and anti-HPV18-E6 (lower left panel), anti-HPV18-E7 (upper right panel) antibodies. As loading control, tubulin levels were monitored using an anti-tubulin antibody (lower right panel).

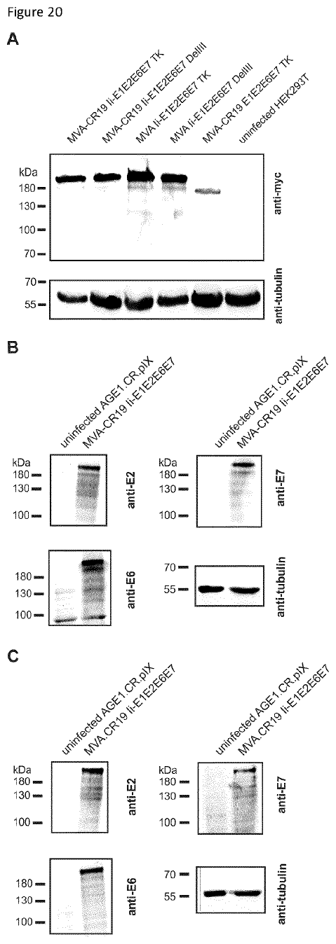

FIG. 20: Expression analysis of MVA-shuttled MfPV3, HPV16 and HPV18 antigen constructs.

A. Western blot analysis of HEK293T cell lysates 24 h following transduction with recombinant MVA or MVA-CR19 containing MfPV3 antigen in DelIII locus or TK locus at an MOI of 10. Antigens were detected anti-myc antibody (upper panel). As loading control, tubulin levels were monitored using an anti-tubulin antibody (lower panel). B. Western blot analysis of AGE1.CR-pIX cell lysates 24 h following transduction with MVA-CR19 containing HPV16 Ii-E1E2E6E7 in DelIII locus at an MOI of 1. Antigens were detected with anti-HPV16-E2 (upper left panel) and anti-HPV16-E6 (lower left panel), anti-HPV16-E7 (upper right panel) antibodies. As loading control, tubulin levels were monitored using an anti-tubulin antibody (lower right panel). C. Western blot analysis of AGE1.CR-pIX cell lysates 24 h following transduction with MVA-CR19 containing HPV18 Ii-E1E2E6E7 in DelIII locus at an MOI of 1. Antigens were detected with anti-HPV18-E2 (upper left panel) and anti-HPV18-E6 (lower left panel), anti-HPV18-E7 (upper right panel) antibodies. As loading control, tubulin levels were monitored using an anti-tubulin antibody (lower right panel).

The Examples illustrate the invention.

### Example 1: Constructs of the Invention and their Performance

### Methods

### Antigen Sequences

Parts of the antigens were synthesized at Geneart/Thermo Fisher (Regensburg, Germany). The gene optimizer algorithm was used to minimize sequence homology and adapt the sequences to human codon usage. All constructs were cloned using standard molecular biology methods. Briefly, antigens were assembled with fusion PCR, type IIs exocutter sites (BsaI-HF v2, New England Biolabs, Ipswich, USA) or NEBuilder HIFI DNA Assembly Kit (New England Biolabs, Ipswich, USA) according to manufacturer's instructions. Constructs were cloned into the different plasmid backbones using AgeI-HF and NotI-HF (New England Biolabs, Ipswich, USA). Sequence correctness was verified by Sanger sequencing. Plasmids were prepared, depending on amount, with alkaline lysis or commercially available kits according to manufacturer's instructions (Plasmid Midi plus, EndoFree Plasmid Mega Kit, Qiagen, Hilden, Germany). The sequence of the antigen Ii-E1E2-p2a-li-E6E7 (FIG. 1) was assembled by connecting the amino acid sequence of li (NM_004355.3) with E1, E2, E6, and E7 of MfPV3 (EF558839.2). Following E2, the porcine teschovirus-1 2A sequence (Liu, Z. et al. Systematic comparison of 2A peptides for cloning multi-genes in a polycistronic vector. Sci. Rep. 7, 2017) was inserted, followed by a second li-sequence. E1 was linked to E2, and E6 to E7, via a GS-linker, respectively. Mutations were introduced into E6 and E7 to inactivate the oncogenic potential: L110Q and deletion of C-terminal ETEV in E6; D24G, L71R, C95A; C297A was introduced into E2 to inactivate DNA-binding. The sequence encoding this fusion protein was optimized and adapted to human codon-usage by the GeneOptimizer-algorithm (Raab, D., Graf, M., Notka, F., Schödl, T. & Wagner, R. The GeneOptimizer Algorithm: using a sliding window approach to cope with the vast sequence space in multiparameter DNA sequence optimization. Syst. Synth. Biol. 4, 215-225, 2010), synthesized by GeneArt/ThermoFisher (Regensburg, Germany) and cloned into pDONR221. The other constructs (FIG. 1) were built by using standard molecular cloning techniques using the above construct as template and cloned into the desired plasmid backbones. DNA for vaccination purposes was produced using the EndoFree Plasmid Mega Kit (Qiagen, Hilden, Germany) according to the manufacturer's instructions.

### Cell Lines, Transfection and Viral Infection

HEK293T cells and A549 cells were maintained and grown in Dulbecco's MEM (DMEM) supplemented with 10% Fetal Calf Serum (FCS) and 1% Penicillin/Streptomycin (Pen/Strep). 9E10 mycl hybridoma cells were cultivated in RPMI supplemented with 10% FCS, 1% Pen/Strep and 2 mM glutamine (Pan). All cell lines were maintained at 37° C. and 5% CO₂ in a non-humidified incubator.

HEK293T cells were transfected using the polyethylenimide (PEI) method (Boussif, O. et al. A versatile vector for gene and oligonucleotide transfer into cells in culture and in vivo: polyethylenimine. Proc. Natl. Acad. Sci. 92, 7297 LP-7301, 1995). For PEI transfection, 4×10⁵ cells were seeded in 6-well plates one day before transfection. The cells were transfected with 2.5 μg plasmid (equimolar amounts, filled with empty vector) and 7.5 μg PEI in DMEM without any supplements. After 6 h incubation, medium was exchanged to DMEM with 10% FCS and 1% Pen/Strep.

Subconfluent A549 cells were infected with Ad19a/64 vectors at an MOI of 30 in DMEM without any supplements. 2 h post infection, medium was exchanged to DMEM with 10% FCS and 1% Pen/Strep.

### Generation of Adenoviral Vectors

E1/E3 deficient adenoviral vectors of serotype Ad19a/64 were generated as previously described (Ruzsics, Z., Lemnitzer, F. & Thirion, C. Engineering Adenovirus Genome by Bacterial Artificial Chromosome (BAC) Technology BT-Adenovirus: Methods and Protocols. in (eds. Chillón, M. & Bosch, A.) 143-158 (Humana Press, 2014). doi: 10.1007/978-1-62703-679-5_11). Briefly, the Genes of Interest (GOI)-(FIG. 1) were cloned into the shuttle vector pO6-19a-HCMV-MCS under control of a CMV promoter. The CMV-GOI-SV40-pA was then transferred via Flp-recombination in E. coli into a BAC vector (Ruzsics, Z. et al, 2014) containing the genome of a replication deficient Ad-based vector deleted in E1/E3 genes. Recombinant viral DNA was released from the purified BAC-DNA by restriction digest with PacI. The obtained linear DNA was transfected into the production cell line disclosed above for virus propagation. Recombinant viruses were released from cells via sodium deoxycholate treatment. Residual free DNA was digested by DNase I. Afterwards, vectors were purified by CsCl gradient ultracentrifugation followed by a buffer exchange to 10 mM Hepes pH 8.0, 2 mM MgCl2 and 4% Sucrose via PD10 columns (GE Healthcare, Chicago, USA). Titration was performed using the RapidTiter method by detection of infected production cells via immunohistochemical staining with anti-hexon antibody (Novus, Adenovirus Antibody (8C4)). Insert integrity was confirmed by PCR amplification of the GOI in DNA extracted from the purified vectors.

### Antibodies and Antibody Purification

The antibody against myc (9E10) was obtained from hybridoma cell supernatants. 9E10 mycl hybridoma cells were seeded at 5×10⁵ cell per ml in RPMI supplemented with 1% FCS, 1% Pen/Strep and 2 mM glutamine. The supernatant was harvested 5 days after seeding and the antibody was purified via a HiTrap Protein G column (GE Healthcare, Chicago, USA). After washing the column with PBS, the antibody was eluted with 0.1 M glycine/HCl (pH 3.2), neutralized with 0.025 volumes of 1 M Tris/HCl (pH 9) and dialyzed against PBS. Other antibodies used were: mouse anti-p2a peptide (3H4, 1:2000, Merck, Darmstadt, Germany), mouse anti-tubulin (DM1α, 1:1000, Santa Cruz, Heidelberg, Germany), mouse anti-ubiquitin-Biotin (eBioP4D1, 1:1000, Invitrogen, Carlsbad, USA), goat anti-mouse-HRP (115-036-003, 1:5000, Jackson, West Grove, USA), goat anti-rabbit-HRP (P0448, 1:2000, Dako, Santa Clara, USA), Streptavidin-HRP (11089153001, 1:5000, Roche, Basel, Swiss), rat anti-mouse-PE (A85-1, 1:100, BD, Franklin Lakes, USA).

### Western Blot Analysis

Western blot analysis was performed as previously described (Kiener, R. et al. Vaccine vectors based on Adenovirus 19a/64 exhibit broad cellular tropism and potently restimulate HCMV-specific T cell responses ex vivo. Sci. Rep. 8, 1474, 2018). Briefly, cells of interest were lysed in TDLB buffer (50 mM Tris, pH 8.0, 150 mM NaCl, 0.1% SDS, 1% Nonident P-40, 0.5% sodium deoxycholate) supplemented with protease inhibitors (Complete Mini, Roche, Basel, Swiss). Total protein concentration of the supernatants was measured by the Bradford method (Protein Assay, BioRad, Feldkirchen, Germany). The proteins were separated on SDS-PAGE under reducing conditions and blotted on a nitrocellulose membrane for western blot analysis. Targets were probed with primary and secondary antibodies as listed above. HRP-labeled secondary antibodies and enhanced chemiluminescence substrate or Femto ECL (Thermo Fisher, Waltham, USA) were used for detection in a Chemilux Pro device (Intas, Göttingen, Germany). For loading control, the membrane was reprobed with an antibody against tubulin.

### Flow Cytometry Analysis of Cell Lines

Intracellular staining of antigens was performed using standard methods (Kiener et al., 2018). Cells were fixed and permeabilized with Cytofix/Cytoperm-Buffer (4% PFA, 1% saponine, in PBS). All washing steps were done with Perm/Wash-Buffer (PBS containing 0.1% saponine). The cells were stained with anti-myc antibody (5 μg/ml, diluted in Perm/Wash-Buffer) and rat anti-mouse-PE (1:100 diluted in Perm/Wash-Buffer) each for 30 min.

The flow cytometry assay was performed using an Attune N×T device (Thermo Fisher, Waltham, USA) with a 488 nm excitation and a 574/26 nm emission filter. Cells were gated on stained, mock-transfected cells. Evaluation of data was performed using Attune N×T software.

### Analysis of Ubiquitination

To analyse ubiquitinylated proteins, 24 h post transfection, cells were treated with 10 μM MG132 proteasome inhibitor for 6 h. Afterwards, cells were harvested in PBS and washed twice. For inactivation of deubiquitination enzymes, 20 mM N-ethylmaleimide from a freshly prepared stock solution were added to the TDLB lysis buffer. Lysates were generated as described above. Before immunoprecipitation, Protein G dynabeads (Thermo Fisher, Waltham, USA) were loaded with 10 μg of pulldown antibody. Using these beads, target protein was immunoprecipitated out of 500 μg cell lysate over night at 4° C. under slow rotation. After washing the beads four times with PBS, SDS-PAGE buffer was added to the beads before heating at 95° C. for 10 min. The samples were used for western blot analyses as described above.

### Animals and Immunizations

Balb/C and CD1 female mice were obtained from Envigo (Horst, The Netherlands) and OF1 mice from Charles River (France). All animals were housed at the Panum Institute, University of Copenhagen. All experiments were initiated after allowing the mice to acclimatize for at least 1 wk. Experiments were approved by the National Animal Experiments Inspectorate (Dyreforsøgstilsynet, license no. 2016-15-0201-01131) and performed according to national guidelines.

DNA immunizations were performed intradermal (i.d.) with 0.5 g DNA coated onto 1.6 μm gold microcarriers (BioRad, Feldkirchen, Germany) using the Helios Genegun System (BioRad, Feldkirchen, Germany). The mice received four DNA immunizations at intervals of one week each. One group received a mixture of four plasmids, 0.5 μg each, into one site. Immunizations with adenoviral vectors were performed intramuscular (i.m.) with 2×10⁷ IFU Ad19a/64 diluted in 50 μL PBS. Mice were anesthetized with isofluorane before Ad19a/64 injections. One group received a mixture of four Ad19a/64, 2×10⁷ IFU each, into one site.

### Flow Cytometry of Splenocytes

Single cell suspensions of splenocytes were obtained by organ harvest in HANKs followed by straining through 70 μm cell strainers. Cells were incubated for 5 hours in 3 μM monensin with or without 1 μg/mL of relevant peptides. The cells were stained against surface markers: APC-Cy7 or BV421 CD8 (53-6.7, 1:200, BioLegend, San Diego, USA), PE-Cy7 CD4 (RM4-5, 1:800, BD), FITC CD44 (IM7, 1:100, BioLegend, San Diego, USA) and PerCP-Cy5.5 B220 (RA3-6B2, 1:200, BioLegend, San Diego, USA). After surface staining, cells were fixed in 1% PFA, permeabilized in 0.5% saponine, and stained intracellularly using APC IFN-γ (XMG1.2, 1:100, BioLegend, San Diego, USA) and PE TNF-α (MP6-XT22, 1:100, BioLegend, San Diego, USA) antibodies.

The peptides used were 16-mers overlapping by 11 amino acids covering the entire Ii-E1E2E6E7 antigen. The peptides were pooled in 5 separate pools containing Ii, E1, E2, E6 and E7 peptides respectively. Peptides were obtained from KareBay, Town, China.

Flow cytometry was performed on the Fortessa 3 (BD Biosciences, Franklin Lakes, USA) flow cytometer and data analysis was performed using FlowJo V10 software. Epitope-specific CD8+ T-cell responses were measured as B220, CD8+ or CD4+, CD44+, IFN-Y′ cells and are presented in total number of cells per organ. The quality of the IFN-γ responses were evaluated by MFI of IFN-γ and fraction of double positive cells (expressing both IFN-γ and TNF) in the IFN-Y CD8+ or CD4+ populations.

### In Vivo Cytotoxicity

The assay was performed similarly to what was previously described (Nielsen, K. N., Steffensen, M. A., Christensen, J. P. & Thomsen, A. R. Priming of CD8 T cells by adenoviral vectors is critically dependent on B7 and dendritic cells but only partially dependent on CD28 ligation on CD8 T cells. J. Immunol. 193, 1223-1232, 2014). Briefly, splenocytes from naïve Balb/C mice were incubated with MfPV3 E1, E2, E6 or E7 peptide pools or no peptide for 30 minutes at 37° C., 5% CO₂, 2.5 μg of each peptide/mL, and subsequently stained with combinations of 0.4 or 5 μM CellTrace CFSE and 0.2 and 2.5 μM CellTraceViolet (CTV; ThermoFisher, Waltham, USA) for 10 minutes at 37° C., 5% CO₂. Pulsed and stained splenocytes were mixed at a 1:1:1:1:1 ratio, and a total of 2.5×10⁷ cells were injected intravenously into Ad19a/64-vaccinated recipient Balb/C mice. 5 hours later, spleens were harvested, and target cells were identified on the Fortessa 3 (BD Biosciences, Franklin Lakes, USA) flow cytometer by CFSE/CTV staining. The percentage of killing was calculated using the following equation:

% targeted killing=100−(% non-peptide pulsed cells in vaccinated mice/% peptide pulsed cells in vaccinated mice/% peptide pulsed cells in non-vaccinated mice/% non-peptide pulsed cells in non-vaccinated mice)*100

### Analysis of E1 (SIINFEKL) Presentation on MHC-I

HEK293T cells were transfected with 2.5-3.5 μg DNA using PEI transfection. 48 hours post transfection, cells were stained with PE anti-H2 KB-SIINFEKL (25-D1.16, 1:160, Invitrogen, Carlsbad, USA) and presence of SIINFEKL-H2Kb presentation on cell surfaces was detected on the LSRII or Fortessa 3 (BD Biosciences, Franklin Lakes, USA) flow cytometers, as a proxy for E1 presentation. All samples were run in biological 6-plicates, and the experiment was repeated at least two times.

### Graphical Representation and Statistical Analysis of Ex Vivo Immunogenicity Data

Non-stimulated samples were used as background controls, and their response values have been subtracted from the peptide-stimulated samples of the corresponding animal before performing statistical analysis and graphical presentation.

Statistical analysis was done using GraphPad Prism 8 software (GraphPad Software, San Diego, USA). Values with background subtracted were used to compare the individual groups using the Mann-Whitney test with Bonferroni correction for multiple comparisons. Positive control groups were not included in the multiple comparison analyses.

In order to aid visual presentation of the results, we applied a threshold for responses based on the average number of B220⁻, CD8⁺, CD44⁺, IFN-γ⁺ counts for unstimulated background samples. All samples with less counts than the average+2×SD of the background samples, are regarded as non-responding and therefore they have manually been adjusted to a value of 100 for the graphical presentation. All statistical analyses were done on non-adjusted values.

The analysis of fraction of double positive (% TNF⁺IFN-γ⁺ out of all IFN-γ⁺) and MFI of IFN-γ⁺ was only performed on responders (IFN-γ⁺ count>avg+2×SD of unstimulated samples). Each symbol represents one mouse. Bars indicate median. Significance levels are marked by * (p<0.05), **(p<0.01), ***(p<0.005).

### Results

### Design of the Antigens

With the aim of generating a therapeutic vaccine capable of eliminating pre-existing papillomavirus infections in non-human primates, antigen constructs comprising E1, E2, E6, and E7 of MfPV3 linked to the intrinsic T-cell adjuvant li were designed. Eight different configurations were conceived (FIG. 1) to select the one eliciting the most potent and broadest response for further development. While in the first composite antigen the order was Ii-E1E2E6E7, the order of E1/E2 and E6/E7 was reversed in Ii_E6E7E1E2 (i) to test the influence of the antigen order, and (ii) to determine the impact of positioning relative to li on the level of transgene expression. In both cases, all proteins were expressed as artificial fusion polypeptides, whereas the individual proteins were separated by a short and flexible GS linker to promote the stability and accumulation of the fusion proteins (Chen, X., Zaro, J. L. & Shen, W.-C. Fusion protein linkers: property, design and functionality. Adv. Drug Deliv. Rev. 65, 1357-1369, 2013) and li was fused to the N-terminus as a T cell adjuvant (Holst, P. J. et al. MHC Class II-Associated Invariant Chain Linkage of Antigen Dramatically Improves Cell-Mediated Immunity Induced by Adenovirus Vaccines. J. Immunol. 180, 3339-3346, 2008). As a reference to assess the T cell adjuvant effects of li, the variant E1E2E6E7 without li was designed. To investigate if the li adjuvant effect was more pronounced when fused to shorter antigens, E1E2 were separated from E6E7 by a p2a peptide in Ii-E1E2-p2a-li-E6E7 (Kim, J. H. et al. High cleavage efficiency of a 2A peptide derived from porcine teschovirus-1 in human cell lines, zebrafish and mice. PLOS One 6, 2011). The nucleotide sequence homology between the two invariant chains was minimized by using divergent codons to hinder homologous recombination. Additionally, each of the four viral proteins was generated on its own as Ii-fusion to serve as benchmark and reference. Because of the lack of antibodies against MfPV3 early proteins, all constructs were modified to contain a C-terminal myc-tag.

Since E6 and E7 are highly potent oncoproteins, only transformation-defective variants should be used in the setting of a vaccine to ensure safety. Based on the homology to HPV16, the oncoprotein E6 was inactivated by introducing a L110Q substitution to prevent binding of E6 to E6BP and therefore binding and degradation of tumor suppressor p53 (Liu, Y. et al. Multiple Functions of Human Papillomavirus Type 16 E6 Contribute to the Immortalization of Mammary Epithelial Cells. 73, 7297-7307, 1999). Additionally, the C-terminal PDZ-domain was deleted (AETEV) to abolish binding of telomerase and other LxxLL proteins (Wieking, B. G. et al. A Non-oncogenic HPV 16 E6/E7 Vaccine Enhances Treatment of HPV Expressing Tumors. Cancer Gene Ther. 19, 667-674, 2013). E7 was inactivated by introducing the substitutions C24G, L71R, and C95A, which inactivate the central LxCxE motif to reduce transforming activity as well as pRB binding, reduce Mi2ß binding, and inhibit dimerization, respectively (Wieking, 2013; Edmonds, C. & Vousden, K. H. A Point Mutational Analysis of Human Papillomavirus Type 16 E7 Protein. 63, 2650-2656, 1989). The conserved DNA binding domain of E2 was modified by C297A to generate polyproteins with an even number of cysteins and reduce DNA binding.

For initial biochemical and immunological characterization, the constructs were cloned into pURVac, a derivative of a DNA vaccine vector with a proven track record in various NHP and clinical trials (Asbach, B. et al. Priming with a Potent HIV-1 DNA Vaccine Frames the Quality of Immune Responses prior to a Poxvirus and Protein Boost. J. Virol. 93, 2019; Sarwar, U. N. et al. Safety and immunogenicity of DNA vaccines encoding Ebolavirus and Marburgvirus wild-type glycoproteins in a phase I clinical trial. J. Infect. Dis. 211, 549-557, 2015; Joseph, S. et al. A Comparative Phase I Study of Combination, Homologous Subtype-C DNA, MVA, and Env gp140 Protein/Adjuvant HIV Vaccines in Two Immunization Regimes. Front. Immunol. 8, 149, 2017; Pantaleo, G. et al. Safety and immunogenicity of a multivalent HIV vaccine comprising envelope protein with either DNA or NYVAC vectors (HVTN 096): a phase 1b, double-blind, placebo-controlled trial. Lancet HIV 6, e737-e749, 2019) where antigens are under the control of a human cytomegalovirus (CMV) promoter in combination with a human T-cell leukemia virus-1 (HTLV-1) regulatory element (Barouch, D. H. et al. A human T-cell leukemia virus type 1 regulatory element enhances the immunogenicity of human immunodeficiency virus type 1 DNA vaccines in mice and nonhuman primates. J. Virol. 79, 8828-8834, 2005) and a bovine growth hormone poly-A terminator.

### Biochemical and Cell Biological Characterization of MfPV3 Antigens

Initially, expression properties of the antigen constructs were evaluated. All antigens were expressed in HEK293T cells with bands of the expected sizes being detected by western blot analysis using an anti-myc antibody (FIG. 2A; FIG. 1). A higher molecular weight band for a non-separated Ii-E1E2-p2a-li-E6E7 could neither be detected in the anti-myc western-blot detecting the C-terminal fragment, nor in the anti-p2a western blot where the N-terminal fragment is detected. This provides evidence that the polyprotein was completely separated at the p2a site. In some cases, especially for proteins with high signal intensities, bands of higher or lower molecular weight as compared to the calculated values could be observed. Based on their sizes, the bands of higher molecular weight may resemble incompletely reduced proteins, which is not completely surprising as li seems to form cysteine linked trimers (Fougeroux, C., Turner, L., Bojesen, A. M., Lavstsen, T. & Holst, P. J. Modified MHC Class II-Associated Invariant Chain Induces Increased Antibody Responses against Plasmodium falciparum Antigens after Adenoviral Vaccination. J. Immunol. 202, 2320-2331, 2019). Bands of lower molecular weight presumably resemble N-terminal degradation products that are most likely cleaved within li as based on the fragment sizes. Partial degradation is expected as li transports some of its linked cargo to endosomal compartments where proteolytic degradation occurs.

Since proteins of different sizes exhibit different blotting conditions, the western blot analysis could not be assumed to be quantitative, so signal intensities may not reflect the real expression levels. Therefore, protein levels were quantified by flow cytometry after intracellular staining with the anti-myc-antibody (FIG. 2B). Expression levels of the different MfPV3 antigens differed only marginally, except for li-E7, which exhibited two- to threefold higher MFI values compared to all other antigens.

MHC Class I-Restricted SIINFEKL Epitope is Processed from E1 Fusion Protein and Abundantly Presented on MHC-I Molecules In Vitro

It has been reported that bovine papillomavirus type 1 E1 is itself an unstable protein which is ubiquitinylated and rapidly degraded in papillomavirus-infected cells (Malcles, M.-H., Cueille, N., Mechali, F., Coux, O. & Bonne-Andrea, C. Regulation of bovine papillomavirus replicative helicase el by the ubiquitin-proteasome pathway. J. Virol. 76, 11350-11358, 2002; Mechali, F., Hsu, C.-Y., Castro, A., Lorca, T. & Bonne-Andrea, C. Bovine papillomavirus replicative helicase E1 is a target of the ubiquitin ligase APC. J. Virol. 78, 2615-2619 (2004). The constitutive degradation might explain why we have been unable to find any studies reporting the detection of E1 expression at the protein level in HPV infected cells and tissues, and why E1 is poorly immunogenic with respect to antibodies (Ewaisha, R., Panicker, G., Maranian, P., Unger, E. R. & Anderson, K. S. Serum Immune Profiling for Early Detection of Cervical Disease. Theranostics 7, 3814-3823, 2017). However, it should be confirmed that, despite being poorly immunogenic in natural HPV infections, E1-derived peptides are presented on MHC-I on infected cells, a prerequisite for being targetable by therapeutic vaccination. To detect presentation of E1-derived peptides, we designed a DNA construct encoding MfPV3 E1 in sequence with the minimal H2-kb-restricted OVA epitope SIINFEKL (Dersh, D., Yewdell, J. W. & Wei, J. A SIINFEKL-Based System to Measure MHC Class I Antigen Presentation Efficiency and Kinetics. Methods Mol. Biol. 1988, 109-122 (2019). Following transfection of the E1-SIINFEKL construct, transgene presentation on MHC-I was assessed using an H2-Kb-SIINFEKL specific antibody (FIG. 2C). Processed and presented SIINFEKL-peptide could be detected on MHC-I on cell surfaces, both when linked to li and when delivered alone. This supports the choice of E1 as a relevant antigen to include in the therapeutic vaccine, as E1-derived peptides can be expected to be presented on the surface of infected cells.

DNA Vaccination of Antigen Constructs Induces CD4⁺ and CD8⁺ T Cell Responses Against MfPV3 Early Antigens

Intradermal (i.d.) DNA immunization of outbred CD1 mice confirmed that all pURVac DNA vaccines encoding E1 and/or E2 induced E1- and E2-specific IFN-γ⁺ CD8⁺ and CD4⁺ T cells measured by ICS and flow cytometry analysis (FIG. 3. More detailed, all DNA vaccine candidates had provided an E1 response on par with the positive control, whereas there was a trend to higher responses for the Ii-E2 positive control. No vaccines induced responses against E6 or E7 in this outbred strain of mice, including the positive controls for these two antigens. Importantly, all vaccines encoding all four antigens as a polyprotein generated responses comparable to those induced by a mix of four vaccines encoding the antigens individually (Ii-E1, Ii-E2, Ii-E6, Ii-E7). This suggests that overall no immunogenicity was lost by delivering the MfPV3 early antigens as polyproteins from a single pURVac DNA vaccine. Altogether, the four different polyprotein encoding DNA vaccines induced T cell responses, which showed no statistical differences regarding the frequencies of E1- and E2-specific IFN-γ⁺ CD8⁺ and CD4+ T cells. This is the case regardless (i) of whether li is fused to E1E2E6E7, (ii) of the order of the early antigens in the polyprotein, and (iii) of the use of a p2a site to separate Ii-E1E2 from Ii-E6E7. The quality of the T-cell responses was comparable across all vaccines, assessed by MFI of IFN-γ and TNF production of IFN-γ T-cells. As expected, Ii gave rise to CD8 responses in most mice, and to CD4 in some mice, as the human li is allogenic in mice. The vaccine with two p2a-separated li-linked antigen sequences (Ii-E1E2-p2a-Ii-E6E7) and Ii-E6E7E1E2 appeared to elicit slightly better CD4 responses against E1 shown by the significant response and significant difference to the negative control group (FIG. 3A).

### Characterization of Adenoviral Vectors

To increase the cellular immune response against the antigens, viral vectors were subsequently used for antigen delivery. Adenoviral vectors from serotype 19a/64 (rAd) had been shown to be a suitable vector for the delivery of MfPV3 antigens that were capable of inducing high CD8⁺ T cell response in cynomolgus macaques (Ragonnaud, E. et al. Replication deficient human adenovirus vector serotype 19a/64: Immunogenicity in mice and female cynomolgus macaques. Vaccine 36, 6212-6222, 2018). A refined panel of rAd vectors was generated comprising a modified set of recombinant MfPV3 antigens. Ii-E6E7E1E2 was excluded because it was not superior to the other polyproteins. The trend toward higher responses against E2 when administered alone led us to speculate whether it could be advantageous to have E2 individually linked to li and expressed as separate domain. Hence, a vaccine encoding an N-terminal li linked to E1, E6 and E7 (Ii-E1E6E7) without E2 as reference, and a version in which the Ii-E1E6E7 antigen was extended by an li-E2 fusion via a self-separating p2a peptide (Ii-E1E6E7-p2a-Ii-E2) was designed. In order to confirm proper expression of the encoded antigens, western blot analysis was performed with adenovirally transduced A549 cells. All vectors readily expressed the antigens with bands resembling the respective fusion proteins being detected at the expected positions (FIG. 4A, FIGS. 1 and 2). As for li-E1E2-p2a-Ii-E6E7, no higher molecular weight fragment could be detected for li-E1E6E7-p2a-li-E2, revealing that the complete separation at the p2a site also worked in the context of adenoviral-vector-delivered antigens. Quantitative comparison of the adenovirally delivered antigens in A549 cells using flow cytometry confirmed similar expression levels of the different MfPV3 antigen constructs with the exception of higher expression of the li-E7 fusion protein (FIG. 4B. The overall picture was quite similar to the expression observed after delivery of pURVac DNA vaccine vectors in HEK293T cells.

### Adenoviral Delivery Induces Potent Cellular Immune Responses Against HPV Early Antigens in Outbred Mice

Intramuscular immunization of outbred CD1 mice confirmed that the Ad19a/64-formulation of the vaccines induced both CD8⁺ and CD4⁺ T cell responses against E1 and E2 antigens (FIGS. 5A and B). Notably, vaccines including li showed a trend towards higher magnitude responses than the vaccine not encoding li (FIGS. 5A and B: E1E2E6E7 compared to other vaccines). A tendency of li-mediated enhancement of responses was observed for both CD4 and CD8 T cell responses for some vaccine configurations, but the difference was only significant for E2-specific CD8 responses induced by Ii-E1E2-p2a-Ii-E6E7 compared to E1E2E6E7 (FIG. 5B, upper panel). This underlines the ability of li to boost T cell responses in the context of adenoviral vaccine delivery. As the Ii-E1E2E6E7 antigen additionally showed a tendency towards higher E1 responses than the Ii-E1E2-p2a-li-E6E7 antigen, follow-on experiments were performed that, however, failed to demonstrate a consistent difference in antigen immunogenicity hierarchy between the constructs (data not shown). No difference in the quality of the T cell responses was detected, assessed by the fraction of double-positive T cells capable of secreting both IFN-γ and TNF and by the MFI values of IFN-γ.

Besides vigorous T-cell responses against E1 and E2 (FIGS. 5C and D), we did indeed see solid CD4+ T-cell responses against E6 (FIG. 5D). A few mice also had CD8+ T-cells reacting towards E6, confirming processing and immunogenicity of our vaccine constructs. As only a few mice had CD8⁺ T-cell responses and these responses were of low magnitude, it could be questioned whether these were true responses or merely due to high background. A representative mouse depicted in FIG. 5C, shows a CD44⁺IFN-γ⁺ population behaving like a true response. Furthermore, the majority of the IFN-γ⁺ cells were producing TNF as well, confirming the activation phenotype.

Overall, the Ad19a/64 delivery of the vaccines efficiently induced T-cells against all the four MfPV3 polyprotein antigens tested, and the immunogenicity of the antigens was enhanced by inclusion of Ii.

### Ii-Fusion Enhanced Ubiquitination and Proteasomal Degradation

The mechanistic basis of the T cell adjuvant effect of li has not been fully clarified yet (Fougeroux et al, 2019), but one of the suggested mechanisms of action is ubiquitination of li leading to proteasomal degradation of the linked antigen and thereby enhanced MHC-I presentation (Esposito, I. et al. MHC class II invariant chain-adjuvanted viral vectored vaccines enhances T cell responses in humans. Sci. Transl. Med. 12, (2020). To investigate if this mechanism could also account for the enhanced CD8⁺ T-cell responses against the li-linked MfPV3 antigens, Ii-E1E2E6E7- and E1E2E6E7-transfected cells were cultivated in absence or presence of the proteasome inhibitor MG132. Anti-ubiquitin western blot analysis of the immunoprecipitated myc-tagged polypeptides revealed higher ubiquitin-depending signal intensity for Ii-E1E2E6E7 compared to E1E2E6E7 without li (FIG. 6A). This effect was even more pronounced when MG132 was present. Myc-specific signals confirmed the fidelity of the immunoprecipitation (IP) procedure, and analysis of the IP supernatants by an anti-tubulin western blot revealed that comparable amounts of cell lysate were used in the IP procedure (FIGS. 6B and C). A higher level of ubiquitination is commonly associated with faster degradation. This could exemplarily be demonstrated by transiently expressing li-E1 and E1, both fused with the C-terminal SIINFEKL peptide, in absence or presence of MG132, respectively (FIGS. 6D and E). Without MG132, Ii-E1-SIINFEKL was hardly detectable whereas a prominent signal could be visualized when MG132 was added. Lower molecular weight signals are indicative of degradation products. This effect was not observed for E1 without li, which suggested that li induced an accelerated proteasomal degradation.

### Adenoviral Delivery Induces Specific Killing of Target Cells In Vivo

To assure that the cellular responses induced by the rAd-delivered antigens had cytotoxic capacity, we immunized inbred Balb/C mice, and 14 days later challenged these mice with peptide-pulsed pre-stained syngeneic target cells. The vaccines induced CD8+ responses against E1 (FIG. 7A), but neither against the other antigens in Balb/C mice (Data not shown), whereas CD4 responses were raised against E1, E2 and E6. Ii again proved its relevance as an adjuvant, as the vaccine without li did not give rise to any detectable E1 specific CD8+ responses in this inbred mouse model (FIG. 7A). The in vivo cytotoxicity assay showed specific killing of E1 labelled cells (FIGS. 7B and C), and the hierarchy of specific killing capability corresponds to the number of IFN-γ+CD8+E1 specific T cells. We can conclude that the designed rAd vaccines induce potent and cytotoxic responses, which supports our hypothesis that this vaccine design will be capable of removing existing MfPV3 infections.

### Example 2: Assessment of Oncogenic Properties of E6 and E7 as Modified in Accordance with the Invention

### Material and Methods

### Cell Lines

NIH-3T3 cells and HEK293T cells were cultivated at 37° C. with 5% CO₂ in DMEM supplemented with 10% fetal calf serum (FCS) and 1% Pen/Strep (PS). The NIH-3T3 cells were maintained between 10% and 70% confluence to prevent maturation and differentiation.

### Production of VSV-G-Pseudotyped Lentiviral Vectors

The HPV16 antigens and HPV16 E6 wt were cloned into pLV-MCS-IRES-Puro, HPV16 E7 wt and GFP were cloned into pLV-MCS-IRES-Neo, respectively, by using AgeI-HF and NotI-HF (NEB, Ipswich, USA) restriction enzymes. Both pLV vectors contain a CMV promoter followed by an MCS, an internal ribosome entry site (IRES) and antibiotic resistance gene against either puromycin or neomycin.

3.9×10⁶ HEK293T cells were seeded in T75 flasks. 24 h post seeding, the medium was changed to DMEM without additives and the cells were transfected with 3.75 μg of pLV containing the antigen and 3.75 μg of lentivirus packaging mix (1:1:1; pMDL-Gag: pVSV-G: pREV) using the PEI method (Boussif, O., Lezoualc′h, F., Zanta, M. A., Mergny, M. D., Scherman, D., Demeneix, B., & Behr, J. P. (1995). A versatile vector for gene and oligonucleotide transfer into cells in culture and in vivo: polyethylenimine. Proceedings of the National Academy of Sciences, 92 (16), 7297 LP-7301. https://doi.org/10.1073/pnas.92.16.7297) in a final volume of 9 ml. 5 h post transfection, 4 ml DMEM containing 10% FCS and 1% PS were added to the flasks. The lentiviral vectors were harvested 48 h post transfection by centrifugation at 1000×g for 5 min, the supernatants were filtered through 0.45 μm syringe filters and stored at −80° C. in aliquots.

### Transduction of NIH-3T3 Cells and Antibiotic Selection

1×10⁵ NIH-3T3 cells were seeded in T25 flasks. 24h post seeding, the medium was aspirated, and the cells were transduced with 2 ml of each supernatant containing the respective lentiviral vector in presence of 10 μg/ml polybrene (TR-1003-G, Sigma Aldrich, St. Louis, USA). After 6 h, 4 ml DMEM containing 10% FCS and 1% PS were added. 72 hours later, transduced cells were selected via 1 mg/ml G418 (CP11.3, Roth, Karlsruhe, Germany) and/or 2 μg/ml puromycin (ant-pr-1, InVivoGen, San Diego, USA), depending on the lentiviral vector used for transduction. The cells were under antibiotic selection for at least 2 weeks, until all untransduced cells had died.

### Antibodies

The following antibodies were used: anti-E2 (TVG-271, 1:200, Santa Cruz Biotechnology, Heidelberg, Germany), anti-E6 (GTX132686, 1:2000, Biozol, Eching, Germany), anti-E7 (NM2, 1:200, Santa Cruz Biotechnology, Heidelberg, Germany), anti-tubulin (DM1a, 1:1000, Santa Cruz Biotechnology, Heidelberg, Germany), goat anti-mouse-HRP (115-036-003, 1:5000, Jackson, West Grove, USA) and goat anti-rabbit-HRP (P0448, 1:2000, Dako, Santa Clara, USA).

### Western Blot Analysis

Western blot analysis was performed as previously described (Kiener, R., Fleischmann, M., Schwegler, C., Ruzsics, Z., Thirion, C., Schrödel, S., Asbach, B., & Wagner, R. (2018). Vaccine vectors based on Adenovirus 19a/64 exhibit broad cellular tropism and potently restimulate HCMV-specific T cell responses ex Scientific Reports, 8 (1), 1474. vivo. https://doi.org/10.1038/s41598-018-19874-1). Briefly, cells of interest were lysed in TDLB buffer (50 mM Tris, pH 8.0, 150 mM NaCl, 0.1% SDS, 1% Nonidet P-40, 0.5% sodium deoxycholate) supplemented with protease inhibitors (Complete Mini, Roche, Basel, Switzerland). Total protein concentration of the supernatants was measured by the Bradford method (Protein Assay, BioRad, Feldkirchen, Germany). The proteins were separated on SDS-PAGE under reducing conditions and blotted on a nitrocellulose membrane for western blot analysis. Targets were probed with primary and secondary antibodies as listed above. HRP-labeled secondary antibodies and enhanced chemiluminescence substrate or Femto ECL (Thermo Fisher, Waltham, USA) were used for detection in a Chemilux Pro device (Intas, Göttingen, Germany). For loading control, the membrane was stripped (stripping buffer (62.5 mM Tris, 2% SDS, 0.1 M B-Mercaptoethanol, pH 6.6) for 30 min at 50° C.) and reprobed with an antibody against tubulin.

### Soft Agar Transformation Assay

The soft agar transformation assay was performed as described previously (Borowicz, S., Van Scoyk, M., Avasarala, S., Karuppusamy Rathinam, M. K., Tauler, J., Bikkavilli, R. K., & Winn, R. A. (2014). The Soft Agar Colony Formation Assay. JoVE, 92, e51998. https://doi.org/doi: 10.3791/51998) with minor modifications as follows. 6-well plates were filled with 2 ml DMEM (without phenol red, low glucose) (D2902, Sigma Aldrich, St. Louis, USA) containing 10% FCS, 1% PS, 3.7 g/l NaHCO₃, 3.8 g/l D-glucose, 0.5% low melt agarose (6351.2, Roth, Karlsruhe, Germany), 1 mg/ml G418 and/or 2 μg/ml puromycin, respectively, and incubated at room temperature until the medium had solidified. 5×10³ of the lentivirally transduced polyclonal NIH-3T3 cells were seeded in 37° C.-prewarmed DMEM containing 10% FCS, 1% PS, 3.7 g/l NaHCO₃, 3.8 g/l D-glucose, 0.35% low melt agarose, 1 mg/ml G418 and/or 2 μg/ml puromycin, respectively, onto the pretreated 6-well plates and incubated at room temperature, until the medium had solidified. The plates were incubated at 37° C. and 5% CO₂ for 21 days. Within this period, the cells were fed twice a week with 200 μl of DMEM, supplemented with 10% FCS and 1% PS. After 21 days, each well was fixed with 100 μl 4% PFA and stained with 0.005% crystal violet in 10% methanol (v/v) for 1 h at room temperature. Pictures were taken on an alphaimager HP (Proteinsimple, San Jose, USA) at an aperture of 5.6, and focus of 2.88. The colonies were counted with ImageJ (Schindelin, J., Arganda-Carreras, I., Frise, E., Kaynig, V., Longair, M., Pietzsch, T., Preibisch, S., Rueden, C., Saalfeld, S., Schmid, B., Tinevez, J.-Y., White, D. J., Hartenstein, V., Eliceiri, K., Tomancak, P., & Cardona, A. (2012). Fiji: an open-source platform for biological-image analysis. Nature Methods, 9 (7), 676-682. https://doi.org/10.1038/nmeth.2019). Statistical analysis was done using GraphPad Prism 8 software (GraphPad Software, San Diego, USA). Individual groups were compared using two-sided, parameterized, unpaired t-test. Each symbol represents one replicate, bars indicate mean with standard error of the mean (SEM). Significance levels are marked by *(p<0.05), **(p<0.005), ns (not significant).

### Results

### Antigen-Expression Did not Cause Anchorage-Independent Growth

Anchorage-independent growth is a hallmark of carcinogenesis, since transformed cells are able to grow independently of attachment to a solid phase (Borowicz et al., 2014). One of the most stringent tests for transformation is the soft agar transformation assay, which characterizes solid-phase-independent growth in soft agar. For this, NIH-3T3 cells were transduced with VSV-G-pseudotyped lentiviral vectors encoding the following HPV16 antigens: Ii-E1E2E6E7, Ii-E1E2-p2a-Ii-E6E7, E1E2E6E7, Ii-E2, Ii-E1E6E7-p2a-li-E2, Ii-E1E6E7, E6 wt, and E7 wt, respectively (FIG. 1A). For control purposes, NIH-3T3 cells were transduced with VSV-G-pseudotyped lentiviral vectors encoding GFP and lentiviral vectors lacking a transgene, respectively. Additionally, one polyclonal NIH-3T3 cell line was generated by transduction with both, HPV16-E6 wt- and HPV16-E7 wt-encoding lentiviral vectors.

After antibiotic selection, transgene expression of polyclonal NIH-3T3 cell lines was verified by western blot analysis. For all cell lines, the expected bands at the correct sizes were detected, except for Ii-E1E6E7-p2a-li-E2 (FIG. 8A). As generation of this cell line failed in 3 attempts, Ii-E1E6E7-p2a-li-E2 was excluded from further analysis. 5×10³ cells were seeded in soft agar as described in the methods section. 3 weeks after seeding, the plates were stained, and colonies were counted with ImageJ. There was no difference in the number of colonies as compared to the negative controls (GFP- and empty-vector-transduced NIH-3T3 cells), except for E7 wt- and E6 wt+E7 wt-transduced cells (FIG. 8B) that readily formed high numbers of colonies in the soft agar. Taken together, this confirms functional inactivation of the transforming potential of the HPV16 antigens, and therefore the antigens can be considered as safe.

### Example 3: Immunogenicity of MfPV3 Ii-E1E2E6E7 in Rhesus Macaque Monkeys

### Materials and Methods

### Cell Lines, Transfection and Viral Infection

AGE1.CR.pIX and AGE1.CR.pIX-T-REx cells grown in suspension were maintained in chemically-defined CD-U6 medium (ProBioGen AG) supplemented with 2 mM L-glutamine (Sigma) and recombinant insulin-like growth factor (LONG-R³ IGF, 50 ng/mL final concentration, Sigma) and were incubated in an orbital shaker (Multitron Pro,Infors HT) at 8% CO₂ with a shaking speed of 180 rpm. For adherent growth AGE1.CR.pIX and AGE1.CR.pIX-T-REx cells were cultivated in Dulbecco's Modified Eagle Medium DMEM/F12 supplemented with 5% Fetal Calf Serum (FCS). Adherent cell lines were maintained at 37° C. and 8% CO₂ in a humidified incubator.

### Generation of Recombinant MVA Vectors

Recombinant MVA vectors were generated as previously described (Jordan, I., Horn, D., Thiele, K., Haag, L., Fiddeke, K., Sandig, V., Virol Sin. 2020 April; 35 (2): 212-226. doi: 10.1007/s12250-019-00176-3.). Briefly, the Genes of Interest (GOI)-(FIG. 1) were cloned into the shuttle vector SP-CR19III-tetO, suitable for integration of the GOI into MVA's deletion site III under control of the MVA E/L-promoter. Recombinant MVA encoding the different GOIs were generated by homologous recombination in adherent AGE1.CR.pIX-T-REx cells that prevent the undesirable expression of the GOI during generation and propagation of the recombinant MVA by taking benefit of the Tet system (s. above). Therefore, the culture monolayers seeded in a six-well plate were infected with MVA or MVA-CR19 with a MOI of 0.05 and transfected with 2 μg of the individual shuttle vector using the Effectene Transfection Reagent (Qiagen, Germany) according to the manufacturer's instructions. The infected/transfected culture was harvested 2-3 days post infection/transfection, sonicated, and used for infection of a cell monolayer in a six-well plate format. Resulting plaques were validated by PCR and an iterative plaque purification procedure was initiated until MVAs without the correct GOI expression cassette were not present any more (usually within 5-8 rounds of plaque purification).

For the propagation of plaque purified MVAs the cell harvest material was sonicated using a Vial Tweeter (set to 20 s of 100% cycle and 90% amplitude, Hielscher, Germany), and AGE1.CR.pIX-T-REx (grown in suspension at 2x 10⁶ cells per ml in a 1:1 mixtures of CD-U4 and CD-VP4 (Merck-Millipore) were inoculated with the individual recombinant MVA vectors at MOI 0.05. Finally, MV As were harvested 48 h-72 h post infection and the TCID₅₀ titer was determined.

### Animals

Ethical approval for use of cynomolgus macaques was granted by the primate research center at the Bogor Agricultural University IACUC (Animal Care and Use Committee). Female cynomolgus macaques between 6 and 10 years old and weighing between 3 and 5 kg were selected for use in this study based on negativity for tuberculosis, Simian T-cell leukemia virus and Simian retrovirus and cervical positivity for MfPV3. The selected animals were acclimatized for a month before vaccination. Animals were housed in groups and fed with monkey chow (Charoen Pokpand, Thailand) twice a day.

All procedures were performed by highly trained veterinarians, animal technicians and caretakers. Before all procedures such as vaccination and blood withdrawal, cervical swabs, animals were anesthetized with Ketamine (10-15 mg/kg) and Xylazine (0.5-1 mg/kg). Before and after the procedures, macaques were monitored for pain, distress, body temperature and weight. Blood collection was limited to the maximum volume allowed over the duration of the experiment. The experiment is still ongoing, but all animals were returned into the colony at the end of the study for use in other experiments or breeding programs.

DNA Purification from Cervical Swabs and MfPV3 Detection by qPCR

Briefly, cervical samples were obtained using a small cervix cytobrush and placed in a preservative solution called TEN buffer (Tris HCl 10 mM pH 7.5; EDTA 5 mM, NaCl 50 mM). DNA was extracted from the cervical samples with the DNA extraction kit (QIAmp DNA blood mini kit, Qiagen, Hilden, Germany) and eluted in 100 mL following the manufacturer's procedure.

Selection of MfPV3 positive cynomolgus macaques, and detection of MfPV3 infection level during the therapeutic vaccination trial was performed at Bogor University (Indonesia) by quantitative PCR (qPCR) with the following set of primers and probe: 5′FAM-aaatggtgcagtgggcatacgacc-3′BHQ1, gggaaccacctgaatggata and ggcgcaatccttcacatatt.

### Vaccinations

Animals were vaccinated on day 0 with either PBS or Ad19a/64 vectored MfPV3 Ii-E1E2E6E7 and with MVA vectored MfPV3 Ii-E1E2E6E7 on day 42. Vaccines were given both i.m., i.rec. and i.vag, 2×10⁸ IFU (Ad19a/64) or TCID50 (MVA) at each route.

### ELISpot

Peripheral blood mononuclear cells (PBMCs) from each vaccinated cynomolgus macaques were purified from blood by Ficoll-Paque (Sigma). Vaccine antigen specific T-cells from the fresh purified PBMCs were measured by IFN-γ production using the Monkey IFN-γ ELISPOTplus kit (MABTECH, #3421M-4HST-2) after 18-20 hours incubation at 37 C and 5% CO2 in presence of 2 mg/mL of the relevant peptide pools. Spot-forming units (SFU) were read on an IRIS instrument and counted using the ELISpot Big emphasis algorithm. Background responses from unstimulated samples were subtracted prior to graphical representation.

### Flow Cytometry of NHP PBMCs

Peripheral blood mononuclear cells (PBMCs) from each vaccinated cynomolgus macaques were purified from blood by Ficoll-Paque (Sigma). Cells were incubated for 5 hours in 20 ug/uL brefeldin A with or without 2 μg/mL of relevant peptides. The cells were stained against surface markers: APC-CD3 (clone SP34-2, BD biosciences), PerCP/Cy5.5-CD4 (Clone L200, BD biosciences), PE-CD8 (Clone RPA-T8, BD biosciences) and fixable viability dye eFlour780 (Thermofisher). After surface staining, cells were fixed and permeabilized in BDcytofix/Cytoperm, and stained intracellularly using FITC-IFN-γ (Clone MD-1, UCy-Tech) and PE/Cy7-TNF-α (Clone mAb11, BD biosciences) antibodies.

The peptides used were 16-mers overlapping by 11 amino acids covering the entire li-E1E2E6E7 antigen. The peptides were pooled in 5 separate pools containing Ii, E1, E2, E6 and E7 peptides respectively. Peptides were obtained from KareBay, Town, China.

Flow cytometry was performed on FACS CANTO flow cytometer and data analysis was performed using FlowJo V10 software. Epitope-specific CD8+ T-cell responses were measured as viable CD3⁺, CD8⁺ or CD4⁺, IFN-γ cells. Background (from unstimulated samples) were subtracted before plotting and analysis. All responses below 0.01% were regarded as below detection limit, and manually adjusted to 0.01 for optimal visual representation.

### Results

Responses after Prime Vaccination Measured by ELISpot

All vaccinated animals respond strongly to the E1 peptide pool (FIG. 10), compared to the PBS (sham/negative control) vaccinated animals. There are E2 responses in all vaccinated animals, albeit at a lower frequency than E1, and E6/E7 responses are seen in two of the vaccinated animals.

This provides evidence that the Ii-E1E2E6E7 Ad19a/64 vaccine is immunogenic in monkeys.

Responses after Boost Vaccination, Measured by Intracellular Staining and Flow Cytometry

Very low T-cell responses are detected prior to boost (FIG. 10, middle panels), but 9 days post MVA boost, the T-cell responses have been re-amplified. All vaccinated monkeys show strong CD8 and CD4 responses against at least one of the vaccine antigens (including E1) in contrast to the PBS controls, and CD8 responses against E2 and E7 are detected in a number of vaccinated animals as well (FIG. 10, bottom panels).

Besides confirming that the viral vectored MfPV3 Ii-E1E2E6E7 vaccine is immunogenic in MfPV3 infected monkeys, this also provides a strong indication that the vaccine can cure the pre-existing MfPV3 infections in the NHPs.

### Clearance of MfPV3 Infection at Day 56 in all Vaccinated Animals

As seen in FIG. 12, only two of the vaccinated animals have detectable MfPV3 vaccination at day 42 (6 weeks after prime) and all vaccinated animals have cleared the infection at day 56 (2 weeks after boost) compared to only 3 out of 6 of the PBS injected negative control animals. Note that one animal in the vaccine-group and one animal of the negative ctrl-group did not have detectable MfPV3 at day 0 (each symbol represents one animal). However, the negative ctrl animal with no detectable infection at day 0, had detectable viremia at day 42, and is therefore regarded as infected, meaning that the clearance rates are 5/5 and 3/6 for the vaccine- and PBS-groups respectively, resulting in a p-value=0.18 (Fishers's Exact test).

Vaccine-Induced T Cell Responses Appear Functional in Animals with Persistent MfPV3 Infection.

Exhaustion of HPV-specific T cells can occur in people with persistent HPV infection, and is characterized by loss of functionality and eventually by elimination of antigen-specific T cells. Thus, it is important that a therapeutic vaccine can induce antigen-specific immune responses in the context of a persistent presence of the infection.

Prime immunization with hAd19a/64 li-E1E2E6E7 did indeed induce IFN-γ producing T cells against the vaccine antigens in all vaccinated animals (FIG. 11A). Notably, there was no observed difference between vaccinated animals with persistent MfPV3 infection and animals where the duration of the HPV infection was not known.

Immunization on day 42 with MVA vectored li-E1E2E6E7 boosted the CD8+ T cell responses against E1 and E2, while the CD8+ T cell responses against E6 and E7 did not seem to be enhanced by boosting (FIG. 11B).

The ability to produce high levels of effector cytokines is a marker of high functionality and absence of exhaustion. Assessment of average IFN-γ production CD8+ T cell revealed, that E1 specific CD8+ T cells had significantly higher levels of IFN-γ compared to E7 (FIG. 11C). This indicates that exhaustion may be more prevalent in the E7-specific T cell population compared to the E1-specific.

### Example 4: Properties of the Different Construct Designs with HPV16 E1, E2, E6 and E7 Antigens, and Tumour Protective Efficacy of HPV16 Ii-E1E2E6E7

### Materials and Methods:

### Animals, Immunizations, Cisplatin Injections and Tumour Challenges

C57BL/6 and CD1 mice were obtained from Envigo (Horst, The Netherlands), OF1 mice from Charles River (France) and HSd-Ola mice from Envigo (UK). All animals were females, 6-8 weeks old, and were housed at the Panum Institute, University of Copenhagen. All experiments were initiated after allowing the mice to acclimatize for at least 1 wk. Experiments were approved by the National Animal Experiments Inspectorate (Dyreforsøgstilsynet, license no. 2016-15-0201-01131) and performed according to national guidelines.

The tumour cell line C3 was developed by transfection of mouse embryonic cells with the HPV16 genome and an activated ras oncogene (Feltkamp et al, Eur J Immunol 1993). Cell line authentication was carried out by flow cytometry detection of HPV16 E7 presence. To assess anti-tumor efficacy in vivo, 1E+06 C3 tumor cell in 200 uL PBS+0.2% BSA were injected subcutaneous (s.c.) into the flank of C57BL/6 mice. Tumor size was measured in length and width three times weekly and the tumor volume was calculated as: length*width²*0.5236 (Janik et al, Cancer Res 1975). Animals were sacrificed by cervical dislocation once the tumor exceeded 1000 mm³ or at the time-points indicated.

For treatment of established tumors (d10 immunizations), mice were assigned to treatment groups based on their tumor size just prior to treatment, to ensure equal average tumor sizes for all treatment groups at the start-point of treatment.

Immunizations with adenoviral vectors were performed intramuscular (i.m.) in thigh in the same site as tumor injection with 2×10⁷ IFU rAd diluted in 50 μL PBS, except for FIG. 18, where mice were immunized in the lower limb, a mixed systemic and cutaneous route, with 2×10⁷ IFU Ad5 or Ad5F35 in 30 uL PBS. Immunization with SLP vaccine (HPV16 E743-77 and HPV16 E641-65, Schafer) was performed s.c. in the opposite flank of tumor injection with 50 μg of each peptide. The SLP vaccine was prepared by dissolving lyophilized peptide to 100 mg/mL in DMSO and further dilution to 0.5 mg/mL in PBS. This was emulsified in a 1:1 ratio with Freunds complete adjuvant (Sigma-Aldrich) to a final concentration of 0.25 mg/mL and a total volume of 200 μL was injected per mouse.

Cisplatin (Sigma-Aldrich) was dissolved to 1 mg/mL in 0.9% NaCl: solution and 3 mg/kg mouse body-weight was injected intra-peritoneal once weekly three times.

Mice were euthanized when tumours exceeded 1000 mm³, necrotic wounds emerged or mobility of the mice was markedly reduced.

### Intracellular Staining and Flow Cytometry Analysis of Splenic, Lymph Node, Blood and Intratumoral Immune Cells

Single cell suspensions of splenocytes and lymph nodes were obtained by organ harvest in HANKs followed by straining through 70 μm cell strainers. Tumor tissue was weighed after harvesting, and processed using the Miltenyi mouse tumor dissociation kit (cat no 130-096-730) and the GentleMACS Dissociator using manufacturer's protocol. Blood was harvested in EDTA Microvette tubes (Sarstedt) and red blood cells were lysed using ACK lysing buffer (Gibco).

Cells were incubated for 5 hours in 3 μM monensin with or without 1 μg/mL of relevant peptides (JPT HPV16 pepmix). The cells were stained against surface markers (all antibodies were from BioLegend, San Diego, USA unless otherwise noted): APC-Cy7 or BV421 CD8 (53-6.7, 1:200, BD Biosciences, Franklin Lakes, USA), PE-Cy7 CD4 (RM4-5, 1:800, BD Biosciences, Franklin Lakes, USA), FITC CD44 (IM7, 1:100), PerCP-Cy5.5 B220 (RA3-6B2, 1:200), Fixable Viability Dye eFluor™ 780 (eBioscience) and BV510 PD-1 (29F.1A12, 1:100). After surface staining, cells were fixed in 1% PFA, permeabilized in 0.5% saponine, and stained intracellularly using APC IFN-γ (XMG1.2, 1:100) and PE TNF-α (MP6-XT22, 1:100) antibodies. Flow cytometry was performed on the Fortessa 3 or Fortessa X20 (BD Biosciences, Franklin Lakes, USA) flow cytometer and data analysis was performed using FlowJo V10 software. HPV16-specific CD8⁺ T-cell responses were measured as B220;, CD8⁺ or CD4⁺, CD44⁺, IFN-γ⁺ cells and are presented in total number of cells per spleen, total number of cells per mL blood, total number of cells per g tumor or as fractions of populations as described in respective Figures. The quality of the IFN-γ responses were evaluated by MFI of IFN-γ and fraction of double positive cells (expressing both IFN-γ and TNF-α) in the IFN-γ+CD8⁺ or CD4⁺ populations.

### Lineage Staining and Flow Cytometry Analysis of Intratumoral Immune Cells

Tumor tissue was weighed after harvesting, and processed using the Miltenyi mouse tumor dissociation kit (cat no 130-096-730) and the GentleMACS Dissociator using manufacturer's protocol, and stained against surface markers (all 1:100, BD Biosciences, Franklin Lakes, USA unless otherwise noted): BV650 CD8 (53-6.7), PerCP/Cy5.5 CD4 (RM4-5), PE/Cy7 CD45.2 (104), APC F4/80 (BM8, BioLegend, San Diego, USA), AF488 CD11c (N418, BioLegend, San Diego, USA), BV421 PD-L1 (10F.9G2, BioLegend, San Diego, USA), BV510 PD1 (29F.1A12, BioLegend, San Diego, USA), AF700 TER119 (TER-119) and Fixable Viability Dye eFluor™ 780 (1:1000, eBioscience). After surface staining, cells were fixed and permeabilized using the eBioscience™ Foxp3/Transcription Factor Staining Buffer Set, and stained intracellularly using PE FoxP3 (FJK-16s, 1:100, Invitrogen).

Flow cytometry was performed on the Fortessa 5 (BD Biosciences, Franklin Lakes, USA) flow cytometer and data analysis was performed using FlowJo V10 software.

### Immune Cell Depletion

CD4⁺ and CD8+ T-cell depletion was done by i.p. injection of 0.25 mg Anti-mouse CD4 (GK1.5; #BE0003-1; BioXCell), 0.1 mg Anti-mouse CD8 (2.43; #BE0061; BioXCell) or 0.1 mg Rat IgG2b isotype control (LTF-2; #BE0090; BioXCell) per mouse in 100 uL PBS. Depletion antibodies were given on day 20, 23 and 26 after C3 tumor challenge. Depletion was verified on day 28 by surface staining and flow cytometry on blood samples, using BV421 CD8 (53-6.7, 1:200, BD Biosciences, Franklin Lakes, USA), PerCP/Cy5.5 CD4 (RM4-5, 1:100, BD Biosciences, Franklin Lakes, USA), PE/Cy7 CD45.2 (104, 1:100 BD Biosciences, Franklin Lakes, USA) and Fixable Viability Dye eFluor™ 780 (1:1000, eBioscience). Flow cytometry was performed on Fortessa 3 (BD Biosciences, Franklin Lakes, USA) flow cytometer and data analysis was performed using FlowJo V10 software.

### Immunohistochemistry Staining of Tumor Tissue

Tumor tissue was carefully harvested at the time of sacrifice and fixated in 4% cold PFA (Alfa Aesar). The fixative was exchanged with 70% ethanol after approximately 24 hours. Fixated tissues were embedded in paraffin and slides were stained with PE and with DAB for CD8a (D4W2Z, 1:600, pH=9, Cell signaling).

Tissues were scanned using Axio Scan.Z1 (Zeiss) and analysed using Zen 3.4 software (cut-off area: 10-180 um². Cut-off circularity: 0.5-1.0).

### Results

Ad19a/64-Vectored Vaccination Induces Robust CD8+ and CD4+ T-Cell Responses in Both Outbred and Inbred Mice which can be Boosted by Ad5

Intramuscular immunization of outbred CD1 mice with a single dose of Ad19a/64-vectored vaccines confirmed immunogenicity of the different antigen constructs in vivo, as CD8+ T-cell responses were detected against E1 (FIG. 13A) and CD4+ responses were measured against E1 (FIGS. 13B) and E2 (FIG. 13C).

To enhance immune responses further, a heterologous prime-boost immunization was applied delivering human Ad5 encoded Ii-E1E2E6E7 as a booster vaccine. This increased CD8+ T-cell responses to E1 (FIG. 13D as compared to FIG. 13A) and induced some responses against E7 as well (FIG. 13E). CD4+ T-cell responses were consistently detected against E1 and E2 and sporadically detected against E6 and E7 (data not shown). We never detected CD8+ T-cell responses to E2 or E6 in CD1 mice, potentially reflecting limited MHC diversity in the supposedly outbred CD1 mouse model due to a rather small number of founder mice (Yalcin et al. 2010). We therefore chose to evaluate the li-E1E2E6E7 vaccine in a Ad19a/64-Ad5 prime boost regimen in selected other outbred mouse strains derived independently from CD1 (OF1 and Hsd-Ola) (Yalcin et al. 2010). Here, we detected vaccine induced CD8+ responses against all four antigens in at least one of the mouse strains (FIG. 13F-I) and CD4+ responses against E1, E2 and E7 (data not shown).

Lastly, we wanted to confirm immunogenicity in the C57BL/6, which is the standard mouse model in the field of HPV16 therapeutic vaccines. We found responding CD8+ T-cells against E1 for all antigen designs (FIG. 13J) and detected E7-specific CD8+ T-cells as well (FIG. 13K), and CD4+ responses against E1 (data not shown). The Ii-E1E2E6E7 gave significantly stronger E7 responses than the Ii-E1E6E7-p2a-li-E2 vaccine, and li-E1E2E6E7 also seemed to give stronger E7 responses than the vaccine not encoding Ii (p=0.077). This advantage of the Ii-E1E2E6E7 was further supported when looking at the integrated mean fluorescence intensity (iMFI) of IFN-γ positive cells combining the magnitude and quality of the induced T-cell response (Shoostari et al. 2010). In this analysis, Ii-E1E2E6E7 significantly enhanced the response against E7 compared to the vaccine not encoding li and the vaccine encoding E7 directly linked to the p2a-li (FIG. 13M). Furthermore, the E1 response was by trend higher for Ii-E1E2E6E7 compared to the non-li vaccine as well (FIG. 13L, p=0.06). We did not detect any E2 or E6-specific CD8+ T-cell responses in spite of a previously reported E6 epitope (aa48-57, EVYDFAFRDL) (Peng et al. 2004). This could be due to immunodominance, as responses against the E6 epitope have previously only been described for vaccines encoding E6 alone, and the E6 epitope has been reported as less immunogenic than the dominant E7 epitope (aa49-57, RAHYNIVTF) (Feltkamp et al. 1993). Based on the superior CD8+ T-cell responses in the C57BL/6 mice, and the lack of inferiority and positive trends in the outbred mouse models, we decided to move forward with the li-E1E2E6E7 antigen design.

Lastly, we confirmed that heterologous Ad5-vectored boosting after Ad19a/64 priming induced a re-call vaccine specific response compared to Ad19a/64 priming alone, whereas homologous boosting with Ad19a/64 did not (FIG. 13N-P). A negative control group of Ad19a/64 and Ad5 vectors containing no antigen were included to confirm that the responses were vaccine specific.

Ad-Vector Vaccination Shortly after Tumor Inoculation Provides Single-Agent Tumor Control and Increased Survival

To investigate the therapeutic effect of our HPV vaccine, C57BL/6 mice were injected with syngeneic C3 tumor cells. The C3 cell line was established by transfection of mouse embryonic cells with the HPV16 genome and an activated ras oncogene (Feltkamp et al. 1993), and the tumors can express all HPV16 antigens (Schmitt and Pawlita et al. 2011), in contrast to the commonly used TC-1 tumor model containing only HPV16 E6 and E7 (Lin et al. 1996). It is worth mentioning that the C3 tumor cell line is generally slower growing and more treatment resistant than the TC-1 model, as shown by Sluis et al. 2015 (cf. their Supplementary FIG. 1), making it more challenging to show therapeutic effect in the C3 model.

In the first experiments, mice were treated with A19a/64 vectors encoding Ii-E1E2E6E7 or an irrelevant antigen (neg ctrl) 2 days after tumor challenge. On day 20, another treatment with Ad5 vectors containing the same antigens was done to boost responses. Ii-E1E2E6E7 treatment significantly reduced tumor growth (FIG. 14A), completely cleared tumors in 3 out of 10 mice (FIG. 14B) and significantly increased survival (FIG. 14C, 3/10 vs 10/10 dead at day 41), indicating that the treatment is effective against small tumors.

Ad-Vectored Vaccination Provides Single-Agent Control of Established Tumors and Works in Synergy with Cisplatin Co-Treatment

Having confirmed that the early treatment of C3 tumors with therapeutic vaccination had good effect as vaccine regimen monotherapy, we went on to investigate the potency on established tumors. Once palpable tumors were present (day 10), mice were treated with therapeutic vaccination, which showed a reduction of tumor growth in some animals (FIGS. 15A and B) and significantly increased survival (FIG. 15C). Cisplatin is standard treatment of HPV+ cancers in the clinic and has previously shown synergy with HPV-targeting therapeutic vaccines (Sluis et al. 2015). Combining the therapeutic vaccination with three once-weekly cisplatin injections, as previously described by Nejad et al. 2016, dramatically increased the tumor control (FIGS. 15D and E) and survival (FIG. 15F) compared to cisplatin treatment and control vaccination, and compared to therapeutic vaccination alone (data not shown).

Immunohistochemical staining of tumor tissues revealed that the therapeutic vaccination significantly increased the number of CD8+ T-cells (FIGS. 15G and 15I) and even more importantly the level of infiltration of these CD8+ T-cells into the central tumor parenchyma (FIGS. 15H and 15I). Co-treatment with cisplatin did not seem to have any obvious effect on the infiltration of CD8+ T-cells visualized by immunohistochemistry (data not shown). To further investigate the cause of the observed therapeutic effect, we harvested tumors and lymphoid organs at the time-points where the therapeutic effect had only just started to kick in for the respective treatment combinations. Organ havest was performed on day 24 for mice treated with vaccine and cisplatin combined (FIG. 15D) and on day 30 for mice treated with the vaccine alone (FIG. 15A). Analysis of the tumor infiltrating CD8+ T-cells showed that the vaccinated mice had more tumor-reacting CD8+ T-cells producing IFN-Y when incubated with tumor cells ex vivo at both time points (FIG. 15J). Looking at the splenic responses, the vaccine specifically induced E1- and E7-specific CD8+ T-cell responses in these tumor bearing mice (FIG. 15K), corresponding to the non-tumor challenged mice previously analyzed (FIG. 13). Cisplatin treatment alone (neg ctrl+cisplatin, d24, FIG. 15K) seemed to induce some anti-HPV responding CD8+ T-cells, primarily to E7 and in some mice towards E1, but the level of IFN-γ per cell (FIG. 15L), and the amount of IFN-Y producing CD8+ T-cells, also capable of producing TNF was clearly inferior compared to li-E1E2E6E7 treated mice (FIG. 15M).

Surface staining analysis revealed that cisplatin treatment did not seem to have an effect on the amount of CD8+ T-cells in the tumor, supporting the immunohistochemistry findings (data not shown), but that the therapeutic vaccination significantly increased the proportion of antigen-presenting CD11c positive cells in the tumor both on day 24 and day 30 (FIG. 15N). We also saw a tendency of higher levels of dendritic cells (DCs) in the tumor of mice who received cisplatin treatment, similarly to what was reported by Nejad et al. 2016, but this difference could also be attributed to the fact that the analysis was done on day 24 and day 30, respectively.

### Tumor Control is Mediated by CD8+ T-Cells

As the CD8+ T-cells seemed to be associated with therapeutic vaccination and tumor-therapeutic effect, we wanted to confirm whether CD8+ T-cells were in fact the principal effector cell type. Mice with palpable tumors (day 10) were treated with the therapeutic vaccine and cisplatin and subsequently treated with antibody-cell depleting antibodies from day 20 to primarily address the effector phase of the anti-tumor response. We saw that CD8 depletion completely abolished the anti-tumor effect of the therapeutic vaccination (FIG. 16A). In contrast, we saw a tendency of CD4+ T-cell depletion leading to increased tumor control (FIG. 16A), potentially indicating an immune suppressive phenotype of CD4+ T-cells in the tumor microenvironment. Indeed, we found that the tumor infiltrating CD4+ population was largely of the FoxP3+subtype, indicating a substantial presence of regulatory T-cells (Tregs) and this was increased by the therapeutic vaccination (although only significant in the cisplatin co-treatment setting) (FIG. 16B).

The Ad-Vectored Ii-E1E2E6E7 Vaccine Provides Enhanced Survival Compared to Two E6/E7 Synthetic Long Reference Peptides in Combination with Cisplatin

Previous studies have shown efficacy against C3 tumors by immunization with an E7 peptide, E743.77, formulated in Incomplete Freund's Adjuvant (IFA) (Sluis et al. 2015; Nejad et al. 2016; Sluis et al. 2015; Zwaveling et al. 2002). This peptide includes a well-established E7 epitope which is immunodominant in C57BL/6 mice (Feltkamp et al. 1993). Another epitope is also reported in E634, although it is less dominant. Although the previously published studies on peptide vaccines against HPV+C3 tumors includes only the E743-77 peptide, we chose to include an E641-65 peptide in our vaccination comparison as well. The two peptides are also part of a complex synthetic long peptide vaccine (under the clinical development name ISA101), consisting of 13 peptides covering the entire HPV16 E6 and E7 (Melief et al. 2020) and were selected (i) due to their compatibility with the inbred mouse strain and (ii) for reference purposes (Sluis et al. 2015; Nejad et al. 2016; Sluis et al. 2015; Zwaveling et al. 2002).

Mice were treated with cisplatin and therapeutic vaccination by either the two peptides formulated in IFA or our Ad-vectored Ii-E1E2E6E7 once palpable tumors were present (day 10). The Ii-E1E2E6E7 vaccine reduced the tumor growth to a larger extent than the peptide vaccine (FIG. 17A), and led to significantly better survival (FIG. 17B). CD8+ T-cell responses against E1 were detected after vaccination with Ii-E1E2E6E7 and expectedly not after vaccination with the two E6/E7 peptides (FIG. 17C). Both vaccines induced a similar level of E7 specific CD8+ T-cells (FIG. 17D) and no detectable CD4+ T-cell responses (data not shown).

Vaccination with Ad5F35 Vectored HPV16 Ii-E1E2E6E7 Induces CD8+ T-Cells of Similar Magnitude and Quality as Ad5 in Mice

To confirm that Ad5F35 is a useful alternative to Ad5 as a booster vector, inbred C57BL/6 mice were immunized with 2×10⁷ IFU of either an Ad5 or an Ad5F35 vector encoding HPV16 Ii-E1E2E6E7. Splenic CD8+ T-cell responses against HPV16 E1 and E7 were analysed 14 days after immunization by intracellular cytokine staining and flow cytometry.

As shown in FIG. 18, Ad5F35 is capable of generating E1 and E7 reactive CD8+ T-cells and the magnitude (FIGS. 18A and B) and quality, assessed by intensity of IFN-γ signal (FIGS. 18E and F) and capability of producing both TNF-α and IFN-γ (FIGS. 18C and D), was similar to the responses induced by and Ad5 vectored vaccine. This provides evidence, that Ad5F35 can replace Ad5 in the therapeutic prime-boost immunization regimen, without affecting the therapeutic efficacy.

### Example 5: Additional Viral Vectors of the Invention and their Expression Control

### Material and Methods

### Cell Lines

HEK293T cells and A549 cells were maintained and grown in Dulbecco's MEM (DMEM) supplemented with 10% Fetal Calf Serum (FCS) and 1% Penicillin/Streptomycin (Pen/Strep). 9E10 mycl hybridoma cells were cultivated in RPMI supplemented with 10% FCS, 1% Pen/Strep and 2 mM glutamine (Pan). These cell lines were maintained at 37° C. and 5% CO2 in a non-humidified incubator.

Adherent AGE1.CR.pIX cells were cultivated in Dulbecco's Modified Eagle Medium DMEM/F12 supplemented with 5% Fetal Calf Serum (FCS) and were maintained at 37° C. and 8% CO2 in a non-humidified incubator.

Subconfluent A549 cells were infected with Ad19a/64 vectors at an MOI of 100 in DMEM without any supplements. 2 h post infection, medium was exchanged to DMEM with 10% FCS and 1% Pen/Strep.

Subconfluent HEK293T cells were infected with MVA and MVA-CR19 vectors at an MOI of 10 in DMEM without any supplements. 2 h post infection, medium was exchanged to DMEM with 10% FCS and 1% Pen/Strep.

Subconfluent AGE1.CR.pIX cells were infected with MVA and MVA-CR19 vectors at an MOI of 1 in DMEM/F12 without any supplements. 2 h post infection, medium was exchanged to DMEM with DMEM/F12 supplemented with 5% FCS.

### Generation of Adenoviral Vectors

E1/E3 deficient adenoviral vectors of serotype Ad19a/64 were generated as previously described (Ruzsics, Z., Lemnitzer, F. & Thirion, C. Engineering Adenovirus Genome by Bacterial Artificial Chromosome (BAC) Technology BT-Adenovirus: Methods and Protocols. in (eds. Chillón, M. & Bosch, A.) 143-158 (Humana Press, 2014). doi: 10.1007/978-1-62703-679-5_11). Briefly, the Genes of Interest (GOI)-(FIG. 1) were cloned into the shuttle vector pO6-19a-HCMV-MCS under control of a CMV promoter. The CMV-GOI-SV40-pA was then transferred via Flp-recombination in E. coli into a BAC vector (Ruzsics, Z. et al, 2014) containing the genome of a replication deficient Ad-based vector deleted in E1/E3 genes. Recombinant viral DNA was released from the purified BAC-DNA by restriction digest with PacI. The obtained linear DNA was transfected into the production cell line disclosed above for virus propagation. Recombinant viruses were released from cells via sodium deoxycholate treatment. Residual free DNA was digested by DNase I. Afterwards, vectors were purified by CsCl gradient ultracentrifugation followed by a buffer exchange to 10 mM Hepes pH 8.0, 2 mM MgCl2 and 4% Sucrose via PD10 columns (GE Healthcare, Chicago, USA). Titration was performed using the RapidTiter method by detection of infected production cells via immunohistochemical staining with anti-hexon antibody (Novus, Adenovirus Antibody (8C4)). Insert integrity was confirmed by PCR amplification of the GOI in DNA extracted from the purified vectors.

### Generation of Recombinant MVA Vectors

Recombinant MVA vectors were generated as previously described (Jordan, I., Horn, D., Thiele, K., Haag, L., Fiddeke, K., Sandig, V., Virol Sin. 2020 April; 35 (2): 212-226. doi: 10.1007/s12250-019-00176-3.). Briefly, the Genes of Interest (GOI)-(FIG. 1) were cloned into the shuttle vector SP-CR19III-tetO, suitable for integration of the GOI into MVA's deletion site III (DelIII) under control of the MVA E/L-promoter or cloned into the shuttle vector LZAW1-tetO, suitable for integration of the GOI into MVA's thymidine kinase locus (TK) under the control of the MVA SSP-promoter. Recombinant MVA encoding the different GOIs were generated by homologous recombination in adherent AGE1.CR.pIX-T-REX cells that prevent the undesirable expression of the GOI during generation and propagation of the recombinant MVA by taking benefit of the Tet system (s. above). Therefore, the culture monolayers seeded in a six-well plate were infected with MVA or MVA-CR19 with a MOI of 0.05 and transfected with 2 μg of the individual shuttle vector using the Effectene Transfection Reagent (Qiagen, Germany) according to the manufacturer's instructions. The infected/transfected culture was harvested 2-3 days post infection/transfection, sonicated, and used for infection of a cell monolayer in a six-well plate format. Resulting plaques were validated by PCR and an iterative plaque purification procedure was initiated until MVAs without the correct GOI expression cassette were not present any more (usually within 5-8 rounds of plaque purification).

For the propagation of plaque purified MVAs the cell harvest material was sonicated using a Vial Tweeter (set to 20 s of 100% cycle and 90% amplitude, Hielscher, Germany), and AGE1.CR.pIX-T-REx (grown in suspension at 2x 10⁶ cells per ml in a 1:1 mixtures of CD-U4 and CD-VP4 (Merck-Millipore) were inoculated with the individual recombinant MVA vectors at MOI 0.05. Finally, MVAs were harvested 48 h-72 h post infection and the TCID50 titer was determined.

### Antibodies and Antibody Purification

The antibody against myc (9E10) was obtained from hybridoma cell supernatants. 9E10 mycl hybridoma cells were seeded at 5×10⁵ cell per ml in RPMI supplemented with 1% FCS, 1% Pen/Strep and 2 mM glutamine. The supernatant was harvested 5 days after seeding and the antibody was purified via a HiTrap Protein G column (GE Healthcare, Chicago, USA). After washing the column with PBS, the antibody was eluted with 0.1 M glycine/HCl (pH 3.2), neutralized with 0.025 volumes of 1 M Tris/HCl (pH 9) and dialyzed against PBS.

Other antibodies used were: anti-HPV16-E2 (TVG-271, 1:200, Santa Cruz Biotechnology, Heidelberg, Germany), anti-HPV16-E6 (GTX132686, 1:2000, Biozol, Eching, Germany), anti-HPV16-E7 (NM2, 1:200, Santa Cruz Biotechnology, Heidelberg, Germany), anti-HPV18-E2 (2E7, 1:1000, Abcam, Cambridge, England), anti-HPV18-E6 (C1P5, 1:200, Santa Cruz Biotechnology, Heidelberg, Germany), anti-HPV18-E7 (F-7, 1:200, Santa Cruz Biotechnology, Heidelberg, Germany), anti-tubulin (DM1a, 1:1000, Santa Cruz Biotechnology, Heidelberg, Germany), goat anti-mouse-HRP (115-036-003, 1:5000, Jackson, West Grove, USA) and goat anti-rabbit-HRP (P0448, 1:2000, Dako, Santa Clara, USA.

### Western Blot Analysis

Western blot analysis was performed as previously described (Kiener, R. et al. Vaccine vectors based on Adenovirus 19a/64 exhibit broad cellular tropism and potently restimulate HCMV-specific T cell responses ex vivo. Sci. Rep. 8, 1474, 2018). Briefly, cells of interest were lysed in TDLB buffer (50 mM Tris, pH 8.0, 150 mM NaCl, 0.1% SDS, 1% Nonident P-40, 0.5% sodium deoxycholate) supplemented with protease inhibitors (Complete Mini, Roche, Basel, Swiss). Total protein concentration of the supernatants was measured by the Bradford method (Protein Assay, BioRad, Feldkirchen, Germany). The proteins were separated on SDS-PAGE under reducing conditions and blotted on a nitrocellulose membrane for western blot analysis. Targets were probed with primary and secondary antibodies as listed above. HRP-labeled secondary antibodies and enhanced chemiluminescence substrate or Femto ECL (Thermo Fisher, Waltham, USA) were used for detection in a Chemilux Pro device (Intas, Göttingen, Germany). For loading control, the membrane was reprobed with an antibody against tubulin.

### Results

Based on the initial results of MfPV3 antigens a refined panel of rAd vectors was generated comprising a set of recombinant HPV16 and HPV18 antigens. In order to confirm proper expression of the encoded antigens, western blot analysis was performed with adenovirally transduced A549 cells. All vectors readily expressed the antigens with bands resembling the respective fusion proteins being detected with E2-, E6- and E7-directed antibodies at the expected positions (HPV16 antigens FIG. 19A, HPV18 antigens FIG. 19B). Especially for HPV16 antigens, degradation products could be observed, indicated by lower molecular weight bands.

Selected antigens of MfPV3 (Ii-E1E2E6E7, E1E2E6E7), HPV16 and HPV18 (both Ii-E1E2E6E7) were cloned into shuttle vectors for integration into DelIII locus or TK locus of MVA or MVA-CR19, respectively. Recombinant poxviral vectors were generated by homologous recombination and proper expression of the encoded antigens was verified by western blot analysis (FIG. 20). All vectors readily expressed the antigens with bands resembling the respective fusion proteins being detected with myc-directed antibody (MfPV3, FIG. 20A) or type-specific E2-, E6- and E7-directed antibodies at the expected positions (HPV16 antigens FIG. 20B, HPV18 antigens FIG. 20C). Especially for HPV16 and HPV18 antigens, degradation products could be observed, indicated by lower molecular weight bands.

1. A nucleic acid comprising nucleic acid sequences encoding papilloma virus proteins E1, E6, and E7 and optionally further comprising nucleic acid sequence encoding papilloma virus protein E2, wherein said nucleic acid molecule encodes a single polyprotein.

2. (canceled)

3. The nucleic acid of claim 1, wherein the order of the nucleic acids encoding said papilloma virus proteins is, from 5′ to 3′,
(a) to the extent E2 is present
(i) E1, E2, E6, E7; or
(ii) E1, E6, E7, E2;
or
(b) E1, E6, E7.

4. The nucleic acid of claim 1, comprising or consisting wherein said nucleic acid sequences encoding papilloma virus proteins E6 and/or E7 are modified as compared to the wild-type counterparts by point mutations and/or deletions, wherein said point mutations and/or deletions
(i) abolish colony forming in a soft agar assay; and
(ii) said modified proteins E6 and/or E7 maintain at least 70% of predicted high-affinity epitopes (IC50<50 nM) and at least 50%, and optionally at least 55% or at least 58%, of predicted low-affinity epitopes (50 nM<IC50<5 μM) of the predicted epitopes of the respective wild-type protein.

5. The nucleic acid of claim 1, wherein said papilloma virus is a primate papilloma virus, optionally a human papilloma virus (HPV) or a macaque papilloma virus, wherein said HPV is optionally selected from HPV16, HPV18, HPV45, HPV31, HPV33, HPV35, HPV52, HPV58, HPV6 and HPV11, or said macaque papilloma virus, is optionally MfPV3.

6. The nucleic acid of claim 4, wherein the following modifications as compared to the wild-type are present in the encoded proteins:
(i) in E6: a modification which reduces or abolishes interactions mediated by the PDZ domain comprised in wild-type E6, wherein;
a. when the virus is HPV16 E6: the deletion is L117X₁;
b. when the virus is HPV18 E6: the deletion is L112X₁;
C. when the virus is MfPV3 E6: the deletion is L110X₁;
and
(ii) in E7:
a. when the virus is HPV 16 E7: the deletions are C24X₂, L67X₃, and C91X₄;
b. when the virus is HPV18 E7: the deletions are C27X₂, L74X₃, and C98X₄;
c. when the virus is MfPV3 E7: the deletions are C24X₂, L71X₃, and C95X₄:
wherein X₁ to X₄ are independently a proteinogenic amino acid different from the respective amino acid they substitute.

7. The nucleic acid of claim 6, wherein said nucleic acid furthermore comprises one or both mutations encoding D21X₅ and C58X₆ in HPV 16 E7, D24X₅ and C65X₆ in HPV 18 E7, and D21X5 and C62X₆ in MfPV3 E7, wherein X₅ and X₆ are independently a proteinogenic amino acid different from the respective amino acid they substitute.

8. The nucleic acid of claim 6, wherein
(i) X₁ is selected from Q, Y, and N;
(ii) X₂ is selected from G, A, V, L, and I;
(iii) X₃ is selected from R, K, and H;
(iv) X₄ is selected from A, G, V, L, and I;
(v) X₅ is selected from G, A, V, L, and I;
(vi) X₆ is selected from G, A, V, L, and I; and/or
(vii) said modification which reduces or abolishes interactions mediated by the PDZ domain is a deletion of 1 to 10 C-terminal amino acids.

9. The nucleic acid of claim 1, wherein the total number of Cys residues in said polyprotein is an even number.

10. The nucleic acid of claim 9, wherein if said number is otherwise not even,
(i) a sequence encoding
a. HPV16 E2 with a C300X₇ mutation;
b. HPV18 E2 with a C301X₇ mutation; or
c. MfPV3 E2 with a C297X₇ mutation
is comprised in said nucleic acid as sequence encoding E2, wherein X₇ is a proteinogenic amino acid other than C, optionally selected from A, S, G, M, T, Y, Q, N, V, L, I, F, W, H, K, R, Q, N, and P;
(ii) or a codon encoding Cys is inserted in said nucleic acid.

11. (canceled)

12. (canceled)

13. (canceled)

14. (canceled)

15. The nucleic acid of claim 1, wherein said nucleic acid comprises at least one nucleic acid sequence encoding a self-cleaving peptide, wherein said nucleic acid sequence encoding a self-cleaving peptide
(i) precedes at least one nucleic acid sequence encoding a genetic adjuvant; or
(ii) is located between and adjacent to two said nucleic acid sequences encoding a papilloma virus protein.

16. (canceled)

17. A viral vector comprising the nucleic acid of claim 1.

18. The viral vector of claim 17, wherein said vector is a DNA viral vector, optionally wherein said DNA viral vector is
(i) an adenoviral vector, optionally deficient with regard to adenoviral E1 and E3 encoding sequences, said vector optionally selected from Ad19a/64, Ad5, Ad5 with fiber replacements such as Ad5F35, Ad26, Chimp63, and chAdOx1: or
(ii) a Poxvirus vector, optionally selected from MVA, MVA-CR19, SCV, Vaccinia, and Fowlpox.

19. A polyprotein encoded by the nucleic acid of of claim 1 or cleavage products derived therefrom.

20. A pharmaceutical composition, optionally a vaccine, comprising at least one pharmaceutically active agent selected from the group consisting of:
(i) the nucleic acid of claim 1;
(ii) a viral vector comprising the nucleic acid of claim 1;
(iii) a polyprotein encoded by the nucleic acid of claim 1, or cleavage products thereof; and
(iv) a cell transduced with the nucleic acid of (i) or the vector of (ii), optionally wherein said cell is an ex vivo or in vitro cell and/or a dendritic cell or a monocyte.

21. The pharmaceutical composition of claim 20, wherein said vector, said nucleic acid, said polyprotein or said cell is the only pharmaceutically active agent.

22. The pharmaceutical composition of claim 20, wherein said pharmaceutical composition comprises two or more pharmaceutically active agents, wherein a second pharmaceutically active agent is selected from the group consisting of:
(i) a second nucleic acid as defined in claim 20(i);
(ii) a second viral vector as defined in claim 20(ii);
(iii) a second polyprotein as defined in claim 20(iii) or cleavage products thereof;
(iv) a second cell as defined in claim 20(iv);
(v) a protein-based vaccine, optionally against papilloma virus; and
(vi) a chemotherapeutic, optionally cisplatin.

23. (canceled)

24. (canceled)

25. (canceled)

26. A method of treating or preventing a papilloma virus infection, low-grade squamous intraepithelial lesions (LSIL), high-grade squamous intraepithelial lesions (HSIL), nasal polyposis or papilloma virus-associated malignant conditions such as cervical cancer, head and neck squamous cell carcinoma (HNSCC), and oropharyngeal squamous cell carcinoma (OPSCC), said method comprising administering to a subject in need thereof, optionally as a vaccine, at least one of (i) a nucleic acid of claim 1, (ii) a viral vector comprising the nucleic acid of claim 1, (iii) a polyprotein encoded by the nucleic acid of claim 1, or cleavage products thereof, and (iv) a cell transduced with the nucleic acid of (i) or the vector of (ii).

27. An in vitro or ex vivo method of stimulating and/or expanding T cells, said method comprising bringing into contact in vitro or ex vivo T cells with the nucleic acid of claim 1.

28. The nucleic acid of claim 8, wherein said deletion of 1 to 10 C-terminal amino acids is optionally selected from ΔETQL in HPV16 E6, ΔETQV in HPV18 E6, and ΔETEV in MfPV3 E6; or said modification is optionally one, two, three or four point mutations in the 10 C-terminal amino acids, optionally within the four C-terminal amino acids.
